(19) United States
(12) Patent Application Publication
(10) Pub. No.: US 2024/0279678 A1
(43) Pub. Date: Aug. 22, 2024

(54) ON DEMAND EXPRESSION OF EXOGENOUS FACTORS IN LYMPHOCYTES TO TREAT HIV

(71) Applicant: American Gene Technologies International Inc., Rockville, MD (US)

(72) Inventors: Haishan Li, Rockville, MD (US); Tyler Lahusen, Rockville, MD (US); Charles David Pauza, Rockville, MD (US)

(21) Appl. No.: 17/908,509

(22) PCT Filed: Mar. 3, 2021
(86) PCT No.: PCT/US2021/020721

§ 371 Date: Aug. 31, 2022

Related U.S. Application Data
(60) Provisional application No. 62/984,716, filed on Mar. 3, 2020.

(51) Int. Cl. C12N 15/86; A61K 35/17; A61K 39/00; A61K 39/21; C07K 14/73; C07K 16/10; C12N 5/0783

(52) U.S. Cl. CPC C12N 15/86; A61K 35/17; A61K 39/21; A61K 39/461; A61K 39/464838; C07K 14/70514; C07K 16/1045; C12N 5/0636; C07K 2317/76; C12N 2740/16043; C12N 2740/16052; C12N 2830/008

(57) ABSTRACT

The present disclosure relates generally to immunization and immunotherapy for the treatment or inhibition of HIV. In embodiments, a viral vectors are disclosed that comprise therapeutic cargo portions comprising a nucleotide sequence that encodes at least one soluble exogenous factor capable of inhibiting HIV infection, and a T cell-responsive promoter that regulates expression of the nucleotide sequence.

### CROSS-REFERENCE TO RELATED APPLICATION

This application is a national stage application of PCT/US2021/020721, filed on Mar. 3, 2021, which claims priority to: U.S. Provisional Patent Application No. 62/984,716, filed Mar. 3, 2020, entitled “ON DEMAND EXPRESSION OF EXOGENOUS FACTORS IN LYMPHOCYTES TO TREAT HIV,” the disclosures of which are incorporated herein by reference.

### FIELD

The present disclosure relates generally to the field of immunotherapy for the treatment and inhibition of HIV. In particular, the disclosed methods of treatment and inhibition relate to the administration of viral vectors and systems for the delivery of gene products and genetic cargo for the treatment and inhibition of HIV.

### BACKGROUND

Combination antiretroviral therapy (cART) (also known as Highly Active Antiretroviral Therapy or HAART) limits HIV-1 replication and retards disease progression, but drug toxicities and the emergence of drug-resistant viruses are challenges for long-term control in HIV-infected persons. Additionally, traditional anti-retroviral therapy, while successful at delaying the onset of AIDS or death, has yet to provide a functional cure. Alternative treatment strategies are needed.

Intense interest in immunotherapy for HIV infection has been precipitated by emerging data indicating that the immune system has a major, albeit usually insufficient, role in limiting HIV replication. Virus-specific T-helper cells, which are critical to maintenance of cytolytic T cell (CTL) function, likely play a role. Viremia is also influenced by neutralizing antibodies, but they are generally low in magnitude in HIV infection and do not keep up with evolving viral variants in vivo.

Together these data indicate that increasing the strength and breadth of HIV-specific cellular immune responses might have a clinical benefit through so-called HIV immunotherapy. Some studies have tested vaccines against HIV, but success has been limited to date. Additionally, there has been interest in augmenting HIV immunotherapy by utilizing gene therapy techniques, but as with other immunotherapy approaches, success has been limited. Accordingly, there remains a need for improved treatments of HIV.

### SUMMARY

In an aspect, viral vector is provided comprising a therapeutic cargo portion, wherein the therapeutic cargo portion comprises a nucleotide sequence that encodes at least one soluble exogenous factor capable of inhibiting HIV infection; and a T cell-responsive promoter that regulates expression of the nucleotide sequence. In embodiments, the at least one soluble exogenous factor comprises an anti-HIV antibody. In embodiments, the anti-HIV antibody is a VRC01 antibody or a 3BNC117 antibody.

In embodiments, the at least one soluble exogenous factor comprises a soluble CD4 protein or a fragment thereof. In embodiments, the soluble CD4 or a fragment thereof comprises a dimeric soluble CD4. In embodiments, the dimeric soluble CD4 comprises a sequence having at least 80%, at least 85%, at least 90%, at least 95%, or at least 98% sequence identity to SEQ ID NO: 9, SEQ ID NO: 76, or SEQ ID NO: 77.

In embodiments, the T cell-responsive promoter comprises a CMV promoter, an IFN-α promoter, an IFN-β promoter, an IFN-γ promoter, an EF-1α promoter, an IL-2 promoter, a CD69 promoter, or a fragment thereof. In embodiments, the T cell-responsive promoter comprises an IL-2 promoter.

In embodiments, the therapeutic cargo portion further comprises a secretory signal that is operably linked to the nucleotide sequence that encodes the at least one soluble exogenous factor. In embodiments, the secretory signal comprises an antibody secretory signal or an IL-2 secretory signal.

In embodiments, the nucleotide sequence comprises a sequence having at least 80%, at least 85%, at least 90%, at least 95%, or at least 98% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87. In embodiments, the nucleotide sequence comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87.

In embodiments, the therapeutic cargo portion further comprises at least one small RNA that targets any one or more of Vif, Tat, and CCR5. In embodiments, the at least one small RNA comprises a sequence having at least 80%, at least 85%, at least 90%, at least 95%, or at least 98% sequence identity to SEQ ID NO: 62, SEQ ID NO: 63, or SEQ ID NO: 64. In embodiments, the at least one small RNA comprises SEQ ID NO: 62, SEQ ID NO: 63, or SEQ ID NO: 64.

In embodiments, the at least one small RNA comprises any two of Vif, Tat, and CCR5. In embodiments, the at least one small RNA comprises Vif, Tat, and CCR5. In embodiments, the at least one small RNA comprises a microRNA cluster that includes Vif, Tat, and CCR5. In embodiments, the at least one soluble exogenous factor comprises soluble CD4 or a fragment thereof. In embodiments, the soluble CD4 or fragment thereof comprises a dimeric soluble CD4. In embodiments, the T cell-responsive promoter comprises a CMV promoter, an IFN-α promoter, an IFN-β promoter, an IFN-γ promoter, an EF-1α promoter, an IL-2 promoter, a CD69 promoter, or a fragment thereof. In embodiments, the therapeutic cargo portion further comprises a secretory signal that is operably linked to the nucleotide sequence that encodes the at least one soluble exogenous factor. In embodiments, the at least one small RNA comprises a sequence having at least 80%, at least 85%, at least 90%, at least 95%, or at least 98% sequence identity to SEQ ID NO: 65. In embodiments, the at least one small RNA comprises SEQ ID NO: 65.

In an aspect, a lentiviral particle produced by a packaging cell and capable of infecting a target cell is provided, the lentiviral particle comprising an envelope protein capable of infecting the target cell; and any of the viral vectors described herein.

In an aspect, a modified cell comprising a lymphocyte infected with a lentiviral particle is provided, wherein the lentiviral particle comprises an envelope protein capable of infecting the lymphocyte; and any of the viral vectors described herein. In embodiments, the lymphocyte comprises a T cell, a B cell, an NKT cell, or an NK cell. In embodiments, the lymphocyte is a T cell, and the T cell comprises a CD4 T cell, a CD8 T cell, or a γδ T cell. In embodiments, the lymphocyte is a T cell, and the T cell comprises a CD4 T cell.

In an aspect, a viral delivery system is provided comprising at least one helper plasmid comprising nucleotide sequences for expressing a functional protein derived from each of a Gag, Pol, and Rev gene: an envelope plasmid comprising a DNA sequence for expressing an envelope protein capable of infecting a target cell; and any of the viral vectors described herein. In embodiments, the at least one helper plasmid comprises first and second helper plasmids, wherein the first helper plasmid encodes nucleotide sequences for expressing functional proteins derived from the Gag and the Pol genes, and the second helper plasmid encodes a nucleotide sequence for expressing a protein derived from the Rev gene

In an aspect, a method of treating HIV is provided, the method comprising contacting peripheral blood mononuclear cells (PBMC) isolated from a subject with a therapeutically effective amount of a stimulatory agent, wherein the contacting is carried out ex vivo; transducing the PBMC ex vivo with a lentiviral particle, wherein the lentiviral particle comprises an envelope protein capable of infecting the PBMC; and any of the viral vectors described herein; and culturing the transduced PBMC for at least 1 day. In embodiments, the method further comprises infusing the transduced PBMC into the subject. In embodiments, the stimulatory agent comprises a Gag peptide or an HIV vaccine.

### BRIEF DESCRIPTION OF THE DRAWINGS

In this Disclosure:

FIG. 1 depicts an exemplary 3-vector lentiviral vector system.

FIG. 2 depicts an exemplary 4-vector lentiviral vector system.

FIGS. 3A-3C depict vectors encode different soluble exogenous factors in a circular form. FIG. 3A depicts a lentiviral vector encoding the exogenous factor VRC01. FIG. 3B depicts a lentiviral vector encoding the exogenous factor sCD4. FIG. 3C depicts a lentiviral vector encoding the exogenous factor sCD4-IgG1 Fc.

FIG. 4 depicts vectors that encode various soluble exogenous factors in a linear form.

FIG. 5 depicts a schematic of a protocol for exogenously expressing the VRC01 antibody in CD4 T cells and then challenging the CD4 T cells with HIV.

FIG. 6 depicts flow cytometry data showing effect of T cell produced 3BNC117 antibody on HIV infection in vitro.

FIG. 7 depicts a schematic of a protocol for exogenously expressing sCD4 in CD4 T cells and then challenging the CD4 T cells with HIV.

FIGS. 8A and 8B depict flow cytometry data showing effect of HIV inhibition by CD4 T cells transduced with a lentivirus vector expressing sCD4.

FIG. 9 depicts a schematic of a protocol for exogenously expressing an HIV antibody in CD4 T cells.

FIG. 10 depicts flow cytometry data showing the effect of peptide stimulation of CD4 T cells following transduction with lentiviral vectors encoding the HIV antibodies VRC01 (AGT111) and 3BNC117 (AGT112).

FIG. 11 depicts a schematic of a protocol for stimulating CD4 T cells followed by transduction with a lentiviral vector encoding HIV antibodies.

FIG. 12 depicts flow cytometry data showing intracellular antibody accumulation in CD4 T cells when the cells are stimulated followed by transduction with lentiviral vectors encoding the VRC01 (AGT111) and 3BNC117 (AGT112) antibodies.

FIG. 13A depicts flow cytometry data showing the effect of T cell produced VRC01 antibody on HIV infection in vitro.

FIG. 13B depicts graphing data showing VRC01 antibody expression in T cells and the effect of the VRC01 antibody expression on HIV replication.

FIG. 14 depicts expression of VRC01 in the C8166 T cell line in cells transduced with a lentiviral vector encoding VRC01.

FIG. 15 depicts a schematic of a protocol for transducing the C8166 cell line with a lentiviral vector encoding a HIV antibody followed by challenging the cells with HIV.

FIG. 16 depicts flow cytometry data showing infection rates of HIV in C8166 cells that are transduced with a lentiviral vector that encodes the VRC01 antibody (AGT111).

FIG. 17 depicts antibody expression in culture after C8166 cells were transduced with a lentivirus encoding a VRC01 antibody (AGT113).

FIG. 18 depicts flow cytometry data showing effect on HIV infection when the VRC01 antibody is expressed in the C8166 T cell line.

FIG. 19 depicts flow cytometry data showing effect on HIV infection when sCD4 is expressed in the C8166 T cell line.

FIG. 20 depicts VRC01 expression in CD4 T cells after mitogen stimulated CD4 T cells were transduced with a lentiviral vector encoding the VRC01 antibody (AGT113).

FIG. 21 depicts flow cytometry data showing the effect of peptide stimulation of CD4 T cells after transduction with a lentiviral vector encoding VRC01 (AGT113).

FIG. 22 depicts a schematic of a protocol for stimulating CD4 T cells and transducing them lentiviral vectors encoding the HIV antibodies VRC01 and 3BNC117, followed by challenging the cells with HIV.

FIG. 23 depicts flow cytometry showing infection rates of CD4 T cells transduced with a lentiviral vector encoding VRC01 (AGT113) and treated with HIV.

FIG. 24 depicts flow cytometry comparing HIV infection rates of CD4 T cells transduced with lentiviral vectors encoding (i) soluble CD4 (AGT116) and (ii) soluble CD4 and IgG1 Fc (AGT117).

FIG. 25 depicts flow cytometry comparing infection rates of CD4 T cells transduced with lentiviral vectors encoding (i) a microRNA cluster that encodes microRNA targeting Vif, Tat, and CCR5 (AGT103) and (ii) a microRNA cluster that encodes microRNA targeting Vif, Tat, and CCR5, and soluble CD4 (AGT118).

FIG. 26 depicts flow cytometry showing expression levels of sCD4 in CD4 T cells using vectors encoding EF-1α, the IFNγ, and the IL-2 promoters.

FIG. 27 depicts flow cytometry comparing HIV infection rates of CD4 T cells transduced with the lentiviral vectors AGT117 (SEQ ID NO: 10), AGT124 (SEQ ID NO: 88), and AGT125 (SEQ ID NO: 89).

FIGS. 28A and 28B depict a schematic showing an expected mechanism of inhibiting HIV infection of T cells using sCD4.

FIG. 29 depicts relative expression levels in C8166 T cells of a lentivirus encoding a fusion protein comprised of soluble CD4 and different versions of the Fc region from human IgG1: Version 1: (SEQ ID NO: 9 (sCD4(D1+D2)-IgG1 Fc): Version 2 (SEQ ID NO: 76 (sCD4-IgG1 Fc (with antibody secretory signal) version 2); and Version 3 (SEQ ID NO: 77 (sCD4-IgG Fc (with antibody secretory signal) version 3).

FIG. 30 depicts binding of cell-free (supernatant) of CD4-IgG version 2 (SEQ ID NO: 76) (sCD4-IgGv2) and version 3 (SEQ ID NO: 77) (sCD4-IgGv3) to CD4-negative monocytoid cells (THP-1) that express Fc Receptor Gamma II.

FIGS. 31A-31G depict flow cytometry analysis of HIV infection of C8166 cells alone or after transduction with CD4-IgG version 1 (SEQ ID NO: 9) (sCD4-IgGv1) or version 2 (SEQ ID NO: 76) (sCD4-IgGv2) lentivirus vectors. Two different virus strains are compared; both versions protected cells from infection with version 2 conferring higher protection on the cells. FIG. 31A shows GFP expression in C8166 cells in which no virus was introduced. FIG. 31B shows GFP expression in C8166 cells in which an HXB2-GFP virus was introduced. FIG. 31C shows GFP expression in C8166 cells in which an HXB2-GFP virus was introduced along with version 1 CD4-IgG (SEQ ID NO: 9). FIG. 31D shows GFP expression in C8166 cells in which an HXB2-GFP virus introduced along with version 2 CD4-IgG (SEQ ID NO: 76). FIG. 31E shows GFP expression in C8166 cells in which an NL4-GFP vector was introduced. FIG. 31F shows GFP expression in C8166 cells in which an NL4-GFP vector was introduced along with version 1 CD4-IgG (SEQ ID NO: 9). FIG. 31G shows GFP expression in C8166 cells in which an NL4-GFP vector was introduced along with version 2 CD4-IgG (SEQ ID NO: 76).

### DETAILED DESCRIPTION

### Overview

Disclosed herein are methods and compositions for treating and/or inhibiting human immunodeficiency virus (HIV) disease to achieve a functional cure. The methods and compositions include lentiviral vectors and related viral vector technology, as described below.

### Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000): Ausubel et al., Short Protocols in Molecular Biology: A Compendium of Methods from Current Protocols in Molecular Biology, Wiley, John & Sons, Inc. (2002): Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, reference to any of “AGT 103,” “AGT111,” “AGT112,” “AGT113,” “AGT114,” “AGT115,” “AGT116,” “AGT117,” “AGT118,” “AGT119,” “AGT120,” “AGT121,” “AGT122,” “AGT123,” “AGT124,” and “AGT125” refers to the vectors disclosed in Table 1.

As used herein, the terms “administration of” or “administering” an active agent means providing an active agent to the subject in need of treatment in a form that can be introduced into that individual's body in a therapeutically useful form and therapeutically effective amount.

Throughout this specification and claims, the word “comprise,” or variations such as “comprises” or “comprising,” will be understood to imply the inclusion of a stated integer or group of integers but not the exclusion of any other integer or group of integers. Further, as used herein, the term “includes” means includes without limitation. The terms, “expression,” “expressed,” or “encodes” refer to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

The term “functional cure,” as referenced herein, refers to a state or condition wherein HIV+ individuals who previously required ongoing HIV therapies such as cART or HAART, may survive with low or undetectable virus replication using lower doses, intermittent doses, or discontinued dosing of such HIV therapies. An individual may be said to have been “functionally cured” while still requiring adjunct therapy to maintain low level virus replication and slow or eliminate disease progression. A possible outcome of a functional cure is the eventual eradication of all or virtually all HIV such that no recurrence is detected within a specified time frame, for example, 1 month, 3 months, 6 months, 1 year, 3 years, and 5 years, and all other time frames as may be defined.

The term “in vivo” refers to processes that occur in a living organism. The term “ex vivo” refers to processes that occur outside of a living organism. For example, in vivo treatment refers to treatment that occurs within a patient's body, while ex vivo treatment is one that occurs outside of a patient's body, but still uses or accesses or interacts with tissues from that patient. Thereafter, an ex vivo treatment step may include a subsequent in vivo treatment step.

The term “miRNA” refers to a microRNA, and also may be referred to herein as “miR”. The term “microRNA cluster” refers to at least two microRNAs that are situate on a vector in close proximity to each other and are co-expressed.

The term “packaging cell line” refers to any cell line that can be used to express a lentiviral particle.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of ordinary skill in the art) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules. BLAST protein searches can be performed with the XBLAST program, score=50, word length=3 to obtain amino acid sequences homologous to the protein molecules. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

As used herein, the term “SEQ ID NO” is synonymous with the term “Sequence ID No.”

As used herein, “small RNA” refers to RNAs that are generally less than about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, for example through pathways that result in the destruction of the target gene mRNA.

As used herein, the phrase “exogenous factor” refers to any nucleotide sequence or amino acid sequence that is capable of being expressed in a host cell and that is derived from a source other than the host cell. In embodiments, the amino acid sequence is capable of being expressed as a protein. In embodiments, the protein is an antibody.

As used herein, the term “stimulatory agent” refers to any exogenous agent that can stimulate an immune response, and includes, without limitation, vaccines (e.g., nucleic acid vaccines, carbohydrate vaccines, and peptides vaccines), including HIV vaccines, and HIV or HIV-related nucleic acids and peptides. A stimulatory agent can preferably stimulate a T cell response.

As used herein, the term “subject” refers to a subject that has an HIV infection or to a subject that is not infected with HIV but is seeking protection from a potential future HIV infection. Subject can include a human patient but also includes other mammals. The terms “subject,” “individual,” “host,” and “patient” may be used interchangeably herein.

As used herein, the phrase “T cell-responsive promoter” is any promoter that can be regulated by T cell receptor signaling and its cognate intracellular signaling pathway.

The term “therapeutically effective amount” refers to a sufficient quantity of the active agents, in a suitable composition, and in a suitable dosage form to treat or inhibit the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” is synonymous with a lentiviral vector.

The term “treatment” or “treating” generally refers to an intervention in an attempt to alter the natural course of the subject being treated, and can be performed either for prophylaxis or during the course of clinical pathology. Desirable effects include, but are not limited to, inhibiting occurrence or recurrence of disease, alleviating symptoms, suppressing, diminishing or inhibiting any direct or indirect pathological consequences of the disease, ameliorating or palliating the disease state, and causing remission or improved prognosis.

As used herein, the term “VRC01” refers to a human IgG1 monoclonal antibody, which targets the CD4 binding site on the HIV envelope gp120. The phrase “VRC01 antibody” is used interchangeably with the term “VRC01.”

As used herein, the term “3BNC117” refers to a human IgG1 monoclonal antibody, which targets the CD4 binding site on the HIV envelope gp160. The term “3BNC” and phrase “3BNC117 antibody” are used interchangeably with the term “3BNC117”.

As used herein, the term “fragment” refers to a portion of a nucleotide sequence that has been separated from a gene or a portion of an amino acid sequence that has been separated from a protein. The portion of the nucleotide or amino acid sequence can be separated from the gene or protein, respectively, using synthetic means (e.g., in a laboratory setting). Alternatively, the portion of the nucleotide or amino acid sequence can be separated from the gene or protein, respectively, through naturally occurring spontaneous processes.

As used herein, the term “enhancer” is a DNA sequence that is capable of being bound by a protein, and that, when bound by a protein, increases the chances that a particular gene will be transcribed.

As used herein, the phrase “soluble exogenous factor” refers to an “exogenous factor” that is capable of being secreted from cells and functioning in the extracellular space.

As used herein, the phrase “secretory signal,” refers to a peptide that is operably linked to a protein that is destined for export from the cell. The “secretory signal” functions to direct the protein to the export machinery within the cell resulting in secretion of the protein.

As used herein, the term “promoter” is a DNA sequence to which proteins are capable of binding and that, when bound, can result in initiation of transcription.

### Description of Aspects and Embodiments of the Disclosure

In an aspect, a viral vector is provided comprising a therapeutic cargo portion, wherein the therapeutic cargo portion comprises a nucleotide sequence that encodes at least one soluble exogenous factor capable of inhibiting HIV infection; and a T cell-responsive promoter that regulates expression of the nucleotide sequence. In embodiments, the viral vector comprises one or more plasmid DNA.

In an aspect, a viral vector is provided comprising a therapeutic cargo portion, wherein the therapeutic cargo portion comprises (i) a first nucleotide sequence that encodes at least one exogenous factor and (ii) a second nucleotide sequence that encodes at least one small RNA that targets at least one HIV gene; and a T cell-responsive promoter that regulates the expression of the first nucleotide sequence and the second nucleotide sequence.

In embodiments, the at least one soluble exogenous factor comprises an anti-HIV antibody. In embodiments, the anti-HIV antibody comprises at least one of a VRC01 antibody or a 3BNC117 antibody. In further embodiments, the anti-HIV antibody comprises at least one of a PG9 antibody, a PG16 antibody, a PG141-145 antibody, a CH01-04 antibody, a PGDM1400 antibody, a CAP256-VRC26.25 antibody, a VRC38 antibody, a PCT64 antibody, a PGT121 antibody, a PGT128 antibody, a PGT135 antibody, a 10-1074 antibody, a PCDN-33A antibody, a PGDM12 antibody, a PGDM21 antibody, a VRC29.03 antibody, a BF520.1 antibody, a VRC41.01 antibody, a BG18 antibody, a DH270.1 antibody, a DH270.6 antibody, a 10E8VLS antibody, a PGV04 antibody, a 8ANC131 antibody, a CH103 antibody, a CH235 antibody, a N6 antibody, a IOMA antibody, a N49-P7 antibody, a VRC07-523LS antibody, a N6LS antibody, a PGT151-158 antibody, a BANC195 antibody, a 35022 antibody, a N123-VRC34.01 antibody, a ACS202 antibody, a VRC-PG05 antibody, a SF12 antibody, a 10E8 antibody, or a Dh511 antibody. In embodiments, the anti-HIV antibody is any present or future anti-HIV antibody understood in the art.

In embodiments, the anti-HIV antibody binds to envelope glycoprotein GP120 (gp120) on the surface of an HIV envelope. In embodiments, the anti-HIV antibody binds to envelop glycoprotein GP160 (gp160) on the surface of an HIV envelope.

In embodiments, the anti-HIV antibody binds to the VIV2 loop on an HIV envelope glycoprotein. In embodiments, the anti-HIV antibody binds to a V3 loop on an HIV envelope glycoprotein. In embodiments, the anti-HIV antibody binds to a CD4 binding site on an HIV envelope glycoprotein. In embodiments, the anti-HIV antibody binds to a Gp120/gp41 interface on an HIV envelope glycoprotein. In embodiments, the anti-HIV antibody binds to a silent face gp120 on an HIV envelope glycoprotein. In embodiments, the anti-HIV antibody binds to a MPER epitope on an HIV envelope glycoprotein.

In embodiments, the anti-HIV antibody comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 69, SEQ ID NO: 71, SEQ ID NO: 72, SEQ ID NO: 74, SEQ ID NO: 75, or SEQ ID NO: 86. In embodiments, the anti-HIV antibody comprises SEQ ID NO: 69, SEQ ID NO: 71, SEQ ID NO: 72, SEQ ID NO: 74, SEQ ID NO: 75, or SEQ ID NO: 86.

In embodiments, the at least one exogenous factor comprises a soluble CD4 protein or a fragment thereof. In embodiments, the soluble CD4 comprises monomeric soluble CD4. In embodiments, the soluble CD4 comprises dimeric soluble CD4. In embodiments, the dimeric soluble CD4 comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 9, SEQ ID NO: 76, or SEQ ID NO: 77. In embodiments, the dimeric soluble CD4 comprises SEQ ID NO: 9, SEQ ID NO: 76, or SEQ ID NO: 77.

In embodiments, the at least one soluble exogenous factor is capable of binding to the envelope of HIV resulting in inhibiting binding of HIV to the surface of a lymphocyte. In embodiments the lymphocyte comprises a T cell, a B cell, an NK cell, an NKT cell. In embodiments, the lymphocyte is a T cell and the T cell comprises a CD8 T cell, a CD4 T cell, or a γδ T cell. In embodiments, the soluble factor binds to an envelope glycoprotein on the surface of the HIV envelope. In embodiments, the envelope glycoprotein is GP120. In embodiments, the envelope glycoprotein is GP160. In embodiments, the envelope glycoprotein is any envelope glycoprotein on the surface of HIV known in the art.

In embodiments, the nucleotide sequence that encodes the at least one soluble exogenous factor comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87. In embodiments, the nucleotide sequence comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87.

In embodiments, the T cell-responsive promoter comprises a CMV promoter, an EF-1a promoter, an IFN-γ promoter, an IL-2 promoter, a CD69 promoter, or a fragment thereof.

In embodiments, the CMV promoter comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 13. In embodiments, the CMV promoter comprises SEQ ID NO: 13.

In embodiments, the EF-1α promoter comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 14. In embodiments, the EF-1α promoter comprises SEQ ID NO: 14.

In embodiments, the IFN-γ promoter comprises a sequence that is at least 80%, at least 81%, at least 82%, at least 83%, at least 84% at least 85%, at least 86%, at least 87%, at least 88%, at least 89%, at least 90%, at least 91%, at least 92%, at least 93%, at least 94% at least 95%, at least 96%, at least 97%, at least 98%, or at least 99% identical to SEQ ID NO: 15. In embodiments, the IFN-γ promoter comprises SEQ ID NO: 15.

In embodiments, the IL-2 promoter comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 66. In embodiments, the IL-2 promoter comprises SEQ ID NO: 66.

In embodiments, the CD69 promoter comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 67 (CD69 promoter (1050)+CNS2) enhancer) or SEQ ID NO: 68 (CD69 promoter (625)+CNS2 enhancer). In embodiments, the CD69 promoter comprises SEQ ID NO: 67 or SEQ ID NO: 68.

In embodiments, the T cell-responsive promoter comprises a constitutive promoter. In embodiments, the T cell-responsive promoter comprises a tissue-specific promoter. In embodiments, the T cell-responsive promoter comprises an inducible promoter.

In embodiments, the T cell-responsive promoter comprises at least one of an IFN-α promoter, an IFN-β promoter, a SV40 promoter, a PGK1 promoter, a CAG promoter, a Ubc promoter, an H1 promoter, or a U6 promoter.

In further embodiments, the T cell-responsive promoter comprises at least one of a FOXP3 promoter, a IL2RA promoter, a CTLA4 promoter, a IKZF2 promoter, a CD40LG promoter, a THEMIS promtoer, a SATB1 promoter, a LAIR2 promoter, a METTL7A promoter, a RTKN2 promoter, a TCF7 promoter, an ANK3 promoter, a NELL2 promoter, an ANXA1 promoter, a TGFB1 promoter, a TIGIT promoter, a TNFRSF10B promoter, a LAG3 promoter, a GZMA promoter, an IL10 promoter, a FGL2 promoter, an ENTPD1 promoter, a CCR6 promoter, a CCR9 promoter, a CCR10 promoter, a MAF promoter, a TBX21 promoter, a RORC promoter, an AHR promoter, a PRDM1 promoter, a GATA3 promoter, an IFNG promoter, a TNFA promoter, a GZMB promoter, a FURIN promoter, an IL12A promoter, an ICOS promoter, a LGALS1 promoter, a CCR7 promoter, a CCL5 promoter, a CCL3 promoter, a CCL4 promoter, a CCR1 promoter, an ICAM1 promoter, a CCR3 promoter, a CCR8 promoter, a CCR2 promoter, a CCR5 promoter, a CXCR6 promoter, a CXCR3 promoter, a CXCR4 promoter, a CXCR5 promoter, a CCR9 promoter, a CCR10 promoter, a FER promoter, a PECAM1 promoter, a CCR4 promoter, an ITGA4 promoter, a SELPLG promoter, a RUNX1 promoter, a STAT5 promoter, a FOXP3 promoter, a H3K27ac promoter, a hPGK promoter, or a RPBSA promoter.

In embodiments, the T cell-responsive promoter is any present or future T cell-responsive promoter understood in the art that is inducible by HIV, an HIV gene, or other HIV structural feature. In embodiments, the HIV gene, protein, or structural feature comprises at least one of: Gag, Pol, Tat, Rev, Nef, Vif, Vpr, Vpu, Tev, LTR, TAR, RRE, PE, SLIP, CRS, and INS.

In embodiments, the viral vector further comprises at least one enhancer that is operably linked to the T cell-responsive promoter. In embodiments, the at least one enhancer comprises one enhancer, two enhancers, three enhancers, four enhancers, five enhancers, or any greater number. In embodiments, the at least one enhancer comprises more than five enhancers.

In embodiments, the enhancer is provided in a promoter/enhancer combination. In embodiments, the promoter/enhancer combination comprises a sequence that is at least 80%, at least 81%, at least 82%, at least 83%, at least 84% at least 85%, at least 86%, at least 87%, at least 88%, at least 89%, at least 90%, at least 91%, at least 92%, at least 93%, at least 94% at least 95%, at least 96%, at least 97%, at least 98%, or at least 99% identical to SEQ ID NO: 16. In embodiments, the promoter/enhancer combination comprises SEQ ID NO: 16.

In embodiments, the therapeutic cargo portion further comprises a secretory signal that is operably linked to the nucleotide sequence that encodes the at least one soluble exogenous factor. In embodiments, the secretory signal is an IL-2 secretory signal. In embodiments, the nucleotide sequence that encodes the IL-2 secretory signal is at least 80%, at least 81%, at least 82%, at least 83%, at least 84%, at least 85%, at least 86%, at least 87%, at least 88%, at least 89% at least 90%, at least 91%, at least 92%, at least 93%, at least 94% at least 95%, at least 96%, at least 97%, at least 98%, or at least 99% identical to SEQ ID NO: 11. In embodiments, the nucleotide sequence that encodes the IL-2 secretory signal comprises SEQ ID NO: 11.

In embodiments, the secretory signal is an antibody secretory signal. In embodiments, the nucleotide sequence that encodes the antibody secretory signal is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 12. In embodiments, the nucleotide sequence that encodes that antibody secretory signal comprises SEQ ID NO: 12.

In embodiments, the secretory signal comprises an APO secretory signal, an ARSF secretory signal, an ART4 secretory signal, an ARTN secretory signal, an AZGP1 secretory signal, a BSGAT1 secretory signal, a BDNF secretory signal, a BMP secretory signal, a BTN secretory signal, a C1Q secretory signal, a C1R secretory signal, a C3 secretory signal, a CA10 secretory signal, a CALCA secretory signal, a CALCB secretory signal, a CCK secretory signal, a CCL secretory signal, a CD14 secretory signal, a CD163 secretory signal, a CD6 secretory signal, a CEACAM16 secretory signal, a CEL secretory signal, a CGA secretory signal, a CGB secretory signal, a CKLFCLEC secretory signal, a COL secretory signal, a CPA secretory signal, a CPB secretory signal, a CSF secretory signal, a CSHCSN secretory signal, a CTRB2 secretory signal, a CXCL secretory signal, a DEF secretory signal, a DPP4 secretory signal, a F10 secretory signal, a F11 secretory signal, a F12 secretory signal, a F13 secretory signal, a F2 secretory signal, a F3 secretory signal, a F5 secretory signal, a F7 secretory signal, a F8 secretory signal, a F9 secretory signal, a FGF secretory signal, a FGFBP secretory signal, a FSHB secretory signal, a GCG secretory signal, a GZM secretory signal, a HSPG2 secretory signal, a IFNA secretory signal, an IFNB secretory signal, an IFNE secretory signal, an IFNG secretory signal, an IFNK secretory signal, an IFNL secretory signal, an IFNW1 secretory signal, an IGF secretory signal, an IL secretory signal, an INS secretory signal, an INSL secretory signal, a KLK secretory signal, a LALB secretory signal, a LBP secretory signal, a LIF secretory signal, a LTF secretory signal, a LYGMBL2 secretory signal, a MMP secretory signal, a MUC secretory signal, a NDNF secretory signal, a NGFN secretory signal, a NPPA secretory signal, a NRP1 secretory signal, a NRP2 secretory signal, a PLAG2G secretory signal, a PLAC1 secretory signal, a PLAT secretory signal, a PLAU secretory signal, a PPIA secretory signal, a PRL secretory signal, a PROC secretory signal, a PRSS secretory signal, a PTH secretory signal, a RNAS secretory signal, a SDC4 secretory signal, a SERPINA secretory signal, a SFTPA secretory signal, a TNFRS secretory signal, a TSLP secretory signal, a TRH secretory signal, a TTR secretory signal, a UTS secretory signal, a VIP secretory signal, a VTN secretory signal, a VWA secretory signal, or a WIF secretory signal. In embodiments, the secretory signal comprises any known or future secretory signal as understood in the art.

In embodiments, the secretory signal comprises any secretory signal capable of facilitating the secretion of an exogenous factor that can target HIV. In embodiments, the exogenous factor can target any HIV gene, protein, or structural feature. In embodiments, the HIV gene, protein, or structural feature can comprise any of the following: Gag, Pol, Tat, Rev, Nef. Vif. Vpr, Vpu, Tev, LTR, TAR, RRE, PE, SLIP, CRS, and INS.

In embodiments, the at least one HIV gene is Vif. In embodiments, the at least one HIV gene is Tat. In embodiments, the at least one HIV gene is Vif and Tat. In embodiments, the at least one HIV gene comprises any one or more HIV genes known in the art. In embodiments, the at least one HIV gene comprises at least one of Gag, Pol, Tat, Rev, Nef, Vif, Vpr. Vpu, and Tev.

In embodiments, the therapeutic cargo portion further comprises a nucleotide sequence that encodes at least one small RNA that targets CCR5. In embodiments, the therapeutic cargo portion comprises at least one small RNA that targets CCR5 and at least one HIV gene. In embodiments, the at least one HIV gene is Vif. In embodiments, the at least one HIV gene is Tat. In embodiments, the at least one HIV gene is Vif and Tat. In embodiments, the at least one HIV gene is any one or more HIV genes known in the art. In embodiments, the at least one HIV gene comprises at least one of Gag, Pol, Tat, Rev, Nef, Vif. Vpr, Vpu, and Tev.

In embodiments, the at least one small RNA is a at least one microRNA, at least one shRNA, or at least one siRNA. In embodiments, the at least one small RNA is any known or future small RNA understood in the art.

In embodiments, the at least one small RNA comprises a microRNA that targets CCR5. In embodiments, the microRNA that targets CCR5 comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 62. In embodiments, the microRNA that targets CCR5 comprises SEQ ID NO: 62.

In embodiments, the at least one small RNA comprises a small RNA that targets CCR5.

In embodiments, the at least one small RNA comprises a microRNA that targets Vif. In embodiments, the microRNA that targets Vif comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 63. In embodiments, the microRNA that targets Vif comprises SEQ ID NO: 63.

In embodiments, the at least one small RNA comprises a small RNA that targets Vif.

In embodiments, the at least one small RNA comprises a microRNA that targets Tat. In embodiments, the microRNA that targets Tat comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 64. In embodiments, the microRNA that targets Tat comprises SEQ ID NO: 64.

In embodiments, the at least one small RNA comprises a small RNA that targets Tat.

In embodiments, the at least one small RNA comprises small RNAs that target Vif, Tat, and CCR5. In embodiments, the small RNAs comprise a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 65. In embodiments, the microRNA cluster comprises SEQ ID NO: 65.

In embodiments, the at least one small RNA comprises a small RNA that targets Vif, a small RNA that targets Tat, and a small RNA that targets CCR5. In embodiments, the at least one small RNA is a microRNA cluster.

In an aspect, lentiviral particle is provided. The lentiviral particle variously comprises an envelope protein capable of infecting the target cell; and any of the viral vectors described herein. In embodiments, the lentiviral particle produced by a packaging cell and capable of infecting a target cell.

In embodiments, the target cell is a lymphocyte. In embodiments, the lymphocyte is a T cell, a B cell, an NKT cell, or an NK cell. In embodiments, the T cell is a CD4 T cell, a CD8 T cell, or a γδ T cell.

In an aspect, a modified cell comprising a lymphocyte infected with a lentiviral particle is provided. In embodiments, the lentiviral particle variously comprises an envelope protein capable of infecting the lymphocyte; and any of the viral vectors described herein. In embodiments, the lymphocyte is a T cell, B cell, NKT cell, or NK cell. In embodiments, the lymphocyte is a T cell, and the T cell is a CD4 T cell, a CD8 T cell, or a γδ T cell.

In an aspect, a viral delivery system is provided. In embodiments, the viral delivery system variously comprises at least one helper plasmid comprising nucleotide sequences for expressing a functional protein derived from each of a Gag, Pol, and Rev gene: an envelope plasmid comprising a DNA sequence for expressing an envelope protein capable of infecting a target cell; and any of the viral vectors described herein. In embodiments, the at least one helper plasmid comprises first and second helper plasmids, wherein the first helper plasmid encodes nucleotide sequences for expressing functional proteins derived from the Gag and the Pol genes, and the second helper plasmid encodes a nucleotide sequence for expressing a protein derived from the Rev gene.

In an aspect, a method of treating HIV is provided. In embodiments, the method variously comprises contacting peripheral blood mononuclear cells (PBMC) isolated from a subject with a therapeutically effective amount of a stimulatory agent, wherein the contacting is carried out ex vivo: transducing the PBMC ex vivo with a lentiviral particle, wherein the lentiviral particle comprises an envelope protein capable of infecting the PBMC; and any of the viral vectors described herein; and culturing the transduced PBMC for at least one day.

In embodiments, the method further comprises infusing the transduced PBMC into a subject.

In embodiments, the stimulatory agent is derived from HIV. In embodiments, the stimulatory agent is a peptide derived from HIV. In further embodiments, the peptide comprises a Gag peptide. In embodiments, the stimulatory agent comprises an Env peptide.

In another aspect, the method comprises administering two stimulatory agents, a first stimulatory agent and a second stimulatory agent. In embodiments, the first stimulatory agent and second stimulatory agent are the same stimulatory agent. In embodiments, the first stimulatory agent and the second stimulatory agent are each a Gag peptide. In embodiments, the first stimulatory agent and the second stimulatory agent are each an Env peptide. In embodiments, the first stimulatory agent and second stimulatory agent are different stimulatory agents. In embodiments, the first stimulatory agent is administered ex vivo. In embodiments, the second stimulatory agent is administered in vivo. In embodiments, the method comprises administering a first simulatory agent, transducing the cells with any lentiviral vector described herein, and administering a second stimulatory agent.

In embodiments, the peptide activates at least one lymphocyte. In embodiments, the at least one type of lymphocyte is a T cell, B cell, NKT cell, or NK cell. In embodiments, the lymphocyte is a T cell. In embodiments, the T cell is a CD4 T cell, a CD8 T cell, or a γδ T cell. In embodiments, the lymphocytes that are activated are MHC class I restricted lymphocytes. In embodiments, the lymphocytes that are activated are MHC class II restricted lymphocytes.

In embodiments, the transduced PBMC can be cultured for more than 1 day. In embodiments, the transduced PBMC are cultured for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days 32 days, 33 days, 34 days, 35 days, or greater. In embodiments, the transduced PBMC are cultured for more than 35 days.

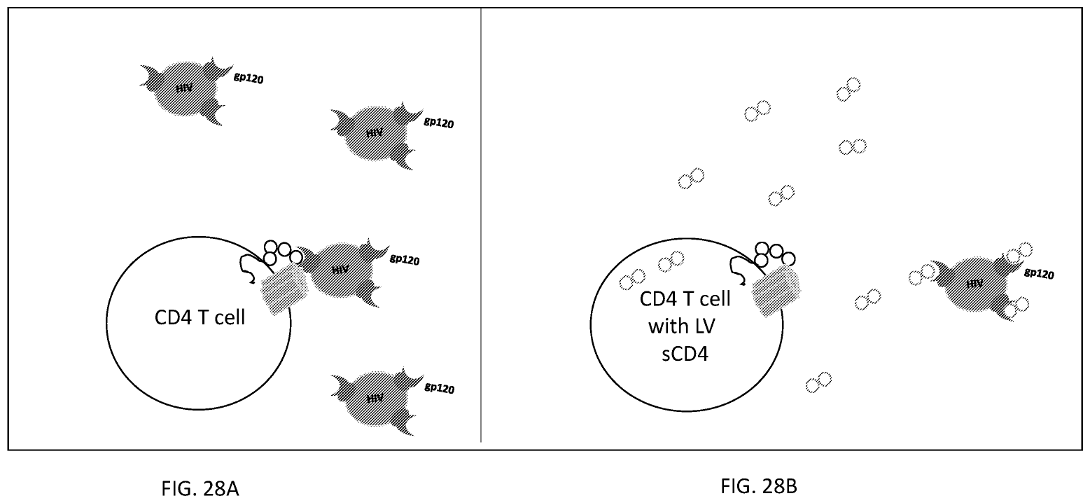

In an aspect, a mechanism of inhibiting HIV infection of CD4 T cells is provided. In embodiments, the mechanism of inhibiting HIV infection is provided in FIGS. 28A and 28B. In embodiments, as shown in FIG. 28A, HIV binds to CD4 receptors (100) on the surface of a CD4 T cell (140). In embodiments, when soluble CD4 (also referred to herein as sCD4) (110) is present in the extracellular space, it binds to an envelope protein (eg. gp120) (120) on the surface of HIV (130) (FIG. 28B). This inhibits binding of HIV to CD4 receptors (100) on the surface of a CD4 T cell (140) (FIG. 28B). In embodiments, sCD4 can be replaced by an anti-HIV antibody. In embodiments, the anti-HIV antibody comprises any anti-HIV antibody disclosed herein or any variant thereof. In embodiments, the anti-HIV antibody comprises any anti-HIV antibody understood in the art or any variant thereof. In embodiments, the sCD4 or anti-HIV antibody is provided to bind any glycoprotein on the surface of HIV that would inhibit HIV from entering the cell.

In an aspect, a method of treating HIV is provided. In embodiments, the method variously comprises obtaining peripheral blood mononuclear cells (PBMC) from a patient. In embodiments, the PBMC are isolated using any suitable technique. In embodiments, the PBMC are contacted with a therapeutically effective amount of a stimulatory agent. In embodiments, contacting the PBMC with the stimulatory agent takes place ex vivo. In embodiments, the stimulatory agent comprises an HIV vaccine. In embodiments, the stimulatory agent comprises a Gag peptide. In embodiments, the stimulatory agent comprises an Env peptide. In embodiments, the stimulatory agent results in the PBMC being more susceptible to transduction. In embodiments, the contacting with a therapeutically effective amount of the stimulatory agent occurs ex vivo. In embodiments, contacting with the stimulatory agent is followed by transduction with a lentiviral particle. In embodiments, the lentiviral particle comprises an envelope protein capable of infecting the PBMC. In embodiments, the lentiviral particle is any lentiviral particle disclosed herein. In embodiments, following transduction, the PBMC are cultured for a time period sufficient to allow for suitable expansion of the PBMC. In embodiments, the time period is at least one day. In embodiments, the PBMC are administered to a patient.

### Human Immunodeficiency Virus (HIV)

Human Immunodeficiency Virus, which is also commonly referred to as “HIV,” is a retrovirus that causes acquired immunodeficiency syndrome (AIDS) in humans. Without treatment, average survival time after infection with HIV is estimated to be 9 to 11 years, depending upon the HIV subtype. Infection with HIV occurs by the transfer of bodily fluids, including but not limited to blood, semen, vaginal fluid, pre-ejaculate, saliva, tears, lymph or cerebro-spinal fluid, or breast milk. HIV may be present in an infected individual as both free virus particles and within infected immune cells.

HIV infects vital cells in the human immune system such as helper T cells, although tropism can vary among HIV subtypes. Immune cells that may be specifically susceptible to HIV infection include but are not limited to CD4+ T cells, macrophages, and dendritic cells. HIV infection leads to low levels of CD4+ T cells through a number of mechanisms, including but not limited to apoptosis of uninfected bystander cells, direct viral killing of infected cells, and killing of infected CD4+ T cells by CD8 cytotoxic lymphocytes that recognize infected cells. When CD4+ T cell numbers decline below a critical level, cell-mediated immunity is lost.

Structurally, HIV is distinct from many other retroviruses. The RNA genome consists of at least seven structural landmarks (LTR, TAR, RRE, PE, SLIP, CRS, and INS), and at least nine genes (Gag, Pol, Env, Tat, Rev, Nef, Vif. Vpr, Vpu, and sometimes a tenth Tev, which is a fusion of Tat, Env, and Rev), encoding 19 proteins. Three of these genes, Gag, Pol, and Env, contain information needed to make the structural proteins for new virus particles.

HIV replicates primarily in CD4 T cells, and causes cellular destruction or dysregulation to reduce host immunity. Because HIV establishes infection as an integrated provirus and may enter a state of latency wherein virus expression in a particular cell decreases below the level for cytopathology affecting that cell or detection by the host immune system, HIV is difficult to treat and has not been eradicated even after prolonged intervals of highly active antiretroviral therapy (HAART). In the vast majority of cases, HIV infection causes fatal disease although survival may be prolonged by HAART.

A major goal in the fight against HIV is to develop strategies for curing disease. Prolonged HAART has not accomplished this goal, so investigators have turned to alternative procedures. Early efforts to improve host immunity by therapeutic immunization (using a vaccine after infection has occurred) had marginal or no impact. Likewise, treatment intensification had moderate or no impact.

Some progress has been made using genetic therapy, but positive results are sporadic and found only among rare human beings carrying defects in one or both alleles of the gene encoding CCR5 (chemokine receptor). However, many investigators are optimistic that genetic therapy holds the best promise for eventually achieving an HIV cure.

As disclosed herein, the methods and compositions are able to achieve a functional cure. The primary obstacles to achieving a functional cure lie in the basic biology of HIV itself. Virus infection deletes CD4 T cells that are critical for nearly all immune functions. Most importantly, HIV infection and depletion of CD4 T cells requires activation of individual cells. Activation is a specific mechanism for individual CD4 T cell clones that recognize pathogens or other molecules, using a rearranged T cell receptor.

In the case of HIV, infection activates a population of HIV-specific T cells that become infected and are consequently depleted before other T cells that are less specific for the virus, which effectively cripples the immune system's defense against the virus. The capacity for HIV-specific T cell responses is rebuilt during prolonged HAART: however, when HAART is interrupted the rebounding virus infection repeats the process and again deletes the virus-specific cells, which promotes disease progression.

A functional cure may be only possible if enough HIV-specific CD4 T cells are protected to allow for a host's native immunity to confront and control HIV once HAART is interrupted.

In various aspects, methods and compositions are provided for improving the effectiveness of genetic therapy to provide a functional cure of HIV disease. In embodiments, methods and compositions are provided for enhancing host immunity against HIV to provide a functional cure. In further embodiments, methods and compositions are provided for enriching HIV-specific CD4 T cells in a patient to achieve a functional cure.

### Gene Therapy

Viral vectors are provided herein to deliver genetic constructs to host cells for the purposes of treating or inhibiting HIV.

These genetic constructs can include, but are not limited to, functional genes or portions of genes to correct or complement existing defects, DNA sequences encoding regulatory proteins, DNA sequences encoding regulatory RNA molecules including antisense, short homology RNA, long non-coding RNA, small interfering RNA or others, and decoy sequences encoding either RNA or proteins designed to compete for critical cellular factors to alter a disease state. Gene therapy as provided herein involves delivering these therapeutic genetic constructs to target cells to provide treatment or alleviation of HIV-related disease.

Gene therapy as provided herein can include, but is not limited to, affinity-enhanced T cell receptors, chimeric antigen receptors on CD4 T cells (or alternatively on CD8 T cells or γδ T cells), modification of signal transduction pathways to avoid cell death cause by viral proteins, increased expression of HIV restriction elements including TREX, SAMHD1, MxA or MxB proteins, APOBEC complexes, TRIM5-alpha complexes, tetherin (BST2), and similar proteins identified as being capable of reducing HIV replication in mammalian cells.

### Immunotherapy

Historically, vaccines have been a go-to weapon against deadly infectious diseases, including smallpox, polio, measles, and yellow fever. Unfortunately, there is no currently approved vaccine for HIV. The HIV virus has unique ways of evading the immune system, and the human body seems incapable of mounting an effective immune response against it. As a result, scientists do not have a clear picture of what is needed to provide protection against HIV. However, immunotherapy may provide a solution that was previously unaddressed by conventional vaccine approaches.

In various aspects and embodiments, immunotherapeutic approaches enrich a population of HIV-specific CD4 T cells for the purpose of increasing the host's anti-HIV immunity. In embodiments, integrating or non-integrating lentivirus vectors are used to transduce a host's immune cells for the purposes of increasing the host's anti-HIV immunity. In further embodiments, a vaccine comprising HIV proteins is provided, including but not limited to a killed particle, a virus-like particle, HIV peptides or peptide fragments, a recombinant viral vector, a recombinant bacterial vector, a purified subunit or plasmid DNA combined with a suitable vehicle and/or biological or chemical adjuvants to increase a host's immune responses. This vaccine may be used to enrich the population of virus-specific T cells or antibodies. Various methods are provided to further enhance through the use of HIV-targeted genetic therapy using lentivirus or other viral vectors.

### Methods

In various aspects, the methods for using viral vectors to achieve a functional cure for HIV disease are provided. The methods variously include immunotherapy to enrich the proportion of HIV-specific CD4 T cells, and lentivirus transduction to enable delivery of exogenous factors capable of inhibiting HIV.

In embodiments, the methods include a first stimulation event to enrich a proportion of HIV-specific CD4 T cells. The first stimulation can include administration of one or more of any agent suitable for enriching a patient's HIV-specific CD4+ T cells including but not limited to a vaccine.

Therapeutic vaccines can include one or more HIV proteins with protein sequences representing the predominant viral types of the geographic region where treatment is occurring. Therapeutic vaccines include purified proteins, inactivated viruses, virally vectored proteins, bacterially vectored proteins, peptides or peptide fragments, virus-like particles (VLPs), biological or chemical adjuvants including cytokines and/or chemokines, vehicles, and methods for immunization. Immunizations may be administered according to standard methods known in the art and HIV patients may continue antiretroviral therapy during the interval of immunization and subsequent ex vivo lymphocyte culture including lentivirus transduction.

In embodiments, the methods include ex vivo stimulation of CD4 T cells from persons or patients previously immunized by therapeutic vaccination, using purified proteins, inactivated viruses, virally vectored proteins, bacterially vectored proteins, biological or chemical adjuvants including cytokines and/or chemokines, vehicles, and methods for stimulation. Ex vivo stimulation may be performed using the same vaccine or immune stimulating compound used for immunization, or it may be performed using a different vaccine or immune stimulating compound than those used for immunization.

In embodiments, peripheral blood mononuclear cells (PBMCs) may be obtained by standard techniques including leukapheresis. In embodiments, the PBMCs are treated ex vivo. In further embodiments, the treatment yields expansion of CD4 T cells. In embodiments, a yield of 1×10¹⁰ CD4 T cells is obtained of which about 0.1%, about 1%, about 5% or about 10% or about 30% may be both HIV-specific in terms of antigen responses, and HIV-resistant by virtue of carrying the therapeutic transgene delivered by the disclosed lentivirus vector. Alternatively, about 1×10⁷, about 1×10⁸, about 1×10⁹, about 1×10¹⁰, about 1×10¹¹, or about 1×10¹² CD4 T cells may be isolated for ex vivo stimulation. Any suitable amount of CD4 T cells are isolated for ex vivo stimulation.

The isolated CD4 T cells can be cultured in appropriate medium throughout stimulation with HIV vaccine antigens, which may include antigens present in the prior therapeutic vaccination. Antiretroviral therapeutic drugs including inhibitors of reverse transcriptase, protease or integrase may be added to inhibit virus re-emergence during prolonged ex vivo culture. CD4 T cell ex vivo stimulation is used to enrich the proportion of HIV-specific CD4 T cells in culture. The same procedure may also be used for analytical objectives wherein smaller blood volumes with peripheral blood mononuclear cells obtained by purification, are used to identify HIV-specific T cells and measure the frequency of this sub-population.

The PBMC fraction may be enriched for HIV-specific CD4 T cells by contacting the cells with HIV proteins matching or complementary to the components of the vaccine previously used for in vivo immunization. Ex vivo stimulation can increase the relative frequency of HIV-specific CD4 T cells by about 5-fold, about 10-fold, about 25-fold, about 50-fold, about 75-fold, about 100-fold, about 125-fold, about 150-fold, about 175-fold, or about 200-fold.

Various methods may additionally include combining in vivo therapeutic immunization and ex vivo stimulation of CD4 T cells with ex vivo lentiviral transduction and culturing.

In various embodiments, an ex vivo stimulated PBMC fraction that has been enriched for HIV-specific CD4 T cells can be transduced with therapeutic lentivirus encoding exogenous factors capable of inhibiting HIV or other vectors and maintained in culture for a sufficient period of time for such transduction, for example from about 1 to about 21 days, including up to about 35 days, or greater than 35 days. In further embodiments, the cells may be cultured for about 1-about 18 days, about 1-about 15 days, about 1-about 12 days, about 1-about 9 days, or about 3-about 7 days. The transduced cells may be cultured for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35 days, or greater than 35 days.

In further embodiments, transduced CD4 T cells are infused back into a patient, such as the original patient from which the CD4 T cells were obtained. Infusion can be performed using any suitable devices and methods. In some embodiments, infusion may be accompanied by pre-treatment with cyclophosphamide or similar compounds to increase the efficiency of engraftment.

In various embodiments, continued virus suppression is provided, including antiretroviral therapy such as cART or HAART. In other embodiments, the antiretroviral therapy is reduced from pre-infusion dosages and/or levels. In some embodiments, reduced or no adjuvant therapy for about 26 weeks may be considered a functional cure for HIV. Other definitions of a functional cure are described herein.

Viral vectors herein may encode at least one, at least two, at least three, at least four, or at least five genes, or at least six genes, or at least seven genes, or at least eight genes, or at least nine genes, or at least ten genes, or at least eleven genes, or at least twelve, or greater, genes of interest. A viral vector herein may encode genes or nucleic acid sequences that include but are not limited to (i) an antibody directed to an HIV antigen associated with HIV disease or a toxin produced by HIV, (ii) cytokines including interleukins that are required for immune cell growth or function and may be therapeutic for immune dysregulation encountered in HIV, (iii) factors that suppress the growth of HIV in vivo including CD8 suppressor factors, (iv) mutations or deletions of chemokine receptor CCR5, mutations or deletions of chemokine receptor CXCR4, or mutations or deletions of chemokine receptor CXCR5, (v) antisense DNA or RNA against specific receptors or peptides associated with HIV or host protein associated with HIV, (vi) small interfering RNA against specific receptors or peptides associated with HIV or host protein associated with HIV, (vii) a exogenous factor such as, for example, a CD4 (e.g., sCD4), that binds to HIV in the extracellular space resulting in inhibition of HIV entry into cells, or (viii) a variety of other therapeutically useful sequences that may be used to treat HIV or AIDS.

Additional examples of HIV-targeted gene therapy for use in the disclosed methods include, but are not limited to, affinity-enhanced T cell receptors, chimeric antigen receptors on CD4 T cells (or alternatively on CD8 T cells or γδ T cells), modification of signal transduction pathways to avoid cell death cause by viral proteins, increased expression of HIV restriction elements including TREX, SAMHD1, MxA or MxB proteins, APOBEC complexes, TRIM5-alpha complexes, tetherin (BST2), and similar proteins identified as being capable of reducing HIV replication in mammalian cells.

In embodiments, a patient may be undergoing cART or HAART concurrently while being treated according to the methods disclosed herein. In other embodiments, a patient may undergo CART or HAART before or after being treated according to the methods disclosed herein. In other embodiments, cART or HAART is maintained throughout treatment and the patient may be monitored for HIV viral burden in blood and frequency of lentivirus-transduced CD4 T cells in blood. Preferably, a patient receiving cART or HAART prior to being treated according is able to discontinue or reduce cART or HAART following treatment.

The frequency of transduced, HIV-specific CD4 T cells, which is a novel surrogate marker for gene therapy effects, may be determined, as discussed in more detail herein.

### Compositions

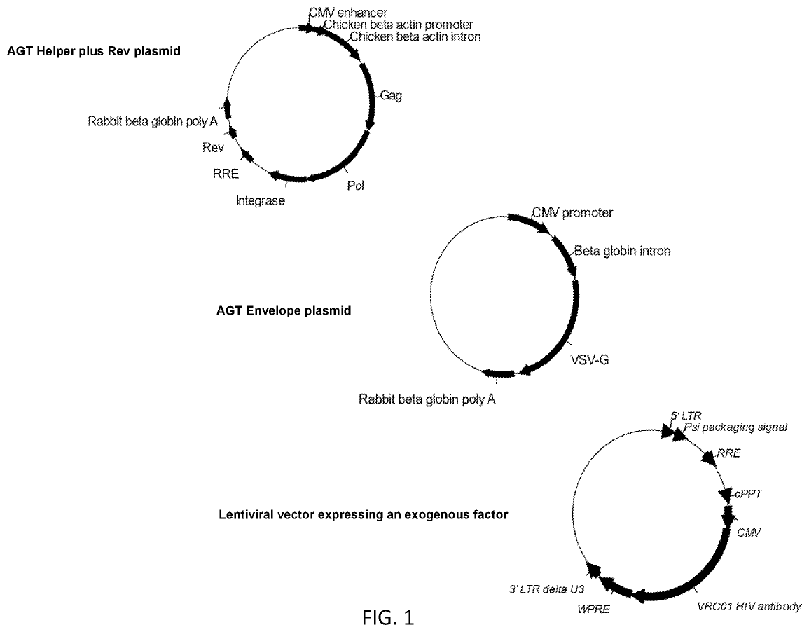

As shown in FIGS. 1-4, an exemplary construct may comprise numerous components. For example, in one embodiment, an exemplary LV construct may comprise the following sections or components:
RSV—a Rous Sarcoma virus long terminal repeat:
   5′LTR—a portion of an HIV long terminal repeat that can be truncated to inhibit replication of the vector after chromosomal integration;
   Psi—a packaging signal that allows for incorporation of the vector RNA genome into viral particles during packaging:
   RRE—a Rev Responsive element can be added to improve expression from the transgene by mobilizing RNA out of the nucleus and into the cytoplasm of cells;
   cPPT—a Poly purine tract that facilitates second strand DNA synthesis prior to integration of the transgene into the host cell chromosome;
   Promoter—a promoter initiates RNA transcription from the integrated transgene to express exogenous factors, small RNA, micro-RNA clusters, or other genetic elements of the construct, and in some embodiments, the vectors may use an EF-1 promoter;
   Anti-VRC01 antibody and anti-3BNC117 as well as other HIV antibodies that block interaction of HIV with the CD4 receptor, thereby inhibiting entry of HIV into CD4 T cells:
   sCD4-blocks interaction of HIV with the CD4 receptor on the surface of T cells, thereby inhibiting entry of HIV into CD4 T cells;
   Anti-CCR5—a micro RNA targeting messenger RNA for the host cell factor CCR5 to reduce its expression on the cell surface:
   Anti-Rev/Tat—a micro RNA targeting HIV genomic or messenger RNA at the junction between HIV Rev and Tat coding regions, which is sometimes designated miRNA Tat or given a similar description in this application;
   Anti-Vif—a micro RNA targeting HIV genomic or messenger RNA within the Vif coding region;
   WPRE—a woodchuck hepatitis virus post-transcriptional regulatory element is an additional vector component that can be used to facilitate RNA transport out of the nucleus; and
   deltaU3 3′LTR—a modified version of a HIV 3′ long terminal repeat where a portion of the U3 region has been deleted to improve safety of the vector.

### Lentiviral Vector System

A lentiviral virion (particle) is provided. In accordance with various aspects and embodiments it may be expressed by a vector system encoding the necessary viral proteins to produce a virion (viral particle). In various embodiments, one vector plasmid containing a nucleic acid sequence encoding the lentiviral Pol proteins is provided for reverse transcription and integration, operably linked to a promoter. In another embodiment, the Pol proteins are expressed by multiple vector plasmids. In other embodiments, vector plasmids containing a nucleic acid sequence encoding the lentiviral Gag proteins for forming a viral capsid, operably linked to a promoter, are provided. In embodiments, this Gag nucleic acid sequence is on a separate vector than at least some of the Pol nucleic acid sequence. In other embodiments, the Gag nucleic acid is on a separate vector from all the Pol nucleic acid sequences that encode Pol proteins.

Numerous modifications can be made to the vectors described herein. In various embodiments such modifications may be used to create particles to further minimize the chance of obtaining wild type revertants. These include, but are not limited to, deletions of the U3 region of the LTR, tat deletions and matrix (MA) deletions. In embodiments, the Gag, Pol and Env vector(s) do not contain nucleotides from the lentiviral genome that package lentiviral RNA, referred to as the lentiviral packaging sequence.

Vector plasmids forming the particle preferably do not contain a nucleic acid sequence from the lentiviral genome that expresses an envelope protein. Preferably, a separate vector plasmid that contains a nucleic acid sequence encoding an envelope protein operably linked to a promoter is used. This env vector also does not contain a lentiviral packaging sequence. In one embodiment, the env nucleic acid sequence encodes a lentiviral envelope protein.

In other embodiments, the envelope protein is not from a lentivirus, but from a different virus. The resultant particle may be referred to as a pseudotyped particle. By appropriate selection of envelopes one can “infect” virtually any cell. For example, one can use an env gene that encodes an envelope protein that targets an endocytic compartment. Examples of viruses from which such env genes and envelope proteins can be derived from include the influenza virus (e.g., the Influenza A virus, Influenza B virus, Influenza C virus, Influenza D virus, Isavirus, Quaranjavirus, and Thogotovirus), the Vesiculovirus (e.g., Indiana vesiculovirus), alpha viruses (e.g., the Semliki forest virus, Sindbis virus, Aura virus, Barmah Forest virus, Bebaru virus, Cabassou virus, Getah virus, Highlands J virus, Trocara virus, Una Virus, Ndumu virus, and Middleburg virus, among others), arenaviruses (e.g., the lymphocytic choriomeningitis virus, Machupo virus, Junin virus and Lassa Fever virus), flaviviruses (e.g., the tick-borne encephalitis virus, Dengue virus, hepatitis C virus, GB virus, Apoi virus, Bagaza virus, Edge Hill virus, Jugra virus, Kadam virus, Dakar bat virus, Modoc virus, Powassan virus, Usutu virus, and Sal Vieja virus, among others), rhabdoviruses (e.g., vesicular stomatitis virus, rabies virus), paramyxoviruses (e.g., mumps or measles) and orthomyxoviruses (e.g., influenza virus) and human coronaviruses (SARS, MERS, SARS-COV-2).

Other envelope proteins that can preferably be used include those derived from endogenous retroviruses (e.g., feline endogenous retroviruses and baboon endogenous retroviruses) and closely related gammaretroviruses (e.g., the Moloney Leukemia Virus, MLV-E, MLV-A, Gibbon Ape Leukemia Virus, GALV, Feline leukemia virus, Koala retrovirus, Trager duck spleen necrosis virus, Viper retrovirus, Chick syncytial virus, Gardner-Arnstein feline sarcoma virus, and Porcine type-C oncovirus, among others). These gammaretroviruses can be used as sources of env genes and envelope proteins for targeting primary cells. The gammaretroviruses are particularly preferred where the host cell is a primary cell.

Envelope proteins can be selected to target a specific desired host cell. For example, targeting specific receptors such as a dopamine receptor can be used for brain delivery. Another target can be vascular endothelium. These cells can be targeted using an envelope protein derived from any virus in the Filoviridae family (e.g., Cuevaviruses, Dianloviruses, Ebolaviruses, and Marburgviruses) or human Coronavirus family. Species of Ebolaviruses include Tai Forest ebolavirus, Zaire ebolavirus, Sudan ebolavirus, Bundibugyo ebolavirus, and Reston ebolavirus.

In addition, in embodiments, glycoproteins can undergo post-transcriptional modifications. For example, in an embodiment, the GP of Ebola, can be modified after translation to become the GP1 and GP2 glycoproteins. In another embodiment, one can use different lentiviral capsids with a pseudotyped envelope (e.g., FIV or SHIV [U.S. Pat. No. 5,654,195]). A SHIV pseudotyped vector can readily be used in animal models such as monkeys.

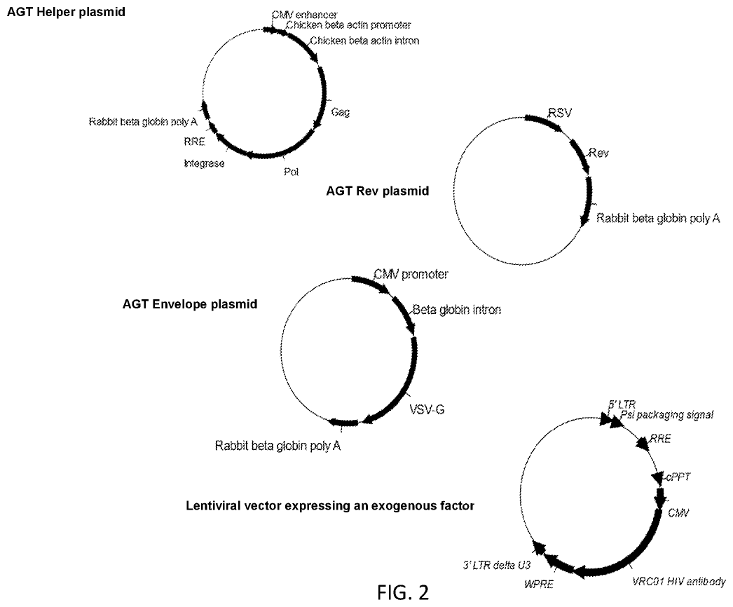

Lentiviral vector systems as provided herein may include at least one helper plasmid comprising at least one of a Gag, Pol, or Rev gene. Each of the Gag, Pol, and Rev genes may be provided on individual plasmids, or one or more genes may be provided together on the same plasmid. In one embodiment, the Gag, Pol, and Rev genes are provided on the same plasmid (e.g., FIG. 1). In another embodiment, the Gag and Pol genes are provided on a first plasmid and the rev gene is provided on a second plasmid (e.g., FIG. 2). Both 3-vector and 4-vector systems may be used to produce a lentivirus as described herein, as well as other suitable vector systems. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle may be produced.

In another aspect, a lentiviral vector system for expressing a lentiviral particle is provided. The system variously includes a lentiviral vector as described herein: an envelope plasmid for expressing an envelope protein optimized for infecting a cell; and at least one helper plasmid for expressing Gag, Pol, and Rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting production of HIV and/or inhibiting HIV from infecting cells.

In another aspect, the lentiviral vector variously includes any of the following elements: hybrid 5′ long terminal repeat (Rous Sarcoma (RSV) promoter (SEQ ID NO: 17)/5′ LTR (SEQ ID NO: 18)), Psi sequence (PSI packaging signal) (SEQ ID NO: 19), RRE (Rev response element (RRE)) (SEQ ID NO: 20), cPPT (Central polypurine tract (cPPT)) (SEQ ID NO: 21), a CMV promoter (SEQ ID NO: 13), Human EF-1α(SEQ ID NO: 14), Interferon gamma (IFNγ) promoter (SEQ ID NO: 15), or the Prothrombin Human Alpha-1 Anti trypsin enhancer/promoter (SEQ ID NO: 16)), Woodchuck Post-Transcriptional Regulatory Element (WPRE) (SEQ ID NO: 22 (Long WPRE sequence) or SEQ ID NO: 23 (Short WPRE sequence)), and 3′ delta LTR (SEQ ID NO: 24). In other aspects, sequence variation, by way of substitution, deletion, addition, or mutation can be used to modify the sequences referenced herein.

In further aspects, a helper plasmid includes any of the following elements: CAG promoter (Helper/Rev; Chicken beta acting (CAG) promoter; Transcription) (SEQ ID NO: 25); HIV component Gag (Helper/Rev; HIV Gag; Viral capsid) (SEQ ID NO: 26); HIV component Pol (Helper/Rev; HIV Pol; Protease and reverse transcriptase) (SEQ ID NO: 27); HIV Int (Helper Rev; HIV Integrase; Integration of viral RNA) (SEQ ID NO: 28); HIV RRE (Helper/Rev; HIV RRE; Binds Rev element) (SEQ ID NO: 29); and HIV Rev (Helper/Rev; HIV Rev; Nuclear export and stabilize viral mRNA) (SEQ ID NO: 30). In further aspects, the helper plasmid may be modified to include a first helper plasmid for expressing the Gag and Pol genes, and a second plasmid for expressing the Rev gene. In further aspects, sequence variation, by way of substitution, deletion, addition, or mutation can be used to modify the sequences referenced herein.

In further aspects, an envelope plasmid includes the following elements: RNA polymerase II promoter (Envelope; CMV promoter) (SEQ ID NO: 31) and vesicular stomatitis virus G glycoprotein (VSV-G) (Envelope; VSV-G; Glycoprotein envelope-cell entry) (SEQ ID NO: 32). In another aspect, sequence variation, by way of substitution, deletion, addition, or mutation can be used to modify the sequences referenced herein.

In further aspects, a helper plasmid includes any of the following elements: CMV enhancer, chicken beta actin promoter, rabbit beta globin intron, HIV component Gag; HIV component Pol; HIV Int; HIV RRE; HIV Rev, and rabbit beta globin poly A.

In aspects, the helper plasmid is modified to include a first helper plasmid for expressing the Gag and Pol genes, and a second and separate plasmid for expressing the rev gene. In further aspects, sequence variation, by way of substitution, deletion, addition, or mutation can be used to modify the sequences referenced herein.

In further aspects, the plasmids used for lentiviral packaging are modified with similar elements: the intron sequences may be removed without loss of vector function. For example, the following elements can replace similar elements in the plasmids that comprise the packaging system: Elongation Factor-1 (EF-1), phosphoglycerate kinase (PGK), and ubiquitin C (UbC) promoters can replace the CMV or CAG promoter. SV40 poly A and bGH poly A can replace the rabbit beta globin poly A. The HIV sequences in the helper plasmid can be constructed from different HIV strains or clades. The VSV-G glycoprotein can be substituted with membrane glycoproteins from human endogenous retroviruses including HERV-W, baboon endogenous retrovirus BaEV, feline endogenous virus (RD114), gibbon ape leukemia virus (GALV), Rabies (FUG), lymphocytic choriomeningitis virus (LCMV), influenza A fowl plague virus (FPV), Ross River alphavirus (RRV), murine leukemia virus 10A1 (MLV), or Ebola virus (EboV).

Of note, lentiviral packaging systems can be acquired commercially (e.g., Lenti-vpak packaging kit from OriGene Technologies, Inc., Rockville, MD), and can also be synthesized using standard techniques. Moreover, it is within the skill of a person skilled in the art to substitute or modify aspects of a lentiviral packaging system to improve any number of relevant factors, including the production efficiency of a lentiviral particle.

### EXAMPLES

### Example 1: Development of a Lentiviral Vector System

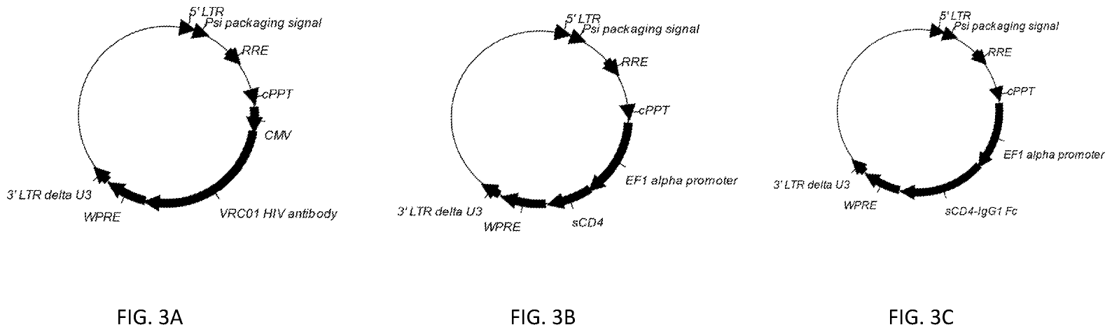

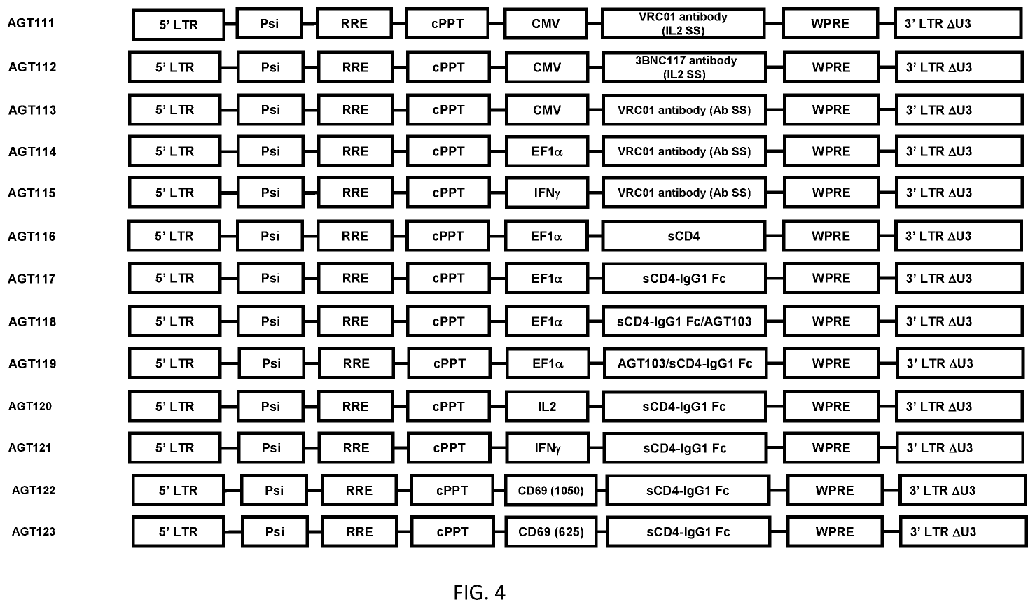

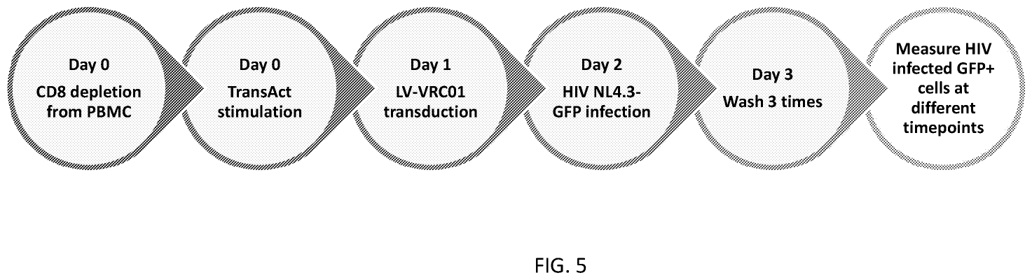

A lentiviral vector system was developed as summarized in FIGS. 1-3 (circularized form) and in FIG. 4 (linear form). Referring first to FIG. 4, representative therapeutic vectors have been designed and produced with the following elements being from left to right: hybrid 5′ long terminal repeat (RSV/5′ LTR) SEQ ID NO: 17 (Rous Sarcoma virus (RSV) promoter) and SEQ ID NO: 18 (5′ Long terminal repeat (LTR)), Psi sequence (RNA packaging site) (SEQ ID NO: 19), RRE (Rev-response element) (SEQ ID NO: 20), cPPT (polypurine tract) (SEQ ID NO: 21), a promoter/promoter enhancer combination (a CMV promoter (SEQ ID NO: 13)), an EF-1α promoter (SEQ ID NO: 14), an IFNγ promoter (SEQ ID NO: 15), a IL-2 promoter (SEQ ID NO: 66), CD69 promoter (SEQ ID NOs: 67 and 68), or a Prothrombin Human Alpha-1 Anti-Trypsin (hAAT) enhancer/promoter (SEQ ID NO: 16)), an exogenous factor (e.g., VRC01 antibody (FIG. 3A), 3BNC117 antibody, sCD4 (FIG. 3B), and sCD4-IgG1 Fc (FIG. 3C)), Woodchuck Post-Transcriptional Regulatory Element (WPRE) (SEQ ID NOS: 22 or 23), and ΔU3 3′ LTR (SEQ ID NO: 24).

A helper plasmid has been designed and produced with the following elements: CAG promoter (SEQ ID NO: 25); HIV component Gag (SEQ ID NO: 26); HIV component Pol (SEQ ID NO: 27); HIV Int (SEQ ID NO: 28); HIV RRE (SEQ ID NO: 29); and HIV Rev (Helper/Rev; HIV Rev; Nuclear export and stabilize viral mRNA) (SEQ ID NO: 30).

An envelope plasmid has been designed and produced with the following elements: RNA polymerase II promoter (Cytomegalovirus (CMV) promoter) (SEQ ID NO: 13) and vesicular stomatitis virus G glycoprotein (VSV-G) (SEQ ID NO: 32).

Lentiviral particles were produced in 293T/17 HEK cells (purchased from American Type Culture Collection, Manassas, VA) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected, and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per cell by quantitative PCR, or by infecting cells and using light (if the vectors encode luciferase or fluorescent protein markers).

A 3-vector system (i.e., a 2-vector lentiviral packaging system) was designed for the production of lentiviral particles. A schematic of the 3-vector system is shown in FIG. 1. Briefly, and with reference to FIG. 1, the top vector depicts a helper plasmid, which, in this case, includes Rev. The middle vector is the envelope plasmid. The bottom vector is the therapeutic vector.

Referring more specifically to the top vector in FIG. 1, the Helper plus Rev plasmid includes a CAG enhancer (Helper/Rev; CMV early (CAG) enhancer; Enhance Transcription) (SEQ ID NO: 33); a CAG promoter (SEQ ID NO: 25); a chicken beta actin intron (Helper/Rev; Chicken beta actin intron; Enhance gene expression) (SEQ ID NO: 34); a HIV Gag (SEQ ID NO: 26); a HIV Pol (SEQ ID NO: 27); a HIV Int (SEQ ID NO: 28); a HIV RRE (SEQ ID NO: 29); a HIV Rev (Helper/Rev; HIV Rev; Nuclear export and stabilize viral mRNA) (SEQ ID NO: 30); and a rabbit beta globin poly A (Helper/Rev: Rabbit beta globin poly A; RNA stability) (SEQ ID NO: 35).

The Envelope plasmid (the middle vector of FIG. 1) includes a CMV promoter (SEQ ID NO: 13); a beta globin intron (Envelope; Beta globin intron; Enhance gene expression) (SEQ ID NO: 36); a VSV-G (SEQ ID NO: 32); and a rabbit beta globin poly A (Envelope; Rabbit beta globin poly A; RNA stability) (SEQ ID NO: 37).

### Synthesis of a 2-Vector Lentiviral Packaging System Including Helper (Plus Rev) and Envelope Plasmids.

### Materials and Methods

Construction of the helper plasmid: The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was SEQ ID NO: 38 and reverse primer was SEQ ID NO: 39. The sequence for the Gag, Pol, Integrase fragment is SEQ ID NO: 40 (Gag, Pol, Integrase fragment).

A DNA fragment containing the Rev, RRE, and rabbit beta globin poly A sequence with XbaI and XmaI flanking restriction sites was synthesized by MWG Operon. The DNA fragment was then inserted into the plasmid at the XbaI and XmaI restriction sites (SEQ ID NO: 41) (DNA Fragment containing Rev, RRE and rabbit beta globin poly A).

The CMV promoter of pCDNA3.1 was replaced with the CAG enhancer/promoter plus a chicken beta actin intron sequence. A DNA fragment containing the CAG enhancer/promoter/intron sequence with MluI and EcoRI flanking restriction sites was synthesized by MWG Operon. The DNA fragment was then inserted into the plasmid at the MluI and EcoRI restriction sites (SEQ ID NO: 42) (DNA fragment containing the CAG enhancer/promoter/intron sequence).

Construction of the VSV-G Envelope plasmid:

The vesicular stomatitis Indiana virus glycoprotein (VSV-G) sequence was synthesized by MWG Operon with flanking EcoRI restriction sites. The DNA fragment was then inserted into the pCDNA3.1 plasmid (Invitrogen) at the EcoRI restriction site and the correct orientation was determined by sequencing using a CMV specific primer (SEQ ID NO: 43) (DNA fragment containing VSV-G).

A 4-vector system (i.e., a 3-vector lentiviral packaging system) has also been designed and produced using the methods and materials described herein. A schematic of the 4-vector system is shown in FIG. 2. Briefly, and with reference to FIG. 2, the top vector of FIG. 2 is a helper plasmid, which, in this case, does not include Rev. The vector second from the top is a separate Rev plasmid. The vector second from the bottom is the envelope plasmid. The bottom vector is the therapeutic vector.

Referring, in part, to the top vector in FIG. 2, the Helper plasmid includes a CAG enhancer (Helper/Rev CMV early (CAG) enhancer: Enhance Transcription) (SEQ ID NO: 33); a CAG promoter (Helper/Rev Chicken beta actin (CAG) promoter) (SEQ ID NO: 25): a chicken beta actin intron (Helper/Rev: Chicken beta actin intron; Enhance gene expression) (SEQ ID NO: 34): a HIV Gag (Helper/Rev; HIV Gag; Viral capsid) (SEQ ID NO: 26); a HIV Pol (Helper/Rev; HIV Pol; Protease and reverse transcriptase) (SEQ ID NO: 27); a HIV Int (Helper Rev; HIV Integrase; Integration of viral RNA) (SEQ ID NO: 28); a HIV RRE (Helper/Rev; HIV RRE; Binds Rev element) (SEQ ID NO: 29); and a rabbit beta globin poly A (Helper/Rev; Rabbit beta globin poly A; RNA stability) (SEQ ID NO: 35).

The Rev plasmid depicted in the vector second from the top in FIG. 2 includes an RSV promoter and a HIV Rev (SEQ ID NO: 45); and a rabbit beta globin poly A (Envelope Rabbit beta globin poly A; RNA stability) (SEQ ID NO: 37).

The Envelope plasmid depicted second from the bottom in FIG. 2 includes a CMV promoter (SEQ ID NO: 13); a beta globin intron (SEQ ID NO: 36); a VSV-G (SEQ ID NO: 32); and a rabbit beta globin poly A (SEQ ID NO: 37).

Synthesis of a 3-Vector Lentiviral Packaging System Including Helper. Rev. and Envelope Plasmids.

### Materials and Methods

Construction of the Helper Plasmid without Rev:

The Helper plasmid without Rev was constructed by inserting a DNA fragment containing the RRE and rabbit beta globin poly A sequence. This sequence was synthesized by MWG Operon with flanking XbaI and XmaI restriction sites. The RRE/rabbit poly A beta globin sequence was then inserted into the Helper plasmid at the XbaI and XmaI restriction sites (SEQ ID NO: 44) (Helper plasmid containing RRE and rabbit beta globin poly A).

### Construction of the Rev Plasmid:

The RSV promoter and HIV Rev sequence was synthesized as a single DNA fragment by MWG Operon with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter (SEQ ID NO: 45) (RSV promoter and HIV Rev).

The plasmids for the 2-vector and 3-vector packaging systems could be modified with similar elements and the intron sequences could potentially be removed without loss of vector function. For example, the following elements could replace similar elements in the 2-vector and 3-vector packaging system:

Promoters: Elongation Factor-1 (Human elongation factor 1 alpha (EF-1α) promoter) (SEQ ID NO: 14), phosphoglycerate kinase (PGK) (Promoter; PGK) (SEQ ID NO: 46), and ubiquitin C (UbC) (Promoter; UbC) (SEQ ID NO: 47) can replace the CMV (SEQ ID NO: 13) or CAG promoter (SEQ ID NO: 48). These sequences can also be further varied by addition, substitution, deletion or mutation.

Poly A sequences: SV40 poly A (Poly A; SV40) (SEQ ID NO: 49) and bGH poly A (Poly A; bGH) (SEQ ID NO: 50) can replace the rabbit beta globin poly A (SEQ ID NO: 35). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (HIV Gag; Bal) (SEQ ID NO: 51); HIV Pol (HIV Pol; Bal) (SEQ ID NO: 52); and HIV Int (HIV Integrase; Bal) (SEQ ID NO: 53) from the Bal strain can be interchanged with the Gag, Pol, and Int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (Envelope; RD114) (SEQ ID NO: 54), gibbon ape leukemia virus (GALV) (Envelope; GALV) (SEQ ID NO: 55), Rabies (FUG) (Envelope FUG) (SEQ ID NO: 56), lymphocytic choriomeningitis virus (LCMV) (Envelope LCMV) (SEQ ID NO: 57), influenza A fowl plague virus (FPV) (Envelope; FPV) (SEQ ID NO: 58), Ross River alphavirus (RRV) (Envelope; RRV) (SEQ ID NO: 59), murine leukemia virus 10A1 (MLV) (Envelope; MLV 10A1) (SEQ ID NO: 60), or Ebola virus (EboV) (Envelope; Ebola) (SEQ ID NO: 61). Sequences for these envelopes are identified in the sequence portion herein. Further, these sequences can also be further varied by addition, substitution, deletion or mutation.

In summary, the 3-vector versus 4-vector systems can be compared and contrasted, in part, as follows. The 3-vector lentiviral vector system contains: 1. Helper plasmid: HIV Gag, Pol, Integrase, and Rev/Tat; 2. Envelope plasmid: VSV-G/FUG envelope; and 3. Therapeutic vector: RSV 5′LTR, Psi Packaging Signal, Gag fragment, RRE, Env fragment, cPPT, WPRE, and 3 delta LTR. The 4-vector lentiviral vector system contains: 1. Helper plasmid: HIV Gag, Pol, and Integrase; 2. Rev plasmid: Rev; 3. Envelope plasmid: VSV-G/FUG envelope; and 4. Therapeutic vector: RSV 5′LTR, Psi Packaging Signal, Gag fragment, RRE, Env fragment, cPPT, WPRE, and 3 delta LTR. Sequences corresponding with the above elements are identified in the sequence listings portion herein.

### Example 2: Construction of Lentiviruses Containing a VRC01 Sequence, a 3BNC117 Sequence, a sCD4 Sequence, or a sCD4-IgG1 Fc Sequence

The heavy (HV) variable and light (LV) variable regions of the anti-HIV neutralizing antibodies were synthesized (Integrated DNA Technologies—IDT) and inserted into a lentivirus plasmid containing the constant regions of the human IgG1 heavy (SEQ ID NO: 70) (IgG1 Heavy Constant Chain) (CH) (Gen Bank: AY623427.1) and light (SEQ ID NO: 73) (IgG1 Light Constant Chain) (CL) (Gen Bank: JQ837832.1) chains. The lentivirus plasmid containing the IgG1 antibody constant regions and the gene fragments of the VRC01 (Gen Bank: GU980702.1) and 3BN117 (Gen Bank: HE584537.1) heavy variable regions were digested with the restriction enzymes XhoI and AgeI (NEB), the plasmid was separated and extracted from a 1% agarose gel (ThermoFisher), and then the plasmid and fragments were ligated with T4 DNA ligase (NEB). The lentivirus plasmid containing the IgG1 antibody constant regions and the gene fragments of the VRC01 (Gen Bank: GU980703.1) and 3BN117 (Gen Bank: HE584538.1) light variable regions were digested with the restriction enzymes BamHI and NotI (NEB). The DNA fragments were separated and extracted from a 1% agarose gel (ThermoFisher), and then the plasmid and fragments were ligated with T4 DNA ligase (NEB). The gene fragments of sCD4 and sCD4-IgG1 Fc were synthesized (IDT) and inserted into a lentivirus plasmid. sCD4 consists of domain 1 and 2 of CD4 (Gen Bank: NM_000616.5) with an antibody secretory sequence and sCD4-IgG1 Fc consists of domain 1 and 2 of CD4 fused to the IgG1 Fc region (Gen Bank: AF237583.1) and an antibody secretory sequence. A lentivirus plasmid and gene fragments of sCD4 and sCD4-IgG1 Fc were digested with BsrGI and NotI (NEB), the plasmid was separated and extracted from a 1% agarose gel, and then the plasmid and fragments were ligated with T4 DNA ligase (NEB). Linear maps of lentiviral vectors containing variations of the promoter and secretory sequence to regulate the expression of anti-HIV neutralizing antibodies and sCD4 are shown in FIG. 4. Table 1 illustrates lentivirus vectors expressing anti-HIV antibodies or sCD4.

TABLE 1

| Vector name | Description of Vector |
|---|---|
| AGT103 (SEQ | Vector encoding inhibitory RNA sequences targeting CCR5 and HIV Vif/Tat |
| ID NO: 78) | with EF-1α promoter (AGT103) |
| AGT111 (SEQ | Vector encoding anti-HIV VRC01 antibody with CMV promoter and IL-2 |
| ID NO: 2) | secretory signal sequence (CMV- VRC01 (IL-2 secretory sequence)-HV-CH- |
| | T2A-LV-CL (AGT111)) |
| AGT112 (SEQ | Vector encoding anti-HIV 3BNC117 antibody with CMV promoter and IL-2 |
| ID NO: 4) | secretory signal sequence (CMV- 3BNC117 (IL-2 secretory sequence)-HV- |
| | CH-T2A-LV-CL (AGT112)) |
| AGT113 (SEQ | Vector encoding anti-HIV VRC01 antibody with CMV promoter and an |
| ID NO: 6) | antibody secretory signal sequence (CMV-VRC01 (Antibody secretory |
| | sequence)-HV-CH-T2A-LV-CL (AGT113)) |
| AGT114 (SEQ | Vector encoding anti-HIV VRC01 antibody with EF-1α promoter and an |
| ID NO: 87) | antibody secretory signal sequence (EF1-VRC01 with Ab signal sequence |
| | (AGT114)) |
| AGT115 (SEQ | Vector encoding anti-HIV VRC01 antibody with IFNγ promoter and an |
| ID NO: 79) | antibody secretory signal sequence (IFNγ promoter, VRC01, antibody |
| | secretion signal sequence (AGT115)) |
| AGT116 (SEQ | Vector encoding soluble CD4 with EF-1α promoter and an antibody |
| ID NO: 8) | secretory signal sequence (EF-1-sCD4(D1 + D2) (AGT116)) |
| AGT117 (SEQ | Vector encoding soluble CD4-IgG1 Fc fusion protein with EF-1α promoter |
| ID NO: 10) | and antibody secretory signal sequence (EF-1α promoter- sCD4(D1 + D2)- |
| | IgG1 Fc fusion protein (truncated SEQ ID NO: 9), antibody secretion |
| | signal(AGT117)) |
| AGT118 (SEQ | Vector encoding soluble CD4-IgG1 Fc fusion protein with EF-1α promoter |
| ID NO: 80) | and antibody secretory signal sequence upstream of inhibitory RNA |
| | sequences targeting CCR5 and HIV Vif/Tat (AGT103) (EF1α promoter, |
| | CD4/IgG1 fusion protein, antibody secretion signal, miR30-CCR5/miR21- |
| | Vif/miR185-Tat microRNA cluster sequence (AGT118)) |
| AGT119 (SEQ | Vector encoding soluble CD4-IgG1 Fc fusion protein with EF-1α promoter |
| ID NO: 81) | and antibody secretory signal sequence downstream of inhibitory RNA |
| | sequences targeting CCR5 and HIV Vif/Tat (AGT103) (EF1α promoter, |
| | miR30-CCR5/miR21-Vif/miR185-Tat microRNA cluster sequence, |
| | CD4/IgGI fusion protein, antibody secretion signal, (AGT119)) |
| AGT120 (SEQ | Vector encoding soluble CD4-IgG1 Fc fusion protein with IL-2 promoter and |
| ID NO: 82) | an antibody secretory signal sequence I(L2 promoter, CD4/IgG1 fusion |
| | protein, antibody secretion signal (AGT120)) |
| AGT121 (SEQ | Vector encoding soluble CD4-IgG1 Fc fusion protein with IFNγ promoter |
| ID NO: 83) | and an antibody secretory signal sequence (IFNγ promoter, CD4/IgG1 fusion |
| | protein, antibody secretion signal (AGT121)) |
| AGT122 (SEQ | Vector encoding soluble CD4-IgG1 Fc fusion protein with CD69 (1050) |
| ID NO: 84) | promoter and an antibody secretory signal sequence (CD69 (1050) promoter, |
| | CD4/IgG1 fusion protein, antibody secretion signal (AGT122)) |
| AGT123 (SEQ | Vector encoding soluble CD4-IgG1 Fc fusion protein with CD69 (625) |
| ID NO: 85) | promoter and an antibody secretory signal sequence (CD69 (625) promoter, |
| | CD4/IgG1 fusion protein, antibody secretion signal (AGT123)) |
| AGT124 (SEQ | Vector encoding Version 2 of soluble CD4-IgG1 Fc fusion protein (SEQ ID |
| ID NO: 88) | NO: 76) (full-length Fc region) with EF-1α promoter and antibody secretory |
| | signal sequence (EF-1a promoter, CD4/IgG1 fusion protein version 2, |
| | antibody secretion signal (AGT124)) |
| AGT125 (SEQ | Vector encoding Version 3 of soluble CD4-IgG1 Fc fusion protein (SEQ ID |
| ID NO: 89) | NO: 77) (full-length Fc region, mutated to remove Fc gamma R II binding |
| | site) with EF-1α promoter and antibody secretory signal sequence (EF-1a |
| | promoter, CD4/IgG1 fusion protein version 3, antibody secretion signal |
| | (AGT125)) |

### Example 3: Impaired Ability of HIV to Infect CD4 T Cells that Express 3BNC117

The HIV antibody 3BNC117 was expressed in CD4 T cells followed by challenging the cells with HIV. The cells were analyzed to determine the frequency of HIV-infected cells.

Method: On day 0), PBMC were depleted of CD8+ T cells and then stimulated with TransAct (CD3/CD28 beads) (MiltenyiBiotec). On day 1, the PBMC were transduced with a lentiviral vectors expressing the broadly neutralizing antibody (bNAb) against HIV (SEQ ID NO:4: AGT112). The components of the vectors are described in Table 2. The AGT112 vector (SEQ ID NO: 4) contains a CMV promoter (SEQ ID NO: 13) that drives expression of a 3BNC117 antibody sequence that contains an IL-2 secretory sequence (SEQ ID NO: 74 (3BNC117 heavy variable chain (with IL-2 secretory sequence)) and SEQ ID NO: 75 (3BNC117 light variable chain (with IL-2 secretory sequence)). The IL-2 secretory sequence is SEQ ID NO: 11.

On day two (2), the PBMC were then infected with HIV NL43-GFP. On day three, cells were washed three times. On day six, HIV-infected GFP positive cells were measured. This protocol is shown in FIG. 5 but here LV-3BNC117 was substituted for LV-VRC01.

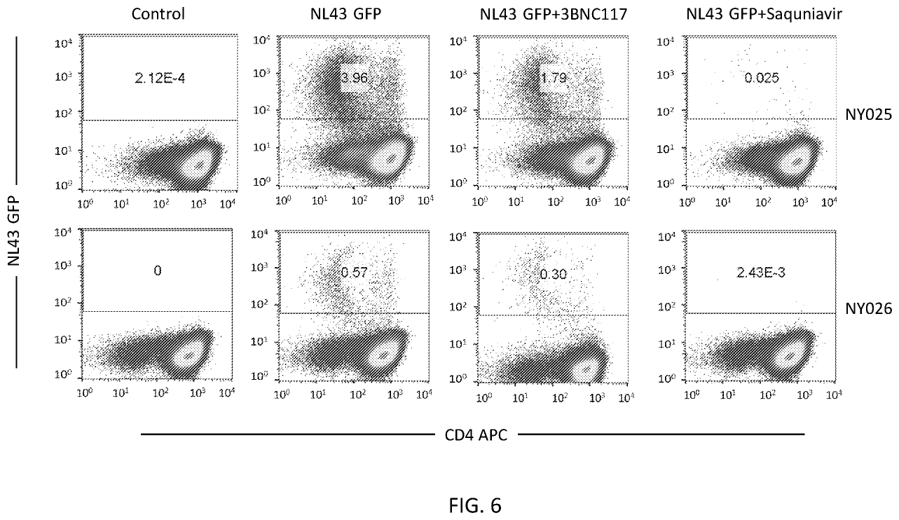

FIG. 6 shows flow cytometry data, which reveals an effect of T cell produced 3BNC117 antibody on HIV infection in vitro. The top and bottom panels of FIG. 6 represent PBMC tested from different donors (NY025 donor and NY026 donor). As shown with both donors, there was a reduction in GFP expression in cells that were transduced with lentiviral vectors encoding 3BNC117 (see column labeled NL43+3BNC117) (1.79% of GFP positive cells with the NY025 donor and 0.30% of GFP positive cells with the NY026 donor) compared to control treated cells (cells not treated with the lentiviral vectors encoding 3BNC117) (see column labeled NL43-GFP) (3.96% of GFP positive cells for the NY025 donor and 0.57% of GFP positive cells for the NY026 donor). The Saquinavir treated cells showed 0.025% and 2.43E-3% GFP positive cells from the NY025 and NY026 donors, respectively.

### Example 4: Impaired Ability of HIV to Infect CD4 T Cells that Express Soluble CD4

Soluble CD4 (sCD4) was expressed in CD4 T cells. The CD4 T cells were then infected with HIV. The cells were analyzed to determine the frequency of HIV infected cells.

Method: On day 0, PBMC were depleted of CD8+ T cell and then stimulated with TransAct (CD3/CD28 beads) (MiltenyiBiotec). On day 1, PBMC were transduced with lentiviral vectors expressing sCD4 (SEQ ID NO: 8 (AGT116) and SEQ ID NO: 10 (AGT117)). The components of the vectors are described in Table 2. The AGT116 vector (SEQ ID NO: 8) contains a sCD4 sequence (SEQ ID NO: 7) and an EF-1α promoter (SEQ ID NO: 14) upstream of the sCD4 sequence. The AGT117 vector (SEQ ID NO: 10) contains a sCD4-IgG1 Fc sequence (SEQ ID NO: 9) and an EF-1α promoter (SEQ ID NO: 14) upstream of the sCD4-IgG1 Fc sequence.

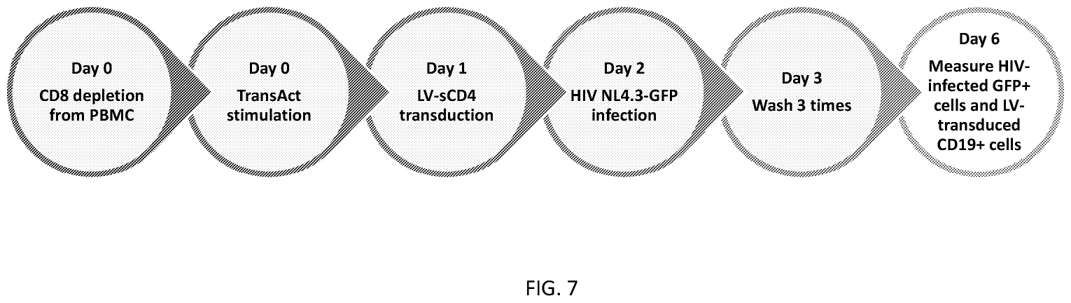

On day 2, the PBMC were infected with HIV NL43-GFP. On day 3, the cells were washed three times. Cells were then cultured for 4 days. On day 6, HIV-infected GFP positive cells were measured. A schematic of this protocol is shown in FIG. 7.

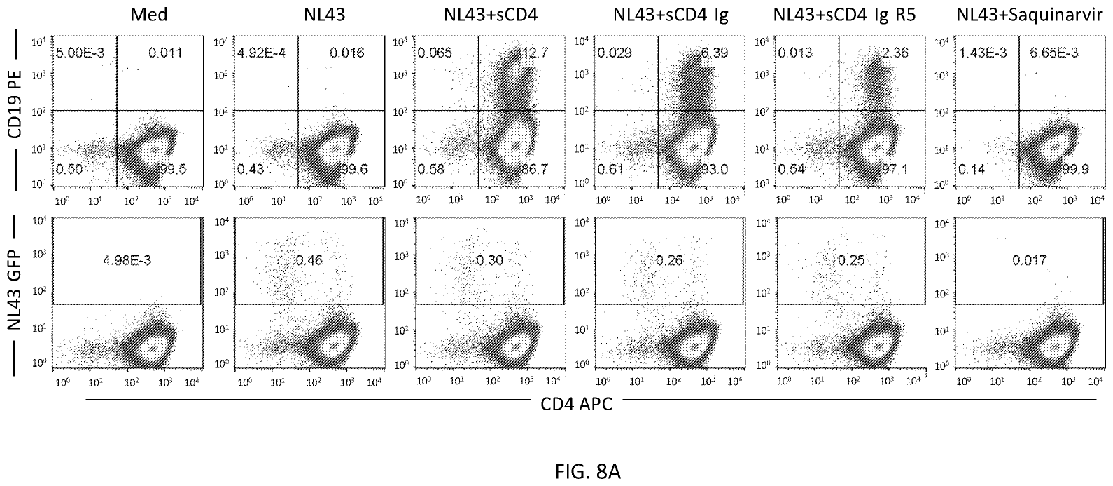

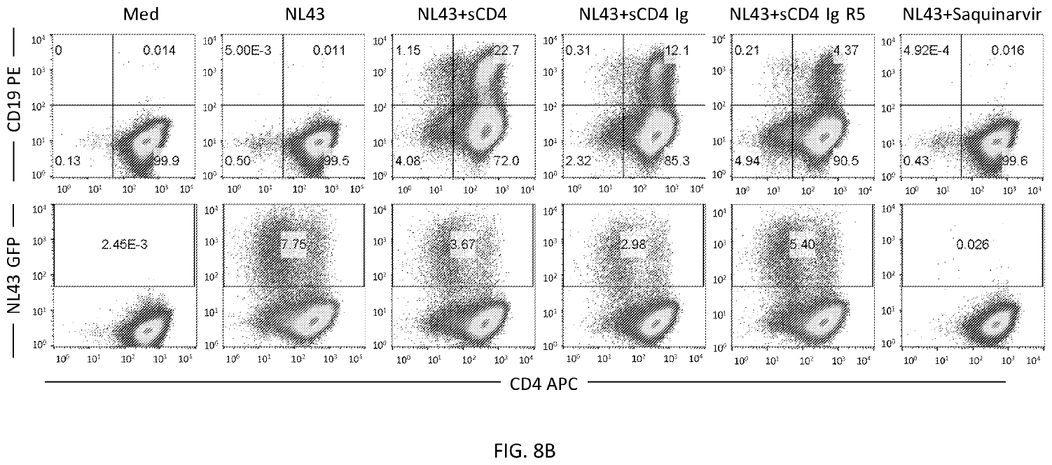

FIGS. 8A and 8B show flow cytometry data, which reveals an effect of T cell produced sCD4 on HIV infection in vitro. As shown in the upper rows of both FIGS. 8A and 8B, the transduction efficiency of sCD4 was low. In FIG. 8A (upper row), the transduction efficiency of AGT116 (SEQ ID NO: 8) (see column labeled NL43-sCD4) was 12.7%, and the transduction efficiency of AGT117 (SEQ ID NO: 10) (see columns labeled NL43-sCD4-Ig and NL43+sCD4-Ig-R5) was 6.39% and 2.36%. In FIG. 8B (upper row), the transduction efficiency of AGT116 (SEQ ID NO: 8) (see column labeled NL43-sCD4) was 22.7% and the transduction efficiency of AGT117 (SEQ ID NO: 10) (see columns labeled NL43-sCD4-Ig and NL43+sCD4-Ig-R5) was 12.1% and 4.37%.

CD4 T cells that expressed sCD4 partially blocked HIV infection (see bottom rows of both FIGS. 8A and 8B). In FIG. 8A (bottom row), in the control treated cells (see column labeled NL43), the percent of GFP positive cells was 0.46%. However, when the cells were treated with AGT116 (SEQ ID NO: 8) (see column labeled NL43+sCD4), the percent of GFP positive cells was 0.30%, and when the cells were treated with (AGT117) (SEQ ID NO: 10) (see columns labeled NL43-sCD4-Ig and NL43+sCD4-Ig-R5), the percent of GFP positive cells was 0.26% and 0.25%. This can be compared to the percent of GFP positive cells in Saquinavir treated cells (see column labeled NL43-Saquinavir) of 0.017%. In FIG. 8B (bottom row), in the control treated cells (see column labeled NL43), the percent of GFP positive cells was 7.75%. However, when the cells were treated with AGT116 (SEQ ID NO: 8) (see column labeled NL43+sCD4), the percent of GFP positive cells was 3.67%, and when the cells were treated with (AGT117) (SEQ ID NO: 10) (see columns labeled NL43-sCD4-Ig and NL43+sCD4-Ig-R5), the percent of GFP positive cells was 2.98% and 5.40%. This can be compared to the percent of GFP positive cells in Saquinavir treated cells (see column labeled NL43-Saquinavir) of 0.026%.

TABLE 2
Description of lentivirus vector components

| Component | Function |
|---|---|
| 5′LTR | Vector packaging and sites for integrase |
| | modification during integration |
| Psi | RNA structure for packaging |
| RRE | Rev-response element promotes transgene |
| | RNA export from nucleus to cytoplasm |
| cPPT | Central polypurine tract is essential |
| | for DNA second strand synthesis during |
| | reverse transcription |
| EF-1α, CMV, | Alternate RNA transcriptional promoters |
| IFNa | to expressing antibodies or sCD4 |
| VRC01, | VRC01 and 3BNC117 are published monoclonal |
| 3BNC117, | antibodies with known HIV neutralizing |
| sCD4, | activity; AGT synthesized the expression |
| sCD4-IgG | constructs from publicly available information |
| | sCD4 and sCD4-IgG are forms of sCD4 that |
| | neutralizes HIV - IgG indicates a fusion |
| | protein that may improve function and |
| | half-life of sCD4 |
| WPRE | Woodchuck hepatitis virus post-transcriptional |
| | regulatory element - improves RNA expression |
| | from the integrated transgene |
| DU3 3′ LTR | Modified 3′ LTR |

### Example 5: Antibody Expression by HIV Gag-Specific CD4 T Cells

The HIV antibodies VRC01 or 3BNC117 were expressed in CD4 T cells. Stimulation of the CD4 T cells with Gag resulted in an increase in antibody expression.

Method: To measure antibody expression by HIV Gag-specific CD4 T cells, HIV positive human peripheral blood mononuclear cells (PBMCs) were separated with Ficoll-Paque PLUS (GE Healthcare, Cat: 17-1440-02). Separated PBMCs (1×10⁷) were stimulated with PepMix™ HIV (GAG) Ultra (Cat: PM-HIV-GAG, JPT Peptide Technologies) in 1 mL medium in a 24-well plate for 18 hours. CD8 T, γδ, NK, and B cells were depleted with PE labeled specific antibodies and anti-PE microbeads. The negatively selected cells were cultured at 2×10⁶/mL in TexMACS GMP medium (Cat: 170-076-309, Miltenyi Biotec) containing IL7 (170-076-111, Miltenyi Biotec), IL15 (170-076-114, Miltenyi Biotec) and saquinavir (Cat: 4658, NIH AIDS Reagent Program). The lentivirus vector AGT111 (SEQ ID NO: 2) encoding anti-HIV antibody VRC01 or AGT112 (SEQ ID NO: 4) encoding anti-HIV antibody 3BNC117 was added 24 hours later at a multiplicity of infection (MOI) of 5.

The AGT111 vector (SEQ ID NO: 2) contains a CMV promoter (SEQ ID NO: 13) that drives expression of a VRC01 antibody sequence that contains an IL-2 secretory sequence (SEQ ID NO: 69 (VRC01 heavy variable chain (with IL-2 secretory signal)) and SEQ ID NO: 72 (VRC01 light variable chain (with IL-2 secretory signal)). The IL-2 secretory sequence is SEQ ID NO: 11.

The AGT112 vector (SEQ ID NO: 4) contains a CMV promoter (SEQ ID NO: 13) that drives expression of a 3BNC117 antibody sequence that contains an IL-2 secretory signal (SEQ ID NO: 74 (3BNC117 heavy variable chain (with IL-2 secretory signal)) and SEQ ID NO: 75 (3BNC117 light variable chain (with IL-2 secretory signal)). The IL-2 secretory signal is SEQ ID NO: 11.

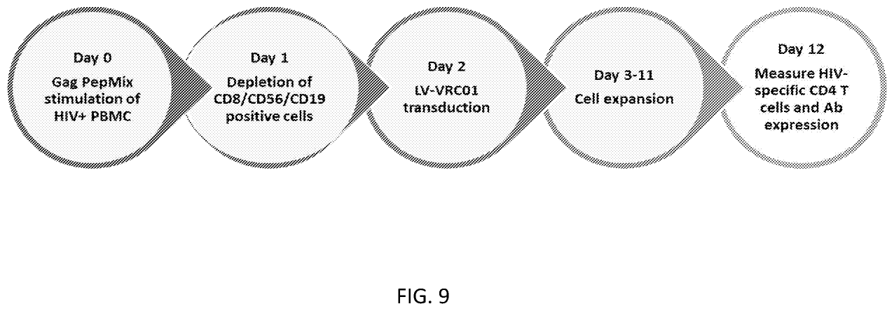

Fresh medium containing IL7, IL15, and saquinavir was added every 2-3 days during cell expansion. The starting concentration of IL7 and IL15 was 10 ng/ml. On day 12-16, 2-3×10⁶ cells were collected for peptide stimulation. The intracellular expression of IFNγ and IgG Fc was detected with a PE anti-IFNγ antibody and an APC anti-IgG1 Fc antibody (Biolegend). A schematic of this protocol is shown in FIG. 9.

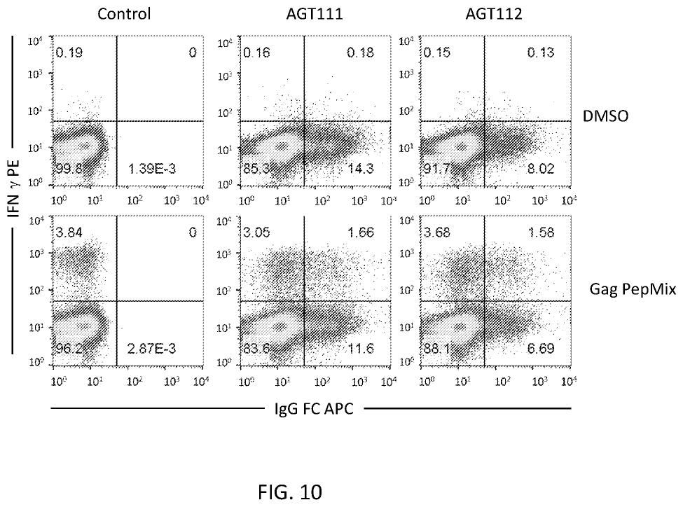

As shown in FIG. 10, antigen-specific CD4 T cells expressed IFNγ and the anti-HIV antibody VRC01 or 3BNC117. This demonstrated inducible antibody expression (induced by peptide) in Gag-specific CD4 T cells (FIG. 10). Also, stimulation with the Gag PepMix (JPT Peptide Technologies) on day 12 (see bottom row labeled Gag PepMix, showing 1.66% of cells transduced with VRC01 and 1.58% of cells transduced with 3BNC117 were positive for IgG Fc) resulted in an approximately 10-fold increase in fluorescence intensity of IgG relative to the cells that were treated with DMSO (see upper row labeled DMSO showing 0.18% of cells transduced with VRC01 and 0.13% of cells transduced with 3BNC117 were positive for IgG Fc) (FIG. 10).

### Example 6: Antibody Expression by CD3/CD28 Bead-Stimulated CD4 T Cells

Mitogen-Stimulated CD4 T cells that were transduced with lentiviral vectors encoding a 3BNC117 HIV antibody resulted intracellular antibody accumulation.

Method: To measure antibody expression in primary CD4 T cells, PBMCs were purified from whole blood and the CD4+ T cell subset was enriched by negative selection using magnetic beads. 1×10⁶ CD4 T cells were cultured in 2 mL of RPMI 1640 medium (Thermo Fisher Scientific) containing 10% FBS (Gemini Bio) and 1% Pen-Strep (Thermo Fisher Scientific) in a 37° C. incubator at 5% CO₂ and supplemented with recombinant human IL-2 (30) U/mL) (Thermo Fisher Scientific) and TransAct (CD3/CD28 microbeads) (Miltenyi Biotec). Cells were cultured for 1 day before adding lentivirus vector AGT111 (SEQ ID NO: 2) encoding anti-HIV antibody VRC01 or AGT112 (SEQ ID NO: 4) encoding anti-HIV antibody 3BNC117, at a MOI of 5.

The AGT111 vector (SEQ ID NO: 2) contains a CMV promoter (SEQ ID NO: 13) that drives expression of a VRC01 antibody sequence that contains an IL-2 secretory signal (SEQ ID NO: 69 (VRC01 heavy variable chain (with IL-2 secretory signal)) and SEQ ID NO: 72 (VRC01 light variable chain (with IL-2 secretory signal)). The IL-2 secretory signal is SEQ ID NO: 11.

The AGT112 vector (SEQ ID NO: 4) contains a CMV promoter (SEQ ID NO: 13) that drives expression of a 3BNC117 antibody sequence that contains an IL-2 secretory signal (SEQ ID NO: 74 (3BNC117 heavy variable chain (with IL-2 secretory signal)) and SEQ ID NO: 75 (3BNC117 light variable chain (with IL-2 secretory sequence)). The IL-2 secretory signal is SEQ ID NO: 11.

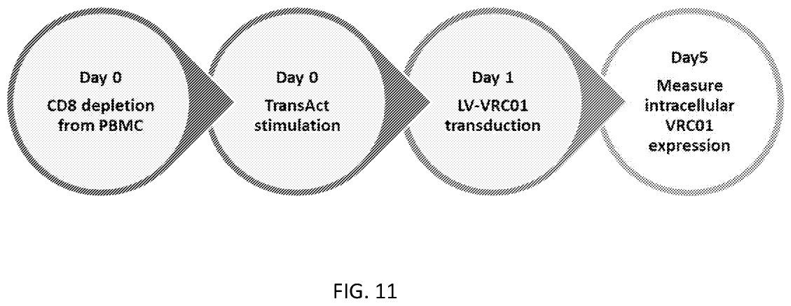

One day after transduction, the medium was removed and replaced with fresh medium plus IL-2. Cells were cultured for an additional 3 days, CD4 T cells were washed and collected to measure the efficiency of transduction. Transduced cells were identified by cell surface staining of CD3 and CD4 glycoproteins that are characteristic of helper T cells and tested to measure intracellular IgG Fc for VRC01 expression. A schematic of this protocol is shown in FIG. 11.

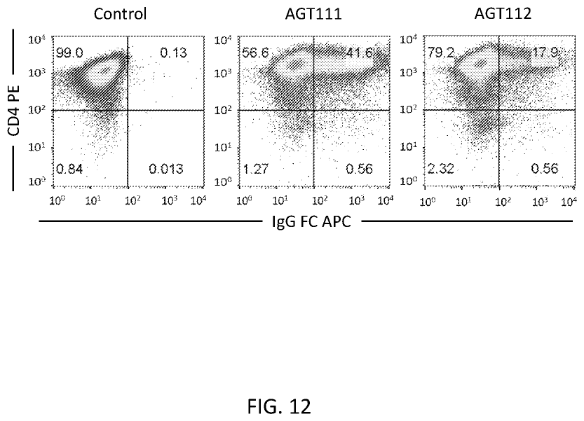

As shown in FIG. 12, CD4 T cells were stained with a PE-labeled anti-CD4 antibody and transduced cells were identified with an APC-labeled antibody against human IgG Fc (Biolegend). In AGT111 (SEQ ID NO: 2) transduced cells (see column labeled AGT111), 41.6% of total cells in the culture were positive for both CD4 and IgG Fc. In AGT112 (SEQ ID NO: 4) 17.9% of total cells in the culture were positive for both CD4 and IgG Fc. In the control treated cells (see column labeled Control) only 0.13% of the cells were positive for both CD4 and IgG. These data demonstrated that antibodies can be expressed by stimulated primary CD4 T cells.

### Example 7: Anti-HIV Antibody Production Protects CD4 T Cells from HIV Infection

Expression of the anti-HIV antibody VRC01 protects CD4 T cells from HIV infection.

Method: CD4 T cells were separated by negative selection and stimulated for 1 day with TransAct (CD3/CD28 beads) (Miltenyi Biotec) plus IL-2 (30 U/mL) (Thermo Fisher Scientific) and then transduced with lentivirus vector AGT111 (SEQ ID NO: 2) at various MOI.

The AGT111 vector (SEQ ID NO: 2) contains a CMV promoter (SEQ ID NO: 13) that drives expression of a VRC01 antibody sequence that contains an IL-2 secretory signal (SEQ ID NO: 69 (VRC01 heavy variable chain (with IL-2 secretory signal)) and SEQ ID NO: 72 (VRC01 light variable chain (with IL-2 secretory signal)). The IL-2 secretory signal is SEQ ID NO: 11.

One day later, cells were infected with 1 MOI of HIV recombinant strain NL43 that expresses GFP. After 24 hours, CD3/CD28 beads, lentivirus and HIV were removed by washing 3 times with PBS and cultured in RPMI 1640 medium (Thermo Fisher Scientific) containing 10% FBS (Gemini Bio) and 1% Pen-Strep (Thermo Fisher Scientific) with IL-2 (30 U/mL) in a 37° C. incubator at 5% CO₂ for 7 days with medium supplementation as needed. As a control, HIV infected cells were treated with 200 nM saquinavir. At the end of the culture, cells were collected and analyzed by flow cytometry for GFP expression and with an APC anti-CD4 antibody. If the CD4 cell expresses GFP, it was infected by HIV.

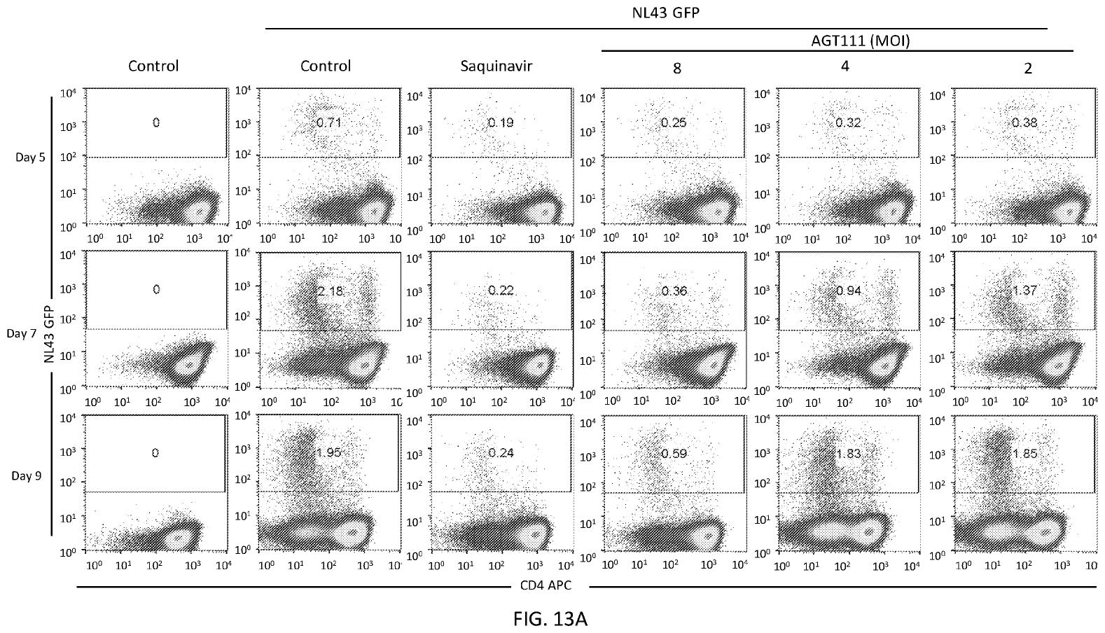

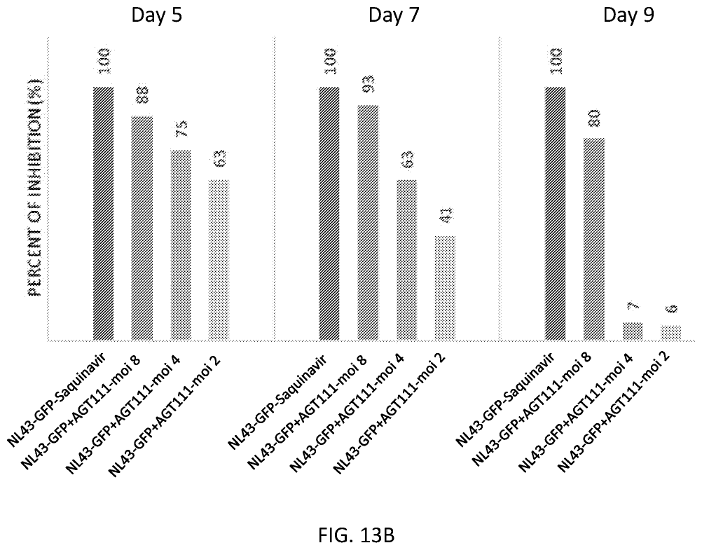

As shown in FIG. 13A, higher proportions of GFP+ cells indicate that more HIV was produced in the primary CD4 T cells. Higher levels of HIV produced in the primary T cell culture are associated with less protection afforded by AGT111. In the cells treated with NL43-GFP but without any vector, the percentage of GFP positive cells was 0.71%, 2.18%, and 1.95% at days 5, 7, and 9, respectively. In the AGT111 (SEQ ID NO: 2) treated cells, at 8 MOI, the percentage of GFP positive cells was 0.25%, 0.36%, and 0.59% at days, 5, 7, and 9, respectively. In the AGT111 (SEQ ID NO: 2) treated cells, at 4 MOI, the percentage of GFP positive cells was 0.32%, 0.94%, and 1.83% at days, 5, 7, and 9, respectively. In the AGT111 (SEQ ID NO: 2) treated cells, at 2 MOI, the percentage of GFP positive cells was 0.38%, 1.37%, and 1.85% at days, 5, 7, and 9, respectively. This can be compared to cells treated with Saquinavir in which the percentage of GFP positive cells was 0.19%, 0.22%, and 0.24% at days 5, 7, and 9, respectively.

FIG. 13B shows the percent inhibition of cells treated with AGT 111 (SEQ ID NO:2) on different days and at different MOI. On day 5, at an MOI of 8, 4, and 2, AGT111 protected 88%, 75%, and 63% of the cells, respectively. On day 7, at an MOI of 8, 4, and 2, AGT111 protected 93%, 63%, and 41% of the cells, respectively. On day 9, at an MOI of 8, 4, and 2, AGT111 protected 80%, 7%, and 6% of the cells, respectively.

### Example 8: Antibody Expression by a Highly HIV Permissive T Cell Leukemia Cell Line

C8166 is a T cell leukemia cell line that is highly permissive for HIV infection. Transduction of the C8166 T cell leukemia cell line with a lentivirus encoding an HIV antibody results in production of the HIV antibody.

Method: C8166 cells were cultured in RPMI 1640 medium (Thermo Fisher Scientific) containing 10% FBS and then transduced with lentivirus vector AGT111 (SEQ ID NO: 2) at a MOI of 5. The AGT111 vector (SEQ ID NO: 2) contains a CMV promoter (SEQ ID NO: 13) that drives expression of a VRC01 antibody sequence that contains an IL-2 secretory signal (SEQ ID NO: 69 (VRC01 heavy variable chain (with IL-2 secretory signal)) and SEQ ID NO: 72 (VRC01 light variable chain (with IL-2 secretory signal)). The IL-2 secretory sequence is SEQ ID NO: 11.

After 72 hours, cells were collected in 12×75 mm FACs tubes and centrifuged at 1000 rpm for 3 minutes. The cells were washed with PBS and centrifuged at 1000 rpm for 3 minutes. 0.2 mL of fixation solution from the BD Fixation/Permeabilization kit was added to the tube and the cells were kept at 4° C. for 15 minutes. The cells were washed 2 times with BD Perm/Wash buffer and 0.1 mL was added to each tube with 2.5 μL of PE anti-human IgG1 Fc antibody (Biolegend). The tubes were kept at 4° C. for 20 minutes and then washed 2 times with PBS. The cells were resuspended in 0.7 mL of PBS and detected on a FACS Calibur flow cytometer.

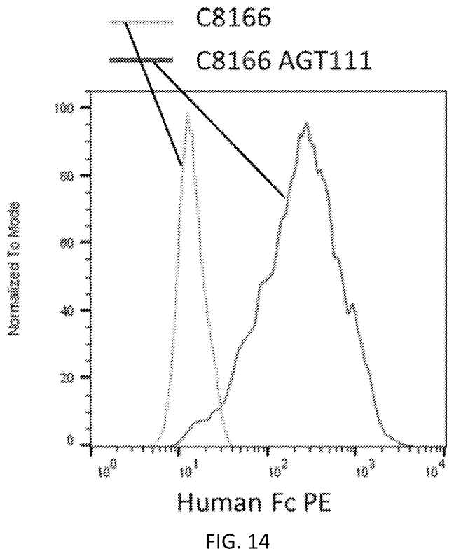

As shown in FIG. 14, antibody expression was detected in AGT111-transduced C8166 cells.

### Example 9: Anti-HIV Antibody Production Protects a Highly Permissive Cell from HIV Infection

Transduction of a lentivirus encoding the VRC01 antibody in the C8166 T cell line resulted in inhibition of HIV NL43 Infection.

Method: C8166 cells were cultured in RPMI 1640 medium (Thermo Fisher Scientific) containing 10% FBS (Gemini Bio) and then transduced without or with lentivirus vector AGT111 (SEQ ID NO: 2) at a MOI of 5 on day 0. The AGT111 vector (SEQ ID NO: 2) contains a CMV promoter (SEQ ID NO: 13) that drives expression of a VRC01 antibody sequence that contains an IL-2 secretory signal (SEQ ID NO: 69 (VRC01 heavy variable chain (with IL-2 secretory signal)) and SEQ ID NO: 72 (VRC01 light variable chain (with IL-2 secretory signal)). The IL-2 secretory signal is SEQ ID NO: 11.

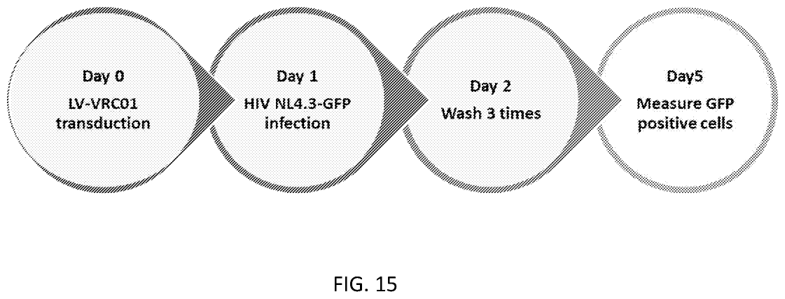

On day 3, cells were infected with 1 MOI of HIV recombinant strain NL43 that expresses GFP. On day 7, cells were collected to measure GFP positive HIV infected cells by flow cytometry. If the C8166 cell expresses GFP, it was infected by HIV. Higher proportions of GFP+C8166 cells indicate that more HIV was produced. A schematic of this protocol is shown in FIG. 15.

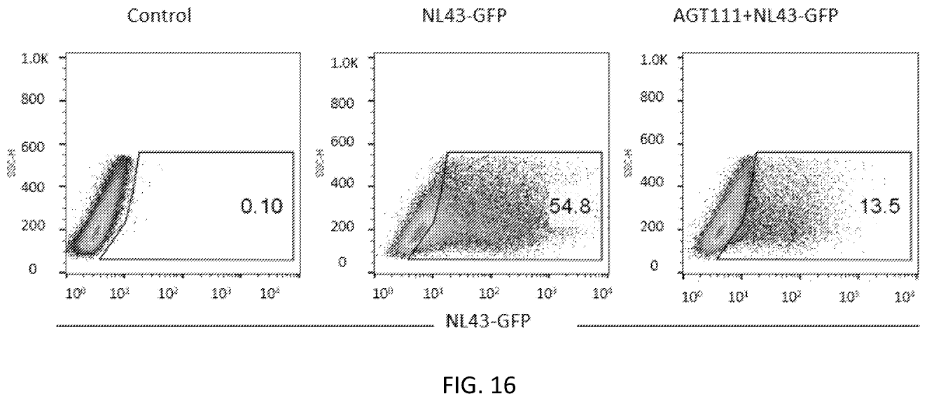

As shown in FIG. 16, AGT111 protected C8166 cells against HIV infection. In AGT111 (SEQ ID NO: 2) transduced cells (see column labeled AGT111+NL43-GFP), 13.5% of C8166 cells were HIV positive. In cells that were not transduced with a lentivirus vector (see column labeled NL43-GFP), 54.8% of the C8166 cells were HIV positive. Therefore, there was a 75.4% decrease in HIV infection with AGT111.

### Example 10: Antibody Secretion from a Highly HIV Permissive T Cell Leukemia Cell Line

Transduction of the C8166 cell line with a lentivirus encoding the VRC01 antibody results in antibody secretion of the antibody.

Method: C8166 cells at 2×10⁵ cells/mL were seeded in a 24 well plate in RPMI 1640 medium (Thermo Fisher Scientific) containing 10% FBS (Gemini Bio) and transduced with or without lentivirus AGT113 (SEQ ID NO: 6) encoding the anti-HIV VRC01 antibody at a MOI of 5. The AGT113 vector (SEQ ID NO: 6) contains a CMV promoter (SEQ ID NO: 13) that drives expression of a VRC01 antibody sequence that contains an antibody secretory signal (SEQ ID NO: 86 (VRC01 heavy variable chain (with antibody secretory signal)) and SEQ ID NO: 71 (VRC01 light variable chain (with antibody secretory signal)). The antibody secretory signal is SEQ ID NO: 12.

On day 4, the cells were centrifuged at 2000 rpm for 5 minutes and the medium was removed. Antibody expression was determined with the Easy Titer IgG Fc antibody detection kit following the manufacturer's instructions (Thermo Fisher Scientific).

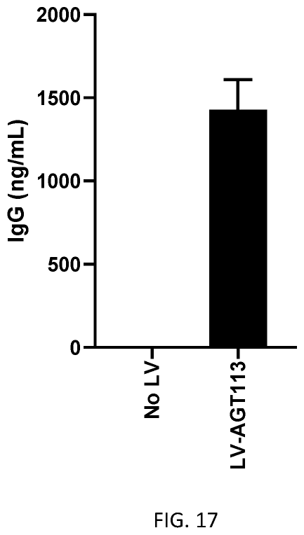

As shown in FIG. 17, transduction of C8166 cells with a lentivirus encoding VRC01 (SEQ ID NO: 6) (see bar labeled LV-AGT113 in FIG. 17) resulted in a concentration of antibody in the cell culture media of approximately 1,500 ng/ml. This is compared to a negligible amount of antibody in the culture media that contained the control treated cells (see bar labeled No LV).

### Example 11: Anti-HIV Antibody Production Protected a Highly Permissive Cell Line from HIV Infection

Transduction of the C8166 cell line with a lentivirus encoding the VRC01 antibody resulted in protection of the cell line from HIV infection.

Method: C8166 cells were cultured in RPMI 1640 medium (Thermo Fisher Scientific) containing 10% FBS (Gemini Bio) and then transduced without or with lentivirus AGT113 (SEQ ID NO: 6) at a MOI of 5 on day 0. The AGT113 vector (SEQ ID NO: 6) contains a CMV promoter (SEQ ID NO: 13) that drives expression of a VRC01 antibody sequence that contains an antibody secretory signal (SEQ ID NO: 86 (VRC01 heavy variable chain (with antibody secretory signal) SEQ ID NO: 71 (VRC01 light variable chain (with antibody secretory signal)). The IL-2 antibody secretory signal is SEQ ID NO: 12.

On day 3, cells were infected with 1 MOI of HIV recombinant strain NL43 that itself expresses GFP. On day 7, cells were collected to measure GFP positive HIV infected cells by flow cytometry. If the C8166 cell expresses GFP, it was infected by HIV. Higher proportions of GFP+C8166 cells indicate that more HIV was produced.

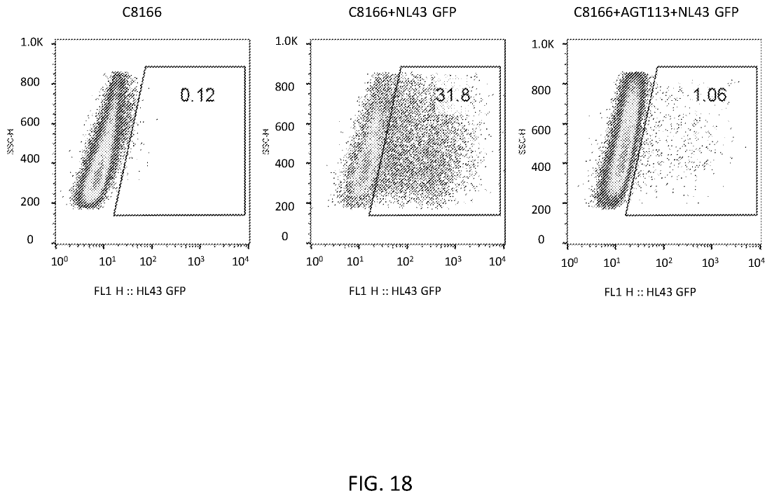

As shown in FIG. 18, AGT113 (SEQ ID NO: 6) protected C8166 cells against HIV infection. In AGT113 (SEQ ID NO: 6) transduced cells (see column labeled C8166+AGT113+NL43-GFP), 1.06% of C8166 cells were HIV positive. In cells not transduced with a lentivirus (see column labeled C8166+NL43-GFP), 31.8% were HIV. Therefore, there was a 96.7% decrease in HIV infection with AGT113.

### Example 12: Soluble CD4 Production Protected a Highly Permissive Cell Line from HIV Infection

Transduction of C8166 T cells with a lentivirus encoding soluble CD4 resulted in protection from HIV infection.

Method: C8166 cells were transduced without or with the lentivirus AGT117 (SEQ ID NO: 10) encoding sCD4-IgG1 Fc at MOI 5. The AGT117 vector (SEQ ID NO: 10) contains an EF-1α promoter (SEQ ID NO: 14) that drives expression of a sCD4-IgG1 Fc (SEQ ID NO: 9) (sCD4(D1+D2)-IgG1 Fc). On day 5, cells were infected with HIV NL43 carrying GFP. On day 7, cells were collected to measure GFP positive HIV infected cells.

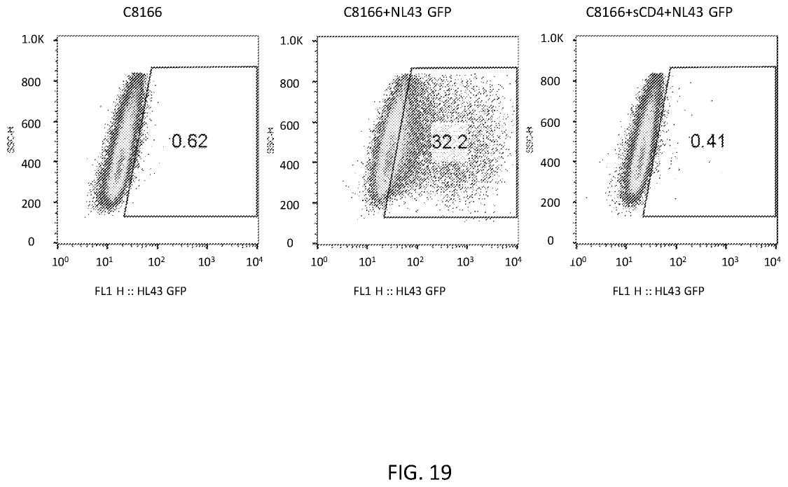

As shown in FIG. 19, GFP fluorescence intensity was significantly reduced in cells transduced with a lentivirus encoding sCD4 and treated with NL43 GFP (AGT117 (SEQ ID NO: 10)) (see column labeled C8166+sCD4+NL43-GFP that shows 0.41% GFP positive cells) compared to GFP fluorescence intensity in cells only treated with NL43-GFP (see column labeled C8166+NL43-GFP that shows 32.2% GFP positive cells). This shows that sCD4 production protected a highly permissive cell line from HIV infection.

### Example 13: Antibody Expression within CD3/CD28 Bead-Stimulated CD4 T Cells

Mitogen-Stimulated CD4 T cells that were transduced with lentiviral vectors encoding a VRC01 HIV antibody resulted intracellular antibody accumulation.

Method: To measure antibody expression in primary CD4 T cells, peripheral blood mononuclear cells (PBMC) were purified from whole blood and the CD4+ T cell subset was enriched by negative selection using magnetic beads. 1×10⁶ CD4 T cells were cultured in 2 mL of RPMI 1640 medium (Thermo Fisher Scientific) containing 10% FBS (Gemini Bio) and 1% Pen-Strep (Thermo Fisher Scientific) in a 37° C. incubator at 5% C02 and supplemented with recombinant human IL-2 (30 U/ml) (Thermo Fisher Scientific) and TransAct (CD3/CD28 microbeads) (Miltenyi Bio). Cells were cultured for 1 day before adding lentivirus AGT113 (SEQ ID NO: 6) at a MOI of 5.

The AGT113 vector (SEQ ID NO: 6) contains a CMV promoter (SEQ ID NO: 13) that drives expression of a VRC01 antibody sequence that contains an antibody secretory signal (SEQ ID NO: 86 (VRC01 heavy variable chain (with antibody secretory signal) and SEQ ID NO: 71 (VRC01 light variable chain (with antibody secretory signal)). The antibody secretory signal is SEQ ID NO: 12.

1 day after transduction the medium was removed and replaced with fresh medium plus IL-2. Cells were cultured for an additional 3 days, CD4 T cells were washed and collected to measure the efficiency of transduction. Transduced cells were tested to measure intracellular IgG Fc for VRC01 expression with a PE anti-human IgG Fc antibody (Biolegend).

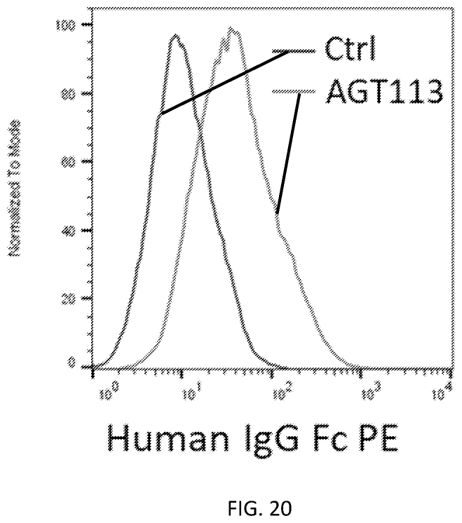

As shown in FIG. 20, IFNγ positive, antigen-specific CD4 T cells expressed the VRC01 antibody.

### Example 14: Antibody Expression within HIV Gag-Specific CD4 T Cells

To measure antibody expression in HIV Gag-specific CD4 T cells, HIV positive human PBMCs were separated with Ficoll-Paque PLUS (GE Healthcare, Cat: 17-1440-02).

Method: PBMCs (1×10⁷) were stimulated with PepMix™ HIV (GAG) Ultra (Cat: PM-HIV-GAG, JPT Peptide Technologies) in 1 mL medium in a 24-well plate for 18 hours. CD8 T γδ, NK and B cells were depleted with PE labeled specific antibodies and anti-PE microbeads. The negatively selected cells were cultured at 2×10⁶/mL in TexMACS GMP medium (Cat: 170-076-309, Miltenyi Biotec) containing IL7 (170-076-111, Miltenyi Biotec), IL15 (170-076-114, Miltenyi Biotec) and saquinavir (Cat: 4658, NIH AIDS Reagent Program). Lentivirus AGT113 (SEQ ID NO: 6) was added 24 hours later at a MOI of 5. The AGT113 vector (SEQ ID NO: 6) contains a CMV promoter (SEQ ID NO: 13) that drives expression of a VRC01 antibody sequence that contains an antibody secretory signal (SEQ ID NO: 86 (VRC01 heavy variable chain (with antibody secretory signal) and SEQ ID NO: 71 (VRC01 light variable chain (with antibody secretory signal)). The antibody secretory signal is SEQ ID NO: 12.

Fresh medium containing IL7, IL15, and saquinavir was added every 2-3 days during cell expansion. The starting concentration of IL7 and IL15 was 10 ng/ml. On day 12-16, 2-3×10⁶ cells were collected for peptide stimulation. The intracellular expression of IFNγ and VRC01 antibody was detected with a PE anti-IFNγ antibody and an APC anti-IgG1 Fc antibody (Biolegend).

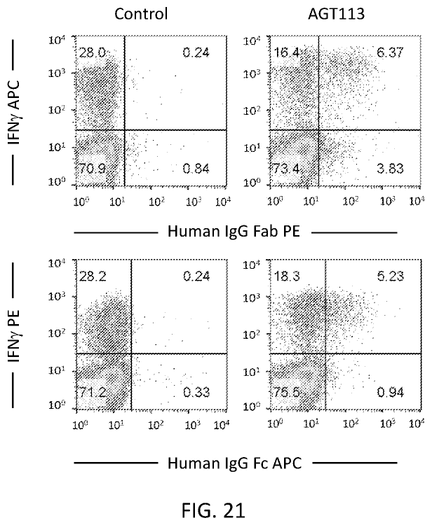

As shown in FIG. 21, IFNγ positive, antigen-specific CD4 T cells expressed the VRC01 antibody contained (see column labeled AGT113—the upper right quadrants of both the top row (6.37% positive cells) and the bottom row (5.23% positive cells), relative to the control treated cells (see column labeled control—the upper right quadrants of both the top row (0.24% positive cells) and the bottom row (0.24% positive cells).

### Example 15: Anti-HIV Antibody Production Protects CD4 T Cells Against HIV Infection

Transduction of a lentivirus encoding the VRC01 antibody in primary CD4 T cells inhibits HIV NL43-GFP infection.

Method: CD4 T cells were separated by negative selection and stimulated for 1 day with TransAct (CD3/CD28 beads) (Miltenyi Biotec) plus IL-2 (30 U/mL) (Thermo Fisher Scientific) and then transduced with lentivirus AGT113 (SEQ ID NO: 6) at a MOI of 5. The AGT113 vector (SEQ ID NO: 6) contains a CMV promoter (SEQ ID NO: 13) that drives expression of a VRC01 antibody sequence that contains an antibody secretory signal (SEQ ID NO: 86 (VRC01 heavy variable chain (with antibody secretory signal) and SEQ ID NO: 71 (VRC01 light variable chain (with antibody secretory signal)). The antibody secretory signal is SEQ ID NO: 12.

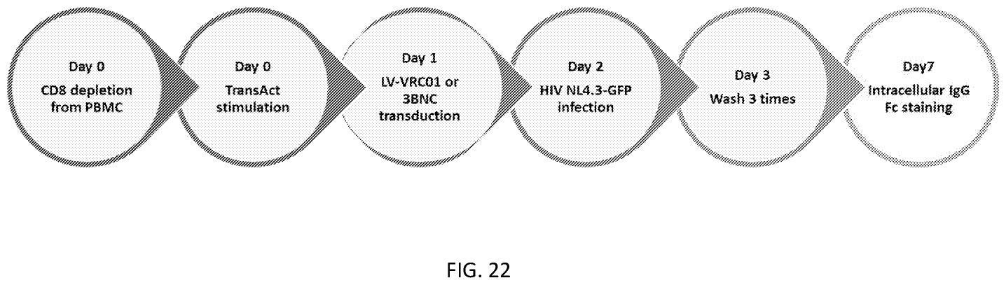

One day later, cells were infected with 1 MOI of HIV recombinant strain NL43 that expresses GFP. After 24 hours, CD3/CD28 beads, lentivirus and HIV were removed by washing 3 times with PBS and cultured in RPMI 1640 medium (Thermo Fisher Scientific) containing 10% FBS (Gemini Bio) and 1% Pen-Strep (Thermo Fisher Scientific) with IL-2 (30 U/mL) in a 37° C. incubator at 5% C02 for 7 days with medium supplementation as needed. At the end of the culture, cells were collected and analyzed by flow cytometry. If the CD4 cell expresses GFP, it was infected by HIV. A schematic of this protocol is shown in FIG. 22.

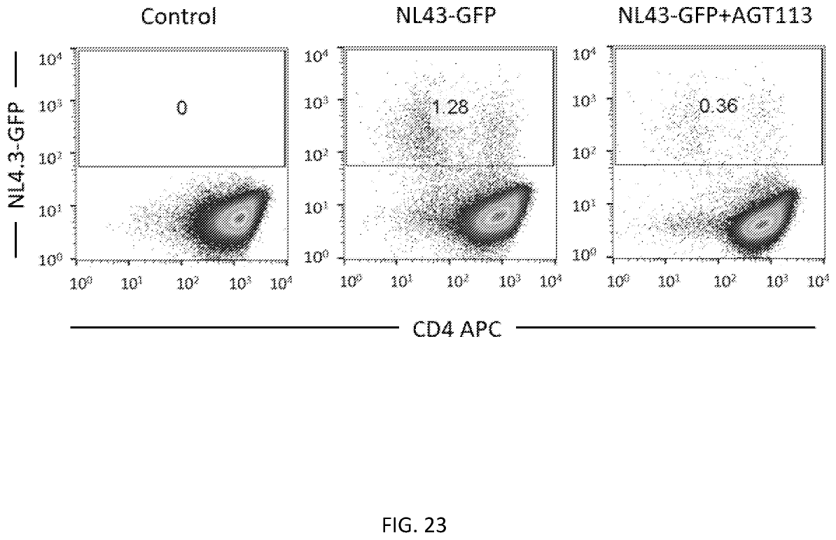

As shown in FIG. 23, AGT113 protected primary CD4 T cells from HIV infection. In AGT113 transduced cells (SEQ ID NO: 6) (see column labeled NL43-GFP-AGT113), 0.36% of cells were HIV positive. In un-transduced cells (see column labeled NL43-GFP), 1.28% of cells were HIV positive.

### Example 16: Production of Soluble CD4-IgG Fc Fusion Protein Protects CD4 T Cells Against HIV Infection

Soluble CD4-IgG Fc fusion protein protects CD4 T cells against HIV infection.

Method (I): CD4 T cells were separated by negative selection and stimulated for 1 day with TransAct (CD3/CD28 beads) (Miltenyi Biotec) plus IL-2 (30 U/mL) (Thermo Fisher Scientific) and then transduced with lentivirus AGT116 (SEQ ID NO: 8) or AGT117 (SEQ ID NO: 10) at a MOI of 5. The AGT116 vector (SEQ ID NO: 8) contains an EF-1α promoter (SEQ ID NO: 14) that drives expression sCD4 (SEQ ID NO: 7) (sCD4(D1+D2). The AGT117 vector (SEQ ID NO: 10) contains an EF-1α promoter (SEQ ID NO: 14) that drives expression of sCD4-IgG1 FC (SEQ ID NO: 9) (sCD4(D1+D2)-IgG1 Fc).

One day later, cells were infected with 1 MOI of HIV recombinant strain NL43 that expresses GFP. After 24 hours, CD3/CD28 beads, lentivirus and HIV were removed by washing 3 times with PBS and cultured in RPMI 1640 medium (Thermo Fisher Scientific) containing 10% FBS (Gemini Bio) and 1% Pen-Strep (Thermo Fisher Scientific) with IL-2 (30 U/mL) in a 37° C. incubator at 5% C02 for 7 days with medium supplementation as needed. At the end of the culture, cells were collected and analyzed by flow cytometry. If the CD4 T cell expresses GFP, it had been infected by the recombinant HIV.

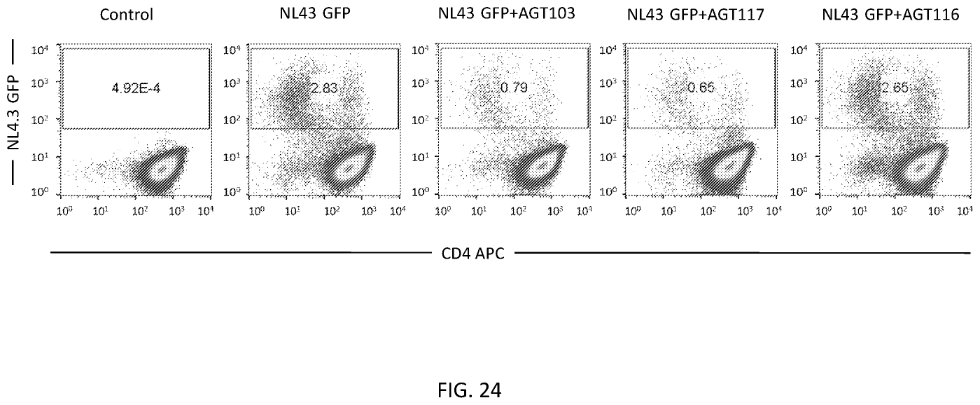

As shown in FIG. 24, AGT117 (SEQ ID NO: 10), which encodes soluble CD4-IgG Fc fusion protein, protected primary CD4 T cells from HIV infection. Specifically, in cells treated with AGT117 (see column labeled NL43-GFP+AGT117), 0.65% of cells were HIV positive. This can be compared to control treated cells (see column labeled NL43-GFP) in which 2.83% were HIV positive. Thus, there was a 77% decrease in HIV infection with AGT117. However, the lentivirus AGT116 (SEQ ID NO: 8), which encodes soluble CD4 demonstrated a low level of HIV inhibition (see column labeled NL43-GFP+AGT116 showing that 2.65% of cells were HIV positive).

Method (II): CD4 T cells were separated by negative selection and stimulated for 1 day with TransAct (CD3/CD28 beads) (Miltenyi Biotec) plus IL-2 (30 U/mL) (Thermo Fisher Scientific) and then transduced with lentivirus AGT117 (SEQ ID NO:10) or AGT124 (SEQ ID NO: 88) or AGT125 (SEQ ID NO:89) at a MOI of 5. The AGT117 vector (SEQ ID NO: 10) contains an EF-1α promoter (SEQ ID NO: 14) that drives expression CD4-IgG where the Fc region is truncated (SEQ ID NO: 7). The AGT124 vector (SEQ ID NO: 88) contains an EF-1α promoter (SEQ ID NO: 14) that drives expression of sCD4-IgG1 where the Fc region is intact and uses the wild-type sequence (SEQ ID NO: 76). The AGT125 vector (SEQ ID NO: 89) contains an EF-1α promoter (SEQ ID NO: 14) that drives expression of sCD4-IgG1 where the Fc region was mutated to remove the binding site for Fc gamma Receptor II (SEQ ID NO: 77).

One day later, cells were infected with 1 MOI of HIV recombinant strain NL43 that expresses GFP. After 24 hours, CD3/CD28 beads, lentivirus and HIV were removed by washing 3 times with PBS and cultured in RPMI 1640 medium (Thermo Fisher Scientific) containing 10% FBS (Gemini Bio) and 1% Pen-Strep (Thermo Fisher Scientific) with IL-2 (30) U/mL) in a 37° C. incubator at 5% C02 for 7 days with medium supplementation as needed. At the end of the culture, cells were collected and analyzed by flow cytometry. If the CD4 T cell expresses GFP, it had been infected by the recombinant HIV.

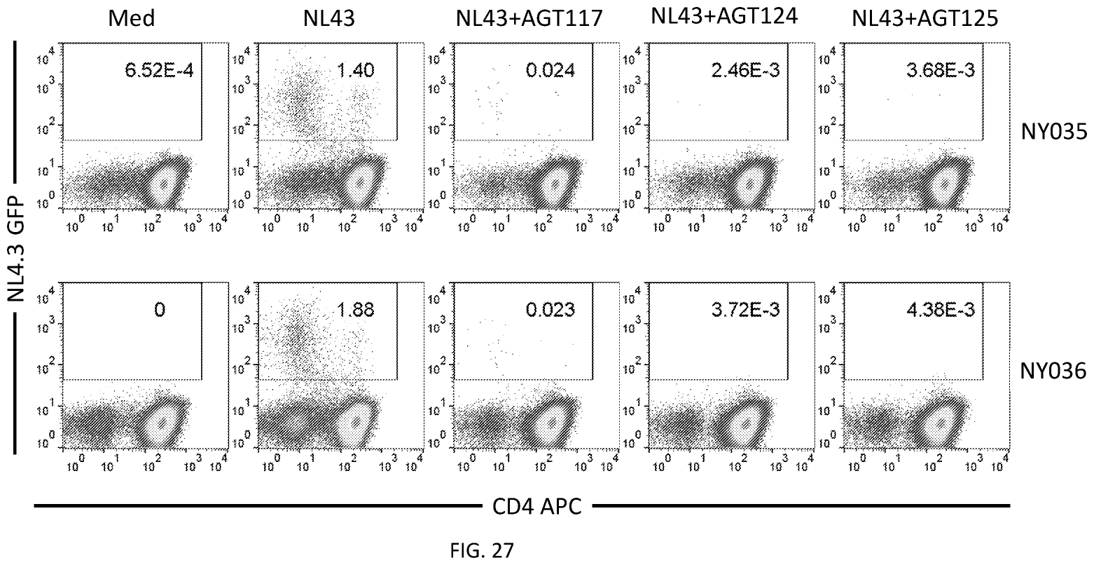

As shown in FIG. 27. NL43-GFP virus infection without lentivirus vector addition produced and average of 2% infected cells, using data from two different blood donors (NY035 and NY036). Transduction of T cells with AGT117 protected against HIV infection and reduced the proportion of infected cells to 0.024%. Transduction with AGT124 protected CD4 T cells from infection and reduced the number of infected cells to 0.003% of control levels and transduction with AGT125 reduced infection of CD4 T cells to 0.004% of control levels. While AGT117, AGT124, and AGT125 vectors all provided protection for CD4 T cells against HIV infection, AGT124 and AGT125 were more potent compared to AGT117.

### Example 17: A Lentivirus Vector Encoding Soluble CD4-IgG Fc Fusion Protein and Inhibitory RNA Against CCR5 and HIV Vif and Tat Protects CD4 T Cells from HIV Infection

Expression of both a soluble sCD4-IgG Fc and inhibitory RNA against CCR Vif and Tat confers better protection against HIV that expression of soluble sCD4-IgG Fc alone.

Method: CD4 T cells were separated by negative selection and stimulated for 1 day with TransAct (CD3/CD28 beads) (Miltenyi Biotec) plus IL-2 (30 U/mL) (Thermo Fisher Scientific) and then transduced with lentivirus AGT103 (SEQ ID NO: 78) or AGT118 (SEQ ID NO: 80) at a MOI of 5. The AGT103 vector (SEQ ID NO: 78) contains an EF-1α promoter (SEQ ID NO: 14) that drives expression of the miR30-CCR5/miR21-Vif/mir185-Tat microRNA cluster sequence (SEQ ID NO: 65) (miR30-CCR5/miR21-Vif/miR185-Tat microRNA cluster sequence). The miR30-CCR5 sequence is SEQ ID NO: 62. The miR21-Vif sequence is SEQ ID NO: 63. The miR185-Tat sequence is SEQ ID NO: 64. The AGT118 vector (SEQ ID NO: 80) contains an EF-1α promoter (SEQ ID NO: 14) that drives expression of sCD4(D1+D2)-IgG1 Fc (SEQ ID NO: 9) and the miR30-CCR5/miR21-Vif/miR185-Tat microRNA cluster sequence (SEQ ID NO: 65).

One day later, cells were infected with 1 MOI of HIV recombinant strain NL43 that expresses GFP. After 24 hours, CD3/CD28 beads, lentivirus and HIV were removed by washing 3 times with PBS and cultured in RPMI 1640 medium (Thermo Fisher Scientific) containing 10% FBS (Gemini Bio) and 1% Pen-Strep (Thermo Fisher Scientific) with IL-2 (30 U/mL) in a 37° C. incubator at 5% C02 for 7 days with medium supplementation as needed. At the end of the culture, cells were collected and analyzed by flow cytometry. If the CD4 cell expresses GFP, it was infected by HIV.

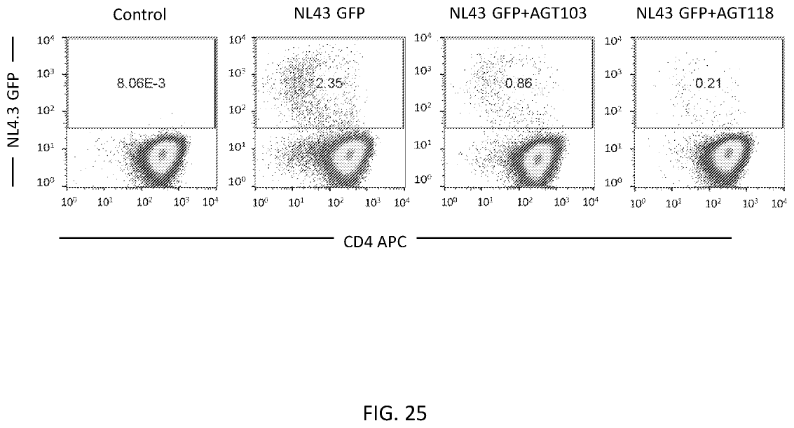

As shown in FIG. 25. AGT118 (SEQ ID NO: 80) further improved the protective effect of AGT103 that encodes an inhibitory RNA against CCR5 and HIV Vif and Tat. In AGT118-transduced cells (see column labeled NL43-GFP+AGT118). 0.21% of cells were HIV positive. In AGT103 transduced cells (see column labeled NL43-GFP+AGT103). 0.86% of cells were HIV positive. In control treated cells (see column labeled NL43-GFP). 2.35% of cells were HIV positive. Therefore, there was a 91.1% decrease in HIV infection with AGT118 and a 75.6% decrease with AGT103.

### Example 18: EF-1α, IL-2, and IFNγ Promoter Regulated CD4-IgG1 Fc Fusion Protein Expression by PHA/Ionomycin Stimulated CD4 T Cells

T cell activation results can induce expression of CD4-IgG1 Fc using the IL-2 promoter.

Method: To measure CD4-IgG1 Fc fusion protein expression in primary CD4 T cells, PBMCs were purified from whole blood and the CD4+ T cell subset was enriched by negative selection using magnetic beads. 1×10⁶ CD4 T cells were cultured in 2 mL of RPMI 1640 medium (Thermo Fisher Scientific) containing 10% FBS (Gemini Bio) and 1% Pen-Strep (Thermo Fisher Scientific) in a 37° C. incubator at 5% CO₂ and supplemented with recombinant human IL-2 (30) U/mL) (Thermo Fisher Scientific) and TransAct (CD3/CD28 microbeads) (Miltenyi Biotec).

Cells were cultured for 1 day before adding 5 MOI of lentivirus vectors AGT117 (SEQ ID NO: 10), AGT120 (SEQ ID NO: 82), and AGT121 (SEQ ID NO: 83). Each of the AGT117 vector, the AGT120 vector, and the AGT121 vector encodes a sCD4-IgG1 Fc sequence (SEQ ID NO: 9) (sCD4(D1+D2)-IgG1 Fc). The AGT117 vector contains an EF-1α promoter (SEQ ID NO: 14) upstream of the sCD4 (D1+D2)-IgG1 sequence. The AGT120 vector contains an IL-2 promoter (SEQ ID NO: 66) upstream of the sCD4 (D1+D2)-IgG1 sequence. The AGT121 vector contains an IFNγ promoter (SEQ ID NO: 15) upstream of the sCD4 (D1+D2)-IgG1 sequence.

One day after transduction, the medium was removed and replaced with fresh medium plus IL-2. Cells were cultured for an additional 6 days and then the medium was replaced without IL-2 for 16 hours. Next, the cells were stimulated with PMA (20 ng/ml) (Millipore Sigma) and ionomycin (1 μg/mL) (Millipore Sigma) for 24 hours. The cells were washed and collected to measure the intracellular expression of CD4-IgG Fc fusion protein with a PE anti-human IgG1 Fc antibody (Cat. No. 12-4998-82, Thermo Fisher Scientific).

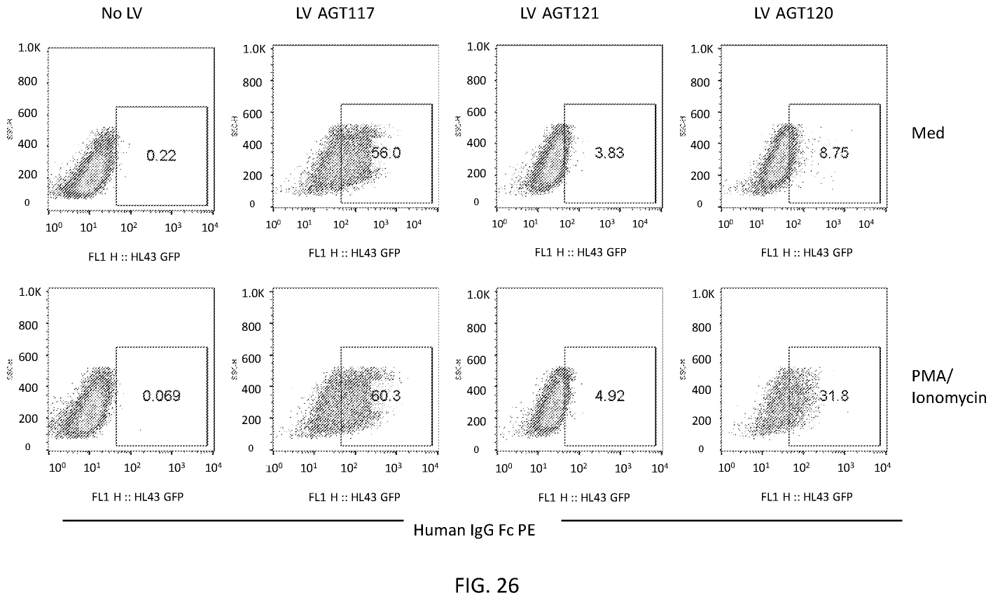

As shown in FIG. 26, CD4 T cells expressing CD4-IgG1 Fc were detected with a PE-labeled antibody against human IgG Fc. PMA/ionomycin stimulates T cell activity by the PKC pathway. In AGT117 (SEQ ID NO: 10) transduced cells, where the EF-1α promoter is regulating expression, 56% of cells expressed CD4-IgG1 Fc (see column labeled LV-AGT117, top panel). The percent expression of CD4-IgG1 Fc increased to 60.3% with PMA/ionomycin treatment (see column labeled LV-AGT117, bottom panel). In AGT121 (SEQ ID NO: 83) transduced cells, where the IFNγ promoter is regulating expression, 3.83% of cells expressed CD4-IgG1 Fc (see column labeled LV-AGT121, top panel). The percent expression of CD4-IgG1 Fc increased to 4.92% with PMA/ionomycin treatment (see column labeled LV-AGT121, bottom panel). In AGT120 (SEQ ID NO: 82) transduced cells, where the IL-2 promoter is regulating expression, 8.75% of cells expressed CD4-IgG1 Fc (see column labeled LV-AGT120, top panel). The percent expression of CD4-IgG1 Fc increased to 31.8% with PMA/ionomycin treatment (see column labeled LV-AGT120, bottom panel). These data demonstrate that CD4-IgG1 Fc can be induced in CD4 T cells by the T cell activating compounds PMA/ionomycin to increase IL-2 promoter activity.

### Example 19: Materials and Methods Utilized in Examples Described Herein

### Cloning of Promoters in a Lentivirus Plasmid:

DNA fragments of the EF-1α(Gen Bank: J04617.1), IL-2 (Gen Bank: M13879.1), IFNγ (Gen Bank: AF330164.1), or CD69 promoter (Gen Bank: Z38109.1) with flanking ClaI and EcoRI restriction enzyme sites was synthesized by Integrated DNA Technologies. The promoter fragments and lentivirus plasmid were digested with ClaI/EcoRI restriction enzymes (New England Biolabs). The digested lentivirus plasmid was electrophoresed on a 1% agarose gel (Thermo Fisher Scientific), excised, and extracted from the gel with the PureLink DNA gel extraction kit (Thermo Fisher Scientific). The DNA concentration was determined and then mixed with the digested DNA fragment using a vector to insert ratio of 3:1. The mixture was ligated with T4 DNA ligase (New England Biolabs) for 16 hours at room temperature and then 3 μL of the ligation mix was added to 23 μL of STBL3 competent bacterial cells (Thermo Fisher Scientific). Transformation was carried out by heat-shock at 42° C. Bacterial cells were streaked onto agar plates containing 100 μg/mL ampicillin and then colonies were expanded in LB broth (VWR). To check for insertion of the DNA fragments, plasmid DNA was extracted from harvested bacteria cultures with the PureLink DNA plasmid mini prep kit (Thermo Fisher Scientific). The inserted DNA fragments were verified by DNA sequencing (Eurofins Genomics). The lentivirus plasmids containing a verified promoter sequence were then used to insert anti-HIV antibody sequences or CD4-IgG1 Fc.

### Cloning of the Anti-HIV Antibodies in a Lentivirus Plasmid:

A DNA fragment of the VRC01 anti-HIV immunoglobulin heavy chain variable region (Gen Bank: GU980702.1) or 3BNC117 (Gen Bank: HE584537.1) with flanking XhoI and NheI restriction enzyme sites and the light chain variable region of VRC01 (Gen Bank: GU980703.1) or 3BNC117 (Gen Bank: HE584538.1) with flanking EcoRI and NotI restriction enzyme sites was synthesized by Integrated DNA Technologies. The VRC01 or 3BNC117 heavy variable fragment was digested with XhoI/NheI restriction enzymes (New England Biolabs) and inserted into the lentivirus plasmid before inserting the light variable fragment. The lentivirus plasmid containing heavy and light constant regions was digested with either XhoI/NheI or EcoRI/NotI restriction enzymes. The digested product was electrophoresed on a 1% agarose gel (Thermo Fisher Scientific), excised, and extracted from the gel with the PureLink DNA gel extraction kit (Thermo Fisher Scientific). The DNA concentration was determined and then mixed with the digested DNA fragment using a vector to insert ratio of 3:1. The mixture was ligated with T4 DNA ligase (New England Biolabs) for 16 hours at room temperature and then 3 μL of the ligation mix was added to 23 μL of STBL3 competent bacterial cells (Thermo Fisher Scientific). Transformation was carried out by heat-shock at 42° C. Bacterial cells were streaked onto agar plates containing 100 μg/mL ampicillin and then colonies were expanded in LB broth (VWR). To check for insertion of the DNA fragments, plasmid DNA was extracted from harvested bacteria cultures with the PureLink DNA plasmid mini prep kit (Thermo Fisher Scientific). The inserted DNA fragments were verified by DNA sequencing (Eurofins Genomics). The lentivirus plasmid containing a verified sequence was then used to package lentiviral particles in 293T cells to test for their ability to express either the VRC01 or 3BNC117 antibody by detection with an APC-labelled anti-IgG1 or anti-IgG Fc antibody (Biolegend) on a flow cytometer.

### Cloning of CD4-IgG1 Fe in a Lentivirus Plasmid:

DNA fragments of CD4 fused with the human immunoglobulin heavy chain containing the hinge and Fc regions were synthesized by Integrated DNA Technologies with flanking BsrGI and NotI restriction enzyme sites. The CD4-IgG1 Fc fragment and lentivirus plasmid was digested with BsrGI/NotI restriction enzymes (New England Biolabs). The digested plasmid was electrophoresed on a 1% agarose gel (Thermo Fisher Scientific), excised, and extracted from the gel with the PureLink DNA gel extraction kit (Thermo Fisher Scientific). The DNA concentration was determined and then mixed with the digested DNA fragment using a vector to insert ratio of 3:1. The mixture was ligated with T4 DNA ligase (New England Biolabs) for 16 hours at room temperature and then 3 μL of the ligation mix was added to 23 μL of STBL3 competent bacterial cells (Thermo Fisher Scientific). Transformation was carried out by heat-shock at 42° ° C. Bacterial cells were streaked onto agar plates containing 100 μg/mL ampicillin and then colonies were expanded in LB broth (VWR). To check for insertion of the DNA fragments, plasmid DNA was extracted from harvested bacteria cultures with the PureLink DNA plasmid mini prep kit (Thermo Fisher Scientific). The inserted DNA fragments were verified by DNA sequencing (Eurofins Genomics). The lentivirus plasmid containing a verified sequence was then used to package lentiviral particles in 293T cells to test for their ability to express CD4-IgG1 Fc by detection with a PE-labelled anti-IgG Fc antibody (Cat. No. 12-4998-82, Thermo Fisher Scientific) on a flow cytometer.

### Example 20: Soluble CD4-IgG Fc Expression in a Leukemic T Cell Line

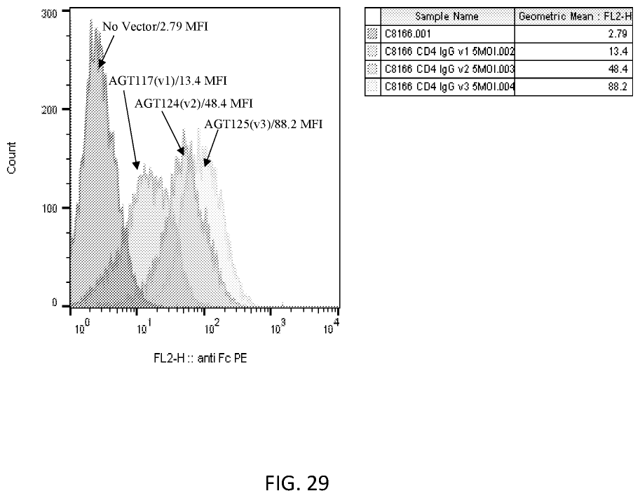

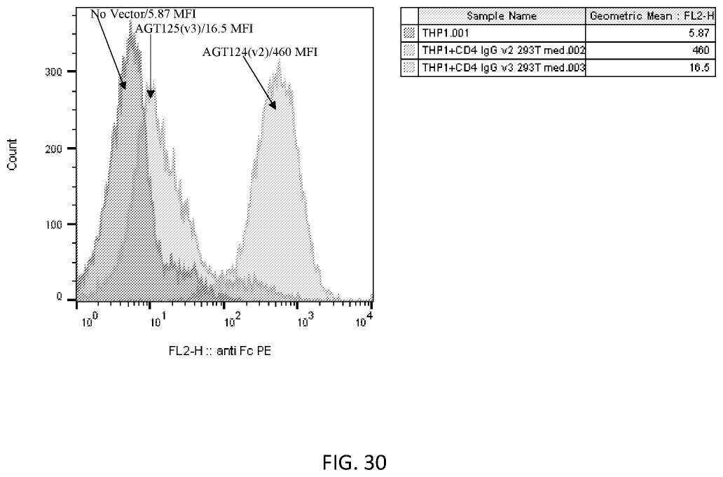

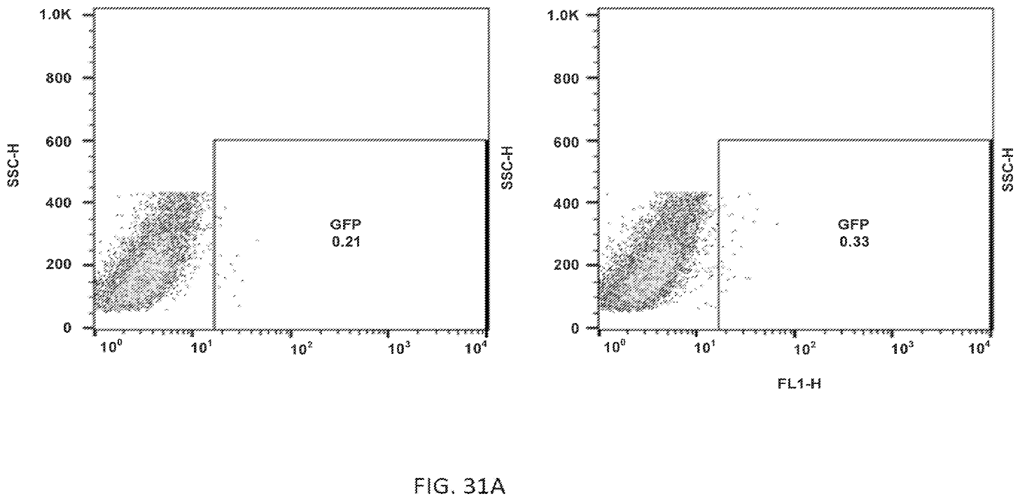

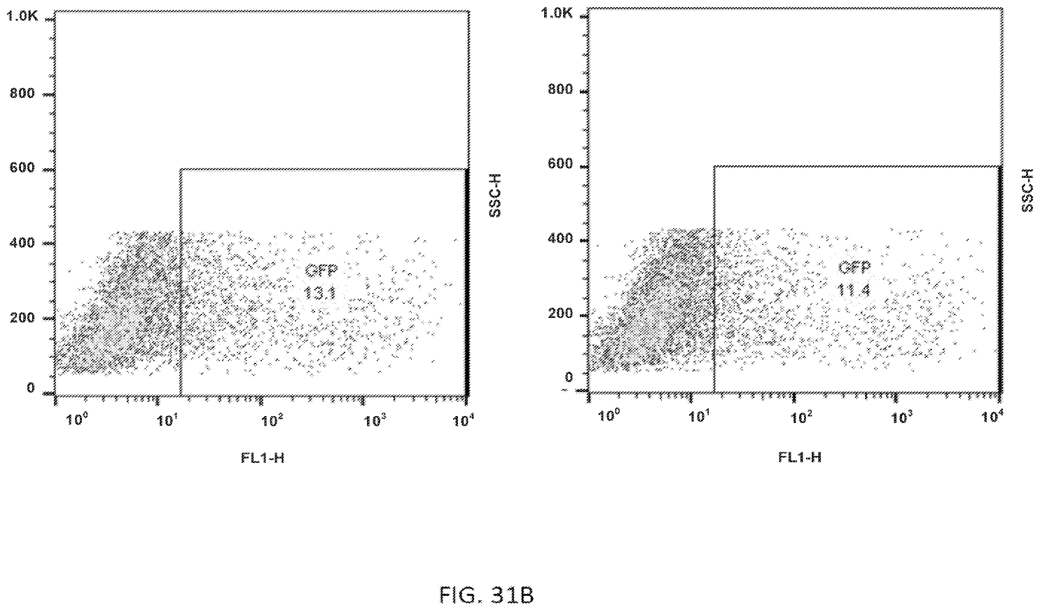

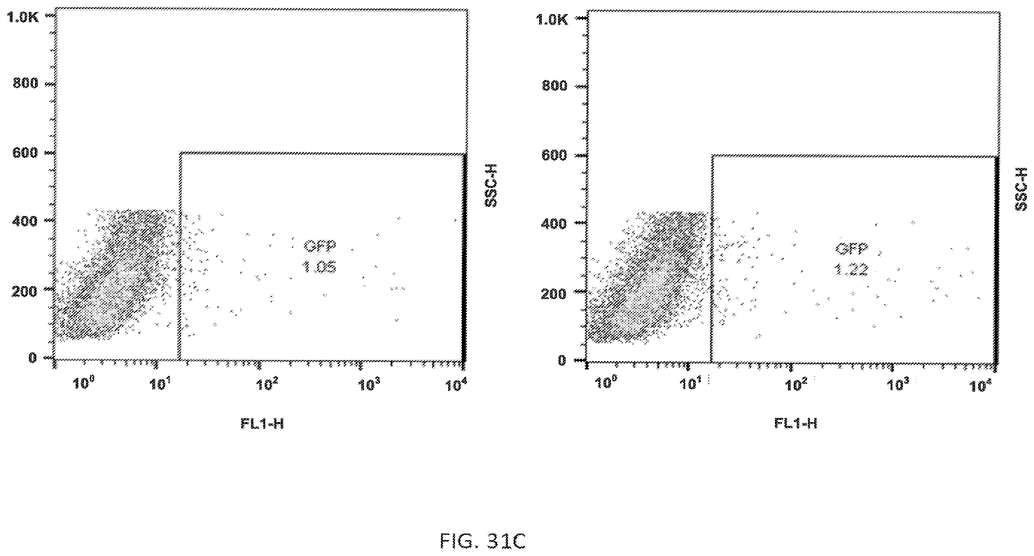

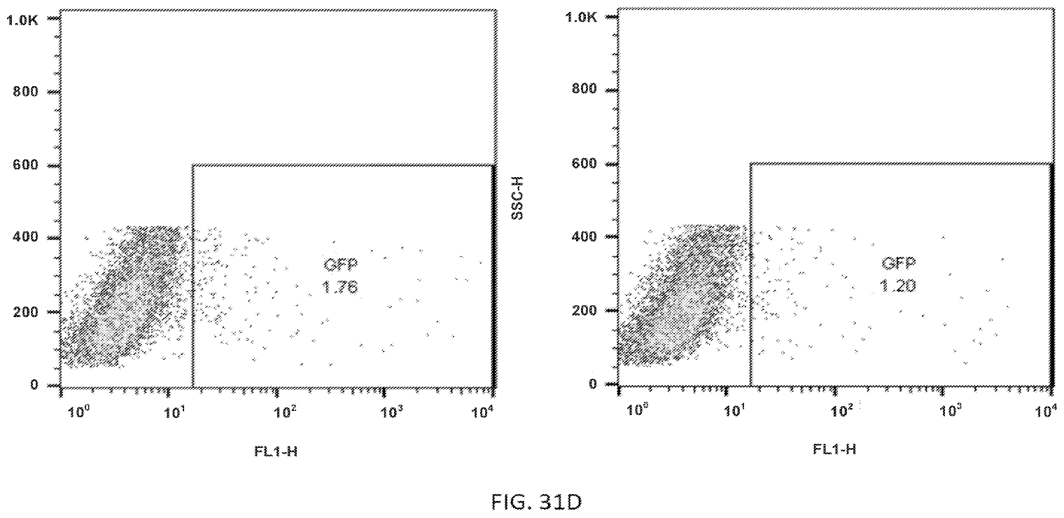

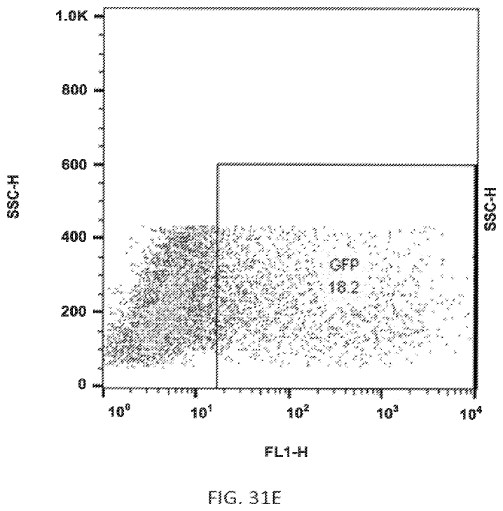

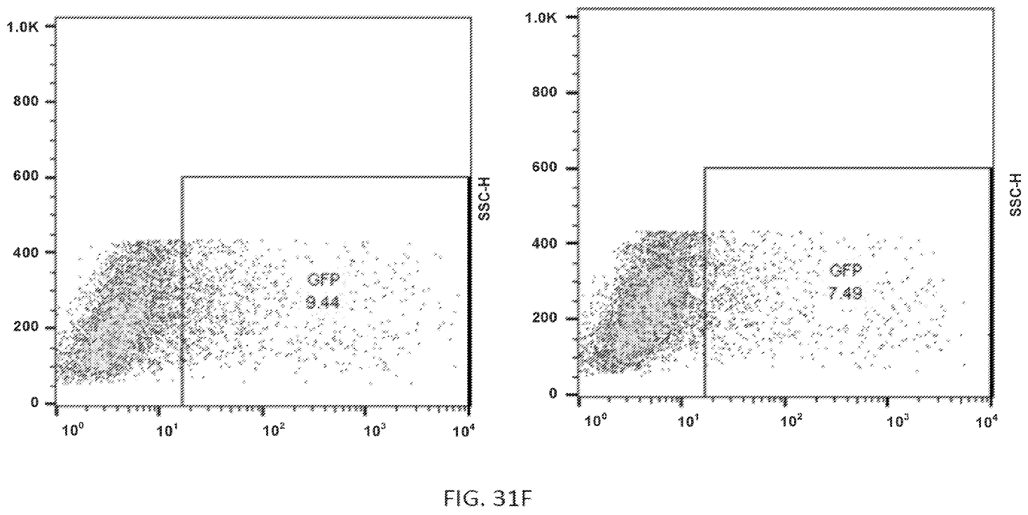

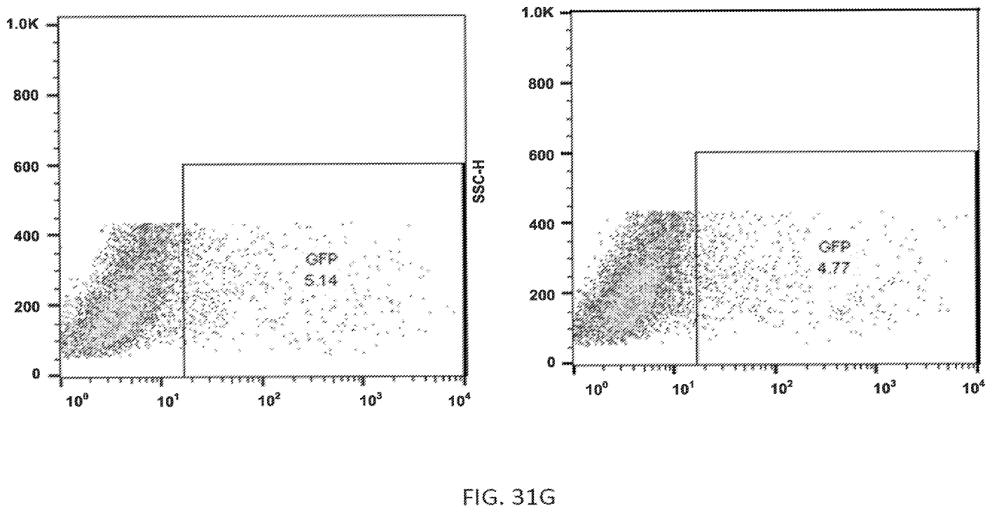

C8166 is a T cell leukemia cell line that is permissive for lentivirus vector modification. Transduction of the C8166 T cell leukemia cell line with a lentivirus encoding fusion protein comprised of soluble CD4 and the Fc region from human IgG1. Three distinct versions of the Fc region were tested. Version 1 (SEQ ID: 9) is a truncated Fc sequence continuing the amino terminal sequence up to the hinge region. Version 2 (SEQ ID: 76) contains the complete IgG1 Fc region with the accepted wild-type sequence. Version 3 (SEQ ID: 77) contains the complete IgG1 Fc region with mutations to disable complement binding and binding to the cell surface Fc Gamma Receptor Type II. Binding to Fc gamma receptors may have inhibitory effects on antibody production in lymph nodes where we expect the soluble CD4-IgG1 Fc molecule to have highest expression. The Version 3 preserves the function of inhibiting HIV but is reduced in binding to the Fc Gamma Receptor II. FIG. 29 shows the relative expression levels for Version 1 (SEQ ID NO: 9) and its corresponding lentivirus vector (SEQ ID NO: 10; AGT117), Version 2 (SEQ ID NO: 76) and its corresponding lentivirus vector (SEQ ID NO: 88; AGT124), or Version 3 (SEQ ID NO: 77) and its corresponding lentivirus vector (SEQ ID NO: 89; ATG125), in C8166 cells. FIG. 30 the relative binding of secreted Version 1, 2, or 3 proteins to CD4-negative, Fc Gamma Receptor III-expressing THP-1 cells, a monocytoid cell line. FIGS. 31A-31G show potency of Version 1 or 2 for inhibiting HIV infection using the NL4 and HXB2 strains of HIV-1 and C8166 cells as targets.

Method: C8166 cells were cultured in RPMI 1640 medium (Thermo Fisher Scientific) containing 10% FBS and then transduced with MOI 5 of lentivirus vector encoding CD4-IgG1 Fc versions 1 (AGT117), 2 (AGT 124), or 3 (AGT125).

After 72 hours, cells were collected in 12×75 mm FACs tubes and centrifuged at 1000 rpm for 3 minutes. The cells were washed with PBS and centrifuged at 1000 rpm for 3 minutes. 0.2 mL of fixation solution from the BD Fixation/Permeabilization kit were added to each tube and the cells were maintained at 4° C. for 15 minutes. The cells were washed 2 times with BD Perm/Wash buffer and 0.1 mL was added to each tube with 2.5 μL of PE anti-human IgG1 Fc antibody (Biolegend). The tubes were kept at 4° C. for 20 minutes and then washed 2 times with PBS. The cells were resuspended in 0.7 mL of PBS and detected on a FACS Calibur flow cytometer.

As shown in FIG. 29, CD4-IgG1 Fc expression was detected in AGT117-transduced C8166 cells. The levels of protein expression are proportional to the Mean Fluorescence Intensity (MFI) after staining. Expression of Version 3 was highest with 88.2 MFI and was similar to Version 2 with 48.4 MFI (logarithmic scale). Version 1 expression was lower at 13.4 MFI and non-transduced cells (background) was 2.79 MFI. Accordingly, Version 2 and Version 3 expression levels were high and roughly similar.

Next, cell-free culture supernatant was collected from cells transduced with AGT124 or AGT125 vectors. Supernatants were overlayed on THP-1 cells for 30 minutes, then cells were fixed and stained as described above except there was no permeabilization step since we are testing for cell surface binding. The THP-1 cell line was used because it expresses the Fc Gamma Receptor III and will bind antibodies containing the natural Fc sequence of human IgG1. As shown in FIG. 30 AGT124, being the natural or wild-type version of the Fc region, demonstrated the highest level of binding to THP-1 cells with MFI 460 compared to AGT125 with MFI 16.5 that was close to the background of untreated control THP-1 cells (MFI 5.87). The result confirms that site-directed mutagenesis of the Fc region in AGT125 eliminated the binding site for Fc Gamma Receptor III.

As shown in FIGS. 31A-31G, both versions AGT117 and AGT124 of CD4-IgG1 Fc vectors were potent inhibitors of HIV-1 infection. C8166 cells were transduced with AGT117 or AGT124 vectors for 3 days then challenged with HIV using either the HXB2 strain engineered to also express Green Fluorescence Protein or the NL4 virus strain also expressing GFP. Infectious HIV was overlayed on transduced cells for 1 day, removed by washing, and the cells were fixed and examined by flow cytometry to detect the level of GFP expression as a measure of infection efficiency.

FIG. 31A shows that, in two (2) replicates, when no virus was introduced into the C8166 cells, the percentage of GFP positive cells was 0.21% and 0.33% (average of 0.27% GFP positive cells). FIG. 31B shows that, in two (2) replicates, when HXB2-GFP virus was introduced into C8166 cells, the percentage of GFP positive cells was 13.1% and 11.4% (average of 12.25% GFP positive cells). FIG. 31C shows that, in two (2) replicates, when HXBc2-GFP virus was introduced into C8166 cells along with version 1 of CD4-IgG (SEQ ID NO: 9), the percentage of GFP positive cells was 1.05% and 1.22% (average of 1.14% GFP positive cells). FIG. 31D shows that, in two (2) replicates, when HXB2-GFP virus was introduced into C8166 cells along with version 2 of the CD4-IgG (SEQ ID NO: 76), the percentage of GFP positive cells was 1.76% and 1.20% (average of 1.48% GFP positive cells).

FIG. 31E shows that when NL4-GFP virus was introduced into C8166 cells, the percentage of GFP positive cells was 18.2%. FIG. 31F shows that, in two (2) replicates, when NL4-GFP virus was introduced into C8166 cells along with version 1 of CD4-IgG (SEQ ID NO: 9), the percentage of GFP positive cells was 9.44% and 7.49% (average of 8.47% GFP positive cells). FIG. 31G shows that, in two (2) replicates, when NL4-GFP virus was introduced into C8166 cells along with version 2 of CD4-IgG (SEQ ID NO: 76), the percentage of GFP positive cells was 5.14% and 4.77% (average of 4.96% GFP positive cells).

Using HXB2-GFP virus challenge, both AGT117 (91% inhibition of virus infection) and AGT124 (88% inhibition of virus infection) proved potent antiviral agents. Using NL4-GFP virus challenge demonstrated an advantage of AGT124 (73% virus inhibition) compared to AGT117 (53% virus inhibition).

### Example 21: Comparing Inducible Expression Using CD69 Promoter 1050 Versus CD69 Promoter 625 to Express CD4-IgG1 Fc in Gag-Specific CD4+ T Cells

Two versions of the CD69 gene promoter are tested to measure strength of gene expression and inducibility in primary, antigen specific CD4 T cells. AGT122 (SEQ ID NO: 84) uses the CD69 1050 promoter (SEQ ID: 67) (CD69 promoter ((1050)+CNS2 enhancer) to express CD4-IgG1 Fc (SEQ ID NO: 9) and AGT123 (SEQ ID NO: 85) uses the CD69 625 promoter (SEQ ID: 68) (CD69 promoter (625)+CNS2 enhancer) to express CD4-IgG1 Fc (SEQ ID NO: 9). Expression levels are compared to AGT120 (SEQ ID NO: 82) that uses the IL-2 promoter (SEQ ID NO: 66) to express CD4-IgG1 Fc (SEQ ID NO: 9).

Methods: Peripheral blood mononuclear cells (PBMC) obtained from an HIV+ donor are purified and stimulated overnight with 152 overlapping peptides representing the HIV-1 Gag polyprotein sequence. The following day cells expressing CD8, CD56 or CD19 are removed by magnetic bead depletion and the remaining cells, highly enriched for CD4+ T cells, are transduced with MOI 10 of AGT122 (SEQ ID NO: 84), AGT123 (SEQ ID NO: 85), or the control AGT120 (SEQ ID NO: 82). Transduced cells are cultured for 8 days under static conditions, then harvested, washed and cryopreserved.

Cryopreserved cells are thawed, suspended in medium and washed three times to remove DMSO, then cultured in RPMI complete medium with 10% fetal bovine serum. After 1 day, the cells are restimulated with the same peptide used before or treated with a mock solution containing excipients but no peptides. Six hours after peptide stimulation cell-free fluids and cells are harvested.

Cell free fluids are tested by ELISA for the presence of CD4-IgG1 Fc. Cells are collected in 12×75 mm FACs tubes and centrifuged at 1000 rpm for 3 minutes. The cells are washed with PBS and centrifuged at 1000 rpm for 3 minutes. 0.2 mL of fixation solution from the BD Fixation/Permeabilization kit are added to each tube and the cells were maintained at 4° C. for 15 minutes. The cells are washed 2 times with BD Perm/Wash buffer and 0.1 mL is added to each tube with 2.5 μL of PE anti-human IgG1 Fc antibody (Biolegend). The tubes are kept at 4° C. for 20 minutes and then washed 2 times with PBS. The cells are resuspended in 0.7 mL of PBS and detected on a FACS Calibur flow cytometer.

### Example 22: Comparing Inducible Expression Using Different Promoters to Express Soluble Exogenous Factors

A promoter can be cloned into a lentiviral plasmid as described in Example 19. Soluble CD4-IgG1 Fc can be cloned into a lentiviral plasmid as described in Example 19. Using this method, multiple lentiviral plasmids can be synthesized in which different promoters are used to express soluble CD4-IgG1 Fc.

Methods: Peripheral blood mononuclear cells (PBMC) obtained from an HIV+ donor are purified and stimulated overnight with 152 overlapping peptides representing the HIV-1 Gag polyprotein sequence. The following day cells expressing CD8, CD56 or CD19 are removed by magnetic bead depletion and the remaining cells, highly enriched for CD4+ T cells, are transduced with the previously synthesized lentiviral vectors. Transduced cells are cultured for 8 days under static conditions, then harvested, washed and cryopreserved.

Cryopreserved cells are thawed, suspended in medium and washed three times to remove DMSO, then cultured in RPMI complete medium with 10% fetal bovine serum. After 1 day, the cells are restimulated with the same peptide used before or treated with a mock solution containing excipients but no peptides. Six hours after peptide stimulation cell-free fluids and cells are harvested.

Cell free fluids are tested by ELISA for the presence of CD4-IgG1 Fc. Cells are collected in 12×75 mm FACs tubes and centrifuged at 1000 rpm for 3 minutes. The cells are washed with PBS and centrifuged at 1000 rpm for 3 minutes. 0.2 mL of fixation solution from the BD Fixation/Permeabilization kit are added to each tube and the cells were maintained at 4° C. for 15 minutes. The cells are washed 2 times with BD Perm/Wash buffer and 0.1 mL is added to each tube with 2.5 μL of PE anti-human IgG1 Fc antibody (Biolegend). The tubes are kept at 4° ° C. for 20 minutes and then washed 2 times with PBS. The cells are resuspended in 0.7 mL of PBS and detected on a FACS Calibur flow cytometer.

### Sequences

The following sequences are referred to herein:

| SEQ ID | | |
|---|---|---|
| NO: | Description | Sequence |
| 1 | VRC01 (IL-2 | ATGTACAGGATGCAACTCCTGTCTTGCATTGCACTAAGTCTTGCACTTGTCA |
| | secretory | CGCAGGTGCAGCTGGTGCAGTCTGGGGGTCAGATGAAGAAGCCTGGCGAGT |
| | sequence)-HV- | CGATGAGAATTTCTTGTCGGGCTTCTGGATATGAATTTATTGATTGTACGCT |
| | CH-T2A-LV- | AAATTGGATTCGTCTGGCCCCCGGAAAAAGGCCTGAGTGGATGGGATGGCT |
| | CL | GAAGCCTCGGGGGGGGGCCGTCAACTACGCACGTCCACTTCAGGGCAGAGT |
| | | GACCATGACACGAGACGTTTATTCCGACACAGCCTTTTTGGAGCTGCGCTCG |
| | | TTGACAGTAGACGACACGGCCGTCTACTTTTGTACTAGGGGAAAAAACTGT |
| | | GATTACAATTGGGACTTCGAACACTGGGGCCGGGGCACCCCGGTCATCGTC |
| | | TCATCAGCTAGCACCAAGGGCCCATCGGTCTTCCCCCTGGCACCCTCCTCCA |
| | | AGAGCACCTCTGGGGGCACAGCGGCCCTGGGCTGCCTGGTCAAGGACTACT |
| | | TCCCCGAACCGGTGACGGTGTCGTGGAACTCAGGCGCCCTGACCAGCGGCG |
| | | TGCACACCTTCCCGGCTGTCCTACAGTCCTCAGGACTCTACTCCCTCAGCAG |
| | | CGTGGTGACCGTGCCCTCCAGCAGCTTGGGCACCCAGACCTACATCTGCAA |
| | | CGTGAATCACAAGCCCAGCAACACCAAGGTGGACAAGAAAGTTGAGCCCA |
| | | AATCTTGTGACAAAACTCACACATGCCCACCGTGCCCAGCACCTGAACTCCT |
| | | GGGGGGACCGTCAGTCTTCCTCTTCCCCCCAAAACCCAAGGACACCCTCAT |
| | | GATCTCCCGGACCCCTGAGGTCACATGCGTGGTGGTGGACGTGAGCCACGA |
| | | AGACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGG |
| | | TCAGCGTCCTCACCGTCCTGCACCAGGACTGGCTGAATGGCAAGGAGTACA |
| | | AGTGCAAGGTCTCCAACAAAGCCCTCCCAGCCCCCATCGAGAAAACCATCT |
| | | CCAAAGCCAAAGGGCAGCCCCGAGAACCACAGGTGTACACCCTGCCCCCAT |
| | | CCCGGGATGAGCTGACCAAGAACCAGGTCAGCCTGACCTGCCTGGTCAAAG |
| | | GCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTT |
| | | CCTCTACAGCAAGCTCACCGTGGACAAGAGCAGGTGGCAGCAGGGGAACG |
| | | TCTTCTCATGCTCCGTGATGCATGAGGCTCTGCACAACCACTACACGCAGAA |
| | | GAGCCTCTCCCTGTCTCCGGGTAAACGTAGACGAAAGCGCGGAAGCGGAGA |
| | | GGGCAGAGGAAGTCTGCTAACATGCGGTGACGTCGAGGAGAATCCTGGACC |
| | | TGGATCCATGTACAGGATGCAACTCCTGTCTTGCATTGCACTAAGTCTTGCA |
| | | CTTGTCACGGAAATTGTGTTGACACAGTCTCCAGGCACCCTGTCTTTGTCTC |
| | | CAGGGGAAACAGCCATCATCTCTTGTCGGACCAGTCAGTATGGTTCCTTAGC |
| | | CTGGTATCAACAGAGGCCCGGCCAGGCCCCCAGGCTCGTCATCTATTCGGG |
| | | CTCTACTCGGGCCGCTGGCATCCCAGACAGGTTCAGCGGCAGTCGGTGGGG |
| | | GCCAGACTACAATCTCACCATCAGCAACCTGGAGTCGGGAGATTTTGGTGT |
| | | TTATTATTGCCAGCAGTATGAATTTTTTGGCCAGGGGACCAAGGTCCAGGTC |
| | | GACATTAAGCGAGAATTCGTGGCTGCACCATCTGTCTTCATCTTCCCGCCAT |
| | | CTGATGAGCAGTTGAAATCTGGAACTGCCTCTGTTGTGTGCCTGCTGAATAA |
| | | CTTCTATCCCAGAGAGGCCAAAGTACAGTGGAAGGTGGATAACGCCCTCCA |
| | | ATCGGGTAACTCCCAGGAGAGTGTCACAGAGCAGGACAGCAAGGACAGCA |
| | | CCTACAGCCTCAGCAGCACCCTGACGCTGAGCAAAGCAGACTACGAGAAAC |
| | | ACAAAGTCTACGCCTGCGAAGTCACCCATCAGGGCCTGAGCTCGCCCGTCA |
| | | CAAAGAGCTTCAACAGGGGAGAGTGTTAG |
| 2 | CMV-VRC01 | ACTAGTATTATGCCCAGTACATGACCTTATGGGACTTTCCTACTTGGCAGTA |
| | (IL-2 secretory | CATCTACGTATTAGTCATCGCTATTACCATGGTGATGCGGTTTTGGCAGTAC |
| | sequence)-HV- | ATCAATGGGCGTGGATAGCGGTTTGACTCACGGGGATTTCCAAGTCTCCAC |
| | CH-T2A-LV- | CCCATTGACGTCAATGGGAGTTTGTTTTGGCACCAAAATCAACGGGACTTTC |
| | CL (AGT111) | CAAAATGTCGTAACAACTCCGCCCCATTGACGCAAATGGGCGGTAGGCGTG |
| | | TACGGTGGGAGGTTTATATAAGCAGAGCTCGTTTAGTGAACCGTCAGATCG |
| | | CCTGGAGACGCCATCCACGCTGTTTTGACCTCCATAGAAGATTCTAGATCTC |
| | | GAGGCCACCATGTACAGGATGCAACTCCTGTCTTGCATTGCACTAAGTCTTG |
| | | CACTTGTCACGCAGGTGCAGCTGGTGCAGTCTGGGGGTCAGATGAAGAAGC |
| | | CTGGCGAGTCGATGAGAATTTCTTGTCGGGCTTCTGGATATGAATTTATTGA |
| | | TTGTACGCTAAATTGGATTCGTCTGGCCCCCGGAAAAAGGCCTGAGTGGAT |
| | | GGGATGGCTGAAGCCTCGGGGGGGGGCCGTCAACTACGCACGTCCACTTCA |
| | | GGGCAGAGTGACCATGACACGAGACGTTTATTCCGACACAGCCTTTTTGGA |
| | | GCTGCGCTCGTTGACAGTAGACGACACGGCCGTCTACTTTTGTACTAGGGG |
| | | AAAAAACTGTGATTACAATTGGGACTTCGAACACTGGGGCCGGGGCACCCC |
| | | GGTCATCGTCTCATCAGCTAGCACCAAGGGCCCATCGGTCTTCCCCCTGGCA |
| | | CCCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCTG |
| | | ACCAGCGGCGTGCACACCTTCCCGGCTGTCCTACAGTCCTCAGGACTCTACT |
| | | CCCTCAGCAGCGTGGTGACCGTGCCCTCCAGCAGCTTGGGCACCCAGACCT |
| | | ACATCTGCAACGTGAATCACAAGCCCAGCAACACCAAGGTGGACAAGAAA |
| | | GTTGAGCCCAAATCTTGTGACAAAACTCACACATGCCCACCGTGCCCAGCA |
| | | CCTGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGACG |
| | | TGAGCCACGAAGACCCTGAGGTCAAGTTCAACTGGTACGTGGACGGCGTGG |
| | | AGGTGCATAATGCCAAGACAAAGCCGCGGGAGGAGCAGTACAACAGCACG |
| | | TACCGTGTGGTCAGCGTCCTCACCGTCCTGCACCAGGACTGGCTGAATGGC |
| | | AAGGAGTACAAGTGCAAGGTCTCCAACAAAGCCCTCCCAGCCCCCATCGAG |
| | | AAAACCATCTCCAAAGCCAAAGGGCAGCCCCGAGAACCACAGGTGTACAC |
| | | CCTGCCCCCATCCCGGGATGAGCTGACCAAGAACCAGGTCAGCCTGACCTG |
| | | CCTGGTCAAAGGCTTCTATCCCAGCGACATCGCCGTGGAGTGGGAGAGCAA |
| | | TGGGCAGCCGGAGAACAACTACAAGACCACGCCTCCCGTGCTGGACTCCGA |
| | | CGGCTCCTTCTTCCTCTACAGCAAGCTCACCGTGGACAAGAGCAGGTGGCA |
| | | GCAGGGGAACGTCTTCTCATGCTCCGTGATGCATGAGGCTCTGCACAACCA |
| | | CTACACGCAGAAGAGCCTCTCCCTGTCTCCGGGTAAACGTAGACGAAAGCG |
| | | CGGAAGCGGAGAGGGCAGAGGAAGTCTGCTAACATGCGGTGACGTCGAGG |
| | | AGAATCCTGGACCTGGATCCATGTACAGGATGCAACTCCTGTCTTGCATTGC |
| | | ACTAAGTCTTGCACTTGTCACGGAAATTGTGTTGACACAGTCTCCAGGCACC |
| | | CTGTCTTTGTCTCCAGGGGAAACAGCCATCATCTCTTGTCGGACCAGTCAGT |
| | | ATGGTTCCTTAGCCTGGTATCAACAGAGGCCCGGCCAGGCCCCCAGGCTCG |
| | | TCATCTATTCGGGCTCTACTCGGGCCGCTGGCATCCCAGACAGGTTCAGCGG |
| | | CAGTCGGTGGGGGCCAGACTACAATCTCACCATCAGCAACCTGGAGTCGGG |
| | | AGATTTTGGTGTTTATTATTGCCAGCAGTATGAATTTTTTGGCCAGGGGACC |
| | | AAGGTCCAGGTCGACATTAAGCGAGAATTCGTGGCTGCACCATCTGTCTTC |
| | | ATCTTCCCGCCATCTGATGAGCAGTTGAAATCTGGAACTGCCTCTGTTGTGT |
| | | GCCTGCTGAATAACTTCTATCCCAGAGAGGCCAAAGTACAGTGGAAGGTGG |
| | | ATAACGCCCTCCAATCGGGTAACTCCCAGGAGAGTGTCACAGAGCAGGACA |
| | | GCAAGGACAGCACCTACAGCCTCAGCAGCACCCTGACGCTGAGCAAAGCA |
| | | GACTACGAGAAACACAAAGTCTACGCCTGCGAAGTCACCCATCAGGGCCTG |
| | | AGCTCGCCCGTCACAAAGAGCTTCAACAGGGGAGAGTGTTAG |
| 3 | 3BNC117 (IL-2 | ATGTACAGGATGCAACTCCTGTCTTGCATTGCACTAAGTCTTGCACTTGTCA |
| | secretory | CGCAGGTCCAATTGTTACAGTCTGGGGCAGCGGTGACGAAGCCCGGGGCCT |
| | sequence)-HV- | CAGTGAGAGTCTCCTGCGAGGCTTCTGGATACAACATTCGTGACTACTTTAT |
| | CH-T2A-LV- | TCATTGGTGGCGACAGGCCCCAGGACAGGGCCTTCAGTGGGTGGGATGGAT |
| | CL | CAATCCTAAGACAGGTCAGCCAAACAATCCTCGTCAATTTCAGGGTAGAGT |
| | | CAGTCTGACTCGACACGCGTCGTGGGACTTTGACACATTTTCCTTTTACATG |
| | | GACCTGAAGGCACTAAGATCGGACGACACGGCCGTTTATTTCTGTGCGCGA |
| | | CAGCGCAGCGACTATTGGGATTTCGACGTCTGGGGCAGTGGAACCCAGGTC |
| | | ACTGTCTCGTCAGCGTCGACCAAGGGCCCAGCTAGCACCAAGGGCCCATCG |
| | | GTCTTCCCCCTGGCACCCTCCTCCAAGAGCACCTCTGGGGGCACAGCGGCCC |
| | | TGGGCTGCCTGGTCAAGGACTACTTCCCCGAACCGGTGACGGTGTCGTGGA |
| | | ACTCAGGCGCCCTGACCAGCGGCGTGCACACCTTCCCGGCTGTCCTACAGTC |
| | | CTCAGGACTCTACTCCCTCAGCAGCGTGGTGACCGTGCCCTCCAGCAGCTTG |
| | | GGCACCCAGACCTACATCTGCAACGTGAATCACAAGCCCAGCAACACCAAG |
| | | GTGGACAAGAAAGTTGAGCCCAAATCTTGTGACAAAACTCACACATGCCCA |
| | | CCGTGCCCAGCACCTGAACTCCTGGGGGGACCGTCAGTCTTCCTCTTCCCCC |
| | | CAAAACCCAAGGACACCCTCATGATCTCCCGGACCCCTGAGGTCACATGCG |
| | | TGGTGGTGGACGTGAGCCACGAAGACCCTGAGGTCAAGTTCAACTGGTACG |
| | | TGGACGGCGTGGAGGTGCATAATGCCAAGACAAAGCCGCGGGAGGAGCAG |
| | | TACAACAGCACGTACCGTGTGGTCAGCGTCCTCACCGTCCTGCACCAGGAC |
| | | TGGCTGAATGGCAAGGAGTACAAGTGCAAGGTCTCCAACAAAGCCCTCCCA |
| | | GCCCCCATCGAGAAAACCATCTCCAAAGCCAAAGGGCAGCCCCGAGAACC |
| | | ACAGGTGTACACCCTGCCCCCATCCCGGGATGAGCTGACCAAGAACCAGGT |
| | | CAGCCTGACCTGCCTGGTCAAAGGCTTCTATCCCAGCGACATCGCCGTGGA |
| | | GTGGGAGAGCAATGGGCAGCCGGAGAACAACTACAAGACCACGCCTCCCG |
| | | TGCTGGACTCCGACGGCTCCTTCTTCCTCTACAGCAAGCTCACCGTGGACAA |
| | | GAGCAGGTGGCAGCAGGGGAACGTCTTCTCATGCTCCGTGATGCATGAGGC |
| | | TCTGCACAACCACTACACGCAGAAGAGCCTCTCCCTGTCTCCGGGTAAACG |
| | | TAGACGAAAGCGCGGAAGCGGAGAGGGCAGAGGAAGTCTGCTAACATGCG |
| | | GTGACGTCGAGGAGAATCCTGGACCTGGATCCATGTACAGGATGCAACTCC |
| | | TGTCTTGCATTGCACTAAGTCTTGCACTTGTCACGGACATCCAGATGACCCA |
| | | GTCTCCATCCTCCCTGTCTGCCTCTGTGGGAGATACCGTCACTATCACTTGC |
| | | CAGGCAAACGGCTACTTAAATTGGTATCAACAGAGGCGAGGGAAAGCCCC |
| | | AAAACTCCTGATCTACGATGGGTCCAAATTGGAAAGAGGGGTCCCATCAAG |
| | | GTTCAGTGGAAGAAGATGGGGGCAAGAATATAATCTGACCATCAACAATCT |
| | | GCAGCCCGAAGACATTGCAACATATTTTTGTCAAGTGTATGAGTTTGTCGTC |
| | | CCTGGGACCAGACTGGATTTGAAACGTACGGTGGCTGCACCAGAATTCGTG |
| | | GCTGCACCATCTGTCTTCATCTTCCCGCCATCTGATGAGCAGTTGAAATCTG |
| | | GAACTGCCTCTGTTGTGTGCCTGCTGAATAACTTCTATCCCAGAGAGGCCAA |
| | | AGTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAG |
| | | TCACCCATCAGGGCCTGAGCTCGCCCGTCACAAAGAGCTTCAACAGGGGAG |
| | | AGTGTTAG |
| 4 | CMV- | ACTAGTATTATGCCCAGTACATGACCTTATGGGACTTTCCTACTTGGCAGTA |
| | 3BNC117 (IL-2 | CATCTACGTATTAGTCATCGCTATTACCATGGTGATGCGGTTTTGGCAGTAC |
| | secretory | ATCAATGGGCGTGGATAGCGGTTTGACTCACGGGGATTTCCAAGTCTCCAC |
| | sequence)-HV- | CCCATTGACGTCAATGGGAGTTTGTTTTGGCACCAAAATCAACGGGACTTTC |
| | CH-T2A-LV- | CAAAATGTCGTAACAACTCCGCCCCATTGACGCAAATGGGCGGTAGGCGTG |
| | CL (AGT112) | TACGGTGGGAGGTCTATATAAGCAGAGCTCGTTTAGTGAACCGTCAGATCG |
| | | CCTGGAGACGCCATCCACGCTGTTTTGACCTCCATAGAAGATTCTAGATCTC |
| | | GAGGCCACCATGTACAGGATGCAACTCCTGTCTTGCATTGCACTAAGTCTTG |
| | | CACTTGTCACGCAGGTCCAATTGTTACAGTCTGGGGCAGCGGTGACGAAGC |
| | | CCGGGGCCTCAGTGAGAGTCTCCTGCGAGGCTTCTGGATACAACATTCGTG |
| | | ACTACTTTATTCATTGGTGGCGACAGGCCCCAGGACAGGGCCTTCAGTGGG |
| | | TGGGATGGATCAATCCTAAGACAGGTCAGCCAAACAATCCTCGTCAATTTC |
| | | AGGGTAGAGTCAGTCTGACTCGACACGCGTCGTGGGACTTTGACACATTTTC |
| | | CTTTTACATGGACCTGAAGGCACTAAGATCGGACGACACGGCCGTTTATTTC |
| | | TGTGCGCGACAGCGCAGCGACTATTGGGATTTCGACGTCTGGGGCAGTGGA |
| | | ACCCAGGTCACTGTCTCGTCAGCGTCGACCAAGGGCCCAGCTAGCACCAAG |
| | | GGCCCATCGGTCTTCCCCCTGGCACCCTCCTCCAAGAGCACCTCTGGGGGCA |
| | | CAGCGGCCCTGGGCTGCCTGGTCAAGGACTACTTCCCCGAACCGGTGACGG |
| | | TGTCGTGGAACTCAGGCGCCCTGACCAGCGGCGTGCACACCTTCCCGGCTG |
| | | TCCTACAGTCCTCAGGACTCTACTCCCTCAGCAGCGTGGTGACCGTGCCCTC |
| | | CAGCAGCTTGGGCACCCAGACCTACATCTGCAACGTGAATCACAAGCCCAG |
| | | CAACACCAAGGTGGACAAGAAAGTTGAGCCCAAATCTTGTGACAAAACTCA |
| | | CACATGCCCACCGTGCCCAGCACCTGAACTCCTGGGGGGACCGTCAGTCTT |
| | | CCTCTTCCCCCCAAAACCCAAGGACACCCTCATGATCTCCCGGACCCCTGAG |
| | | GTCACATGCGTGGTGGTGGACGTGAGCCACGAAGACCCTGAGGTCAAGTTC |
| | | AACTGGTACGTGGACGGCGTGGAGGTGCATAATGCCAAGACAAAGCCGCG |
| | | GGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACA |
| | | AAGCCCTCCCAGCCCCCATCGAGAAAACCATCTCCAAAGCCAAAGGGCAGC |
| | | CCCGAGAACCACAGGTGTACACCCTGCCCCCATCCCGGGATGAGCTGACCA |
| | | AGAACCAGGTCAGCCTGACCTGCCTGGTCAAAGGCTTCTATCCCAGCGACA |
| | | TCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCA |
| | | CCGTGGACAAGAGCAGGTGGCAGCAGGGGAACGTCTTCTCATGCTCCGTGA |
| | | TGCATGAGGCTCTGCACAACCACTACACGCAGAAGAGCCTCTCCCTGTCTCC |
| | | GGGTAAACGTAGACGAAAGCGCGGAAGCGGAGAGGGCAGAGGAAGTCTGC |
| | | TAACATGCGGTGACGTCGAGGAGAATCCTGGACCTGGATCCATGTACAGGA |
| | | TGCAACTCCTGTCTTGCATTGCACTAAGTCTTGCACTTGTCACGGACATCCA |
| | | GATGACCCAGTCTCCATCCTCCCTGTCTGCCTCTGTGGGAGATACCGTCACT |
| | | ATCACTTGCCAGGCAAACGGCTACTTAAATTGGTATCAACAGAGGCGAGGG |
| | | AAAGCCCCAAAACTCCTGATCTACGATGGGTCCAAATTGGAAAGAGGGGTC |
| | | CCATCAAGGTTCAGTGGAAGAAGATGGGGGCAAGAATATAATCTGACCATC |
| | | AACAATCTGCAGCCCGAAGACATTGCAACATATTTTTGTCAAGTGTATGAGT |
| | | TTGTCGTCCCTGGGACCAGACTGGATTTGAAACGTACGGTGGCTGCACCAG |
| | | AATTCGTGGCTGCACCATCTGTCTTCATCTTCCCGCCATCTGATGAGCAGTT |
| | | GAAATCTGGAACTGCCTCTGTTGTGTGCCTGCTGAATAACTTCTATCCCAGA |
| | | GAGGCCAAAGTACAGTGGAAGGTGGATAACGCCCTCCAATCGGGTAACTCC |
| | | CAGGAGAGTGTCACAGAGCAGGACAGCAAGGACAGCACCTACAGCCTCAG |
| | | CAGCACCCTGACGCTGAGCAAAGCAGACTACGAGAAACACAAAGTCTACG |
| | | CCTGCGAAGTCACCCATCAGGGCCTGAGCTCGCCCGTCACAAAGAGCTTCA |
| | | ACAGGGGAGAGTGTTAG |
| 5 | VRC01 | ATGGGATGGTCATGTATCATCCTTTTTCTAGTAGCAACTGCAACTGGTGTAC |
| | (Antibody | ATTCCCAGGTGCAGCTGGTGCAGTCTGGGGGTCAGATGAAGAAGCCTGGCG |
| | secretory | AGTCGATGAGAATTTCTTGTCGGGCTTCTGGATATGAATTTATTGATTGTAC |
| | sequence)-HV- | GCTAAATTGGATTCGTCTGGCCCCCGGAAAAAGGCCTGAGTGGATGGGATG |
| | CH-T2A-LV- | GCTGAAGCCTCGGGGGGGGGCCGTCAACTACGCACGTCCACTTCAGGGCAG |
| | CL | AGTGACCATGACACGAGACGTTTATTCCGACACAGCCTTTTTGGAGCTGCGC |
| | | TCGTTGACAGTAGACGACACGGCCGTCTACTTTTGTACTAGGGGAAAAAAC |
| | | TGTGATTACAATTGGGACTTCGAACACTGGGGCCGGGGCACCCCGGTCATC |
| | | GTCTCATCAGCTAGCACCAAGGGCCCATCGGTCTTCCCCCTGGCACCCTCCT |
| | | CCAAGAGCACCTCTGGGGGCACAGCGGCCCTGGGCTGCCTGGTCAAGGACT |
| | | ACTTCCCCGAACCGGTGACGGTGTCGTGGAACTCAGGCGCCCTGACCAGCG |
| | | GCGTGCACACCTTCCCGGCTGTCCTACAGTCCTCAGGACTCTACTCCCTCAG |
| | | CAGCGTGGTGACCGTGCCCTCCAGCAGCTTGGGCACCCAGACCTACATCTG |
| | | CAACGTGAATCACAAGCCCAGCAACACCAAGGTGGACAAGAAAGTTGAGC |
| | | CCAAATCTTGTGACAAAACTCACACATGCCCACCGTGCCCAGCACCTGAAC |
| | | TCCTGGGGGGACCGTCAGTCTTCCTCTTCCCCCCAAAACCCAAGGACACCCT |
| | | CATGATCTCCCGGACCCCTGAGGTCACATGCGTGGTGGTGGACGTGAGCCA |
| | | CGAAGACCCTGAGGTCAAGTTCAACTGGTACGTGGACGGCGTGGAGGTGCA |
| | | TAATGCCAAGACAAAGCCGCGGGAGGAGCAGTACAACAGCACGTACCGTG |
| | | TGGTCAGCGTCCTCACCGTCCTGCACCAGGACTGGCTGAATGGCAAGGAGT |
| | | ACAAGTGCAAGGTCTCCAACAAAGCCCTCCCAGCCCCCATCGAGAAAACCA |
| | | TCTCCAAAGCCAAAGGGCAGCCCCGAGAACCACAGGTGTACACCCTGCCCC |
| | | CATCCCGGGATGAGCTGACCAAGAACCAGGTCAGCCTGACCTGCCTGGTCA |
| | | AAGGCTTCTATCCCAGCGACATCGCCGTGGAGTGGGAGAGCAATGGGCAGC |
| | | CGGAGAACAACTACAAGACCACGCCTCCCGTGCTGGACTCCGACGGCTCCT |
| | | TCTTCCTCTACAGCAAGCTCACCGTGGACAAGAGCAGGTGGCAGCAGGGGA |
| | | ACGTCTTCTCATGCTCCGTGATGCATGAGGCTCTGCACAACCACTACACGCA |
| | | GAAGAGCCTCTCCCTGTCTCCGGGTAAACGTAGACGAAAGCGCGGAAGCGG |
| | | AGAGGGCAGAGGAAGTCTGCTAACATGCGGTGACGTCGAGGAGAATCCTG |
| | | GACCTGGATCCATGGGATGGTCATGTATCATCCTTTTTCTAGTAGCAACTGC |
| | | AACTGGTGTACATTCCGAAATTGTGTTGACACAGTCTCCAGGCACCCTGTCT |
| | | TTGTCTCCAGGGGAAACAGCCATCATCTCTTGTCGGACCAGTCAGTATGGTT |
| | | CCTTAGCCTGGTATCAACAGAGGCCCGGCCAGGCCCCCAGGCTCGTCATCT |
| | | ATTCGGGCTCTACTCGGGCCGCTGGCATCCCAGACAGGTTCAGCGGCAGTC |
| | | GGTGGGGGCCAGACTACAATCTCACCATCAGCAACCTGGAGTCGGGAGATT |
| | | TTGGTGTTTATTATTGCCAGCAGTATGAATTTTTTGGCCAGGGGACCAAGGT |
| | | CCAGGTCGACATTAAGCGAGAATTCGTGGCTGCACCATCTGTCTTCATCTTC |
| | | CCGCCATCTGATGAGCAGTTGAAATCTGGAACTGCCTCTGTTGTGTGCCTGC |
| | | TGAATAACTTCTATCCCAGAGAGGCCAAAGTACAGTGGAAGGTGGATAACG |
| | | CCCTCCAATCGGGTAACTCCCAGGAGAGTGTCACAGAGCAGGACAGCAAGG |
| | | ACAGCACCTACAGCCTCAGCAGCACCCTGACGCTGAGCAAAGCAGACTACG |
| | | AGAAACACAAAGTCTACGCCTGCGAAGTCACCCATCAGGGCCTGAGCTCGC |
| | | CCGTCACAAAGAGCTTCAACAGGGGAGAGTGTTAG |
| 6 | CMV-VRC01 | ACTAGTATTATGCCCAGTACATGACCTTATGGGACTTTCCTACTTGGCAGTA |
| | (Antibody | CATCTACGTATTAGTCATCGCTATTACCATGGTGATGCGGTTTTGGCAGTAC |
| | secretory | ATCAATGGGCGTGGATAGCGGTTTGACTCACGGGGATTTCCAAGTCTCCAC |
| | sequence)-HV- | CCCATTGACGTCAATGGGAGTTTGTTTTGGCACCAAAATCAACGGGACTTTC |
| | CH-T2A-LV- | CAAAATGTCGTAACAACTCCGCCCCATTGACGCAAATGGGCGGTAGGCGTG |
| | CL (AGT113) | TACGGTGGGAGGTTTATATAAGCAGAGCTCGTTTAGTGAACCGTCAGATCG |
| | | CCTGGAGACGCCATCCACGCTGTTTTGACCTCCATAGAAGATTCTAGATCTC |
| | | GAGGCCACCATGGGATGGTCATGTATCATCCTTTTTCTAGTAGCAACTGCAA |
| | | CTGGTGTACATTCCCAGGTGCAGCTGGTGCAGTCTGGGGGTCAGATGAAGA |
| | | AGCCTGGCGAGTCGATGAGAATTTCTTGTCGGGCTTCTGGATATGAATTTAT |
| | | TGATTGTACGCTAAATTGGATTCGTCTGGCCCCCGGAAAAAGGCCTGAGTG |
| | | GATGGGATGGCTGAAGCCTCGGGGGGGGGCCGTCAACTACGCACGTCCACT |
| | | TCAGGGCAGAGTGACCATGACACGAGACGTTTATTCCGACACAGCCTTTTT |
| | | GGAGCTGCGCTCGTTGACAGTAGACGACACGGCCGTCTACTTTTGTACTAG |
| | | GGGAAAAAACTGTGATTACAATTGGGACTTCGAACACTGGGGCCGGGGCAC |
| | | CCCGGTCATCGTCTCATCAGCTAGCACCAAGGGCCCATCGGTCTTCCCCCTG |
| | | GCACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCGCC |
| | | CTGACCAGCGGCGTGCACACCTTCCCGGCTGTCCTACAGTCCTCAGGACTCT |
| | | ACTCCCTCAGCAGCGTGGTGACCGTGCCCTCCAGCAGCTTGGGCACCCAGA |
| | | CCTACATCTGCAACGTGAATCACAAGCCCAGCAACACCAAGGTGGACAAGA |
| | | AAGTTGAGCCCAAATCTTGTGACAAAACTCACACATGCCCACCGTGCCCAG |
| | | CACCTGAACTCCTGGGGGGACCGTCAGTCTTCCTCTTCCCCCCAAAACCCAA |
| | | GGACACCCTCATGATCTCCCGGACCCCTGAGGTCACATGCGTGGTGGTGGA |
| | | CGTGAGCCACGAAGACCCTGAGGTCAAGTTCAACTGGTACGTGGACGGCGT |
| | | GGAGGTGCATAATGCCAAGACAAAGCCGCGGGAGGAGCAGTACAACAGCA |
| | | CGTACCGTGTGGTCAGCGTCCTCACCGTCCTGCACCAGGACTGGCTGAATG |
| | | GCAAGGAGTACAAGTGCAAGGTCTCCAACAAAGCCCTCCCAGCCCCCATCG |
| | | AGAAAACCATCTCCAAAGCCAAAGGGCAGCCCCGAGAACCACAGGTGTAC |
| | | ACCCTGCCCCCATCCCGGGATGAGCTGACCAAGAACCAGGTCAGCCTGACC |
| | | TGCCTGGTCAAAGGCTTCTATCCCAGCGACATCGCCGTGGAGTGGGAGAGC |
| | | AATGGGCAGCCGGAGAACAACTACAAGACCACGCCTCCCGTGCTGGACTCC |
| | | GACGGCTCCTTCTTCCTCTACAGCAAGCTCACCGTGGACAAGAGCAGGTGG |
| | | CAGCAGGGGAACGTCTTCTCATGCTCCGTGATGCATGAGGCTCTGCACAAC |
| | | CACTACACGCAGAAGAGCCTCTCCCTGTCTCCGGGTAAACGTAGACGAAAG |
| | | CGCGGAAGCGGAGAGGGCAGAGGAAGTCTGCTAACATGCGGTGACGTCGA |
| | | GGAGAATCCTGGACCTGGATCCATGGGATGGTCATGTATCATCCTTTTTCTA |
| | | GTAGCAACTGCAACTGGTGTACATTCCGAAATTGTGTTGACACAGTCTCCAG |
| | | GCACCCTGTCTTTGTCTCCAGGGGAAACAGCCATCATCTCTTGTCGGACCAG |
| | | TCAGTATGGTTCCTTAGCCTGGTATCAACAGAGGCCCGGCCAGGCCCCCAG |
| | | GCTCGTCATCTATTCGGGCTCTACTCGGGCCGCTGGCATCCCAGACAGGTTC |
| | | AGCGGCAGTCGGTGGGGGCCAGACTACAATCTCACCATCAGCAACCTGGAG |
| | | TCGGGAGATTTTGGTGTTTATTATTGCCAGCAGTATGAATTTTTTGGCCAGG |
| | | GGACCAAGGTCCAGGTCGACATTAAGCGAGAATTCGTGGCTGCACCATCTG |
| | | TCTTCATCTTCCCGCCATCTGATGAGCAGTTGAAATCTGGAACTGCCTCTGT |
| | | TGTGTGCCTGCTGAATAACTTCTATCCCAGAGAGGCCAAAGTACAGTGGAA |
| | | GGTGGATAACGCCCTCCAATCGGGTAACTCCCAGGAGAGTGTCACAGAGCA |
| | | GGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGG |
| | | GCCTGAGCTCGCCCGTCACAAAGAGCTTCAACAGGGGAGAGTGTTAG |
| 7 | sCD4(D1 + D2) | ATGGGATGGTCATGTATCATCCTTTTTCTAGTAGCAACTGCAACTGGTGTAC |
| | | ATTCCAAGAAAGTGGTGCTGGGCAAAAAAGGGGATACAGTGGAACTGACC |
| | | TGCACAGCTTCCCAGAAGAAGAGCATACAATTCCACTGGAAAAACTCCAAC |
| | | CAGATAAAGATTCTGGGAAATCAGGGCTCCTTCTTAACTAAAGGTCCATCC |
| | | AAGCTGAATGATCGCGCTGACTCAAGAAGAAGCCTTTGGGACCAAGGAAAC |
| | | TTTCCCCTGATCATCAAGAATCTTAAGATAGAAGACTCAGATACTTACATCT |
| | | GTGAAGTGGAGGACCAGAAGGAGGAGGTGCAATTGCTAGTGTTCGGATTGA |
| | | CTGCCAACTCTGACACCCACCTGCTTCAGGGGCAGAGCCTGACCCTGACCTT |
| | | GGAGAGCCCCCCTGGTAGTAGCCCCTCAGTGCAATGTAGGAGTCCAAGGGG |
| | | TAAAAACATACAGGGGGGGAAGACCCTCTCCGTGTCTCAGCTGGAGCTCCA |
| | | GGATAGTGGCACCTGGACATGCACTGTCTTGCAGAACCAGAAGAAGGTGGA |
| | | GTTCAAAATAGACATCGTGGTGCTAGCTTGA |
| 8 | EF-1- | CCGGTGCCTAGAGAAGGTGGCGCGGGGTAAACTGGGAAAGTGATGTCGTGT |
| | sCD4(D1 + D2) | ACTGGCTCCGCCTTTTTCCCGAGGGTGGGGGAGAACCGTATATAAGTGCAG |
| | (AGT116) | TAGTCGCCGTGAACGTTCTTTTTCGCAACGGGTTTGCCGCCAGAACACAGGT |
| | | AAGTGCCGTGTGTGGTTCCCGCGGGCCTGGCCTCTTTACGGGTTATGGCCCT |
| | | TGCGTGCCTTGAATTACTTCCACGCCCCTGGCTGCAGTACGTGATTCTTGAT |
| | | CCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGC |
| | | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAGCCACCATGGGATGGTCATGTATCA |
| | | TCCTTTTTCTAGTAGCAACTGCAACTGGTGTTCATTCCAAGAAAGTGGTGCT |
| | | GGGCAAAAAAGGGGATACAGTGGAACTGACCTGCACAGCTTCCCAGAAGA |
| | | AGAGCATACAATTCCACTGGAAAAACTCCAACCAGATAAAGATTCTGGGAA |
| | | ATCAGGGCTCCTTCTTAACTAAAGGTCCATCCAAGCTGAATGATCGCGCTGA |
| | | CTCAAGAAGAAGCCTTTGGGACCAAGGAAACTTTCCCCTGATCATCAAGAA |
| | | TCTTAAGATAGAAGACTCAGATACTTACATCTGTGAAGTGGAGGACCAGAA |
| | | GGAGGAGGTGCAATTGCTAGTGTTCGGATTGACTGCCAACTCTGACACCCA |
| | | CCTGCTTCAGGGGCAGAGCCTGACCCTGACCTTGGAGAGCCCCCCTGGTAG |
| | | TAGCCCCTCAGTGCAATGTAGGAGTCCAAGGGGTAAAAACATACAGGGGG |
| | | GGAAGACCCTCTCCGTGTCTCAGCTGGAGCTCCAGGATAGTGGCACCTGGA |
| | | CATGCACTGTCTTGCAGAACCAGAAGAAGGTGGAGTTCAAAATAGACATCG |
| | | TGGTGCTAGCTTGA |
| 9 | sCD4(D1 + D2)- | ATGGGATGGTCATGTATCATCCTTTTTCTAGTAGCAACTGCAACTGGTGTAC |
| | IgG1 Fc | ATTCCAAGAAAGTGGTGCTGGGCAAAAAAGGGGATACAGTGGAACTGACC |
| | | TGCACAGCTTCCCAGAAGAAGAGCATACAATTCCACTGGAAAAACTCCAAC |
| | | CAGATAAAGATTCTGGGAAATCAGGGCTCCTTCTTAACTAAAGGTCCATCC |
| | | AAGCTGAATGATCGCGCTGACTCAAGAAGAAGCCTTTGGGACCAAGGAAAC |
| | | TTTCCCCTGATCATCAAGAATCTTAAGATAGAAGACTCAGATACTTACATCT |
| | | GTGAAGTGGAGGACCAGAAGGAGGAGGTGCAATTGCTAGTGTTCGGATTGA |
| | | CTGCCAACTCTGACACCCACCTGCTTCAGGGGCAGAGCCTGACCCTGACCTT |
| | | GGAGAGCCCCCCTGGTAGTAGCCCCTCAGTGCAATGTAGGAGTCCAAGGGG |
| | | TAAAAACATACAAGGTGGTAAGACCCTCTCCGTGTCTCAGCTGGAGCTCCA |
| | | GGATAGTGGCACCTGGACATGCACTGTCTTGCAGAACCAGAAGAAGGTGGA |
| | | GTTCAAAATAGACATCGTGGTGCTAGCTGCTGCAGATCCGGAGCCCAAGAG |
| | | CTGCGACAAGACCCACACCTGTCCACCATGCCCCGCCCACCTGAACTCCTG |
| | | GGGGGACCGTCAGTCTTCCTCTTCCCCCCAAAACCCAAGGACACCCTCATG |
| | | ATCTCCCGGACCCCTGAGGTCACATGCGTGGTGGTGGACGTGAGCCACGAA |
| | | GACCCTGAGGTCAAGTTCAACTGGTACGTGGACGGCGTGGAGGTGCATAAT |
| | | GCCAAGACAAAGCCGCGGGAGGAGCAGTACAACAGCACGTACCGTGTGGT |
| | | CAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTC |
| | | CAAAGCCAAAGGTGGGACCCGTGGGGTGCGAGGGCCACATGGACAGAGGC |
| | | CGGCTCGGCCCACCCTCTGCCCTGAGAGTGACCGCTGTACCAACCTCTGTCC |
| | | CTACAGGGCAGCCCCGAGAACCACAGGTCTACACCCTGCCCCCATCCCGGG |
| | | AGGAGATGACCAAGAACCAGGTCAGCCTGACCTGCCTGGTCAAAGGCTTCT |
| | | ATCCCAGCGACATCGCCGTGGAGTGGGAGAGCAATGGGCAGCCGGAGAAC |
| | | AACTACAAGACCACGCCTCCCGTGCTGGACTCCGACGGCTCCTTCTTCCTCT |
| | | ATAGCAAGCTCACCGTGGACAAGAGCAGGTGGCAGCAGGGGAACGTCTTCT |
| | | CATGCTCCGTGATGCATGAGGCTCTGCACAACCACTACACGCAGAAGAGCC |
| | | TCTCCCTGTCCCCGGGTAAATGA |

| | | |
|---|---|---|
| 10 | EF-1α | CCGGTGCCTAGAGAAGGTGGCGCGGGGTAAACTGGGAAAGTGATGTCGTGT |
| | promoter- | ACTGGCTCCGCCTTTTTCCCGAGGGTGGGGGAGAACCGTATATAAGTGCAG |
| | sCD4(D1 + D2)- | TAGTCGCCGTGAACGTTCTTTTTCGCAACGGGTTTGCCGCCAGAACACAGGT |
| | IgG1 Fc fusion | AAGTGCCGTGTGTGGTTCCCGCGGGCCTGGCCTCTTTACGGGTTATGGCCCT |
| | protein | TGCGTGCCTTGAATTACTTCCACGCCCCTGGCTGCAGTACGTGATTCTTGAT |
| | (truncated SEQ | CCCGAGCTTCGGGTTGGAAGTGGGTGGGAGAGTTCGAGGCCTTGCGCTTAA |
| | ID NO: 9), | GGAGCCCCTTCGCCTCGTGCTTGAGTTGAGGCCTGGCCTGGGCGCTGGGGC |
| | antibody | CGCCGCGTGCGAATCTGGTGGCACCTTCGCGCCTGTCTCGCTGCTTTCGATA |
| | secretion | AGTCTCTAGCCATTTAAAATTTTTGATGACCTGCTGCGACGCTTTTTTTCTGG |
| | signal(AGT117) | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAGCCACCATGGGATGGTCATGTATCA |
| | | TCCTTTTTCTAGTAGCAACTGCAACTGGTGTACATTCCAAGAAAGTGGTGCT |
| | | GGGCAAAAAAGGGGATACAGTGGAACTGACCTGCACAGCTTCCCAGAAGA |
| | | AGAGCATACAATTCCACTGGAAAAACTCCAACCAGATAAAGATTCTGGGAA |
| | | ATCAGGGCTCCTTCTTAACTAAAGGTCCATCCAAGCTGAATGATCGCGCTGA |
| | | CTCAAGAAGAAGCCTTTGGGACCAAGGAAACTTTCCCCTGATCATCAAGAA |
| | | TCTTAAGATAGAAGACTCAGATACTTACATCTGTGAAGTGGAGGACCAGAA |
| | | GGAGGAGGTGCAATTGCTAGTGTTCGGATTGACTGCCAACTCTGACACCCA |
| | | CCTGCTTCAGGGGCAGAGCCTGACCCTGACCTTGGAGAGCCCCCCTGGTAG |
| | | TAGCCCCTCAGTGCAATGTAGGAGTCCAAGGGGTAAAAACATACAAGGTGG |
| | | TAAGACCCTCTCCGTGTCTCAGCTGGAGCTCCAGGATAGTGGCACCTGGAC |
| | | ATGCACTGTCTTGCAGAACCAGAAGAAGGTGGAGTTCAAAATAGACATCGT |
| | | GGTGCTAGCTGCTGCAGATCCGGAGCCCAAGAGCTGCGACAAGACCCACAC |
| | | CTGTCCACCATGCCCCGCCCACCTGAACTCCTGGGGGGACCGTCAGTCTTCC |
| | | TCTTCCCCCCAAAACCCAAGGACACCCTCATGATCTCCCGGACCCCTGAGGT |
| | | CACATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGGG |
| | | AGGAGCAGTACAACAGCACGTACCGTGTGGTCAGCGTCCTCACCGTCCTGC |
| | | ACCAGGACTGGCTGAATGGCAAGGAGTACAAGTGCAAGGTCTCCAACAAA |
| | | GCCCTCCCAGCCCCCATCGAGAAAACCATCTCCAAAGCCAAAGGTGGGACC |
| | | CGTGGGGTGCGAGGGCCACATGGACAGAGGCCGGCTCGGCCCACCCTCTGC |
| | | CCTGAGAGTGACCGCTGTACCAACCTCTGTCCCTACAGGGCAGCCCCGAGA |
| | | ACCACAGGTCTACACCCTGCCCCCATCCCGGGAGGAGATGACCAAGAACCA |
| | | GGTCAGCCTGACCTGCCTGGTCAAAGGCTTCTATCCCAGCGACATCGCCGTG |
| | | GAGTGGGAGAGCAATGGGCAGCCGGAGAACAACTACAAGACCACGCCTCC |
| | | CGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAG |
| | | GCTCTGCACAACCACTACACGCAGAAGAGCCTCTCCCTGTCCCCGGGTAAA |
| | | TGA |
| 11 | IL-2 secretory | ATGTACAGGATGCAACTCCTGTCTTGCATTGCACTAAGTCTTGCACTTGTCA |
| | signal | CG |
| 12 | Antibody | ATGGGATGGTCATGTATCATCCTTTTTCTAGTAGCAACTGCAACTGGTGTAC |
| | secretory signal | ATTCC |
| 13 | Cytomegalovirus | ACTAGTATTATGCCCAGTACATGACCTTATGGGACTTTCCTACTTGGCAGTA |
| | (CMV) | CATCTACGTATTAGTCATCGCTATTACCATGGTGATGCGGTTTTGGCAGTAC |
| | promoter | ATCAATGGGCGTGGATAGCGGTTTGACTCACGGGGATTTCCAAGTCTCCAC |
| | | CCCATTGACGTCAATGGGAGTTTGTTTTGGCACCAAAATCAACGGGACTTTC |
| | | CAAAATGTCGTAACAACTCCGCCCCATTGACGCAAATGGGCGGTAGGCGTG |
| | | TACGGTGGGAGGTTTATATAAGCAGAGCTCGTTTAGTGAACCGTCAGATCG |
| | | CCTGGAGACGCCATCCACGCTGTTTTGACCTCCATAGAAGA |
| 14 | Human | CCGGTGCCTAGAGAAGGTGGCGCGGGGTAAACTGGGAAAGTGATGTCGTGT |
| | elongation | ACTGGCTCCGCCTTTTTCCCGAGGGTGGGGGAGAACCGTATATAAGTGCAG |
| | factor 1 alpha | TAGTCGCCGTGAACGTTCTTTTTCGCAACGGGTTTGCCGCCAGAACACAGGT |
| | (EF-1α) | AAGTGCCGTGTGTGGTTCCCGCGGGCCTGGCCTCTTTACGGGTTATGGCCCT |
| | promoter | TGCGTGCCTTGAATTACTTCCACGCCCCTGGCTGCAGTACGTGATTCTTGAT |
| | | CCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGC |
| | | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGA |
| 15 | Interferon | TGTATTTCTACTGGGCAGTGCTGATCTAGAGCAATTTGAAACTTGTGGTAGA |
| | gamma (IFNγ) | TATTTTACTAACCAACTCTGATGAAGGACTTCCTCACCAAATTGTTCTTTTA |
| | promoter | ACCGCATTCTTTCCTTGCTTTCTGGTCATTTGCAAGAAAAATTTTAAAAGGC |
| | | TGCCCCTTTGTAAAGGTTTGAGAGGCCCTAGAATTTCGTTTTTCACTTGTTCC |
| | | CAACCACAAGCAAATGATCAATGTGCTTTGTGAATGAAGAGTCAACATTTT |
| | | ACCAGGGCGAAGTGGGGAGGTACAAAAAAATTTCCAGTCCTTGAATGGTGT |
| | | GAAGTAAAAGTGCCTTCAAAGAATCCCACCAGAATGGCACAGGTGGGCATA |
| | | ATGGGTCTGTCTCATCGTCAAAGGACCCAAGGAGTCTAAAGGAAACTCTAA |
| | | CTACAACACCCAAATGCCACAAAACCTTAGTTATTAATACAAACTATCATCC |
| | | CTGCCTATCTGTCACCATCTCATCTTAAAAAACTTGTGAAAATACGTAATCC |
| | | TCAGGAGACTTCAATTAGGTATAAATACCAGCAGCCAGAGGAGGTGCAGCA |
| | | CATTGTTCTGATCATCTGAAGATCAGCTATTAGAAGAGAAAGATCAG |
| 16 | Prothrombin/ | GCGAGAACTTGTGCCTCCCCGTGTTCCTGCTCTTTGTCCCTCTGTCCTACTTA |
| | Human alpha-1- | GACTAATATTTGCCTTGGGTACTGCAAACAGGAAATGGGGGAGGGACAGGA |
| | anti trypsin | GTAGGGCGGAGGGTAGCCCGGGGATCTTGCTACCAGTGGAACAGCCACTAA |
| | (hAAT) | GGATTCTGCAGTGAGAGCAGAGGGCCAGCTAAGTGGTACTCTCCCAGAGAC |
| | enhancer/promoter | TGTCTGACTCACGCCACCCCCTCCACCTTGGACACAGGACGCTGTGGTTTCT |
| | | GAGCCAGGTACAATGACTCCTTTCGGTAAGTGCAGTGGAAGCTGTACACTG |
| | | CCCAGGCAAAGCGTCCGGGCAGCGTAGGCGGGCGACTCAGATCCCAGCCA |
| | | GTGGACTTAGCCCCTGTTTGCTCCTCCGATAACTGGGGTGACCTTGGTTAAT |
| | | ATTCACCAGCAGCCTCCCCCGTTGCCCCTCTGGATCCACTGCTTAAATACGG |
| | | ACGAGGACAGGGCCCTGTCTCCTCAGCTTCAGGCACCACCACTGACCTGGG |
| | | ACAGTGAAT |
| 17 | Rous Sarcoma | GTAGTCTTATGCAATACTCTTGTAGTCTTGCAACATGGTAACGATGAGTTAG |
| | virus (RSV) | CAACATGCCTTACAAGGAGAGAAAAAGCACCGTGCATGCCGATTGGTGGAA |
| | promoter | GTAAGGTGGTACGATCGTGCCTTATTAGGAAGGCAACAGACGGGTCTGACA |
| | | TGGATTGGACGAACCACTGAATTGCCGCATTGCAGAGATATTGTATTTAAGT |
| | | GCCTAGCTCGATACAATAAACG |
| 18 | 5′ Long | GGTCTCTCTGGTTAGACCAGATCTGAGCCTGGGAGCTCTCTGGCTAACTAGG |
| | terminal repeat | GAACCCACTGCTTAAGCCTCAATAAAGCTTGCCTTGAGTGCTTCAAGTAGTG |
| | (LTR) | TGTGCCCGTCTGTTGTGTGACTCTGGTAACTAGAGATCCCTCAGACCCTTTT |
| | | AGTCAGTGTGGAAAATCTCTAGCA |
| 19 | Psi Packaging | TACGCCAAAAATTTTGACTAGCGGAGGCTAGAAGGAGAGAG |
| | signal | |
| 20 | Rev response | AGGAGCTTTGTTCCTTGGGTTCTTGGGAGCAGCAGGAAGCACTATGGGCGC |
| | element (RRE) | AGCCTCAATGACGCTGACGGTACAGGCCAGACAATTATTGTCTGGTATAGT |
| | | GCAGCAGCAGAACAATTTGCTGAGGGCTATTGAGGCGCAACAGCATCTGTT |
| | | GCAACTCACAGTCTGGGGCATCAAGCAGCTCCAGGCAAGAATCCTGGCTGT |
| | | GGAAAGATACCTAAAGGATCAACAGCTCC |
| 21 | Central | TTTTAAAAGAAAAGGGGGGATTGGGGGGTACAGTGCAGGGGAAAGAATAG |
| | polypurine tract | TAGACATAATAGCAACAGACATACAAACTAAAGAATTACAAAAACAAATT |
| | (cPPT) | ACAAAATTCAAAATTTTA |
| 22 | Long WPRE | AATCAACCTCTGATTACAAAATTTGTGAAAGATTGACTGGTATTCTTAACTA |
| | sequence | TGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGTGT |
| | | GCACTGTGTTTGCTGACGCAACCCCCACTGGTTGGGGCATTGCCACCACCTG |
| | | TCAGCTCCTTTCCGGGACTTTCGCTTTCCCCCTCCCTATTGCCACGGCGGAA |
| | | CTCATCGCCGCCTGCCTTGCCCGCTGCTGGACAGGGGCTCGGCTGTTGGGCA |
| | | CTGACAATTCCGTGGTGTTGTCGGGGAAATCATCGTCCTTTCCTTGGCTGCT |
| | | CGCCTGTGTTGCCACCTGGATTCTGCGCGGGACGTCCTTCTGCTACGTCCCT |
| | | TCGGCCCTCAATCCAGCGGACCTTCCTTCCCGCGGCCTGCTGCCGGCTCTGC |
| | | GGCCTCTTCCGCGTCTTCGCCTTCGCCCTCAGACGAGTCGGATCTCCCTTTG |
| | | GGCCGCCTCCCCGCCT |
| 23 | Short WPRE | AATCAACCTCTGGATTACAAAATTTGTGAAAGATTGACTGATATTCTTAACT |
| | sequence | ATGTTGCTCCTTTTACGCTGTGTGGATATGCTGCTTTAATGCCTCTGTATCAT |
| | | GCTATTGCTTCCCGTACGGCTTTCGTTTTCTCCTCCTTGTATAAATCCTGGTT |
| | | GCTGTCTCTTTATGAGGAGTTGTGGCCCGTTGTCCGTCAACGTGGCGTGGTG |
| | | TGCTCTGTGTTTGCTGACGCAACCCCCACTGGCTGGGGCATTGCCACCACCT |
| | | GTCAACTCCTTTCTGGGACTTTCGCTTTCCCCCTCCCGATCGCCACGGCAGA |
| | | ACTCATCGCCGCCTGCCTTGCCCGCTGCTGGACAGGGGCTAGGTTGCTGGGC |
| | | ACTGATAATTCCGTGGTGTTGTC |
| 24 | 3′ delta LTR | TGGAAGGGCTAATTCACTCCCAACGAAGATAAGATCTGCTTTTTGCTTGTAC |
| | | TGGGTCTCTCTGGTTAGACCAGATCTGAGCCTGGGAGCTCTCTGGCTAACTA |
| | | GGGAACCCACTGCTTAAGCCTCAATAAAGCTTGCCTTGAGTGCTTCAAGTA |
| | | GTGTGTGCCCGTCTGTTGTGTGACTCTGGTAACTAGAGATCCCTCAGACCCT |
| | | TTTAGTCAGTGTGGAAAATCTCTAGCAGTAGTAGTTCATGTCA |
| 25 | Helper/Rev; | GCTATTACCATGGGTCGAGGTGAGCCCCACGTTCTGCTTCACTCTCCCCATC |
| | Chicken beta | TCCCCCCCCTCCCCACCCCCAATTTTGTATTTATTTATTTTTTAATTATTTTGT |
| | actin (CAG) | GCAGCGATGGGGGCGGGGGGGGGGGGGGCGCGCGCCAGGCGGGGCGGGG |
| | promoter; | CGGGGCGAGGGGCGGGGCGGGGCGAGGCGGAGAGGTGCGGCGGCAGCCA |
| | | ATCAGAGCGGCGCGCTCCGAAAGTTTCCTTTTATGGCGAGGCGGCGGCGGC |
| | | GGCGGCCCTATAAAAAGCGAAGCGCGCGGCGGGCG |
| 26 | Transcription | ATGGGTGCGAGAGCGTCAGTATTAAGCGGGGGAGAATTAGATCGATGGGA |
| | Helper/Rev; | AAAAATTCGGTTAAGGCCAGGGGGAAAGAAAAAATATAAATTAAAACATA |
| | HIV Gag; Viral | TAGTATGGGCAAGCAGGGAGCTAGAACGATTCGCAGTTAATCCTGGCCTGT |
| | capsid | TAGAAACATCAGAAGGCTGTAGACAAATACTGGGACAGCTACAACCATCCC |
| | | TTCAGACAGGATCAGAAGAACTTAGATCATTATATAATACAGTAGCAACCC |
| | | TCTATTGTGTGCATCAAAGGATAGAGATAAAAGACACCAAGGAAGCTTTAG |
| | | ACAAGATAGAGGAAGAGCAAAACAAAAGTAAGAAAAAAGCACAGCAAGC |
| | | AGCAGCTGACACAGGACACAGCAATCAGGTCAGCCAAAATTACCCTATAGT |
| | | GCAGAACATCCAGGGGCAAATGGTACATCAGGCCATATCACCTAGAACTTT |
| | | AAATGCATGGGTAAAAGTAGTAGAAGAGAAGGCTTTCAGCCCAGAAGTGA |
| | | TACCCATGTTTTCAGCATTATCAGAAGGAGCCACCCCACAAGATTTAAACA |
| | | CCATGCTAAACACAGTGGGGGGACATCAAGCAGCCATGCAAATGTTAAAAG |
| | | AGACCATCAATGAGGAAGCTGCAGAATGGGATAGAGTGCATCCAGTGCATG |
| | | CAGGGCCTATTGCACCAGGCCAGATGAGAGAACCAAGGGGAAGTGACATA |
| | | GCAGGAACTACTAGTACCCTTCAGGAACAAATAGGATGGATGACACATAAT |
| | | CCACCTATCCCAGTAGGAGAAATCTATAAAAGATGGATAATCCTGGGATTA |
| | | AATAAAATAGTAAGAATGTATAGCCCTACCAGCATTCTGGACATAAGACAA |
| | | GGACCAAAGGAACCCTTTAGAGACTATGTAGACCGATTCTATAAAACTCTA |
| | | AGAGCCGAGCAAGCTTCACAAGAGGTAAAAAATTGGATGACAGAAACCTT |
| | | GTTGGTCCAAAATGCGAACCCAGATTGTAAGACTATTTTAAAAGCATTGGG |
| | | ACCAGGAGCGACACTAGAAGAAATGATGACAGCATGTCAGGGAGTGGGGG |
| | | GACCCGGCCATAAAGCAAGAGTTTTGGCTGAAGCAATGAGCCAAGTAACAA |
| | | ATCCAGCTACCATAATGATACAGAAAGGCAATTTTAGGAACCAAAGAAAGA |
| | | CTGTTAAGTGTTTCAATTGTGGCAAAGAAGGGCACATAGCCAAAAATTGCA |
| | | GGGCCCCTAGGAAAAAGGGCTGTTGGAAATGTGGAAAGGAAGGACACCAA |
| | | ATGAAAGATTGTACTGAGAGACAGGCTAATTTTTTAGGGAAGATCTGGCCT |
| | | TCCCACAAGGGAAGGCCAGGGAATTTTCTTCAGAGCAGACCAGAGCCAACA |
| | | GCCCCACCAGAAGAGAGCTTCAGGTTTGGGGAAGAGACAACAACTCCCTCT |
| | | CAGAAGCAGGAGCCGATAGACAAGGAACTGTATCCTTTAGCTTCCCTCAGA |
| | | TCACTCTTTGGCAGCGACCCCTCGTCACAATAA |
| 27 | Helper/Rev; | ATGAATTTGCCAGGAAGATGGAAACCAAAAATGATAGGGGGAATTGGAGG |
| | HIV Pol; | TTTTATCAAAGTAGGACAGTATGATCAGATACTCATAGAAATCTGCGGACA |
| | Protease and | TAAAGCTATAGGTACAGTATTAGTAGGACCTACACCTGTCAACATAATTGG |
| | reverse | AAGAAATCTGTTGACTCAGATTGGCTGCACTTTAAATTTTCCCATTAGTCCT |
| | transcriptase | ATTGAGACTGTACCAGTAAAATTAAAGCCAGGAATGGATGGCCCAAAAGTT |
| | | AAACAATGGCCATTGACAGAAGAAAAAATAAAAGCATTAGTAGAAATTTGT |
| | | ACAGAAATGGAAAAGGAAGGAAAAATTTCAAAAATTGGGCCTGAAAATCC |
| | | ATACAATACTCCAGTATTTGCCATAAAGAAAAAAGACAGTACTAAATGGAG |
| | | AAAATTAGTAGATTTCAGAGAACTTAATAAGAGAACTCAAGATTTCTGGGA |
| | | AGTTCAATTAGGAATACCACATCCTGCAGGGTTAAAACAGAAAAAATCAGT |
| | | AACAGTACTGGATGTGGGCGATGCATATTTTTCAGTTCCCTTAGATAAAGAC |
| | | TTCAGGAAGTATACTGCATTTACCATACCTAGTATAAACAATGAGACACCA |
| | | GGGATTAGATATCAGTACAATGTGCTTCCACAGGGATGGAAAGGATCACCA |
| | | GCAATATTCCAGTGTAGCATGACAAAAATCTTAGAGCCTTTTAGAAAACAA |
| | | AATCCAGACATAGTCATCTATCAATACATGGATGATTTGTATGTAGGATCTG |
| | | ACTTAGAAATAGGGCAGCATAGAACAAAAATAGAGGAACTGAGACAACAT |
| | | CTGTTGAGGTGGGGATTTACCACACCAGACAAAAAACATCAGAAAGAACCT |
| | | CCATTCCTTTGGATGGGTTATGAACTCCATCCTGATAAATGGACAGTACAGC |
| | | CTATAGTGCTGCCAGAAAAGGACAGCTGGACTGTCAATGACATACAGAAAT |
| | | TAGTGGGAAAATTGAATTGGGCAAGTCAGATTTATGCAGGGATTAAAGTAA |
| | | GGCAATTATGTAAACTTCTTAGGGGAACCAAAGCACTAACAGAAGTAGTAC |
| | | CACTAACAGAAGAAGCAGAGCTAGAACTGGCAGAAAACAGGGAGATTCTA |
| | | AAAGAACCGGTACATGGAGTGTATTATGACCCATCAAAAGACTTAATAGCA |
| | | GAAATACAGAAGCAGGGGCAAGGCCAATGGACATATCAAATTTATCAAGA |
| | | GCCATTTAAAAATCTGAAAACAGGAAAATATGCAAGAATGAAGGGTGCCC |
| | | ACACTAATGATGTGAAACAATTAACAGAGGCAGTACAAAAAATAGCCACA |
| | | GAAAGCATAGTAATATGGGGAAAGACTCCTAAATTTAAATTACCCATACAA |
| | | AAGGAAACATGGGAAGCATGGTGGACAGAGTATTGGCAAGCCACCTGGAT |
| | | TCCTGAGTGGGAGTTTGTCAATACCCCTCCCTTAGTGAAGTTATGGTACCAG |
| | | TTAGAGAAAGAACCCATAATAGGAGCAGAAACTTTCTATGTAGATGGGGCA |
| | | GCCAATAGGGAAACTAAATTAGGAAAAGCAGGATATGTAACTGACAGAGG |
| | | AAGACAAAAAGTTGTCCCCCTAACGGACACAACAAATCAGAAGACTGAGTT |
| | | ACAAGCAATTCATCTAGCTTTGCAGGATTCGGGATTAGAAGTAAACATAGT |
| | | GACAGACTCACAATATGCATTGGGAATCATTCAAGCACAACCAGATAAGAG |
| | | TGAATCAGAGTTAGTCAGTCAAATAATAGAGCAGTTAATAAAAAAGGAAA |
| | | AAGTCTACCTGGCATGGGTACCAGCACACAAAGGAATTGGAGGAAATGAA |
| | | CAAGTAGATGGGTTGGTCAGTGCTGGAATCAGGAAAGTACTA |
| 28 | Helper Rev; | TTTTTAGATGGAATAGATAAGGCCCAAGAAGAACATGAGAAATATCACAGT |
| | HIV Integrase; | AATTGGAGAGCAATGGCTAGTGATTTTAACCTACCACCTGTAGTAGCAAAA |
| | Integration of | GAAATAGTAGCCAGCTGTGATAAATGTCAGCTAAAAGGGGAAGCCATGCAT |
| | viral RNA | GGACAAGTAGACTGTAGCCCAGGAATATGGCAGCTAGATTGTACACATTTA |
| | | GAAGGAAAAGTTATCTTGGTAGCAGTTCATGTAGCCAGTGGATATATAGAA |
| | | GCAGAAGTAATTCCAGCAGAGACAGGGCAAGAAACAGCATACTTCCTCTTA |
| | | AAATTAGCAGGAAGATGGCCAGTAAAAACAGTACATACAGACAATGGCAG |
| | | CAATTTCACCAGTACTACAGTTAAGGCCGCCTGTTGGTGGGCGGGGATCAA |
| | | GCAGGAATTTGGCATTCCCTACAATCCCCAAAGTCAAGGAGTAATAGAATC |
| | | TATGAATAAAGAATTAAAGAAAATTATAGGACAGGTAAGAGATCAGGCTG |
| | | AACATCTTAAGACAGCAGTACAAATGGCAGTATTCATCCACAATTTTAAAA |
| | | GAAAAGGGGGGATTGGGGGGTACAGTGCAGGGGAAAGAATAGTAGACATA |
| | | ATAGCAACAGACATACAAACTAAAGAATTACAAAAACAAATTACAAAAAT |
| | | TCAAAATTTTCGGGTTTATTACAGGGACAGCAGAGATCCAGTTTGGAAAGG |
| | | ACCAGCAAAGCTCCTCTGGAAAGGTGAAGGGGCAGTAGTAATACAAGATA |
| | | ATAGTGACATAAAAGTAGTGCCAAGAAGAAAAGCAAAGATCATCAGGGAT |
| | | TATGGAAAACAGATGGCAGGTGATGATTGTGTGGCAAGTAGACAGGATGA |
| | | GGATTAA |
| 29 | Helper/Rev; | AGGAGCTTTGTTCCTTGGGTTCTTGGGAGCAGCAGGAAGCACTATGGGCGC |
| | HIV RRE; | AGCGTCAATGACGCTGACGGTACAGGCCAGACAATTATTGTCTGGTATAGT |
| | Binds Rev | GCAGCAGCAGAACAATTTGCTGAGGGCTATTGAGGCGCAACAGCATCTGTT |
| | element | GCAACTCACAGTCTGGGGCATCAAGCAGCTCCAGGCAAGAATCCTGGCTGT |
| | | GGAAAGATACCTAAAGGATCAACAGCTCCT |
| 30 | Helper/Rev; | ATGGCAGGAAGAAGCGGAGACAGCGACGAAGAACTCCTCAAGGCAGTCAG |
| | HIV Rev; | ACTCATCAAGTTTCTCTATCAAAGCAACCCACCTCCCAATCCCGAGGGGACC |
| | Nuclear export | CGACAGGCCCGAAGGAATAGAAGAAGAAGGTGGAGAGAGAGACAGAGAC |
| | and stabilize | AGATCCATTCGATTAGTGAACGGATCCTTAGCACTTATCTGGGACGATCTGC |
| | viral mRNA | GGAGCCTGTGCCTCTTCAGCTACCACCGCTTGAGAGACTTACTCTTGATTGT |
| | | AACGAGGATTGTGGAACTTCTGGGACGCAGGGGGTGGGAAGCCCTCAAATA |
| | | TTGGTGGAATCTCCTACAATATTGGAGTCAGGAGCTAAAGAATAG |
| 31 | Envelope; | ACATTGATTATTGACTAGTTATTAATAGTAATCAATTACGGGGTCATTAGTT |
| | CMV promoter | CATAGCCCATATATGGAGTTCCGCGTTACATAACTTACGGTAAATGGCCCGC |
| | | CTGGCTGACCGCCCAACGACCCCCGCCCATTGACGTCAATAATGACGTATG |
| | | TTCCCATAGTAACGCCAATAGGGACTTTCCATTGACGTCAATGGGTGGAGT |
| | | ATTTACGGTAAACTGCCCACTTGGCAGTACATCAAGTGTATCATATGCCAAG |
| | | TACGCCCCCTATTGACGTCAATGACGGTAAATGGCCCGCCTGGCATTATGCC |
| | | CAGTACATGACCTTATGGGACTTTCCTACTTGGCAGTACATCTACGTATTAG |
| | | TCATCGCTATTACCATGGTGATGCGGTTTTGGCAGTACATCAATGGGCGTGG |
| | | ATAGCGGTTTGACTCACGGGGATTTCCAAGTCTCCACCCCATTGACGTCAAT |
| | | GGGAGTTTGTTTTGGCACCAAAATCAACGGGACTTTCCAAAATGTCGTAAC |
| | | AACTCCGCCCCATTGACGCAAATGGGCGGTAGGCGTGTACGGTGGGAGGTC |
| | | TATATAAGC |
| 32 | Envelope; | ATGAAGTGCCTTTTGTACTTAGCCTTTTTATTCATTGGGGTGAATTGCAAGTT |
| | VSV-G; | CACCATAGTTTTTCCACACAACCAAAAAGGAAACTGGAAAAATGTTCCTTC |
| | Glycoprotein | TAATTACCATTATTGCCCGTCAAGCTCAGATTTAAATTGGCATAATGACTTA |
| | envelope-cell | ATAGGCACAGCCTTACAAGTCAAAATGCCCAAGAGTCACAAGGCTATTCAA |
| | entry | GCAGACGGTTGGATGTGTCATGCTTCCAAATGGGTCACTACTTGTGATTTCC |
| | | GCTGGTATGGACCGAAGTATATAACACATTCCATCCGATCCTTCACTCCATC |
| | | TGTAGAACAATGCAAGGAAAGCATTGAACAAACGAAACAAGGAACTTGGC |
| | | TGAATCCAGGCTTCCCTCCTCAAAGTTGTGGATATGCAACTGTGACGGATGC |
| | | CGAAGCAGTGATTGTCCAGGTGACTCCTCACCATGTGCTGGTTGATGAATAC |
| | | ACAGGAGAATGGGTTGATTCACAGTTCATCAACGGAAAATGCAGCAATTAC |
| | | ATATGCCCCACTGTCCATAACTCTACAACCTGGCATTCTGACTATAAGGTCA |
| | | AAGGGCTATGTGATTCTAACCTCATTTCCATGGACATCACCTTCTTCTCAGA |
| | | GGACGGAGAGCTATCATCCCTGGGAAAGGAGGGCACAGGGTTCAGAAGTA |
| | | ACTACTTTGCTTATGAAACTGGAGGCAAGGCCTGCAAAATGCAATACTGCA |
| | | AGCATTGGGGAGTCAGACTCCCATCAGGTGTCTGGTTCGAGATGGCTGATA |
| | | AGGATCTCTTTGCTGCAGCCAGATTCCCTGAATGCCCAGAAGGGTCAAGTA |
| | | TCTCTGCTCCATCTCAGACCTCAGTGGATGTAAGTCTAATTCAGGACGTTGA |
| | | GAGGATCTTGGATTATTCCCTCTGCCAAGAAACCTGGAGCAAAATCAGAGC |
| | | GGGTCTTCCAATCTCTCCAGTGGATCTCAGCTATCTTGCTCCTAAAAACCCA |
| | | GGAACCGGTCCTGCTTTCACCATAATCAATGGTACCCTAAAATACTTTGAGA |
| | | CCAGATACATCAGAGTCGATATTGCTGCTCCAATCCTCTCAAGAATGGTCGG |
| | | AATGATCAGTGGAACTACCACAGAAAGGGAACTGTGGGATGACTGGGCAC |
| | | CATATGAAGACGTGGAAATTGGACCCAATGGAGTTCTGAGGACCAGTTCAG |
| | | GATATAAGTTTCCTTTATACATGATTGGACATGGTATGTTGGACTCCGATCT |
| | | TCATCTTAGCTCAAAGGCTCAGGTGTTCGAACATCCTCACATTCAAGACGCT |
| | | GCTTCGCAACTTCCTGATGATGAGAGTTTATTTTTTGGTGATACTGGGCTAT |
| | | CCAAAAATCCAATCGAGCTTGTAGAAGGTTGGTTCAGTAGTTGGAAAAGCT |
| | | CTATTGCCTCTTTTTTCTTTATCATAGGGTTAATCATTGGACTATTCTTGGTT |
| | | CTCCGAGTTGGTATCCATCTTTGCATTAAATTAAAGCACACCAAGAAAAGA |
| | | CAGATTTATACAGACATAGAGATGA |
| 33 | Helper/Rev; | TAGTTATTAATAGTAATCAATTACGGGGTCATTAGTTCATAGCCCATATATG |
| | CMV early | GAGTTCCGCGTTACATAACTTACGGTAAATGGCCCGCCTGGCTGACCGCCC |
| | (CAG) | AACGACCCCCGCCCATTGACGTCAATAATGACGTATGTTCCCATAGTAACG |
| | enhancer; | CCAATAGGGACTTTCCATTGACGTCAATGGGTGGACTATTTACGGTAAACTG |
| | Enhance | CCCACTTGGCAGTACATCAAGTGTATCATATGCCAAGTACGCCCCCTATTGA |
| | Transcription | CGTCAATGACGGTAAATGGCCCGCCTGGCATTATGCCCAGTACATGACCTT |
| | | ATGGGACTTTCCTACTTGGCAGTACATCTACGTATTAGTCATC |
| 34 | Helper/Rev; | GGAGTCGCTGCGTTGCCTTCGCCCCGTGCCCCGCTCCGCGCCGCCTCGCGCC |
| | Chicken beta | GCCCGCCCCGGCTCTGACTGACCGCGTTACTCCCACAGGTGAGCGGGCGGG |
| | actin intron; | ACGGCCCTTCTCCTCCGGGCTGTAATTAGCGCTTGGTTTAATGACGGCTCGT |
| | Enhance gene | TTCTTTTCTGTGGCTGCGTGAAAGCCTTAAAGGGCTCCGGGAGGGCCCTTTG |
| | expression | TGCGGGGGGGAGCGGCTCGGGGGGTGCGTGCGTGTGTGTGTGCGTGGGGAG |
| | | CGCCGCGTGCGGCCCGCGCTGCCCGGCGGCTGTGAGCGCTGCGGGCGCGGC |
| | | GCGGGGCTTTGTGCGCTCCGCGTGTGCGCGAGGGGAGCGCGGCCGGGGGCG |
| | | GTGCCCCGCGGTGCGGGGGGGCTGCGAGGGGAACAAAGGCTGCGTGCGGG |
| | | GTGTGTGCGTGGGGGGGTGAGCAGGGGGTGTGGGCGCGGCGGTCGGGCTGT |
| | | AACCCCCCCCTGCACCCCCCTCCCCGAGTTGCTGAGCACGGCCCGGCTTCGG |
| | | GTGCGGGGCTCCGTGCGGGGCGTGGCGCGGGGCTCGCCGTGCCGGGCGGGG |
| | | GGTGGCGGCAGGTGGGGGTGCCGGGCGGGGCGGGGCCGCCTCGGGCCGGG |
| | | GAGGGCTCGGGGGAGGGGCGCGGCGGCCCCGGAGCGCCGGCGGCTGTCGA |
| | | GGCGCGGCGAGCCGCAGCCATTGCCTTTTATGGTAATCGTGCGAGAGGGCG |
| | | CAGGGACTTCCTTTGTCCCAAATCTGGCGGAGCCGAAATCTGGGAGGCGCC |
| | | GCCGCACCCCCTCTAGCGGGCGCGGGCGAAGCGGTGCGGCGCCGGCAGGA |
| | | AGGAAATGGGCGGGGAGGGCCTTCGTGCGTCGCCGCGCCGCCGTCCCCTTC |
| | | TCCATCTCCAGCCTCGGGGCTGCCGCAGGGGGACGGCTGCCTTCGGGGGGG |
| | | ACGGGGCAGGGCGGGGTTCGGCTTCTGGCGTGTGACCGGCGG |
| 35 | Helper/Rev; | AGATCTTTTTCCCTCTGCCAAAAATTATGGGGACATCATGAAGCCCCTTGAG |
| | Rabbit beta | CATCTGACTTCTGGCTAATAAAGGAAATTTATTTTCATTGCAATAGTGTGTT |
| | globin poly A; | GGAATTTTTTGTGTCTCTCACTCGGAAGGACATATGGGAGGGCAAATCATTT |
| | RNA stability | AAAACATCAGAATGAGTATTTGGTTTAGAGTTTGGCAACATATGCCATATG |
| | | CTGGCTGCCATGAACAAAGGTGGCTATAAAGAGGTCATCAGTATATGAAAC |
| | | AGCCCCCTGCTGTCCATTCCTTATTCCATAGAAAAGCCTTGACTTGAGGTTA |
| | | GATTTTTTTTATATTTTGTTTTGTGTTATTTTTTTCTTTAACATCCCTAAAATT |
| | | TTCCTTACATGTTTTACTAGCCAGATTTTTCCTCCTCTCCTGACTACTCCCAG |
| | | TCATAGCTGTCCCTCTTCTCTTATGAAGATC |
| 36 | Envelope; Beta | GTGAGTTTGGGGACCCTTGATTGTTCTTTCTTTTTCGCTATTGTAAAATTCAT |
| | globin intron; | GTTATATGGAGGGGGCAAAGTTTTCAGGGTGTTGTTTAGAATGGGAAGATG |
| | Enhance gene | TCCCTTGTATCACCATGGACCCTCATGATAATTTTGTTTCTTTCACTTTCTAC |
| | expression | TCTGTTGACAACCATTGTCTCCTCTTATTTTCTTTTCATTTTCTGTAACTTTTT |
| | | CGTTAAACTTTAGCTTGCATTTGTAACGAATTTTTAAATTCACTTTTGTTTAT |
| | | TTGTCAGATTGTAAGTACTTTCTCTAATCACTTTTTTTTCAAGGCAATCAGGG |
| | | TATATTATATTGTACTTCAGCACAGTTTTAGAGAACAATTGTTATAATTAAA |
| | | TGATAAGGTAGAATATTTCTGCATATAAATTCTGGCTGGCGTGGAAATATTC |
| | | TTATTGGTAGAAACAACTACACCCTGGTCATCATCCTGCCTTTCTCTTTATG |
| | | GTTACAATGATATACACTGTTTGAGATGAGGATAAAATACTCTGAGTCCAA |
| | | ACCGGGCCCCTCTGCTAACCATGTTCATGCCTTCTTCTCTTTCCTACAG |
| 37 | Envelope; | AGATCTTTTTCCCTCTGCCAAAAATTATGGGGACATCATGAAGCCCCTTGAG |
| | Rabbit beta | CATCTGACTTCTGGCTAATAAAGGAAATTTATTTTCATTGCAATAGTGTGTT |
| | globin poly A; | GGAATTTTTTGTGTCTCTCACTCGGAAGGACATATGGGAGGGCAAATCATTT |
| | RNA stability | AAAACATCAGAATGAGTATTTGGTTTAGAGTTTGGCAACATATGCCCATAT |
| | | GCTGGCTGCCATGAACAAAGGTTGGCTATAAAGAGGTCATCAGTATATGAA |
| | | ACAGCCCCCTGCTGTCCATTCCTTATTCCATAGAAAAGCCTTGACTTGAGGT |
| | | TAGATTTTTTTTATATTTTGTTTTGTGTTATTTTTTTCTTTAACATCCCTAAAA |
| | | TTTTCCTTACATGTTTTACTAGCCAGATTTTTCCTCCTCTCCTGACTACTCCC |
| | | AGTCATAGCTGTCCCTCTTCTCTTATGGAGATC |
| 38 | Forward Primer | TAAGCAGAATTCATGAATTTGCCAGGAAGAT |
| 39 | Reverse Primer | CCATACAATGAATGGACACTAGGCGGCCGCACGAAT |
| 40 | Gag, Pol, | GAATTCATGAATTTGCCAGGAAGATGGAAACCAAAAATGATAGGGGGAATT |
| | Integrase | GGAGGTTTTATCAAAGTAAGACAGTATGATCAGATACTCATAGAAATCTGC |
| | fragment | GGACATAAAGCTATAGGTACAGTATTAGTAGGACCTACACCTGTCAACATA |
| | | ATTGGAAGAAATCTGTTGACTCAGATTGGCTGCACTTTAAATTTTCCCATTA |
| | | GTCCTATTGAGACTGTACCAGTAAAATTAAAGCCAGGAATGGATGGCCCAA |
| | | AAGTTAAACAATGGCCATTGACAGAAGAAAAAATAAAAGCATTAGTAGAA |
| | | ATTTGTACAGAAATGGAAAAGGAAGGAAAAATTTCAAAAATTGGGCCTGA |
| | | AAATCCATACAATACTCCAGTATTTGCCATAAAGAAAAAAGACAGTACTAA |
| | | ATGGAGAAAATTAGTAGATTTCAGAGAACTTAATAAGAGAACTCAAGATTT |
| | | CTGGGAAGTTCAATTAGGAATACCACATCCTGCAGGGTTAAAACAGAAAAA |
| | | ATCAGTAACAGTACTGGATGTGGGCGATGCATATTTTTCAGTTCCCTTAGAT |
| | | AAAGACTTCAGGAAGTATACTGCATTTACCATACCTAGTATAAACAATGAG |
| | | ACACCAGGGATTAGATATCAGTACAATGTGCTTCCACAGGGATGGAAAGGA |
| | | TCACCAGCAATATTCCAGTGTAGCATGACAAAAATCTTAGAGCCTTTTAGA |
| | | AAACAAAATCCAGACATAGTCATCTATCAATACATGGATGATTTGTATGTA |
| | | GGATCTGACTTAGAAATAGGGCAGCATAGAACAAAAATAGAGGAACTGAG |
| | | ACAACATCTGTTGAGGTGGGGATTTACCACACCAGACAAAAAACATCAGAA |
| | | AGAACCTCCATTCCTTTGGATGGGTTATGAACTCCATCCTGATAAATGGACA |
| | | GTACAGCCTATAGTGCTGCCAGAAAAGGACAGCTGGACTGTCAATGACATA |
| | | CAGAAATTAGTGGGAAAATTGAATTGGGCAAGTCAGATTTATGCAGGGATT |
| | | AAAGTAAGGCAATTATGTAAACTTCTTAGGGGAACCAAAGCACTAACAGAA |
| | | GTAGTACCACTAACAGAAGAAGCAGAGCTAGAACTGGCAGAAAACAGGGA |
| | | GATTCTAAAAGAACCGGTACATGGAGTGTATTATGACCCATCAAAAGACTT |
| | | AATAGCAGAAATACAGAAGCAGGGGCAAGGCCAATGGACATATCAAATTT |
| | | ATCAAGAGCCATTTAAAAATCTGAAAACAGGAAAGTATGCAAGAATGAAG |
| | | GGTGCCCACACTAATGATGTGAAACAATTAACAGAGGCAGTACAAAAAATA |
| | | GCCACAGAAAGCATAGTAATATGGGGAAAGACTCCTAAATTTAAATTACCC |
| | | ATACAAAAGGAAACATGGGAAGCATGGTGGACAGAGTATTGGCAAGCCAC |
| | | CTGGATTCCTGAGTGGGAGTTTGTCAATACCCCTCCCTTAGTGAAGTTATGG |
| | | TACCAGTTAGAGAAAGAACCCATAATAGGAGCAGAAACTTTCTATGTAGAT |
| | | GGGGCAGCCAATAGGGAAACTAAATTAGGAAAAGCAGGATATGTAACTGA |
| | | CAGAGGAAGACAAAAAGTTGTCCCCCTAACGGACACAACAAATCAGAAGA |
| | | CTGAGTTACAAGCAATTCATCTAGCTTTGCAGGATTCGGGATTAGAAGTAA |
| | | ACATAGTGACAGACTCACAATATGCATTGGGAATCATTCAAGCACAACCAG |
| | | ATAAGAGTGAATCAGAGTTAGTCAGTCAAATAATAGAGCAGTTAATAAAAA |
| | | AGGAAAAAGTCTACCTGGCATGGGTACCAGCACACAAAGGAATTGGAGGA |
| | | AATGAACAAGTAGATAAATTGGTCAGTGCTGGAATCAGGAAAGTACTATTT |
| | | TTAGATGGAATAGATAAGGCCCAAGAAGAACATGAGAAATATCACAGTAA |
| | | TTGGAGAGCAATGGCTAGTGATTTTAACCTACCACCTGTAGTAGCAAAAGA |
| | | AATAGTAGCCAGCTGTGATAAATGTCAGCTAAAAGGGGAAGCCATGCATGG |
| | | ACAAGTAGACTGTAGCCCAGGAATATGGCAGCTAGATTGTACACATTTAGA |
| | | AGGAAAAGTTATCTTGGTAGCAGTTCATGTAGCCAGTGGATATATAGAAGC |
| | | AGAAGTAATTCCAGCAGAGACAGGGCAAGAAACAGCATACTTCCTCTTAAA |
| | | ATTAGCAGGAAGATGGCCAGTAAAAACAGTACATACAGACAATGGCAGCA |
| | | ATTTCACCAGTACTACAGTTAAGGCCGCCTGTTGGTGGGCGGGGATCAAGC |
| | | AGGAATTTGGCATTCCCTACAATCCCCAAAGTCAAGGAGTAATAGAATCTA |
| | | TGAATAAAGAATTAAAGAAAATTATAGGACAGGTAAGAGATCAGGCTGAA |
| | | CATCTTAAGACAGCAGTACAAATGGCAGTATTCATCCACAATTTTAAAAGA |
| | | AAAGGGGGGATTGGGGGGTACAGTGCAGGGGAAAGAATAGTAGACATAAT |
| | | AGCAACAGACATACAAACTAAAGAATTACAAAAACAAATTACAAAAATTC |
| | | AAAATTTTCGGGTTTATTACAGGGACAGCAGAGATCCAGTTTGGAAAGGAC |
| | | CAGCAAAGCTCCTCTGGAAAGGTGAAGGGGCAGTAGTAATACAAGATAAT |
| | | AGTGACATAAAAGTAGTGCCAAGAAGAAAAGCAAAGATCATCAGGGATTA |
| | | TGGAAAACAGATGGCAGGTGATGATTGTGTGGCAAGTAGACAGGATGAGG |
| | | ATTAA |
| 41 | DNA Fragment | TCTAGAATGGCAGGAAGAAGCGGAGACAGCGACGAAGAGCTCATCAGAAC |
| | containing Rev, | AGTCAGACTCATCAAGCTTCTCTATCAAAGCAACCCACCTCCCAATCCCGAG |
| | RRE and rabbit | GGGACCCGACAGGCCCGAAGGAATAGAAGAAGAAGGTGGAGAGAGAGAC |
| | beta globin poly | AGAGACAGATCCATTCGATTAGTGAACGGATCCTTGGCACTTATCTGGGAC |
| | A | GATCTGCGGAGCCTGTGCCTCTTCAGCTACCACCGCTTGAGAGACTTACTCT |
| | | TGATTGTAACGAGGATTGTGGAACTTCTGGGACGCAGGGGGTGGGAAGCCC |
| | | TCAAATATTGGTGGAATCTCCTACAATATTGGAGTCAGGAGCTAAAGAATA |
| | | GAGGAGCTTTGTTCCTTGGGTTCTTGGGAGCAGCAGGAAGCACTATGGGCG |
| | | CAGCGTCAATGACGCTGACGGTACAGGCCAGACAATTATTGTCTGGTATAG |
| | | TGCAGCAGCAGAACAATTTGCTGAGGGCTATTGAGGCGCAACAGCATCTGT |
| | | TGCAACTCACAGTCTGGGGCATCAAGCAGCTCCAGGCAAGAATCCTGGCTG |
| | | TGGAAAGATACCTAAAGGATCAACAGCTCCTAGATCTTTTTCCCTCTGCCAA |
| | | AAATTATGGGGACATCATGAAGCCCCTTGAGCATCTGACTTCTGGCTAATA |
| | | AAGGAAATTTATTTTCATTGCAATAGTGTGTTGGAATTTTTTGTGTCTCTCAC |
| | | TCGGAAGGACATATGGGAGGGCAAATCATTTAAAACATCAGAATGAGTATT |
| | | TGGTTTAGAGTTTGGCAACATATGCCATATGCTGGCTGCCATGAACAAAGG |
| | | TGGCTATAAAGAGGTCATCAGTATATGAAACAGCCCCCTGCTGTCCATTCCT |
| | | TATTCCATAGAAAAGCCTTGACTTGAGGTTAGATTTTTTTTATATTTTGTTTT |
| | | GTGTTATTTTTTTCTTTAACATCCCTAAAATTTTCCTTACATGTTTTACTAGC |
| | | CAGATTTTTCCTCCTCTCCTGACTACTCCCAGTCATAGCTGTCCCTCTTCTCT |
| | | TATGAAGATCCCTCGACCTGCAGCCCAAGCTTGGCGTAATCATGGTCATAG |
| | | CTGTTTCCTGTGTGAAATTGTTATCCGCTCACAATTCCACACAACATACGAG |
| | | CCGGAAGCATAAAGTGTAAAGCCTGGGGTGCCTAATGAGTGAGCTAACTCA |
| | | CATTAATTGCGTTGCGCTCACTGCCCGCTTTCCAGTCGGGAAACCTGTCGTG |
| | | CCAGCGGATCCGCATCTCAATTAGTCAGCAACCATAGTCCCGCCCCTAACTC |
| | | CGCCCATCCCGCCCCTAACTCCGCCCAGTTCCGCCCATTCTCCGCCCCATGG |
| | | CTGACTAATTTTTTTTATTTATGCAGAGGCCGAGGCCGCCTCGGCCTCTGAG |
| | | CTATTCCAGAAGTAGTGAGGAGGCTTTTTTGGAGGCCTAGGCTTTTGCAAAA |
| | | AGCTAACTTGTTTATTGCAGCTTATAATGGTTACAAATAAAGCAATAGCATC |
| | | ACAAATTTCACAAATAAAGCATTTTTTTCACTGCATTCTAGTTGTGGTTTGTC |
| | | CAAACTCATCAATGTATCTTATCAGCGGCCGCCCCGGG |
| 42 | DNA fragment | ACGCGTTAGTTATTAATAGTAATCAATTACGGGGTCATTAGTTCATAGCCCA |
| | containing the | TATATGGAGTTCCGCGTTACATAACTTACGGTAAATGGCCCGCCTGGCTGAC |
| | CAG | CGCCCAACGACCCCCGCCCATTGACGTCAATAATGACGTATGTTCCCATAGT |
| | enhancer/promoter/ | AACGCCAATAGGGACTTTCCATTGACGTCAATGGGTGGACTATTTACGGTA |
| | intron | AACTGCCCACTTGGCAGTACATCAAGTGTATCATATGCCAAGTACGCCCCCT |
| | sequence | ATTGACGTCAATGACGGTAAATGGCCCGCCTGGCATTATGCCCAGTACATG |
| | | ACCTTATGGGACTTTCCTACTTGGCAGTACATCTACGTATTAGTCATCGCTA |
| | | TTACCATGGGTCGAGGTGAGCCCCACGTTCTGCTTCACTCTCCCCATCTCCC |
| | | CCCCCTCCCCACCCCCAATTTTGTATTTATTTATTTTTTAATTATTTTGTGCA |
| | | GCGATGGGGGCGGGGGGGGGGGGGGCGCGCGCCAGGCGGGGGGGGGGGG |
| | | GGCGAGGGGCGGGGCGGGGCGAGGCGGAGAGGTGCGGCGGCAGCCAATCA |
| | | GAGCGGCGCGCTCCGAAAGTTTCCTTTTATGGCGAGGCGGCGGCGGCGGCG |
| | | GCCCTATAAAAAGCGAAGCGCGCGGCGGGCGGGAGTCGCTGCGTTGCCTTC |
| | | GCCCCGTGCCCCGCTCCGCGCCGCCTCGCGCCGCCCGCCCCGGCTCTGACTG |
| | | ACCGCGTTACTCCCACAGGTGAGCGGGCGGGACGGCCCTTCTCCTCCGGGC |
| | | TGTAATTAGCGCTTGGTTTAATGACGGCTCGTTTCTTTTCTGTGGCTGCGTGA |
| | | AAGCCTTAAAGGGCTCCGGGAGGGCCCTTTGTGCGGGGGGGAGCGGCTCGG |
| | | GGGGTGCGTGCGTGTGTGTGTGCGTGGGGAGCGCCGCGTGCGGCCCGCGCT |
| | | GCCCGGCGGCTGTGAGCGCTGCGGGCGCGGCGCGGGGCTTTGTGCGCTCCG |
| | | CGTGTGCGCGAGGGGAGCGCGGCCGGGGGCGGTGCCCCGCGGTGCGGGGG |
| | | GGCTGCGAGGGGAACAAAGGCTGCGTGCGGGGTGTGTGCGTGGGGGGGTG |
| | | AGCAGGGGGTGTGGGCGCGGCGGTCGGGCTGTAACCCCCCCCTGCACCCCC |
| | | CTCCCCGAGTTGCTGAGCACGGCCCGGCTTCGGGTGCGGGGCTCCGTGCGG |
| | | GGCGTGGCGCGGGGCTCGCCGTGCCGGGCGGGGGGTGGCGGCAGGTGGGG |
| | | GTGCCGGGCGGGGCGGGGCCGCCTCGGGCCGGGGAGGGCTCGGGGGAGGG |
| | | GCGCGGCGGCCCCGGAGCGCCGGCGGCTGTCGAGGCGCGGCGAGCCGCAG |
| | | CCATTGCCTTTTATGGTAATCGTGCGAGAGGGCGCAGGGACTTCCTTTGTCC |
| | | CAAATCTGGCGGAGCCGAAATCTGGGAGGCGCCGCCGCACCCCCTCTAGCG |
| | | GGCGCGGGCGAAGCGGTGCGGCGCCGGCAGGAAGGAAATGGGCGGGGAGG |
| | | GCCTTCGTGCGTCGCCGCGCCGCCGTCCCCTTCTCCATCTCCAGCCTCGGGG |
| | | CTGCCGCAGGGGGACGGCTGCCTTCGGGGGGGACGGGGCAGGGCGGGGTT |
| | | CGGCTTCTGGCGTGTGACCGGCGGGAATTC |
| 43 | DNA fragment | GAATTCATGAAGTGCCTTTTGTACTTAGCCTTTTTATTCATTGGGGTGAATTG |
| | containing | CAAGTTCACCATAGTTTTTCCACACAACCAAAAAGGAAACTGGAAAAATGT |
| | VSV-G | TCCTTCTAATTACCATTATTGCCCGTCAAGCTCAGATTTAAATTGGCATAAT |
| | | GACTTAATAGGCACAGCCTTACAAGTCAAAATGCCCAAGAGTCACAAGGCT |
| | | ATTCAAGCAGACGGTTGGATGTGTCATGCTTCCAAATGGGTCACTACTTGTG |
| | | ATTTCCGCTGGTATGGACCGAAGTATATAACACATTCCATCCGATCCTTCAC |
| | | TCCATCTGTAGAACAATGCAAGGAAAGCATTGAACAAACGAAACAAGGAA |
| | | CTTGGCTGAATCCAGGCTTCCCTCCTCAAAGTTGTGGATATGCAACTGTGAC |
| | | GGATGCCGAAGCAGTGATTGTCCAGGTGACTCCTCACCATGTGCTGGTTGAT |
| | | GAATACACAGGAGAATGGGTTGATTCACAGTTCATCAACGGAAAATGCAGC |
| | | AATTACATATGCCCCACTGTCCATAACTCTACAACCTGGCATTCTGACTATA |
| | | AGGTCAAAGGGCTATGTGATTCTAACCTCATTTCCATGGACATCACCTTCTT |
| | | CTCAGAGGACGGAGAGCTATCATCCCTGGGAAAGGAGGGCACAGGGTTCA |
| | | GAAGTAACTACTTTGCTTATGAAACTGGAGGCAAGGCCTGCAAAATGCAAT |
| | | ACTGCAAGCATTGGGGAGTCAGACTCCCATCAGGTGTCTGGTTCGAGATGG |
| | | CTGATAAGGATCTCTTTGCTGCAGCCAGATTCCCTGAATGCCCAGAAGGGTC |
| | | AAGTATCTCTGCTCCATCTCAGACCTCAGTGGATGTAAGTCTAATTCAGGAC |
| | | GTTGAGAGGATCTTGGATTATTCCCTCTGCCAAGAAACCTGGAGCAAAATC |
| | | AGAGCGGGTCTTCCAATCTCTCCAGTGGATCTCAGCTATCTTGCTCCTAAAA |
| | | ACCCAGGAACCGGTCCTGCTTTCACCATAATCAATGGTACCCTAAAATACTT |
| | | TGAGACCAGATACATCAGAGTCGATATTGCTGCTCCAATCCTCTCAAGAAT |
| | | GGTCGGAATGATCAGTGGAACTACCACAGAAAGGGAACTGTGGGATGACT |
| | | GGGCACCATATGAAGACGTGGAAATTGGACCCAATGGAGTTCTGAGGACCA |
| | | GTTCAGGATATAAGTTTCCTTTATACATGATTGGACATGGTATGTTGGACTC |
| | | CGATCTTCATCTTAGCTCAAAGGCTCAGGTGTTCGAACATCCTCACATTCAA |
| | | GACGCTGCTTCGCAACTTCCTGATGATGAGAGTTTATTTTTTGGTGATACTG |
| | | GGCTATCCAAAAATCCAATCGAGCTTGTAGAAGGTTGGTTCAGTAGTTGGA |
| | | AAAGCTCTATTGCCTCTTTTTTCTTTATCATAGGGTTAATCATTGGACTATTC |
| | | TTGGTTCTCCGAGTTGGTATCCATCTTTGCATTAAATTAAAGCACACCAAGA |
| | | AAAGACAGATTTATACAGACATAGAGATGAGAATTC |
| 44 | Helper plasmid | TCTAGAAGGAGCTTTGTTCCTTGGGTTCTTGGGAGCAGCAGGAAGCACTAT |
| | containing RRE | GGGCGCAGCGTCAATGACGCTGACGGTACAGGCCAGACAATTATTGTCTGG |
| | and rabbit beta | TATAGTGCAGCAGCAGAACAATTTGCTGAGGGCTATTGAGGCGCAACAGCA |
| | globin poly A | TCTGTTGCAACTCACAGTCTGGGGCATCAAGCAGCTCCAGGCAAGAATCCT |
| | | GGCTGTGGAAAGATACCTAAAGGATCAACAGCTCCTAGATCTTTTTCCCTCT |
| | | GCCAAAAATTATGGGGACATCATGAAGCCCCTTGAGCATCTGACTTCTGGC |
| | | TAATAAAGGAAATTTATTTTCATTGCAATAGTGTGTTGGAATTTTTTGTGTCT |
| | | CTCACTCGGAAGGACATATGGGAGGGCAAATCATTTAAAACATCAGAATGA |
| | | GTATTTGGTTTAGAGTTTGGCAACATATGCCATATGCTGGCTGCCATGAACA |
| | | AAGGTGGCTATAAAGAGGTCATCAGTATATGAAACAGCCCCCTGCTGTCCA |
| | | TTCCTTATTCCATAGAAAAGCCTTGACTTGAGGTTAGATTTTTTTTATATTTT |
| | | GTTTTGTGTTATTTTTTTCTTTAACATCCCTAAAATTTTCCTTACATGTTTTAC |
| | | TAGCCAGATTTTTCCTCCTCTCCTGACTACTCCCAGTCATAGCTGTCCCTCTT |
| | | CTCTTATGAAGATCCCTCGACCTGCAGCCCAAGCTTGGCGTAATCATGGTCA |
| | | TAGCTGTTTCCTGTGTGAAATTGTTATCCGCTCACAATTCCACACAACATAC |
| | | GAGCCGGAAGCATAAAGTGTAAAGCCTGGGGTGCCTAATGAGTGAGCTAAC |
| | | TCACATTAATTGCGTTGCGCTCACTGCCCGCTTTCCAGTCGGGAAACCTGTC |
| | | GTGCCAGCGGATCCGCATCTCAATTAGTCAGCAACCATAGTCCCGCCCCTA |
| | | ACTCCGCCCATCCCGCCCCTAACTCCGCCCAGTTCCGCCCATTCTCCGCCCC |
| | | ATGGCTGACTAATTTTTTTTATTTATGCAGAGGCCGAGGCCGCCTCGGCCTC |
| | | TGAGCTATTCCAGAAGTAGTGAGGAGGCTTTTTTGGAGGCCTAGGCTTTTGC |
| | | AAAAAGCTAACTTGTTTATTGCAGCTTATAATGGTTACAAATAAAGCAATA |
| | | GCATCACAAATTTCACAAATAAAGCATTTTTTTCACTGCATTCTAGTTGTGG |
| | | TTTGTCCAAACTCATCAATGTATCTTATCACCCGGG |
| 45 | RSV promoter | CAATTGCGATGTACGGGCCAGATATACGCGTATCTGAGGGGACTAGGGTGT |
| | and HIV Rev | GTTTAGGCGAAAAGCGGGGCTTCGGTTGTACGCGGTTAGGAGTCCCCTCAG |
| | | GATATAGTAGTTTCGCTTTTGCATAGGGAGGGGGAAATGTAGTCTTATGCA |
| | | ATACACTTGTAGTCTTGCAACATGGTAACGATGAGTTAGCAACATGCCTTAC |
| | | AAGGAGAGAAAAAGCACCGTGCATGCCGATTGGTGGAAGTAAGGTGGTAC |
| | | GATCGTGCCTTATTAGGAAGGCAACAGACAGGTCTGACATGGATTGGACGA |
| | | ACCACTGAATTCCGCATTGCAGAGATAATTGTATTTAAGTGCCTAGCTCGAT |
| | | ACAATAAACGCCATTTGACCATTCACCACATTGGTGTGCACCTCCAAGCTCG |
| | | AGCTCGTTTAGTGAACCGTCAGATCGCCTGGAGACGCCATCCACGCTGTTTT |
| | | GACCTCCATAGAAGACACCGGGACCGATCCAGCCTCCCCTCGAAGCTAGCG |
| | | ATTAGGCATCTCCTATGGCAGGAAGAAGCGGAGACAGCGACGAAGAACTC |
| | | CTCAAGGCAGTCAGACTCATCAAGTTTCTCTATCAAAGCAACCCACCTCCCA |
| | | ATCCCGAGGGGACCCGACAGGCCCGAAGGAATAGAAGAAGAAGGTGGAGA |
| | | GAGAGACAGAGACAGATCCATTCGATTAGTGAACGGATCCTTAGCACTTAT |
| | | CTGGGACGATCTGCGGAGCCTGTGCCTCTTCAGCTACCACCGCTTGAGAGA |
| | | CTTACTCTTGATTGTAACGAGGATTGTGGAACTTCTGGGACGCAGGGGGTG |
| | | GGAAGCCCTCAAATATTGGTGGAATCTCCTACAATATTGGAGTCAGGAGCT |
| | | AAAGAATAGTCTAGA |
| 46 | Promoter; PGK | GGGGTTGGGGTTGCGCCTTTTCCAAGGCAGCCCTGGGTTTGCGCAGGGACG |
| | | CGGCTGCTCTGGGCGTGGTTCCGGGAAACGCAGCGGCGCCGACCCTGGGTC |
| | | TCGCACATTCTTCACGTCCGTTCGCAGCGTCACCCGGATCTTCGCCGCTACC |
| | | CTTGTGGGCCCCCCGGCGACGCTTCCTGCTCCGCCCCTAAGTCGGGAAGGTT |
| | | CCTTGCGGTTCGCGGCGTGCCGGACGTGACAAACGGAAGCCGCACGTCTCA |
| | | CTAGTACCCTCGCAGACGGACAGCGCCAGGGAGCAATGGCAGCGCGCCGA |
| | | CCGCGATGGGCTGTGGCCAATAGCGGCTGCTCAGCAGGGCGCGCCGAGAGC |
| | | AGCGGCCGGGAAGGGGCGGTGCGGGAGGCGGGGTGTGGGGCGGTAGTGTG |
| | | GGCCCTGTTCCTGCCCGCGCGGTGTTCCGCATTCTGCAAGCCTCCGGAGCGC |
| | | ACGTCGGCAGTCGGCTCCCTCGTTGACCGAATCACCGACCTCTCTCCCCAG |
| 47 | Promoter; UbC | GCGCCGGGTTTTGGCGCCTCCCGCGGGCGCCCCCCTCCTCACGGCGAGCGCT |
| | | GCCACGTCAGACGAAGGGCGCAGGAGCGTTCCTGATCCTTCCGCCCGGACG |
| | | CTCAGGACAGCGGCCCGCTGCTCATAAGACTCGGCCTTAGAACCCCAGTAT |
| | | CAGCAGAAGGACATTTTAGGACGGGACTTGGGTGACTCTAGGGCACTGGTT |
| | | TTCTTTCCAGAGAGCGGAACAGGCGAGGAAAAGTAGTCCCTTCTCGGCGAT |
| | | TCTGCGGAGGGATCTCCGTGGGGCGGTGAACGCCGATGATTATATAAGGAC |
| | | GCGCCGGGTGTGGCACAGCTAGTTCCGTCGCAGCCGGGATTTGGGTCGCGG |
| | | TTCTTGTTTGTGGATCGCTGTGATCGTCACTTGGTGAGTTGCGGGCTGCTGG |
| | | GCTGGCCGGGGCTTTCGTGGCCGCCGGGCCGCTCGGTGGGACGGAAGCGTG |
| | | TGGAGAGACCGCCAAGGGCTGTAGTCTGGGTCCGCGAGCAAGGTTGCCCTG |
| | | AACTGGGGGTTGGGGGGAGCGCACAAAATGGCGGCTGTTCCCGAGTCTTGA |
| | | ATGGAAGACGCTTGTAAGGCGGGCTGTGAGGTCGTTGAAACAAGGTGGGG |
| | | GGCATGGTGGGCGGCAAGAACCCAAGGTCTTGAGGCCTTCGCTAATGCGGG |
| | | AAAGCTCTTATTCGGGTGAGATGGGCTGGGGCACCATCTGGGGACCCTGAC |
| | | GTGAAGTTTGTCACTGACTGGAGAACTCGGGTTTGTCGTCTGGTTGCGGGGG |
| | | CGGCAGTTATGCGGTGCCGTTGGGCAGTGCACCCGTACCTTTGGGAGCGCG |
| | | CGCCTCGTCGTGTCGTGACGTCACCCGTTCTGTTGGCTTATAATGCAGGGTG |
| | | GGGCCACCTGCCGGTAGGTGTGCGGTAGGCTTTTCTCCGTCGCAGGACGCA |
| | | GGGTTCGGGCCTAGGGTAGGCTCTCCTGAATCGACAGGCGCCGGACCTCTG |
| | | GTGAGGGGAGGGATAAGTGAGGCGTCAGTTTCTTTGGTCGGTTTTATGTACC |
| | | TATCTTCTTAAGTAGCTGAAGCTCCGGTTTTGAACTATGCGCTCGGGGTTGG |
| | | CGAGTGTGTTTTGTGAAGTTTTTTAGGCACCTTTTGAAATGTAATCATTTGG |
| | | GTCAATATGTAATTTTCAGTGTTAGACTAGTAAA |
| 48 | CAG promoter | TAGTTATTAATAGTAATCAATTACGGGGTCATTAGTTCATAGCCCATATATG |
| | | GAGTTCCGCGTTACATAACTTACGGTAAATGGCCCGCCTGGCTGACCGCCC |
| | | AACGACCCCCGCCCATTGACGTCAATAATGACGTATGTTCCCATAGTAACG |
| | | CCAATAGGGACTTTCCATTGACGTCAATGGGTGGACTATTTACGGTAAACTG |
| | | CCCACTTGGCAGTACATCAAGTGTATCATATGCCAAGTACGCCCCCTATTGA |
| | | CGTCAATGACGGTAAATGGCCCGCCTGGCATTATGCCCAGTACATGACCTT |
| | | ATGGGACTTTCCTACTTGGCAGTACATCTACGTATTAGTCATCGCTATTACC |
| | | ATGGGTCGAGGTGAGCCCCACGTTCTGCTTCACTCTCCCCATCTCCCCCCCC |
| | | TCCCCACCCCCAATTTTGTATTTATTTATTTTTTAATTATTTTGTGCAGCGAT |
| | | GGGGGCGGGGGGGGGGGGGGCGCGCGCCAGGCGGGGCGGGGCGGGGCGA |
| | | GGGGCGGGGCGGGGCGAGGCGGAGAGGTGCGGCGGCAGCCAATCAGAGCG |
| | | GCGCGCTCCGAAAGTTTCCTTTTATGGCGAGGCGGCGGCGGCGGCGGCCCT |
| | | ATAAAAAGCGAAGCGCGCGGCGGGCG |
| 49 | Poly A; SV40 | GTTTATTGCAGCTTATAATGGTTACAAATAAAGCAATAGCATCACAAATTTC |
| | | ACAAATAAAGCATTTTTTTCACTGCATTCTAGTTGTGGTTTGTCCAAACTCA |
| | | TCAATGTATCTTATCA |
| 50 | Poly A; bGH | GACTGTGCCTTCTAGTTGCCAGCCATCTGTTGTTTGCCCCTCCCCCGTGCCTT |
| | | CCTTGACCCTGGAAGGTGCCACTCCCACTGTCCTTTCCTAATAAAATGAGGA |
| | | AATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGG |
| | | GGATGCGGTGGGCTCTATGG |
| 51 | HIV Gag; Bal | ATGGGTGCGAGAGCGTCAGTATTAAGCGGGGGAGAATTAGATAGGTGGGA |
| | | AAAAATTCGGTTAAGGCCAGGGGGAAAGAAAAAATATAGATTAAAACATA |
| | | TAGTATGGGCAAGCAGGGAACTAGAAAGATTCGCAGTCAATCCTGGCCTGT |
| | | TAGAAACATCAGAAGGCTGCAGACAAATACTGGGACAGCTACAACCATCCC |
| | | TTCAGACAGGATCAGAAGAACTTAGATCATTATATAATACAGTAGCAACCC |
| | | TCTATTGTGTACATCAAAAGATAGAGGTAAAAGACACCAAGGAAGCTTTAG |
| | | ACAAAATAGAGGAAGAGCAAAACAAATGTAAGAAAAAGGCACAGCAAGC |
| | | AGCAGCTGACACAGGAAACAGCGGTCAGGTCAGCCAAAATTTCCCTATAGT |
| | | GCAGAACCTCCAGGGGCAAATGGTACATCAGGCCATATCACCTAGAACTTT |
| | | AAATGCATGGGTAAAAGTAATAGAAGAGAAAGCTTTCAGCCCAGAAGTAA |
| | | TACCCATGTTTTCAGCATTATCAGAAGGAGCCACCCCACAAGATTTAAACA |
| | | CCATGCTAAACACAGTGGGGGGACATCAAGCAGCCATGCAAATGTTAAAAG |
| | | AACCCATCAATGAGGAAGCTGCAAGATGGGATAGATTGCATCCCGTGCAGG |
| | | CAGGGCCTGTTGCACCAGGCCAGATAAGAGATCCAAGGGGAAGTGACATA |
| | | GCAGGAACTACCAGTACCCTTCAGGAACAAATAGGATGGATGACAAGTAAT |
| | | CCACCTATCCCAGTAGGAGAAATCTATAAAAGATGGATAATCCTGGGATTA |
| | | AATAAAATAGTAAGGATGTATAGCCCTACCAGCATTTTGGACATAAGACAA |
| | | GGACCAAAGGAACCCTTTAGAGACTATGTAGACCGGTTCTATAAAACTCTA |
| | | AGAGCCGAGCAAGCTTCACAGGAGGTAAAAAATTGGATGACAGAAACCTT |
| | | GTTGGTCCAAAATGCGAACCCAGATTGTAAGACTATTTTAAAAGCATTGGG |
| | | ACCAGCAGCTACACTAGAAGAAATGATGACAGCATGTCAGGGAGTGGGAG |
| | | GACCCAGCCATAAAGCAAGAATTTTGGCAGAAGCAATGAGCCAAGTAACA |
| | | AATTCAGCTACCATAATGATGCAGAAAGGCAATTTTAGGAACCAAAGAAAG |
| | | ATTGTTAAATGTTTCAATTGTGGCAAAGAAGGGCACATAGCCAGAAACTGC |
| | | AGGGCCCCTAGGAAAAGGGGCTGTTGGAAATGTGGAAAGGAAGGACACCA |
| | | AATGAAAGACTGTACTGAGAGACAGGCTAATTTTTTAGGGAAAATCTGGCC |
| | | TTCCCACAAAGGAAGGCCAGGGAATTTCCTTCAGAGCAGACCAGAGCCAAC |
| | | AGCCCCACCAGCCCCACCAGAAGAGAGCTTCAGGTTTGGGGAAGAGACAA |
| | | CAACTCCCTCTCAGAAGCAGGAGCTGATAGACAAGGAACTGTATCCTTTAG |
| | | CTTCCCTCAGATCACTCTTTGGCAACGACCCCTCGTCACAATAA |
| 52 | HIV Pol; Bal | ATGAATTTGCCAGGAAGATGGAAACCAAAAATGATAGGGGGAATTGGAGG |
| | | TTTTATCAAAGTAAGACAGTATGATCAGATACTCATAGAAATCTGTGGACA |
| | | TAAAGCTATAGGTACAGTATTAATAGGACCTACACCTGTCAACATAATTGG |
| | | AAGAAATCTGTTGACTCAGATTGGTTGCACTTTAAATTTTCCCATTAGTCCT |
| | | ATTGAAACTGTACCAGTAAAATTAAAACCAGGAATGGATGGCCCAAAAGTT |
| | | AAACAATGGCCACTGACAGAAGAAAAAATAAAAGCATTAATGGAAATCTG |
| | | TACAGAAATGGAAAAGGAAGGGAAAATTTCAAAAATTGGGCCTGAAAATC |
| | | CATACAATACTCCAGTATTTGCCATAAAGAAAAAAGACAGTACTAAATGGA |
| | | GAAAATTAGTAGATTTCAGAGAACTTAATAAGAAAACTCAAGACTTCTGGG |
| | | AAGTACAATTAGGAATACACATCCCGCAGGGGTTAAAAAAGAAAAAATCA |
| | | GTAACAGTACTGGATGTGGGTGATGCATATTTTTCAGTTCCCTTAGATAAAG |
| | | AATTCAGGAAGTATACTGCATTTACCATACCTAGTATAAACAATGAAACAC |
| | | CAGGGATCAGATATCAGTACAATGTACTTCCACAGGGATGGAAAGGATCAC |
| | | CAGCAATATTTCAAAGTAGCATGACAAGAATCTTAGAGCCTTTTAGAAAAC |
| | | AAAATCCAGAAATAGTGATCTATCAATACATGGATGATTTGTATGTAGGAT |
| | | CTGACTTAGAAATAGGGCAGCATAGAACAAAAATAGAGGAACTGAGACAA |
| | | CATCTGTTGAGGTGGGGATTTACCACACCAGACAAAAAACATCAGAAAGAA |
| | | CCTCCATTCCTTTGGATGGGTTATGAACTCCATCCTGATAAATGGACAGTAC |
| | | AGCCTATAGTGCTGCCAGAAAAAGACAGCTGGACTGTCAATGACATACAGA |
| | | AGTTAGTGGGAAAATTGAATTGGGCAAGTCAGATTTACCCAGGAATTAAAG |
| | | TAAAGCAATTATGTAGGCTCCTTAGGGGAACCAAGGCATTAACAGAAGTAA |
| | | TACCACTAACAAAAGAAACAGAGCTAGAACTGGCAGAGAACAGGGAAATT |
| | | CTAAAAGAACCAGTACATGGGGTGTATTATGACCCATCAAAAGACTTAATA |
| | | GCAGAAATACAGAAGCAGGGGCAAGGCCAATGGACATATCAAATTTATCA |
| | | AGAGCCATTTAAAAATCTGAAAACAGGAAAATATGCAAGAATGAGGGGTG |
| | | CCCACACTAATGATGTAAAACAATTAACAGAGGCAGTGCAAAAAATAACCA |
| | | CAGAAAGCATAGTAATATGGGGAAAGACTCCTAAATTTAAACTACCCATAC |
| | | AAAAAGAAACATGGGAAACATGGTGGACAGAGTATTGGCAAGCCACCTGG |
| | | ATTCCTGAGTGGGAGTTTGTCAATACCCCTCCCTTAGTGAAATTATGGTACC |
| | | AGTTAGAGAAAGAACCCATAATAGGAGCAGAAACATTCTATGTAGATGGA |
| | | GCAGCTAACCGGGAGACTAAATTAGGAAAAGCAGGATATGTTACTAACAG |
| | | AGGAAGACAAAAAGTTGTCTCCCTAACTGACACAACAAATCAGAAGACTGA |
| | | GTTACAAGCAATTCATCTAGCTTTACAAGATTCAGGATTAGAAGTAAACAT |
| | | AGTAACAGACTCACAATATGCATTAGGAATCATTCAAGCACAACCAGATAA |
| | | AAGTGAATCAGAGTTAGTCAGTCAAATAATAGAACAGTTAATAAAAAAGG |
| | | AAAAGGTCTACCTGGCATGGGTACCAGCGCACAAAGGAATTGGAGGAAAT |
| | | GAACAAGTAGATAAATTAGTCAGTACTGGAATCAGGAAAGTACTA |
| 53 | HIV Integrase; | TTTTTAGATGGAATAGATATAGCCCAAGAAGAACATGAGAAATATCACAGT |
| | Bal | AATTGGAGAGCAATGGCTAGTGATTTTAACCTGCCACCTGTGGTAGCAAAA |
| | | GAAATAGTAGCCAGCTGTGATAAATGTCAGCTAAAAGGAGAAGCCATGCAT |
| | | GGACAAGTAGACTGTAGTCCAGGAATATGGCAACTAGATTGTACACATTTA |
| | | GAAGGAAAAATTATCCTGGTAGCAGTTCATGTAGCCAGTGGATATATAGAA |
| | | GCAGAAGTTATTCCAGCAGAGACAGGGCAGGAAACAGCATACTTTCTCTTA |
| | | AAATTAGCAGGAAGATGGCCAGTAAAAACAATACATACAGACAATGGCAG |
| | | CAATTTCACTAGTACTACAGTCAAGGCCGCCTGTTGGTGGGCGGGGATCAA |
| | | GCAGGAATTTGGCATTCCCTACAATCCCCAAAGTCAGGGAGTAGTAGAATC |
| | | TATAAATAAAGAATTAAAGAAAATTATAGGACAGGTAAGAGATCAGGCTG |
| | | AACATCTTAAAACAGCAGTACAAATGGCAGTATTCATCCACAATTTTAAAA |
| | | GAAAAGGGGGGATTGGGGGGTATAGTGCAGGGGAAAGAATAGTAGACATA |
| | | ATAGCAACAGACATACAAACTAAAGAATTACAAAAACAAATTACAAAAAT |
| | | TCAAAATTTTCGGGTTTATTACAGGGACAGCAGAGATCCACTTTGGAAAGG |
| | | ACCAGCAAAGCTTCTCTGGAAAGGTGAAGGGGCAGTAGTAATACAAGATA |
| | | ATAGTGACATAAAAGTAGTACCAAGAAGAAAAGCAAAGATCATTAGGGAT |
| | | TATGGAAAACAGATGGCAGGTGATGATTGTGTGGCAAGTAGACAGGATGA |
| | | GGATTAG |
| 54 | Envelope; | ATGAAACTCCCAACAGGAATGGTCATTTTATGTAGCCTAATAATAGTTCGG |
| | RD114 | GCAGGGTTTGACGACCCCCGCAAGGCTATCGCATTAGTACAAAAACAACAT |
| | | GGTAAACCATGCGAATGCAGCGGAGGGCAGGTATCCGAGGCCCCACCGAA |
| | | CTCCATCCAACAGGTAACTTGCCCAGGCAAGACGGCCTACTTAATGACCAA |
| | | CCAAAAATGGAAATGCAGAGTCACTCCAAAAAATCTCACCCCTAGCGGGGG |
| | | AGAACTCCAGAACTGCCCCTGTAACACTTTCCAGGACTCGATGCACAGTTCT |
| | | TGTTATACTGAATACCGGCAATGCAGGGCGAATAATAAGACATACTACACG |
| | | GCCACCTTGCTTAAAATACGGTCTGGGAGCCTCAACGAGGTACAGATATTA |
| | | CAAAACCCCAATCAGCTCCTACAGTCCCCTTGTAGGGGCTCTATAAATCAGC |
| | | CCGTTTGCTGGAGTGCCACAGCCCCCATCCATATCTCCGATGGTGGAGGACC |
| | | CCTCGATACTAAGAGAGTGTGGACAGTCCAAAAAAGGCTAGAACAAATTCA |
| | | TAAGGCTATGCATCCTGAACTTCAATACCACCCCTTAGCCCTGCCCAAAGTC |
| | | AGAGATGACCTTAGCCTTGATGCACGGACTTTTGATATCCTGAATACCACTT |
| | | TTAGGTTACTCCAGATGTCCAATTTTAGCCTTGCCCAAGATTGTTGGCTCTG |
| | | TTTAAAACTAGGTACCCCTACCCCTCTTGCGATACCCACTCCCTCTTTAACCT |
| | | ACTCCCTAGCAGACTCCCTAGCGAATGCCTCCTGTCAGATTATACCTCCCCT |
| | | CTTGGTTCAACCGATGCAGTTCTCCAACTCGTCCTGTTTATCTTCCCCTTTCA |
| | | TTAACGATACGGAACAAATAGACTTAGGTGCAGTCACCTTTACTAACTGCA |
| | | CCTCTGTAGCCAATGTCAGTAGTCCTTTATGTGCCCTAAACGGGTCAGTCTT |
| | | CCTCTGTGGAAATAACATGGCATACACCTATTTACCCCAAAACTGGACAGG |
| | | ACTTTGCGTCCAAGCCTCCCTCCTCCCCGACATTGACATCATCCCGGGGGAT |
| | | GAGCCAGTCCCCATTCCTGCCATTGATCATTATATACATAGACCTAAACGAG |
| | | CTGTACAGTTCATCCCTTTACTAGCTGGACTGGGAATCACCGCAGCATTCAC |
| | | CACCGGAGCTACAGGCCTAGGTGTCTCCGTCACCCAGTATACAAAATTATC |
| | | CCATCAGTTAATATCTGATGTCCAAGTCTTATCCGGTACCATACAAGATTTA |
| | | CAAGACCAGGTAGACTCGTTAGCTGAAGTAGTTCTCCAAAATAGGAGGGGA |
| | | CTGGACCTACTAACGGCAGAACAAGGAGGAATTTGTTTAGCCTTACAAGAA |
| | | AAATGCTGTTTTTATGCTAACAAGTCAGGAATTGTGAGAAACAAAATAAGA |
| | | ACCCTACAAGAAGAATTACAAAAACGCAGGGAAAGCCTGGCATCCAACCCT |
| | | CTCTGGACCGGGCTGCAGGGCTTTCTTCCGTACCTCCTACCTCTCCTGGGAC |
| | | CCCTACTCACCCTCCTACTCATACTAACCATTGGGCCATGCGTTTTCAATCG |
| | | ATTGGTCCAATTTGTTAAAGACAGGATCTCAGTGGTCCAGGCTCTGGTTTTG |
| | | ACTCAGCAATATCACCAGCTAAAACCCATAGAGTACGAGCCATGA |
| 55 | Envelope; | ATGCTTCTCACCTCAAGCCCGCACCACCTTCGGCACCAGATGAGTCCTGGGA |
| | GALV | GCTGGAAAAGACTGATCATCCTCTTAAGCTGCGTATTCGGAGACGGCAAAA |
| | | CGAGTCTGCAGAATAAGAACCCCCACCAGCCTGTGACCCTCACCTGGCAGG |
| | | TACTGTCCCAAACTGGGGACGTTGTCTGGGACAAAAAGGCAGTCCAGCCCC |
| | | TTTGGACTTGGTGGCCCTCTCTTACACCTGATGTATGTGCCCTGGCGGCCGG |
| | | TCTTGAGTCCTGGGATATCCCGGGATCCGATGTATCGTCCTCTAAAAGAGTT |
| | | AGACCTCCTGATTCAGACTATACTGCCGCTTATAAGCAAATCACCTGGGGA |
| | | GCCATAGGGTGCAGCTACCCTCGGGCTAGGACCAGGATGGCAAATTCCCCC |
| | | TTCTACGTGTGTCCCCGAGCTGGCCGAACCCATTCAGAAGCTAGGAGGTGT |
| | | GGGGGGCTAGAATCCCTATACTGTAAAGAATGGAGTTGTGAGACCACGGGT |
| | | ACCGTTTATTGGCAACCCAAGTCCTCATGGGACCTCATAACTGTAAAATGG |
| | | GACCAAAATGTGAAATGGGAGCAAAAATTTCAAAAGTGTGAACAAACCGG |
| | | CTGGTGTAACCCCCTCAAGATAGACTTCACAGAAAAAGGAAAACTCTCCAG |
| | | AGATTGGATAACGGAAAAAACCTGGGAATTAAGGTTCTATGTATATGGACA |
| | | CCCAGGCATACAGTTGACTATCCGCTTAGAGGTCACTAACATGCCGGTTGTG |
| | | GCAGTGGGCCCAGACCCTGTCCTTGCGGAACAGGGACCTCCTAGCAAGCCC |
| | | CTCACTCTCCCTCTCTCCCCACGGAAAGCGCCGCCCACCCCTCTACCCCCGG |
| | | CGGCTAGTGAGCAAACCCCTGCGGTGCATGGAGAAACTGTTACCCTAAACT |
| | | CTCCGCCTCCCACCAGTGGCGACCGACTCTTTGGCCTTGTGCAGGGGGCCTT |
| | | CCTAACCTTGAATGCTACCAACCCAGGGGCCACTAAGTCTTGCTGGCTCTGT |
| | | TTGGGCATGAGCCCCCCTTATTATGAAGGGATAGCCTCTTCAGGAGAGGTC |
| | | GCTTATACCTCCAACCATACCCGATGCCACTGGGGGGCCCAAGGAAAGCTT |
| | | ACCCTCACTGAGGTCTCCGGACTCGGGTCATGCATAGGGAAGGTGCCTCTT |
| | | ACCCATCAACATCTTTGCAACCAGACCTTACCCATCAATTCCTCTAAAAACC |
| | | ATCAGTATCTGCTCCCCTCAAACCATAGCTGGTGGGCCTGCAGCACTGGCCT |
| | | CACCCCCTGCCTCTCCACCTCAGTTTTTAATCAGTCTAAAGACTTCTGTGTCC |
| | | AGGTCCAGCTGATCCCCCGCATCTATTACCATTCTGAAGAAACCTTGTTACA |
| | | AGCCTATGACAAATCACCCCCCAGGTTTAAAAGAGAGCCTGCCTCACTTAC |
| | | CCTAGCTGTCTTCCTGGGGTTAGGGATTGCGGCAGGTATAGGTACTGGCTCA |
| | | ACCGCCCTAATTAAAGGGCCCATAGACCTCCAGCAAGGCCTAACCAGCCTC |
| | | CAAATCGCCATTGACGCTGACCTCCGGGCCCTTCAGGACTCAATCAGCAAG |
| | | CTAGAGGACTCACTGACTTCCCTATCTGAGGTAGTACTCCAAAATAGGAGA |
| | | GGCCTTGACTTACTATTCCTTAAAGAAGGAGGCCTCTGCGCGGCCCTAAAA |
| | | GAAGAGTGCTGTTTTTATGTAGACCACTCAGGTGCAGTACGAGACTCCATG |
| | | AAAAAACTTAAAGAAAGACTAGATAAAAGACAGTTAGAGCGCCAGAAAAA |
| | | CCAAAACTGGTATGAAGGGTGGTTCAATAACTCCCCTTGGTTTACTACCCTA |
| | | CTATCAACCATCGCTGGGCCCCTATTGCTCCTCCTTTTGTTACTCACTCTTGG |
| | | GCCCTGCATCATCAATAAATTAATCCAATTCATCAATGATAGGATAAGTGC |
| | | AGTCAAAATTTTAGTCCTTAGACAGAAATATCAGACCCTAGATAACGAGGA |
| | | AAACCTTTAA |
| 56 | Envelope; FUG | ATGGTTCCGCAGGTTCTTTTGTTTGTACTCCTTCTGGGTTTTTCGTTGTGTTTC |
| | | GGGAAGTTCCCCATTTACACGATACCAGACGAACTTGGTCCCTGGAGCCCT |
| | | ATTGACATACACCATCTCAGCTGTCCAAATAACCTGGTTGTGGAGGATGAA |
| | | GGATGTACCAACCTGTCCGAGTTCTCCTACATGGAACTCAAAGTGGGATAC |
| | | ATCTCAGCCATCAAAGTGAACGGGTTCACTTGCACAGGTGTTGTGACAGAG |
| | | GCAGAGACCTACACCAACTTTGTTGGTTATGTCACAACCACATTCAAGAGA |
| | | AAGCATTTCCGCCCCACCCCAGACGCATGTAGAGCCGCGTATAACTGGAAG |
| | | ATGGCCGGTGACCCCAGATATGAAGAGTCCCTACACAATCCATACCCCGAC |
| | | TACCACTGGCTTCGAACTGTAAGAACCACCAAAGAGTCCCTCATTATCATAT |
| | | CCCCAAGTGTGACAGATTTGGACCCATATGACAAATCCCTTCACTCAAGGG |
| | | TCTTCCCTGGCGGAAAGTGCTCAGGAATAACGGTGTCCTCTACCTACTGCTC |
| | | AACTAACCATGATTACACCATTTGGATGCCCGAGAATCCGAGACCAAGGAC |
| | | ACCTTGTGACATTTTTACCAATAGCAGAGGGAAGAGAGCATCCAACGGGAA |
| | | CAAGACTTGCGGCTTTGTGGATGAAAGAGGCCTGTATAAGTCTCTAAAAGG |
| | | AGCATGCAGGCTCAAGTTATGTGGAGTTCTTGGACTTAGACTTATGGATGG |
| | | AACATGGGTCGCGATGCAAACATCAGATGAGACCAAATGGTGCCCTCCAGA |
| | | TCAGTTGGTGAATTTGCACGACTTTCGCTCAGACGAGATCGAGCATCTCGTT |
| | | GTGGAGGAGTTAGTTAAGAAAAGAGAGGAATGTCTGGATGCATTAGAGTCC |
| | | ATCATGACCACCAAGTCAGTAAGTTTCAGACGTCTCAGTCACCTGAGAAAA |
| | | CTTGTCCCAGGGTTTGGAAAAGCATATACCATATTCAACAAAACCTTGATG |
| | | GAGGCTGATGCTCACTACAAGTCAGTCCGGACCTGGAATGAGATCATCCCC |
| | | TCAAAAGGGTGTTTGAAAGTTGGAGGAAGGTGCCATCCTCATGTGAACGGG |
| | | GTGTTTTTCAATGGTATAATATTAGGGCCTGACGACCATGTCCTAATCCCAG |
| | | AGATGCAATCATCCCTCCTCCAGCAACATATGGAGTTGTTGGAATCTTCAGT |
| | | TATCCCCCTGATGCACCCCCTGGCAGACCCTTCTACAGTTTTCAAAGAAGGT |
| | | GATGAGGCTGAGGATTTTGTTGAAGTTCACCTCCCCGATGTGTACAAACAG |
| | | ATCTCAGGGGTTGACCTGGGTCTCCCGAACTGGGGAAAGTATGTATTGATG |
| | | ACTGCAGGGGCCATGATTGGCCTGGTGTTGATATTTTCCCTAATGACATGGT |
| | | GCAGAGTTGGTATCCATCTTTGCATTAAATTAAAGCACACCAAGAAAAGAC |
| | | AGATTTATACAGACATAGAGATGAACCGACTTGGAAAGTAA |
| 57 | Envelope; | ATGGGTCAGATTGTGACAATGTTTGAGGCTCTGCCTCACATCATCGATGAGG |
| | LCMV | TGATCAACATTGTCATTATTGTGCTTATCGTGATCACGGGTATCAAGGCTGT |
| | | CTACAATTTTGCCACCTGTGGGATATTCGCATTGATCAGTTTCCTACTTCTGG |
| | | CTGGCAGGTCCTGTGGCATGTACGGTCTTAAGGGACCCGACATTTACAAAG |
| | | GAGTTTACCAATTTAAGTCAGTGGAGTTTGATATGTCACATCTGAACCTGAC |
| | | CATGCCCAACGCATGTTCAGCCAACAACTCCCACCATTACATCAGTATGGG |
| | | GACTTCTGGACTAGAATTGACCTTCACCAATGATTCCATCATCAGTCACAAC |
| | | TTTTGCAATCTGACCTCTGCCTTCAACAAAAAGACCTTTGACCACACACTCA |
| | | TGAGTATAGTTTCGAGCCTACACCTCAGTATCAGAGGGAACTCCAACTATA |
| | | AGGCAGTATCCTGCGACTTCAACAATGGCATAACCATCCAATACAACTTGA |
| | | CATTCTCAGATCGACAAAGTGCTCAGAGCCAGTGTAGAACCTTCAGAGGTA |
| | | GAGTCCTAGATATGTTTAGAACTGCCTTCGGGGGGAAATACATGAGGAGTG |
| | | GCTGGGGCTGGACAGGCTCAGATGGCAAGACCACCTGGTGTAGCCAGACGA |
| | | GTTACCAATACCTGATTATACAAAATAGAACCTGGGAAAACCACTGCACAT |
| | | ATGCAGGTCCTTTTGGGATGTCCAGGATTCTCCTTTCCCAAGAGAAGACTAA |
| | | GTTCTTCACTAGGAGACTAGCGGGCACATTCACCTGGACTTTGTCAGACTCT |
| | | TCAGGGGTGGAGAATCCAGGTGGTTATTGCCTGACCAAATGGATGATTCTT |
| | | GCTGCAGAGCTTAAGTGTTTCGGGAACACAGCAGTTGCGAAATGCAATGTA |
| | | AATCATGATGCCGAATTCTGTGACATGCTGCGACTAATTGACTACAACAAG |
| | | GCTGCTTTGAGTAAGTTCAAAGAGGACGTAGAATCTGCCTTGCACTTATTCA |
| | | AAACAACAGTGAATTCTTTGATTTCAGATCAACTACTGATGAGGAACCACTT |
| | | GAGAGATCTGATGGGGGTGCCATATTGCAATTACTCAAAGTTTTGGTACCTA |
| | | GAACATGCAAAGACCGGCGAAACTAGTGTCCCCAAGTGCTGGCTTGTCACC |
| | | AATGGTTCTTACTTAAATGAGACCCACTTCAGTGATCAAATCGAACAGGAA |
| | | GCCGATAACATGATTACAGAGATGTTGAGGAAGGATTACATAAAGAGGCA |
| | | GGGGAGTACCCCCCTAGCATTGATGGACCTTCTGATGTTTTCCACATCTGCA |
| | | TATCTAGTCAGCATCTTCCTGCACCTTGTCAAAATACCAACACACAGGCACA |
| | | TAAAAGGTGGCTCATGTCCAAAGCCACACCGATTAACCAACAAAGGAATTT |
| | | GTAGTTGTGGTGCATTTAAGGTGCCTGGTGTAAAAACCGTCTGGAAAAGAC |
| | | GCTGA |
| 58 | Envelope; FPV | ATGAACACTCAAATCCTGGTTTTCGCCCTTGTGGCAGTCATCCCCACAAATG |
| | | CAGACAAAATTTGTCTTGGACATCATGCTGTATCAAATGGCACCAAAGTAA |
| | | ACACACTCACTGAGAGAGGAGTAGAAGTTGTCAATGCAACGGAAACAGTG |
| | | GAGCGGACAAACATCCCCAAAATTTGCTCAAAAGGGAAAAGAACCACTGA |
| | | TCTTGGCCAATGCGGACTGTTAGGGACCATTACCGGACCACCTCAATGCGA |
| | | CCAATTTCTAGAATTTTCAGCTGATCTAATAATCGAGAGACGAGAAGGAAA |
| | | TGATGTTTGTTACCCGGGGAAGTTTGTTAATGAAGAGGCATTGCGACAAAT |
| | | CCTCAGAGGATCAGGTGGGATTGACAAAGAAACAATGGGATTCACATATAG |
| | | TGGAATAAGGACCAACGGAACAACTAGTGCATGTAGAAGATCAGGGTCTTC |
| | | ATTCTATGCAGAAATGGAGTGGCTCCTGTCAAATACAGACAATGCTGCTTTC |
| | | CCACAAATGACAAAATCATACAAAAACACAAGGAGAGAATCAGCTCTGAT |
| | | AGTCTGGGGAATCCACCATTCAGGATCAACCACCGAACAGACCAAACTATA |
| | | TGGGAGTGGAAATAAACTGATAACAGTCGGGAGTTCCAAATATCATCAATC |
| | | TTTTGTGCCGAGTCCAGGAACACGACCGCAGATAAATGGCCAGTCCGGACG |
| | | GATTGATTTTCATTGGTTGATCTTGGATCCCAATGATACAGTTACTTTTAGTT |
| | | TCAATGGGGCTTTCATAGCTCCAAATCGTGCCAGCTTCTTGAGGGGAAAGTC |
| | | CATGGGGATCCAGAGCGATGTGCAGGTTGATGCCAATTGCGAAGGGGAATG |
| | | CTACCACAGTGGAGGGACTATAACAAGCAGATTGCCTTTTCAAAACATCAA |
| | | TAGCAGAGCAGTTGGCAAATGCCCAAGATATGTAAAACAGGAAAGTTTATT |
| | | ATTGGCAACTGGGATGAAGAACGTTCCCGAACCTTCCAAAAAAAGGAAAA |
| | | AAAGAGGCCTGTTTGGCGCTATAGCAGGGTTTATTGAAAATGGTTGGGAAG |
| | | GTCTGGTCGACGGGTGGTACGGTTTCAGGCATCAGAATGCACAAGGAGAAG |
| | | GAACTGCAGCAGACTACAAAAGCACCCAATCGGCAATTGATCAGATAACCG |
| | | GAAAGTTAAATAGACTCATTGAGAAAACCAACCAGCAATTTGAGCTAATAG |
| | | ATAATGAATTCACTGAGGTGGAAAAGCAGATTGGCAATTTAATTAACTGGA |
| | | CCAAAGACTCCATCACAGAAGTATGGTCTTACAATGCTGAACTTCTTGTGGC |
| | | AATGGAAAACCAGCACACTATTGATTTGGCTGATTCAGAGATGAACAAGCT |
| | | GTATGAGCGAGTGAGGAAACAATTAAGGGAAAATGCTGAAGAGGATGGCA |
| | | CTGGTTGCTTTGAAATTTTTCATAAATGTGACGATGATTGTATGGCTAGTAT |
| | | AAGGAACAATACTTATGATCACAGCAAATACAGAGAAGAAGCGATGCAAA |
| | | ATAGAATACAAATTGACCCAGTCAAATTGAGTAGTGGCTACAAAGATGTGA |
| | | TACTTTGGTTTAGCTTCGGGGCATCATGCTTTTTGCTTCTTGCCATTGCAATG |
| | | GGCCTTGTTTTCATATGTGTGAAGAACGGAAACATGCGGTGCACTATTTGTA |
| | | TATAA |
| 59 | Envelope; RRV | AGTGTAACAGAGCACTTTAATGTGTATAAGGCTACTAGACCATACCTAGCA |
| | | CATTGCGCCGATTGCGGGGACGGGTACTTCTGCTATAGCCCAGTTGCTATCG |
| | | AGGAGATCCGAGATGAGGCGTCTGATGGCATGCTTAAGATCCAAGTCTCCG |
| | | CCCAAATAGGTCTGGACAAGGCAGGCACCCACGCCCACACGAAGCTCCGAT |
| | | ATATGGCTGGTCATGATGTTCAGGAATCTAAGAGAGATTCCTTGAGGGTGT |
| | | ACACGTCCGCAGCGTGCTCCATACATGGGACGATGGGACACTTCATCGTCG |
| | | CACACTGTCCACCAGGCGACTACCTCAAGGTTTCGTTCGAGGACGCAGATT |
| | | CGCACGTGAAGGCATGTAAGGTCCAATACAAGCACAATCCATTGCCGGTGG |
| | | GTAGAGAGAAGTTCGTGGTTAGACCACACTTTGGCGTAGAGCTGCCATGCA |
| | | CCTCATACCAGCTGACAACGGCTCCCACCGACGAGGAGATTGACATGCATA |
| | | CACCGCCAGATATACCGGATCGCACCCTGCTATCACAGACGGCGGGCAACG |
| | | TCAAAATAACAGCAGGCGGCAGGACTATCAGGTACAACTGTACCTGCGGCC |
| | | GTGACAACGTAGGCACTACCAGTACTGACAAGACCATCAACACATGCAAGA |
| | | TTGACCAATGCCATGCTGCCGTCACCAGCCATGACAAATGGCAATTTACCTC |
| | | TCCATTTGTTCCCAGGGCTGATCAGACAGCTAGGAAAGGCAAGGTACACGT |
| | | TCCGTTCCCTCTGACTAACGTCACCTGCCGAGTGCCGTTGGCTCGAGCGCCG |
| | | GATGCCACCTATGGTAAGAAGGAGGTGACCCTGAGATTACACCCAGATCAT |
| | | CCGACGCTCTTCTCCTATAGGAGTTTAGGAGCCGAACCGCACCCGTACGAG |
| | | GAATGGGTTGACAAGTTCTCTGAGCGCATCATCCCAGTGACGGAAGAAGGG |
| | | ATTGAGTACCAGTGGGGCAACAACCCGCCGGTCTGCCTGTGGGCGCAACTG |
| | | ACGACCGAGGGCAAACCCCATGGCTGGCCACATGAAATCATTCAGTACTAT |
| | | TATGGACTATACCCCGCCGCCACTATTGCCGCAGTATCCGGGGCGAGTCTG |
| | | ATGGCCCTCCTAACTCTGGCGGCCACATGCTGCATGCTGGCCACCGCGAGG |
| | | AGAAAGTGCCTAACACCGTACGCCCTGACGCCAGGAGCGGTGGTACCGTTG |
| | | ACACTGGGGCTGCTTTGCTGCGCACCGAGGGCGAATGCA |
| 60 | Envelope; MLV | GGATCCACGCCGCTCACGTAAAGGCGGCGACAACCCCTCCGGCCGGAACAG |
| | 10A1 | CATCAGGACCGACATGGAAGGTCCAGCGTTCTCAAAACCCCTTAAAGATAA |
| | | GATTAACCCGTGGAAGTCCTTAATGGTCATGGGGGTCTATTTAAGAGTAGG |
| | | GATGGCAGAGAGCCCCCATCAGGTCTTTAATGTAACCTGGAGAGTCACCAA |
| | | CCTGATGACTGGGCGTACCGCCAATGCCACCTCCCTTTTAGGAACTGTACAA |
| | | GATGCCTTCCCAAGATTATATTTTGATCTATGTGATCTGGTCGGAGAAGAGT |
| | | GGGACCCTTCAGACCAGGAACCATATGTCGGGTATGGCTGCAAATACCCCG |
| | | GAGGGAGAAAGCGGACCCGGACTTTTGACTTTTACGTGTGCCCTGGGCATA |
| | | CCGTAAAATCGGGGTGTGGGGGGCCAAGAGAGGGCTACTGTGGTGAATGG |
| | | GGTTGTGAAACCACCGGACAGGCTTACTGGAAGCCCACATCATCATGGGAC |
| | | CTAATCTCCCTTAAGCGCGGTAACACCCCCTGGGACACGGGATGCTCCAAA |
| | | ATGGCTTGTGGCCCCTGCTACGACCTCTCCAAAGTATCCAATTCCTTCCAAG |
| | | GGGCTACTCGAGGGGGCAGATGCAACCCTCTAGTCCTAGAATTCACTGATG |
| | | CAGGAAAAAAGGCTAATTGGGACGGGCCCAAATCGTGGGGACTGAGACTG |
| | | TACCGGACAGGAACAGATCCTATTACCATGTTCTCCCTGACCCGCCAGGTCC |
| | | TCAATATAGGGCCCCGCATCCCCATTGGGCCTAATCCCGTGATCACTGGTCA |
| | | ACTACCCCCCTCCCGACCCGTGCAGATCAGGCTCCCCAGGCCTCCTCAGCCT |
| | | CCTCCTACAGGCGCAGCCTCTATAGTCCCTGAGACTGCCCCACCTTCTCAAC |
| | | AACCTGGGACGGGAGACAGGCTGCTAAACCTGGTAGAAGGAGCCTATCAG |
| | | GCGCTTAACCTCACCAATCCCGACAAGACCCAAGAATGTTGGCTGTGCTTA |
| | | GTGTCGGGACCTCCTTATTACGAAGGAGTAGCGGTCGTGGGCACTTATACC |
| | | AATCATTCTACCGCCCCGGCCAGCTGTACGGCCACTTCCCAACATAAGCTTA |
| | | CCCTATCTGAAGTGACAGGACAGGGCCTATGCATGGGAGCACTACCTAAAA |
| | | CTCACCAGGCCTTATGTAACACCACCCAAAGTGCCGGCTCAGGATCCTACT |
| | | ACCTTGCAGCACCCGCTGGAACAATGTGGGCTTGTAGCACTGGATTGACTC |
| | | CCTGCTTGTCCACCACGATGCTCAATCTAACCACAGACTATTGTGTATTAGT |
| | | TGAGCTCTGGCCCAGAATAATTTACCACTCCCCCGATTATATGTATGGTCAG |
| | | CTTGAACAGCGTACCAAATATAAGAGGGAGCCAGTATCGTTGACCCTGGCC |
| | | CTTCTGCTAGGAGGATTAACCATGGGAGGGATTGCAGCTGGAATAGGGACG |
| | | GGGACCACTGCCCTAATCAAAACCCAGCAGTTTGAGCAGCTTCACGCCGCT |
| | | ATCCAGACAGACCTCAACGAAGTCGAAAAATCAATTACCAACCTAGAAAAG |
| | | TCACTGACCTCGTTGTCTGAAGTAGTCCTACAGAACCGAAGAGGCCTAGAT |
| | | TTGCTCTTCCTAAAAGAGGGAGGTCTCTGCGCAGCCCTAAAAGAAGAATGT |
| | | TGTTTTTATGCAGACCACACGGGACTAGTGAGAGACAGCATGGCCAAACTA |
| | | AGGGAAAGGCTTAATCAGAGACAAAAACTATTTGAGTCAGGCCAAGGTTGG |
| | | TTCGAAGGGCAGTTTAATAGATCCCCCTGGTTTACCACCTTAATCTCCACCA |
| | | TCATGGGACCTCTAATAGTACTCTTACTGATCTTACTCTTTGGACCCTGCATT |
| | | CTCAATCGATTGGTCCAATTTGTTAAAGACAGGATCTCAGTGGTCCAGGCTC |
| | | TGGTTTTGACTCAACAATATCACCAGCTAAAACCTATAGAGTACGAGCCAT |
| | | GA |
| 61 | Envelope; | ATGGGTGTTACAGGAATATTGCAGTTACCTCGTGATCGATTCAAGAGGACA |
| | Ebola | TCATTCTTTCTTTGGGTAATTATCCTTTTCCAAAGAACATTTTCCATCCCACT |
| | | TGGAGTCATCCACAATAGCACATTACAGGTTAGTGATGTCGACAAACTGGT |
| | | TTGCCGTGACAAACTGTCATCCACAAATCAATTGAGATCAGTTGGACTGAA |
| | | TCTCGAAGGGAATGGAGTGGCAACTGACGTGCCATCTGCAACTAAAAGATG |
| | | GGGCTTCAGGTCCGGTGTCCCACCAAAGGTGGTCAATTATGAAGCTGGTGA |
| | | ATGGGCTGAAAACTGCTACAATCTTGAAATCAAAAAACCTGACGGGAGTGA |
| | | GTGTCTACCAGCAGCGCCAGACGGGATTCGGGGCTTCCCCCGGTGCCGGTA |
| | | TGTGCACAAAGTATCAGGAACGGGACCGTGTGCCGGAGACTTTGCCTTCCA |
| | | CAAAGAGGGTGCTTTCTTCCTGTATGACCGACTTGCTTCCACAGTTATCTAC |
| | | CGAGGAACGACTTTCGCTGAAGGTGTCGTTGCATTTCTGATACTGCCCCAAG |
| | | CTAAGAAGGACTTCTTCAGCTCACACCCCTTGAGAGAGCCGGTCAATGCAA |
| | | CGGAGGACCCGTCTAGTGGCTACTATTCTACCACAATTAGATATCAAGCTAC |
| | | CGGTTTTGGAACCAATGAGACAGAGTATTTGTTCGAGGTTGACAATTTGACC |
| | | TACGTCCAACTTGAATCAAGATTCACACCACAGTTTCTGCTCCAGCTGAATG |
| | | AGACAATATATACAAGTGGGAAAAGGAGCAATACCACGGGAAAACTAATT |
| | | TGGAAGGTCAACCCCGAAATTGATACAACAATCGGGGAGTGGGCCTTCTGG |
| | | GAAACTAAAAAAACCTCACTAGAAAAATTCGCAGTGAAGAGTTGTCTTTCA |
| | | CAGCTGTATCAAACAGAGCCAAAAACATCAGTGGTCAGAGTCCGGCGCGAA |
| | | CTTCTTCCGACCCAGGGACCAACACAACAACTGAAGACCACAAAATCATGG |
| | | CTTCAGAAAATTCCTCTGCAATGGTTCAAGTGCACAGTCAAGGAAGGGAAG |
| | | CTGCAGTGTCGCATCTGACAACCCTTGCCACAATCTCCACGAGTCCTCAACC |
| | | CCCCACAACCAAACCAGGTCCGGACAACAGCACCCACAATACACCCGTGTA |
| | | TAAACTTGACATCTCTGAGGCAACTCAAGTTGAACAACATCACCGCAGAAC |
| | | AGACAACGACAGCACAGCCTCCGACACTCCCCCCGCCACGACCGCAGCCGG |
| | | ACCCCTAAAAGCAGAGAACACCAACACGAGCAAGGGTACCGACCTCCTGG |
| | | ACCCCGCCACCACAACAAGTCCCCAAAACCACAGCGAGACCGCTGGCAACA |
| | | ACAACACTCATCACCAAGATACCGGAGAAGAGAGTGCCAGCAGCGGGAAG |
| | | CTAGGCTTAATTACCAATACTATTGCTGGAGTCGCAGGACTGATCACAGGC |
| | | GGGAGGAGAGCTCGAAGAGAAGCAATTGTCAATGCTCAACCCAAATGCAA |
| | | CCCTAATTTACATTACTGGACTACTCAGGATGAAGGTGCTGCAATCGGACTG |
| | | GCCTGGATACCATATTTCGGGCCAGCAGCCGAGGGAATTTACATAGAGGGG |
| | | CTGATGCACAATCAAGATGGTTTAATCTGTGGGTTGAGACAGCTGGCCAAC |
| | | GAGACGACTCAAGCTCTTCAACTGTTCCTGAGAGCCACAACCGAGCTACGC |
| | | ACCTTTTCAATCCTCAACCGTAAGGCAATTGATTTCTTGCTGCAGCGATGGG |
| | | GCGGCACATGCCACATTTTGGGACCGGACTGCTGTATCGAACCACATGATT |
| | | GGACCAAGAACATAACAGACAAAATTGATCAGATTATTCATGATTTTGTTG |
| | | ATAAAACCCTTCCGGACCAGGGGGACAATGACAATTGGTGGACAGGATGG |
| | | AGACAATGGATACCGGCAGGTATTGGAGTTACAGGCGTTATAATTGCAGTT |
| | | ATCGCTTTATTCTGTATATGCAAATTTGTCTTTTAG |
| 62 | miR30-CCR5 | AGGTATATTGCTGTTGACAGTGAGCGACTGTAAACTGAGCTTGCTCTACTGT |
| | | GAAGCCACAGATGGGTAGAGCAAGCACAGTTTACCGCTGCCTACTGCCTCG |
| | | GACTTCAAGGGGCTT |
| 63 | miR21-Vif | CATCTCCATGGCTGTACCACCTTGTCGGGGGATGTGTACTTCTGAACTTGTG |
| | | TTGAATCTCATGGAGTTCAGAAGAACACATCCGCACTGACATTTTGGTATCT |
| | | TTCATCTGACCA |
| 64 | miR185-Tat | GGGCCTGGCTCGAGCAGGGGGCGAGGGATTCCGCTTCTTCCTGCCATAGCG |
| | | TGGTCCCCTCCCCTATGGCAGGCAGAAGCGGCACCTTCCCTCCCAATGACCG |
| | | CGTCTTCGTC |
| 65 | miR30- | AGGTATATTGCTGTTGACAGTGAGCGACTGTAAACTGAGCTTGCTCTACTGT |
| | CCR5/miR21- | GAAGCCACAGATGGGTAGAGCAAGCACAGTTTACCGCTGCCTACTGCCTCG |
| | Vif/miR185-Tat | GACTTCAAGGGGCTTCCCGGGCATCTCCATGGCTGTACCACCTTGTCGGGGG |
| | microRNA | ATGTGTACTTCTGAACTTGTGTTGAATCTCATGGAGTTCAGAAGAACACATC |
| | cluster | CGCACTGACATTTTGGTATCTTTCATCTGACCAGCTAGCGGGCCTGGCTCGA |
| | sequence | GCAGGGGGCGAGGGATTCCGCTTCTTCCTGCCATAGCGTGGTCCCCTCCCCT |
| | | ATGGCAGGCAGAAGCGGCACCTTCCCTCCCAATGACCGCGTCTTCGTC |
| 66 | IL-2 promoter | ATCTATCTTATTGTATGCAATTAGCTCATTGTGTGGATAAAAAGGTAAAACC |
| | | ATTCTGAAACAGGAAACCAATACACTTCCTGTTTAATCAACAAATCTAAAC |
| | | ATTTATTCTTTTCATCTGTTTACTCTTGCTCTTGTCCACCACAATATGCTATTC |
| | | ACATGTTCAGTGTAGTTTTATGACAAAGAAAATTTTCTGAGTTACTTTTGTA |
| | | TCCCCACCCCCTTAAAGAAAGGAGGAAAAACTGTTTCATACAGAAGGCGTT |
| | | AATTGCATGAATTAGAGCTATCACCTAAGTGTGGGCTAATGTAACAAAGAG |
| | | GGATTTCACCTACATCCATTCAGTCAGTCTTTGGGGGTTTAAAGAAATTCCA |
| | | AAGAGTCATCAGAAGAGGAAAAATGAAGGTAATGTTTTTTCAGACTGGTAA |
| | | AGTCTTTGAAAATATGTGTAATATGTAAAACATTTTGACACCCCCATAATAT |
| | | TTTTCCAGAATTAACAGTATAAATTGCATCTCTTGTTCAAGAGTTCCCTATC |
| | | ACTCTT |
| 67 | CD69 promoter | CAGACACTGAGGCTTGGGTTGGGCGAGGCCATTCAGACACTAAAACCCAGT |
| | (1050) + CNS2 | GCAGTTCTCCCTCAAGTGTGACCTTATATGAAGAATCCGGAGGGAGGTTTCT |
| | enhancer | GAAAGGAAATGATGTAATGTGAGGCAGATGCAAAGTGCGGCAGGAAGGCA |
| | | GGGTGTACAGTCCTTATCACGGCAGCTGCCTTAGTGGTATGTGTTCAAAGGA |
| | | ACCACAAACTTCCGACCTGAGGCAGTTTCCGGTGACAACCTGCTCATCATAT |
| | | TTCAAAATGATTTTTTTCTTTCAGTGAGTGAATGAGGTACTGGAATGTCCTC |
| | | TAGATGATAACTTCCAACCCACCTATGCATAAAATTTAACGTCTTTATTCTA |
| | | AATAAGTGATATTAATAATAAAATTTGGGGCACCAAGATTATTAATCAGAG |
| | | TGGTATTTTGATTTCCCTCCTTAAATCACCATACATAGCTTTCTGCATTCATC |
| | | TTGCGTTGACTGTCATTACTTGTCTGAGTGAGACTGATACCACAGCGATGTT |
| | | TTAAATAATAATCATACCTCAAAAGACTGAAGTCTCAGAGGTATCTGAAGA |
| | | GAATAACCTAGAGCACAGGGGGAGAATTGAAGGAGCTGTTACTGAGGTGA |
| | | CATAAAAGCAGTCTAAATGACAGTAAAATGTGACAAGAAAATTAGCAGGA |
| | | AACAAATGAAACAGATAATTTAAGATAAACAATTTTAGAGCATAGCAAGGA |
| | | AGTTCCAGACCACAAGCTTTCTGTTTCCTGCATTCTTACTTCTTACTACGTGA |
| | | TACATCTAGTCACCAGGGAAGAAGCGAATGACACACTTCCAAAAACCAATT |
| | | CGTAGCTTTCTAAATAAAACCCTTTCTAGCTGGAGAGAGATCCATGAGCAT |
| | | AGAGATCTTAAAATTCATGTTCAGCAATAAATCCTGGGGCCCCAGACAGTG |
| | | TCAGGTGCATAGGGGGTGTTCAGTAAATATCAGTTAAATGTATGCATAAAT |
| | | CGATAAACGGGATTCCTGGAAAATACTACACTCTCCTTCTCCAAATTATCTT |
| | | CATCTCAAAGACAGGAACCTCTAACTTTTAATTCTTTACTTAGATTATGCTG |
| | | TCTCCTAAACTGTTTATGTTTTCTAGAAATTTAAGGCAGGATGTCTCAGAGT |
| | | CTGGGAAAATCCCACTTTCCTCCTGCTACACCTTACAGTTGTGAGAAAGCAC |
| | | ATTTCAGACAACAGGGAAAACCCATACTTCACCACAACAACACACTATACA |
| | | TTGTCTGGTCCACTGGAGCATAAATTAAAGAGAAACAATGTAGTCAAGCAA |
| | | GTAGGCGGCAAGAGGAAGGGGGCGGAGACATCATCAGGGAGTATAAACTC |
| | | TGAGATGCCTCAGAGCCTCAC |
| 68 | CD69 promoter | CAGACACTGAGGCTTGGGTTGGGCGAGGCCATTCAGACACTAAAACCCAGT |
| | (625) + CNS2 | GCAGTTCTCCCTCAAGTGTGACCTTATATGAAGAATCCGGAGGGAGGTTTCT |
| | enhancer | GAAAGGAAATGATGTAATGTGAGGCAGATGCAAAGTGCGGCAGGAAGGCA |
| | | GGGTGTACAGTCCTTATCACGGCAGCTGCCTTAGTGGTATGTGTTCAAAGGA |
| | | ACCACAAACTTCCGACCTGAGGCAGTTTCCGGTGACAACCTGCTCATCATAT |
| | | TTCAAAATGATTTTTTTCTTTCAGTGAGTGAATGAGGTACTGGAATGTCCAA |
| | | GCTTTCTGTTTCCTGCATTCTTACTTCTTACTACGTGATACATCTAGTCACCA |
| | | GGGAAGAAGCGAATGACACACTTCCAAAAACCAATTCGTAGCTTTCTAAAT |
| | | AAAACCCTTTCTAGCTGGAGAGAGATCCATGAGCATAGAGATCTTAAAATT |
| | | CATGTTCAGCAATAAATCCTGGGGCCCCAGACAGTGTCAGGTGCATAGGGG |
| | | GTGTTCAGTAAATATCAGTTAAATGTATGCATAAATCGATAAACGGGATTC |
| | | CTGGAAAATACTACACTCTCCTTCTCCAAATTATCTTCATCTCAAAGACAGG |
| | | AACCTCTAACTTTTAATTCTTTACTTAGATTATGCTGTCTCCTAAACTGTTTA |
| | | TGTTTTCTAGAAATTTAAGGCAGGATGTCTCAGAGTCTGGGAAAATCCCACT |
| | | TTCCTCCTGCTACACCTTACAGTTGTGAGAAAGCACATTTCAGACAACAGGG |
| | | AAAACCCATACTTCACCACAACAACACACTATACATTGTCTGGTCCACTGG |
| | | AGCATAAATTAAAGAGAAACAATGTAGTCAAGCAAGTAGGCGGCAAGAGG |
| | | AAGGGGGCGGAGACATCATCAGGGAGTATAAACTCTGAGATGCCTCAGAG |
| | | CCTCAC |
| 69 | VRC01 Heavy | ATGTACAGGATGCAACTCCTGTCTTGCATTGCACTAAGTCTTGCACTTGTCA |
| | Variable Chain | CGCAGGTGCAGCTGGTGCAGTCTGGGGGTCAGATGAAGAAGCCTGGCGAGT |
| | (with IL-2 | CGATGAGAATTTCTTGTCGGGCTTCTGGATATGAATTTATTGATTGTACGCT |
| | secretory | AAATTGGATTCGTCTGGCCCCCGGAAAAAGGCCTGAGTGGATGGGATGGCT |
| | signal) | GAAGCCTCGGGGGGGGGCCGTCAACTACGCACGTCCACTTCAGGGCAGAGT |
| | | GACCATGACACGAGACGTTTATTCCGACACAGCCTTTTTGGAGCTGCGCTCG |
| | | TTGACAGTAGACGACACGGCCGTCTACTTTTGTACTAGGGGAAAAAACTGT |
| | | GATTACAATTGGGACTTCGAACACTGGGGCCGGGGCACCCCGGTCATCGTC |
| | | TCATCA |
| 70 | IgG1 Heavy | CACCAAGGGCCCATCGGTCTTCCCCCTGGCACCCTCCTCCAAGAGCACCTCT |
| | Constant Chain | GGGGGCACAGCGGCCCTGGGCTGCCTGGTCAAGGACTACTTCCCCGAACCG |
| | | GTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCG |
| | | TGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGTGACA |
| | | AAACTCACACATGCCCACCGTGCCCAGCACCTGAACTCCTGGGGGGACCGT |
| | | CAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGT |
| | | CAAGTTCAACTGGTACGTGGACGGCGTGGAGGTGCATAATGCCAAGACAAA |
| | | GCCGCGGGAGGAGCAGTACAACAGCACGTACCGTGTGGTCAGCGTCCTCAC |
| | | CGTCCTGCACCAGGACTGGCTGAATGGCAAGGAGTACAAGTGCAAGGTCTC |
| | | CAACAAAGCCCTCCCAGCCCCCATCGAGAAAACCATCTCCAAAGCCAAAGG |
| | | GCAGCCCCGAGAACCACAGGTGTACACCCTGCCCCCATCCCGGGATGAGCT |
| | | GACCAAGAACCAGGTCAGCCTGACCTGCCTGGTCAAAGGCTTCTATCCCAG |
| | | CGACATCGCCGTGGAGTGGGAGAGCAATGGGCAGCCGGAGAACAACTACA |
| | | AGACCACGCCTCCCGTGCTGGACTCCGACGGCTCCTTCTTCCTCTACAGCAA |
| | | GCTCACCGTGGACAAGAGCAGGTGGCAGCAGGGGAACGTCTTCTCATGCTC |
| | | CGTGATGCATGAGGCTCTGCACAACCACTACACGCAGAAGAGCCTCTCCCT |
| | | GTCTCCGGGTAAA |
| 71 | VRC01 Light | ATGGGATGGTCATGTATCATCCTTTTTCTAGTAGCAACTGCAACTGGTGTAC |
| | Variable Chain | ATTCCGAAATTGTGTTGACACAGTCTCCAGGCACCCTGTCTTTGTCTCCAGG |
| | (with antibody | GGAAACAGCCATCATCTCTTGTCGGACCAGTCAGTATGGTTCCTTAGCCTGG |
| | secretory | TATCAACAGAGGCCCGGCCAGGCCCCCAGGCTCGTCATCTATTCGGGCTCT |
| | signal) | ACTCGGGCCGCTGGCATCCCAGACAGGTTCAGCGGCAGTCGGTGGGGGCCA |
| | | GACTACAATCTCACCATCAGCAACCTGGAGTCGGGAGATTTTGGTGTTTATT |
| | | ATTGCCAGCAGTATGAATTTTTTGGCCAGGGGACCAAGGTCCAGGTCGACA |
| | | TTAAGCGA |
| 72 | VRC01 Light | ATGTACAGGATGCAACTCCTGTCTTGCATTGCACTAAGTCTTGCACTTGTCA |
| | Variable Chain | CGGAAATTGTGTTGACACAGTCTCCAGGCACCCTGTCTTTGTCTCCAGGGGA |
| | (with IL-2 | AACAGCCATCATCTCTTGTCGGACCAGTCAGTATGGTTCCTTAGCCTGGTAT |
| | secretory | CAACAGAGGCCCGGCCAGGCCCCCAGGCTCGTCATCTATTCGGGCTCTACT |
| | signal) | CGGGCCGCTGGCATCCCAGACAGGTTCAGCGGCAGTCGGTGGGGGCCAGAC |
| | | TACAATCTCACCATCAGCAACCTGGAGTCGGGAGATTTTGGTGTTTATTATT |
| | | GCCAGCAGTATGAATTTTTTGGCCAGGGGACCAAGGTCCAGGTCGACATTA |
| | | AGCGA |
| 73 | IgG1 Light | GTGGCTGCACCATCTGTCTTCATCTTCCCGCCATCTGATGAGCAGTTGAAAT |
| | Constant Chain | CTGGAACTGCCTCTGTTGTGTGCCTGCTGAATAACTTCTATCCCAGAGAGGC |
| | | CAAAGTACAGTGGAAGGTGGATAACGCCCTCCAATCGGGTAACTCCCAGGA |
| | | GAGTGTCACAGAGCAGGACAGCAAGGACAGCACCTACAGCCTCAGCAGCA |
| | | CCCTGACGCTGAGCAAAGCAGACTACGAGAAACACAAAGTCTACGCCTGCG |
| | | AAGTCACCCATCAGGGCCTGAGCTCGCCCGTCACAAAGAGCTTCAACAGGG |
| | | GAGAGTGTTAG |
| 74 | 3BNC117 | ATGTACAGGATGCAACTCCTGTCTTGCATTGCACTAAGTCTTGCACTTGTCA |
| | Heavy Variable | CGCAGGTCCAATTGTTACAGTCTGGGGCAGCGGTGACGAAGCCCGGGGCCT |
| | Chain (with IL- | CAGTGAGAGTCTCCTGCGAGGCTTCTGGATACAACATTCGTGACTACTTTAT |
| | 2 secretory | TCATTGGTGGCGACAGGCCCCAGGACAGGGCCTTCAGTGGGTGGGATGGAT |
| | signal) | CAATCCTAAGACAGGTCAGCCAAACAATCCTCGTCAATTTCAGGGTAGAGT |
| | | CAGTCTGACTCGACACGCGTCGTGGGACTTTGACACATTTTCCTTTTACATG |
| | | GACCTGAAGGCACTAAGATCGGACGACACGGCCGTTTATTTCTGTGCGCGA |
| | | CAGCGCAGCGACTATTGGGATTTCGACGTCTGGGGCAGTGGAACCCAGGTC |
| | | ACTGTCTCGTCAGCGTCGACCAAGGGCCCA |
| 75 | 3BNC117 Light | ATGTACAGGATGCAACTCCTGTCTTGCATTGCACTAAGTCTTGCACTTGTCA |
| | Variable Chain | CGGACATCCAGATGACCCAGTCTCCATCCTCCCTGTCTGCCTCTGTGGGAGA |
| | (with IL-2 | TACCGTCACTATCACTTGCCAGGCAAACGGCTACTTAAATTGGTATCAACAG |
| | secretory | AGGCGAGGGAAAGCCCCAAAACTCCTGATCTACGATGGGTCCAAATTGGAA |
| | signal) | AGAGGGGTCCCATCAAGGTTCAGTGGAAGAAGATGGGGGCAAGAATATAA |
| | | TCTGACCATCAACAATCTGCAGCCCGAAGACATTGCAACATATTTTTGTCAA |
| | | GTGTATGAGTTTGTCGTCCCTGGGACCAGACTGGATTTGAAACGTACGGTG |
| | | GCTGCACCA |
| 76 | sCD4-IgG1 Fc | ATGGGATGGTCATGTATCATCCTTTTTCTAGTAGCAACTGCAACTGGTGTAC |
| | (with antibody | ATTCCAAGAAAGTGGTGCTGGGCAAAAAAGGGGATACAGTGGAACTGACC |
| | secretory | TGCACAGCTTCCCAGAAGAAGAGCATACAATTCCACTGGAAAAACTCCAAC |
| | signal) version | CAGATAAAGATTCTGGGAAATCAGGGCTCCTTCTTAACTAAAGGTCCATCC |
| | 2 | AAGCTGAATGATCGCGCTGACTCAAGAAGAAGCCTTTGGGACCAAGGAAAC |
| | | TTTCCCCTGATCATCAAGAATCTTAAGATAGAAGACTCAGATACTTACATCT |
| | | GTGAAGTGGAGGACCAGAAGGAGGAGGTGCAATTGCTAGTGTTCGGATTGA |
| | | CTGCCAACTCTGACACCCACCTGCTTCAGGGGCAGAGCCTGACCCTGACCTT |
| | | GGAGAGCCCCCCTGGTAGTAGCCCCTCAGTGCAATGTAGGAGTCCAAGGGG |
| | | TAAAAACATACAAGGTGGTAAGACCCTCTCCGTGTCTCAGCTGGAGCTCCA |
| | | GGATAGTGGCACCTGGACATGCACTGTCTTGCAGAACCAGAAGAAGGTGGA |
| | | GTTCAAAATAGACATCGTGGTGCTAGCTGAGCCCAAGAGCTGCGACAAGAC |
| | | CCACACCTGTCCACCATGCCCCGCACCTGAACTCCTGGGGGGACCGTCAGT |
| | | CTTCCTCTTCCCCCCAAAACCCAAGGACACCCTCATGATCTCCCGGACCCCT |
| | | GAGGTCACATGCGTGGTGGTGGACGTGAGCCACGAAGACCCTGAGGTCAAG |
| | | TTCAACTGGTACGTGGACGGCGTGGAGGTGCATAATGCCAAGACAAAGCCG |
| | | CGGGAGGAGCAGTACAACAGCACGTACCGTGTGGTCAGCGTCCTCACCGTC |
| | | CTGCACCAGGACTGGCTGAATGGCAAGGAGTACAAGTGCAAGGTCTCCAAC |
| | | AAAGCCCTCCCAGCCCCCATCGAGAAAACCATCTCCAAAGCCAAAGGGCAG |
| | | CCCCGAGAACCACAGGTGTACACCCTGCCCCCATCCCGGGATGAGCTGACC |
| | | AAGAACCAGGTCAGCCTGACCTGCCTGGTCAAAGGCTTCTATCCCAGCGAC |
| | | ATCGCCGTGGAGTGGGAGAGCAATGGGCAGCCGGAGAACAACTACAAGAC |
| | | CACGCCTCCCGTGCTGGACTCCGACGGCTCCTTCTTCCTCTACAGCAAGCTC |
| | | ACCGTGGACAAGAGCAGGTGGCAGCAGGGGAACGTCTTCTCATGCTCCGTG |
| | | ATGCATGAGGCTCTGCACAACCACTACACGCAGAAGAGCCTCTCCCTGTCT |
| | | CCGGGTAAATGA |
| 77 | sCD4-IgG1 Fc | ATGGGATGGTCATGTATCATCCTTTTTCTAGTAGCAACTGCAACTGGTGTAC |
| | (with antibody | ATTCCAAGAAAGTGGTGCTGGGCAAAAAAGGGGATACAGTGGAACTGACC |
| | secretory | TGCACAGCTTCCCAGAAGAAGAGCATACAATTCCACTGGAAAAACTCCAAC |
| | signal) version | CAGATAAAGATTCTGGGAAATCAGGGCTCCTTCTTAACTAAAGGTCCATCC |
| | 3 | AAGCTGAATGATCGCGCTGACTCAAGAAGAAGCCTTTGGGACCAAGGAAAC |
| | | TTTCCCCTGATCATCAAGAATCTTAAGATAGAAGACTCAGATACTTACATCT |
| | | GTGAAGTGGAGGACCAGAAGGAGGAGGTGCAATTGCTAGTGTTCGGATTGA |
| | | CTGCCAACTCTGACACCCACCTGCTTCAGGGGCAGAGCCTGACCCTGACCTT |
| | | GGAGAGCCCCCCTGGTAGTAGCCCCTCAGTGCAATGTAGGAGTCCAAGGGG |
| | | TAAAAACATACAAGGTGGTAAGACCCTCTCCGTGTCTCAGCTGGAGCTCCA |
| | | GGATAGTGGCACCTGGACATGCACTGTCTTGCAGAACCAGAAGAAGGTGGA |
| | | GTTCAAAATAGACATCGTGGTGCTAGCTGAGCCCAAGAGCTGCGACAAGAC |
| | | CCACACCTGTCCACCATGCCCCGCACCTGAAGCTGCAGGGGGACCGTCAGT |
| | | CTTCCTCTTCCCCCCAAAACCCAAGGACACCCTCATGATCTCCCGGACCCCT |
| | | GAGGTCACATGCGTGGTGGTGGACGTGAGCCACGAAGACCCTGAGGTCAAG |
| | | TTCAACTGGTACGTGGACGGCGTGGAGGTGCATAATGCCAAGACAAAGCCG |
| | | CGGGAGGAGCAGTACAACAGCACGTACCGTGTGGTCAGCGTCCTCACCGTC |
| | | CTGCACCAGGACTGGCTGAATGGCAAGGAGTACAAGTGCGCTGTCTCCAAC |
| | | AAAGCCCTCCCAGCCCCCATCGAGAAAACCATCTCCAAAGCCAAAGGGCAG |
| | | CCCCGAGAACCACAGGTGTACACCCTGCCCCCATCCCGGGATGAGCTGACC |
| | | AAGAACCAGGTCAGCCTGACCTGCCTGGTCAAAGGCTTCTATCCCAGCGAC |
| | | ATCGCCGTGGAGTGGGAGAGCAATGGGCAGCCGGAGAACAACTACAAGAC |
| | | CACGCCTCCCGTGCTGGACTCCGACGGCTCCTTCTTCCTCTACAGCAAGCTC |
| | | ACCGTGGACAAGAGCAGGTGGCAGCAGGGGAACGTCTTCTCATGCTCCGTG |
| | | ATGCATGAGGCTCTGCACAACCACTACACGCAGAAGAGCCTCTCCCTGTCT |
| | | CCGGGTAAATGA |
| 78 | AGT103 | CCGGTGCCTAGAGAAGGTGGCGCGGGGTAAACTGGGAAAGTGATGTCGTGT |
| | | ACTGGCTCCGCCTTTTTCCCGAGGGTGGGGGAGAACCGTATATAAGTGCAG |
| | | TAGTCGCCGTGAACGTTCTTTTTCGCAACGGGTTTGCCGCCAGAACACAGGT |
| | | AAGTGCCGTGTGTGGTTCCCGCGGGCCTGGCCTCTTTACGGGTTATGGCCCT |
| | | TGCGTGCCTTGAATTACTTCCACGCCCCTGGCTGCAGTACGTGATTCTTGAT |
| | | CCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGC |
| | | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAAGGTATATTGCTGTTGACAGTGAGC |
| | | GACTGTAAACTGAGCTTGCTCTACTGTGAAGCCACAGATGGGTAGAGCAAG |
| | | CACAGTTTACCGCTGCCTACTGCCTCGGACTTCAAGGGGCTTCCCGGGCATC |
| | | TCCATGGCTGTACCACCTTGTCGGGGGATGTGTACTTCTGAACTTGTGTTGA |
| | | ATCTCATGGAGTTCAGAAGAACACATCCGCACTGACATTTTGGTATCTTTCA |
| | | TCTGACCAGCTAGCGGGCCTGGCTCGAGCAGGGGGCGAGGGATTCCGCTTC |
| | | TTCCTGCCATAGCGTGGTCCCCTCCCCTATGGCAGGCAGAAGCGGCACCTTC |
| | | CCTCCCAATGACCGCGTCTTCGTC |
| 79 | IFNγ promoter, | TGTATTTCTACTGGGCAGTGCTGATCTAGAGCAATTTGAAACTTGTGGTAGA |
| | VRC01, | TATTTTACTAACCAACTCTGATGAAGGACTTCCTCACCAAATTGTTCTTTTA |
| | antibody | ACCGCATTCTTTCCTTGCTTTCTGGTCATTTGCAAGAAAAATTTTAAAAGGC |
| | secretion signal | TGCCCCTTTGTAAAGGTTTGAGAGGCCCTAGAATTTCGTTTTTCACTTGTTCC |
| | sequence | CAACCACAAGCAAATGATCAATGTGCTTTGTGAATGAAGAGTCAACATTTT |
| | (AGT115) | ACCAGGGCGAAGTGGGGAGGTACAAAAAAATTTCCAGTCCTTGAATGGTGT |
| | | GAAGTAAAAGTGCCTTCAAAGAATCCCACCAGAATGGCACAGGTGGGCATA |
| | | ATGGGTCTGTCTCATCGTCAAAGGACCCAAGGAGTCTAAAGGAAACTCTAA |
| | | CTACAACACCCAAATGCCACAAAACCTTAGTTATTAATACAAACTATCATCC |
| | | CTGCCTATCTGTCACCATCTCATCTTAAAAAACTTGTGAAAATACGTAATCC |
| | | TCAGGAGACTTCAATTAGGTATAAATACCAGCAGCCAGAGGAGGTGCAGCA |
| | | CATTGTTCTGATCATCTGAAGATCAGCTATTAGAAGAGAAAGATCAGCTCG |
| | | AGGCCACCATGGGATGGTCATGTATCATCCTTTTTCTAGTAGCAACTGCAAC |
| | | TGGTGTACATTCCCAGGTGCAGCTGGTGCAGTCTGGGGGTCAGATGAAGAA |
| | | GCCTGGCGAGTCGATGAGAATTTCTTGTCGGGCTTCTGGATATGAATTTATT |
| | | GATTGTACGCTAAATTGGATTCGTCTGGCCCCCGGAAAAAGGCCTGAGTGG |
| | | ATGGGATGGCTGAAGCCTCGGGGGGGGGCCGTCAACTACGCACGTCCACTT |
| | | CAGGGCAGAGTGACCATGACACGAGACGTTTATTCCGACACAGCCTTTTTG |
| | | GAGCTGCGCTCGTTGACAGTAGACGACACGGCCGTCTACTTTTGTACTAGG |
| | | GGAAAAAACTGTGATTACAATTGGGACTTCGAACACTGGGGCCGGGGCACC |
| | | CCGGTCATCGTCTCATCAGCTAGCACCAAGGGCCCATCGGTCTTCCCCCTGG |
| | | CACCCTCCTCCAAGAGCACCTCTGGGGGCACAGCGGCCCTGGGCTGCCTGG |
| | | TCAAGGACTACTTCCCCGAACCGGTGACGGTGTCGTGGAACTCAGGCGCCC |
| | | TGACCAGCGGCGTGCACACCTTCCCGGCTGTCCTACAGTCCTCAGGACTCTA |
| | | CTCCCTCAGCAGCGTGGTGACCGTGCCCTCCAGCAGCTTGGGCACCCAGAC |
| | | CTACATCTGCAACGTGAATCACAAGCCCAGCAACACCAAGGTGGACAAGAA |
| | | AGTTGAGCCCAAATCTTGTGACAAAACTCACACATGCCCACCGTGCCCAGC |
| | | ACCTGAACTCCTGGGGGGACCGTCAGTCTTCCTCTTCCCCCCAAAACCCAAG |
| | | GACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGTG |
| | | GAGGTGCATAATGCCAAGACAAAGCCGCGGGAGGAGCAGTACAACAGCAC |
| | | GTACCGTGTGGTCAGCGTCCTCACCGTCCTGCACCAGGACTGGCTGAATGG |
| | | CAAGGAGTACAAGTGCAAGGTCTCCAACAAAGCCCTCCCAGCCCCCATCGA |
| | | GAAAACCATCTCCAAAGCCAAAGGGCAGCCCCGAGAACCACAGGTGTACA |
| | | CCCTGCCCCCATCCCGGGATGAGCTGACCAAGAACCAGGTCAGCCTGACCT |
| | | GCCTGGTCAAAGGCTTCTATCCCAGCGACATCGCCGTGGAGTGGGAGAGCA |
| | | ATGGGCAGCCGGAGAACAACTACAAGACCACGCCTCCCGTGCTGGACTCCG |
| | | ACGGCTCCTTCTTCCTCTACAGCAAGCTCACCGTGGACAAGAGCAGGTGGC |
| | | AGCAGGGGAACGTCTTCTCATGCTCCGTGATGCATGAGGCTCTGCACAACC |
| | | ACTACACGCAGAAGAGCCTCTCCCTGTCTCCGGGTAAACGTAGACGAAAGC |
| | | GCGGAAGCGGAGAGGGCAGAGGAAGTCTGCTAACATGCGGTGACGTCGAG |
| | | GAGAATCCTGGACCTGGATCCATGGGATGGTCATGTATCATCCTTTTTCTAG |
| | | TAGCAACTGCAACTGGTGTACATTCCGAAATTGTGTTGACACAGTCTCCAGG |
| | | CACCCTGTCTTTGTCTCCAGGGGAAACAGCCATCATCTCTTGTCGGACCAGT |
| | | CAGTATGGTTCCTTAGCCTGGTATCAACAGAGGCCCGGCCAGGCCCCCAGG |
| | | CTCGTCATCTATTCGGGCTCTACTCGGGCCGCTGGCATCCCAGACAGGTTCA |
| | | GCGGCAGTCGGTGGGGGCCAGACTACAATCTCACCATCAGCAACCTGGAGT |
| | | CGGGAGATTTTGGTGTTTATTATTGCCAGCAGTATGAATTTTTTGGCCAGGG |
| | | GACCAAGGTCCAGGTCGACATTAAGCGAGAATTCGTGGCTGCACCATCTGT |
| | | CTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAG |
| | | GACAGCAAGGACAGCACCTACAGCCTCAGCAGCACCCTGACGCTGAGCAA |
| | | AGCAGACTACGAGAAACACAAAGTCTACGCCTGCGAAGTCACCCATCAGGG |
| | | CCTGAGCTCGCCCGTCACAAAGAGCTTCAACAGGGGAGAGTGTTAG |
| 80 | EF1α promoter, | CCGGTGCCTAGAGAAGGTGGCGCGGGGTAAACTGGGAAAGTGATGTCGTGT |
| | CD4/IgG1 | ACTGGCTCCGCCTTTTTCCCGAGGGTGGGGGAGAACCGTATATAAGTGCAG |
| | fusion protein, | TAGTCGCCGTGAACGTTCTTTTTCGCAACGGGTTTGCCGCCAGAACACAGGT |
| | antibody | AAGTGCCGTGTGTGGTTCCCGCGGGCCTGGCCTCTTTACGGGTTATGGCCCT |
| | secretion signal, | TGCGTGCCTTGAATTACTTCCACGCCCCTGGCTGCAGTACGTGATTCTTGAT |
| | miR30- | CCCGAGCTTCGGGTTGGAAGTGGGTGGGAGAGTTCGAGGCCTTGCGCTTAA |
| | CCR5/miR21- | GGAGCCCCTTCGCCTCGTGCTTGAGTTGAGGCCTGGCCTGGGCGCTGGGGC |
| | Vif/miR185-Tat | CGCCGCGTGCGAATCTGGTGGCACCTTCGCGCCTGTCTCGCTGCTTTCGATA |
| | microRNA | AGTCTCTAGCCATTTAAAATTTTTGATGACCTGCTGCGACGCTTTTTTTCTGG |
| | cluster | CAAGATAGTCTTGTAAATGCGGGCCAAGATCTGCACACTGGTATTTCGGTTT |
| | sequence | TTGGGGCCGCGGGCGGCGACGGGGCCCGTGCGTCCCAGCGCACATGTTCGG |
| | (AGT118) | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAGCCACCATGGGATGGTCATGTATCA |
| | | TCCTTTTTCTAGTAGCAACTGCAACTGGTGTACATTCCAAGAAAGTGGTGCT |
| | | GGGCAAAAAAGGGGATACAGTGGAACTGACCTGCACAGCTTCCCAGAAGA |
| | | AGAGCATACAATTCCACTGGAAAAACTCCAACCAGATAAAGATTCTGGGAA |
| | | ATCAGGGCTCCTTCTTAACTAAAGGTCCATCCAAGCTGAATGATCGCGCTGA |
| | | CTCAAGAAGAAGCCTTTGGGACCAAGGAAACTTTCCCCTGATCATCAAGAA |
| | | TCTTAAGATAGAAGACTCAGATACTTACATCTGTGAAGTGGAGGACCAGAA |
| | | GGAGGAGGTGCAATTGCTAGTGTTCGGATTGACTGCCAACTCTGACACCCA |
| | | CCTGCTTCAGGGGCAGAGCCTGACCCTGACCTTGGAGAGCCCCCCTGGTAG |
| | | TAGCCCCTCAGTGCAATGTAGGAGTCCAAGGGGTAAAAACATACAAGGTGG |
| | | TAAGACCCTCTCCGTGTCTCAGCTGGAGCTCCAGGATAGTGGCACCTGGAC |
| | | ATGCACTGTCTTGCAGAACCAGAAGAAGGTGGAGTTCAAAATAGACATCGT |
| | | GGTGCTAGCTGCTGCAGATCCGGAGCCCAAGAGCTGCGACAAGACCCACAC |
| | | CTGTCCACCATGCCCCGCCCACCTGAACTCCTGGGGGGACCGTCAGTCTTCC |
| | | TCTTCCCCCCAAAACCCAAGGACACCCTCATGATCTCCCGGACCCCTGAGGT |
| | | CACATGCGTGGTGGTGGACGTGAGCCACGAAGACCCTGAGGTCAAGTTCAA |
| | | CTGGTACGTGGACGGCGTGGAGGTGCATAATGCCAAGACAAAGCCGCGGG |
| | | AGGAGCAGTACAACAGCACGTACCGTGTGGTCAGCGTCCTCACCGTCCTGC |
| | | ACCAGGACTGGCTGAATGGCAAGGAGTACAAGTGCAAGGTCTCCAACAAA |
| | | GCCCTCCCAGCCCCCATCGAGAAAACCATCTCCAAAGCCAAAGGTGGGACC |
| | | CGTGGGGTGCGAGGGCCACATGGACAGAGGCCGGCTCGGCCCACCCTCTGC |
| | | CCTGAGAGTGACCGCTGTACCAACCTCTGTCCCTACAGGGCAGCCCCGAGA |
| | | ACCACAGGTCTACACCCTGCCCCCATCCCGGGAGGAGATGACCAAGAACCA |
| | | GGTCAGCCTGACCTGCCTGGTCAAAGGCTTCTATCCCAGCGACATCGCCGTG |
| | | GAGTGGGAGAGCAATGGGCAGCCGGAGAACAACTACAAGACCACGCCTCC |
| | | CGTGCTGGACTCCGACGGCTCCTTCTTCCTCTATAGCAAGCTCACCGTGGAC |
| | | AAGAGCAGGTGGCAGCAGGGGAACGTCTTCTCATGCTCCGTGATGCATGAG |
| | | GCTCTGCACAACCACTACACGCAGAAGAGCCTCTCCCTGTCCCCGGGTAAA |
| | | TGAGCGGCCGCTCGAGCATGCATCTAGTCAAGGTATATTGCTGTTGACAGT |
| | | GAGCGACTGTAAACTGAGCTTGCTCTACTGTGAAGCCACAGATGGGTAGAG |
| | | CAAGCACAGTTTACCGCTGCCTACTGCCTCGGACTTCAAGGGGCTTCCCGGG |
| | | CATCTCCATGGCTGTACCACCTTGTCGGGGGATGTGTACTTCTGAACTTGTG |
| | | TTGAATCTCATGGAGTTCAGAAGAACACATCCGCACTGACATTTTGGTATCT |
| | | TTCATCTGACCAGCTAGCGGGCCTGGCTCGAGCAGGGGGCGAGGGATTCCG |
| | | CTTCTTCCTGCCATAGCGTGGTCCCCTCCCCTATGGCAGGCAGAAGCGGCAC |
| | | CTTCCCTCCCAATGACCGCGTCTTCGTCG |
| 81 | EF1α promoter, | CCGGTGCCTAGAGAAGGTGGCGCGGGGTAAACTGGGAAAGTGATGTCGTGT |
| | miR30- | ACTGGCTCCGCCTTTTTCCCGAGGGTGGGGGAGAACCGTATATAAGTGCAG |
| | CCR5/miR21- | TAGTCGCCGTGAACGTTCTTTTTCGCAACGGGTTTGCCGCCAGAACACAGGT |
| | Vif/miR185-Tat | AAGTGCCGTGTGTGGTTCCCGCGGGCCTGGCCTCTTTACGGGTTATGGCCCT |
| | microRNA | TGCGTGCCTTGAATTACTTCCACGCCCCTGGCTGCAGTACGTGATTCTTGAT |
| | cluster | CCCGAGCTTCGGGTTGGAAGTGGGTGGGAGAGTTCGAGGCCTTGCGCTTAA |
| | sequence, | GGAGCCCCTTCGCCTCGTGCTTGAGTTGAGGCCTGGCCTGGGCGCTGGGGC |
| | CD4/IgG1 | CGCCGCGTGCGAATCTGGTGGCACCTTCGCGCCTGTCTCGCTGCTTTCGATA |
| | fusion protein, | AGTCTCTAGCCATTTAAAATTTTTGATGACCTGCTGCGACGCTTTTTTTCTGG |
| | antibody | CAAGATAGTCTTGTAAATGCGGGCCAAGATCTGCACACTGGTATTTCGGTTT |
| | secretion signal, | TTGGGGCCGCGGGCGGCGACGGGGCCCGTGCGTCCCAGCGCACATGTTCGG |
| | (AGT119) | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAAGGTATATTGCTGTTGACAGTGAGC |
| | | GACTGTAAACTGAGCTTGCTCTACTGTGAAGCCACAGATGGGTAGAGCAAG |
| | | CACAGTTTACCGCTGCCTACTGCCTCGGACTTCAAGGGGCTTCCCGGGCATC |
| | | TCCATGGCTGTACCACCTTGTCGGGGGATGTGTACTTCTGAACTTGTGTTGA |
| | | ATCTCATGGAGTTCAGAAGAACACATCCGCACTGACATTTTGGTATCTTTCA |
| | | TCTGACCAGCTAGCGGGCCTGGCTCGAGCAGGGGGCGAGGGATTCCGCTTC |
| | | TTCCTGCCATAGCGTGGTCCCCTCCCCTATGGCAGGCAGAAGCGGCACCTTC |
| | | CCTCCCAATGACCGCGTCTTCGTCGCGGCCGCGCCACCATGGGATGGTCATG |
| | | TATCATCCTTTTTCTAGTAGCAACTGCAACTGGTGTACATTCCAAGAAAGTG |
| | | GTGCTGGGCAAAAAAGGGGATACAGTGGAACTGACCTGCACAGCTTCCCAG |
| | | AAGAAGAGCATACAATTCCACTGGAAAAACTCCAACCAGATAAAGATTCTG |
| | | GGAAATCAGGGCTCCTTCTTAACTAAAGGTCCATCCAAGCTGAATGATCGC |
| | | GCTGACTCAAGAAGAAGCCTTTGGGACCAAGGAAACTTTCCCCTGATCATC |
| | | AAGAATCTTAAGATAGAAGACTCAGATACTTACATCTGTGAAGTGGAGGAC |
| | | CAGAAGGAGGAGGTGCAATTGCTAGTGTTCGGATTGACTGCCAACTCTGAC |
| | | ACCCACCTGCTTCAGGGGCAGAGCCTGACCCTGACCTTGGAGAGCCCCCCT |
| | | GGTAGTAGCCCCTCAGTGCAATGTAGGAGTCCAAGGGGTAAAAACATACAA |
| | | GGTGGTAAGACCCTCTCCGTGTCTCAGCTGGAGCTCCAGGATAGTGGCACC |
| | | TGGACATGCACTGTCTTGCAGAACCAGAAGAAGGTGGAGTTCAAAATAGAC |
| | | ATCGTGGTGCTAGCTGCTGCAGATCCGGAGCCCAAGAGCTGCGACAAGACC |
| | | CACACCTGTCCACCATGCCCCGCCCACCTGAACTCCTGGGGGGACCGTCAG |
| | | TCTTCCTCTTCCCCCCAAAACCCAAGGACACCCTCATGATCTCCCGGACCCC |
| | | TGAGGTCACATGCGTGGTGGTGGACGTGAGCCACGAAGACCCTGAGGTCAA |
| | | GTTCAACTGGTACGTGGACGGCGTGGAGGTGCATAATGCCAAGACAAAGCC |
| | | GCGGGAGGAGCAGTACAACAGCACGTACCGTGTGGTCAGCGTCCTCACCGT |
| | | CCTGCACCAGGACTGGCTGAATGGCAAGGAGTACAAGTGCAAGGTCTCCAA |
| | | CAAAGCCCTCCCAGCCCCCATCGAGAAAACCATCTCCAAAGCCAAAGGTGG |
| | | GACCCGTGGGGTGCGAGGGCCACATGGACAGAGGCCGGCTCGGCCCACCCT |
| | | CTGCCCTGAGAGTGACCGCTGTACCAACCTCTGTCCCTACAGGGCAGCCCC |
| | | GAGAACCACAGGTCTACACCCTGCCCCCATCCCGGGAGGAGATGACCAAGA |
| | | ACCAGGTCAGCCTGACCTGCCTGGTCAAAGGCTTCTATCCCAGCGACATCG |
| | | CCGTGGAGTGGGAGAGCAATGGGCAGCCGGAGAACAACTACAAGACCACG |
| | | CCTCCCGTGCTGGACTCCGACGGCTCCTTCTTCCTCTATAGCAAGCTCACCG |
| | | TGGACAAGAGCAGGTGGCAGCAGGGGAACGTCTTCTCATGCTCCGTGATGC |
| | | ATGAGGCTCTGCACAACCACTACACGCAGAAGAGCCTCTCCCTGTCCCCGG |
| | | GTAAATGA |
| 82 | IL2 promoter, | ATCTATCTTATTGTATGCAATTAGCTCATTGTGTGGATAAAAAGGTAAAACC |
| | CD4/IgG1 | ATTCTGAAACAGGAAACCAATACACTTCCTGTTTAATCAACAAATCTAAAC |
| | fusion protein, | ATTTATTCTTTTCATCTGTTTACTCTTGCTCTTGTCCACCACAATATGCTATTC |
| | antibody | ACATGTTCAGTGTAGTTTTATGACAAAGAAAATTTTCTGAGTTACTTTTGTA |
| | secretion signal | TCCCCACCCCCTTAAAGAAAGGAGGAAAAACTGTTTCATACAGAAGGCGTT |
| | (AGT120) | AATTGCATGAATTAGAGCTATCACCTAAGTGTGGGCTAATGTAACAAAGAG |
| | | GGATTTCACCTACATCCATTCAGTCAGTCTTTGGGGGTTTAAAGAAATTCCA |
| | | AAGAGTCATCAGAAGAGGAAAAATGAAGGTAATGTTTTTTCAGACTGGTAA |
| | | AGTCTTTGAAAATATGTGTAATATGTAAAACATTTTGACACCCCCATAATAT |
| | | TTTTCCAGAATTAACAGTATAAATTGCATCTCTTGTTCAAGAGTTCCCTATC |
| | | ACTCTTGAATTCGCCACCATGGGATGGTCATGTATCATCCTTTTTCTAGTAG |
| | | CAACTGCAACTGGTGTACATTCCAAGAAAGTGGTGCTGGGCAAAAAAGGGG |
| | | ATACAGTGGAACTGACCTGCACAGCTTCCCAGAAGAAGAGCATACAATTCC |
| | | ACTGGAAAAACTCCAACCAGATAAAGATTCTGGGAAATCAGGGCTCCTTCT |
| | | TAACTAAAGGTCCATCCAAGCTGAATGATCGCGCTGACTCAAGAAGAAGCC |
| | | TTTGGGACCAAGGAAACTTTCCCCTGATCATCAAGAATCTTAAGATAGAAG |
| | | ACTCAGATACTTACATCTGTGAAGTGGAGGACCAGAAGGAGGAGGTGCAAT |
| | | TGCTAGTGTTCGGATTGACTGCCAACTCTGACACCCACCTGCTTCAGGGGCA |
| | | GAGCCTGACCCTGACCTTGGAGAGCCCCCCTGGTAGTAGCCCCTCAGTGCA |
| | | ATGTAGGAGTCCAAGGGGTAAAAACATACAAGGTGGTAAGACCCTCTCCGT |
| | | GTCTCAGCTGGAGCTCCAGGATAGTGGCACCTGGACATGCACTGTCTTGCA |
| | | GAACCAGAAGAAGGTGGAGTTCAAAATAGACATCGTGGTGCTAGCTGCTGC |
| | | AGATCCGGAGCCCAAGAGCTGCGACAAGACCCACACCTGTCCACCATGCCC |
| | | CGCCCACCTGAACTCCTGGGGGGACCGTCAGTCTTCCTCTTCCCCCCAAAAC |
| | | CCAAGGACACCCTCATGATCTCCCGGACCCCTGAGGTCACATGCGTGGTGG |
| | | TGGACGTGAGCCACGAAGACCCTGAGGTCAAGTTCAACTGGTACGTGGACG |
| | | GCGTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGCTG |
| | | AATGGCAAGGAGTACAAGTGCAAGGTCTCCAACAAAGCCCTCCCAGCCCCC |
| | | ATCGAGAAAACCATCTCCAAAGCCAAAGGTGGGACCCGTGGGGTGCGAGG |
| | | GCCACATGGACAGAGGCCGGCTCGGCCCACCCTCTGCCCTGAGAGTGACCG |
| | | CTGTACCAACCTCTGTCCCTACAGGGCAGCCCCGAGAACCACAGGTCTACA |
| | | CCCTGCCCCCATCCCGGGAGGAGATGACCAAGAACCAGGTCAGCCTGACCT |
| | | GCCTGGTCAAAGGCTTCTATCCCAGCGACATCGCCGTGGAGTGGGAGAGCA |
| | | ATGGGCAGCCGGAGAACAACTACAAGACCACGCCTCCCGTGCTGGACTCCG |
| | | ACGGCTCCTTCTTCCTCTATAGCAAGCTCACCGTGGACAAGAGCAGGTGGC |
| | | AGCAGGGGAACGTCTTCTCATGCTCCGTGATGCATGAGGCTCTGCACAACC |
| | | ACTACACGCAGAAGAGCCTCTCCCTGTCCCCGGGTAAATGA |
| 83 | IFNγ promoter, | TGTATTTCTACTGGGCAGTGCTGATCTAGAGCAATTTGAAACTTGTGGTAGA |
| | CD4/IgG1 | TATTTTACTAACCAACTCTGATGAAGGACTTCCTCACCAAATTGTTCTTTTA |
| | fusion protein, | ACCGCATTCTTTCCTTGCTTTCTGGTCATTTGCAAGAAAAATTTTAAAAGGC |
| | antibody | TGCCCCTTTGTAAAGGTTTGAGAGGCCCTAGAATTTCGTTTTTCACTTGTTCC |
| | secretion signal | CAACCACAAGCAAATGATCAATGTGCTTTGTGAATGAAGAGTCAACATTTT |
| | (AGT121) | ACCAGGGCGAAGTGGGGAGGTACAAAAAAATTTCCAGTCCTTGAATGGTGT |
| | | GAAGTAAAAGTGCCTTCAAAGAATCCCACCAGAATGGCACAGGTGGGCATA |
| | | ATGGGTCTGTCTCATCGTCAAAGGACCCAAGGAGTCTAAAGGAAACTCTAA |
| | | CTACAACACCCAAATGCCACAAAACCTTAGTTATTAATACAAACTATCATCC |
| | | CTGCCTATCTGTCACCATCTCATCTTAAAAAACTTGTGAAAATACGTAATCC |
| | | TCAGGAGACTTCAATTAGGTATAAATACCAGCAGCCAGAGGAGGTGCAGCA |
| | | CATTGTTCTGATCATCTGAAGATCAGCTATTAGAAGAGAAAGATCAGGAAT |
| | | TCGCCACCATGGGATGGTCATGTATCATCCTTTTTCTAGTAGCAACTGCAAC |
| | | TGGTGTACATTCCAAGAAAGTGGTGCTGGGCAAAAAAGGGGATACAGTGG |
| | | AACTGACCTGCACAGCTTCCCAGAAGAAGAGCATACAATTCCACTGGAAAA |
| | | ACTCCAACCAGATAAAGATTCTGGGAAATCAGGGCTCCTTCTTAACTAAAG |
| | | GTCCATCCAAGCTGAATGATCGCGCTGACTCAAGAAGAAGCCTTTGGGACC |
| | | AAGGAAACTTTCCCCTGATCATCAAGAATCTTAAGATAGAAGACTCAGATA |
| | | CTTACATCTGTGAAGTGGAGGACCAGAAGGAGGAGGTGCAATTGCTAGTGT |
| | | TCGGATTGACTGCCAACTCTGACACCCACCTGCTTCAGGGGCAGAGCCTGA |
| | | CCCTGACCTTGGAGAGCCCCCCTGGTAGTAGCCCCTCAGTGCAATGTAGGA |
| | | GTCCAAGGGGTAAAAACATACAAGGTGGTAAGACCCTCTCCGTGTCTCAGC |
| | | TGGAGCTCCAGGATAGTGGCACCTGGACATGCACTGTCTTGCAGAACCAGA |
| | | AGAAGGTGGAGTTCAAAATAGACATCGTGGTGCTAGCTGCTGCAGATCCGG |
| | | AGCCCAAGAGCTGCGACAAGACCCACACCTGTCCACCATGCCCCGCCCACC |
| | | TGAACTCCTGGGGGGACCGTCAGTCTTCCTCTTCCCCCCAAAACCCAAGGAC |
| | | ACCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGAG |
| | | GTGCATAATGCCAAGACAAAGCCGCGGGAGGAGCAGTACAACAGCACGTA |
| | | CCGTGTGGTCAGCGTCCTCACCGTCCTGCACCAGGACTGGCTGAATGGCAA |
| | | GGAGTACAAGTGCAAGGTCTCCAACAAAGCCCTCCCAGCCCCCATCGAGAA |
| | | AACCATCTCCAAAGCCAAAGGTGGGACCCGTGGGGTGCGAGGGCCACATG |
| | | GACAGAGGCCGGCTCGGCCCACCCTCTGCCCTGAGAGTGACCGCTGTACCA |
| | | ACCTCTGTCCCTACAGGGCAGCCCCGAGAACCACAGGTCTACACCCTGCCC |
| | | CCATCCCGGGAGGAGATGACCAAGAACCAGGTCAGCCTGACCTGCCTGGTC |
| | | AAAGGCTTCTATCCCAGCGACATCGCCGTGGAGTGGGAGAGCAATGGGCAG |
| | | CCGGAGAACAACTACAAGACCACGCCTCCCGTGCTGGACTCCGACGGCTCC |
| | | TTCTTCCTCTATAGCAAGCTCACCGTGGACAAGAGCAGGTGGCAGCAGGGG |
| | | AACGTCTTCTCATGCTCCGTGATGCATGAGGCTCTGCACAACCACTACACGC |
| | | AGAAGAGCCTCTCCCTGTCCCCGGGTAAATGA |
| 84 | CD69 (1050) | CAGACACTGAGGCTTGGGTTGGGCGAGGCCATTCAGACACTAAAACCCAGT |
| | promoter, | GCAGTTCTCCCTCAAGTGTGACCTTATATGAAGAATCCGGAGGGAGGTTTCT |
| | CD4/IgG1 | GAAAGGAAATGATGTAATGTGAGGCAGATGCAAAGTGCGGCAGGAAGGCA |
| | fusion protein, | GGGTGTACAGTCCTTATCACGGCAGCTGCCTTAGTGGTATGTGTTCAAAGGA |
| | antibody | ACCACAAACTTCCGACCTGAGGCAGTTTCCGGTGACAACCTGCTCATCATAT |
| | secretion signal | TTCAAAATGATTTTTTTCTTTCAGTGAGTGAATGAGGTACTGGAATGTCCTC |
| | (AGT122) | TAGATGATAACTTCCAACCCACCTATGCATAAAATTTAACGTCTTTATTCTA |
| | | AATAAGTGATATTAATAATAAAATTTGGGGCACCAAGATTATTAATCAGAG |
| | | TGGTATTTTGATTTCCCTCCTTAAATCACCATACATAGCTTTCTGCATTCATC |
| | | TTGCGTTGACTGTCATTACTTGTCTGAGTGAGACTGATACCACAGCGATGTT |
| | | TTAAATAATAATCATACCTCAAAAGACTGAAGTCTCAGAGGTATCTGAAGA |
| | | GAATAACCTAGAGCACAGGGGGAGAATTGAAGGAGCTGTTACTGAGGTGA |
| | | CATAAAAGCAGTCTAAATGACAGTAAAATGTGACAAGAAAATTAGCAGGA |
| | | AACAAATGAAACAGATAATTTAAGATAAACAATTTTAGAGCATAGCAAGGA |
| | | AGTTCCAGACCACAAGCTTTCTGTTTCCTGCATTCTTACTTCTTACTACGTGA |
| | | TACATCTAGTCACCAGGGAAGAAGCGAATGACACACTTCCAAAAACCAATT |
| | | CGTAGCTTTCTAAATAAAACCCTTTCTAGCTGGAGAGAGATCCATGAGCAT |
| | | AGAGATCTTAAAATTCATGTTCAGCAATAAATCCTGGGGCCCCAGACAGTG |
| | | TCAGGTGCATAGGGGGTGTTCAGTAAATATCAGTTAAATGTATGCATAAAT |
| | | CGATAAACGGGATTCCTGGAAAATACTACACTCTCCTTCTCCAAATTATCTT |
| | | CATCTCAAAGACAGGAACCTCTAACTTTTAATTCTTTACTTAGATTATGCTG |
| | | TCTCCTAAACTGTTTATGTTTTCTAGAAATTTAAGGCAGGATGTCTCAGAGT |
| | | CTGGGAAAATCCCACTTTCCTCCTGCTACACCTTACAGTTGTGAGAAAGCAC |
| | | ATTTCAGACAACAGGGAAAACCCATACTTCACCACAACAACACACTATACA |
| | | TTGTCTGGTCCACTGGAGCATAAATTAAAGAGAAACAATGTAGTCAAGCAA |
| | | GTAGGCGGCAAGAGGAAGGGGGCGGAGACATCATCAGGGAGTATAAACTC |
| | | TGAGATGCCTCAGAGCCTCACGAATTCGCCACCATGGGATGGTCATGTATC |
| | | ATCCTTTTTCTAGTAGCAACTGCAACTGGTGTACATTCCAAGAAAGTGGTGC |
| | | TGGGCAAAAAAGGGGATACAGTGGAACTGACCTGCACAGCTTCCCAGAAG |
| | | AAGAGCATACAATTCCACTGGAAAAACTCCAACCAGATAAAGATTCTGGGA |
| | | AATCAGGGCTCCTTCTTAACTAAAGGTCCATCCAAGCTGAATGATCGCGCTG |
| | | ACTCAAGAAGAAGCCTTTGGGACCAAGGAAACTTTCCCCTGATCATCAAGA |
| | | ATCTTAAGATAGAAGACTCAGATACTTACATCTGTGAAGTGGAGGACCAGA |
| | | AGGAGGAGGTGCAATTGCTAGTGTTCGGATTGACTGCCAACTCTGACACCC |
| | | ACCTGCTTCAGGGGCAGAGCCTGACCCTGACCTTGGAGAGCCCCCCTGGTA |
| | | GTAGCCCCTCAGTGCAATGTAGGAGTCCAAGGGGTAAAAACATACAAGGTG |
| | | GTAAGACCCTCTCCGTGTCTCAGCTGGAGCTCCAGGATAGTGGCACCTGGA |
| | | CATGCACTGTCTTGCAGAACCAGAAGAAGGTGGAGTTCAAAATAGACATCG |
| | | TGGTGCTAGCTGAGCCCAAGAGCTGCGACAAGACCCACACCTGTCCACCAT |
| | | GCCCCGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGT |
| | | GGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACA |
| | | ACAGCACGTACCGTGTGGTCAGCGTCCTCACCGTCCTGCACCAGGACTGGC |
| | | TGAATGGCAAGGAGTACAAGTGCAAGGTCTCCAACAAAGCCCTCCCAGCCC |
| | | CCATCGAGAAAACCATCTCCAAAGCCAAAGGGCAGCCCCGAGAACCACAG |
| | | GTGTACACCCTGCCCCCATCCCGGGATGAGCTGACCAAGAACCAGGTCAGC |
| | | CTGACCTGCCTGGTCAAAGGCTTCTATCCCAGCGACATCGCCGTGGAGTGG |
| | | GAGAGCAATGGGCAGCCGGAGAACAACTACAAGACCACGCCTCCCGTGCT |
| | | GGACTCCGACGGCTCCTTCTTCCTCTACAGCAAGCTCACCGTGGACAAGAG |
| | | CAGGTGGCAGCAGGGGAACGTCTTCTCATGCTCCGTGATGCATGAGGCTCT |
| | | GCACAACCACTACACGCAGAAGAGCCTCTCCCTGTCTCCGGGTAAATGA |
| 85 | CD69 (625) | CAGACACTGAGGCTTGGGTTGGGCGAGGCCATTCAGACACTAAAACCCAGT |
| | promoter, | GCAGTTCTCCCTCAAGTGTGACCTTATATGAAGAATCCGGAGGGAGGTTTCT |
| | CD4/IgG1 | GAAAGGAAATGATGTAATGTGAGGCAGATGCAAAGTGCGGCAGGAAGGCA |
| | fusion protein, | GGGTGTACAGTCCTTATCACGGCAGCTGCCTTAGTGGTATGTGTTCAAAGGA |
| | antibody | ACCACAAACTTCCGACCTGAGGCAGTTTCCGGTGACAACCTGCTCATCATAT |
| | secretion signal | TTCAAAATGATTTTTTTCTTTCAGTGAGTGAATGAGGTACTGGAATGTCCAA |
| | (AGT123) | GCTTTCTGTTTCCTGCATTCTTACTTCTTACTACGTGATACATCTAGTCACCA |
| | | GGGAAGAAGCGAATGACACACTTCCAAAAACCAATTCGTAGCTTTCTAAAT |
| | | AAAACCCTTTCTAGCTGGAGAGAGATCCATGAGCATAGAGATCTTAAAATT |
| | | CATGTTCAGCAATAAATCCTGGGGCCCCAGACAGTGTCAGGTGCATAGGGG |
| | | GTGTTCAGTAAATATCAGTTAAATGTATGCATAAATCGATAAACGGGATTC |
| | | CTGGAAAATACTACACTCTCCTTCTCCAAATTATCTTCATCTCAAAGACAGG |
| | | AACCTCTAACTTTTAATTCTTTACTTAGATTATGCTGTCTCCTAAACTGTTTA |
| | | TGTTTTCTAGAAATTTAAGGCAGGATGTCTCAGAGTCTGGGAAAATCCCACT |
| | | TTCCTCCTGCTACACCTTACAGTTGTGAGAAAGCACATTTCAGACAACAGGG |
| | | AAAACCCATACTTCACCACAACAACACACTATACATTGTCTGGTCCACTGG |
| | | AGCATAAATTAAAGAGAAACAATGTAGTCAAGCAAGTAGGCGGCAAGAGG |
| | | AAGGGGGCGGAGACATCATCAGGGAGTATAAACTCTGAGATGCCTCAGAG |
| | | CCTCACGAATTCGCCACCATGGGATGGTCATGTATCATCCTTTTTCTAGTAG |
| | | CAACTGCAACTGGTGTACATTCCAAGAAAGTGGTGCTGGGCAAAAAAGGGG |
| | | ATACAGTGGAACTGACCTGCACAGCTTCCCAGAAGAAGAGCATACAATTCC |
| | | ACTGGAAAAACTCCAACCAGATAAAGATTCTGGGAAATCAGGGCTCCTTCT |
| | | TAACTAAAGGTCCATCCAAGCTGAATGATCGCGCTGACTCAAGAAGAAGCC |
| | | TTTGGGACCAAGGAAACTTTCCCCTGATCATCAAGAATCTTAAGATAGAAG |
| | | ACTCAGATACTTACATCTGTGAAGTGGAGGACCAGAAGGAGGAGGTGCAAT |
| | | TGCTAGTGTTCGGATTGACTGCCAACTCTGACACCCACCTGCTTCAGGGGCA |
| | | GAGCCTGACCCTGACCTTGGAGAGCCCCCCTGGTAGTAGCCCCTCAGTGCA |
| | | ATGTAGGAGTCCAAGGGGTAAAAACATACAAGGTGGTAAGACCCTCTCCGT |
| | | GTCTCAGCTGGAGCTCCAGGATAGTGGCACCTGGACATGCACTGTCTTGCA |
| | | GAACCAGAAGAAGGTGGAGTTCAAAATAGACATCGTGGTGCTAGCTGAGCC |
| | | CAAGAGCTGCGACAAGACCCACACCTGTCCACCATGCCCCGCACCTGAACT |
| | | CCTGGGGGGACCGTCAGTCTTCCTCTTCCCCCCAAAACCCAAGGACACCCTC |
| | | ATGATCTCCCGGACCCCTGAGGTCACATGCGTGGTGGTGGACGTGAGCCAC |
| | | GAAGACCCTGAGGTCAAGTTCAACTGGTACGTGGACGGCGTGGAGGTGCAT |
| | | AATGCCAAGACAAAGCCGCGGGAGGAGCAGTACAACAGCACGTACCGTGT |
| | | GGTCAGCGTCCTCACCGTCCTGCACCAGGACTGGCTGAATGGCAAGGAGTA |
| | | CAAGTGCAAGGTCTCCAACAAAGCCCTCCCAGCCCCCATCGAGAAAACCAT |
| | | CTCCAAAGCCAAAGGGCAGCCCCGAGAACCACAGGTGTACACCCTGCCCCC |
| | | ATCCCGGGATGAGCTGACCAAGAACCAGGTCAGCCTGACCTGCCTGGTCAA |
| | | AGGCTTCTATCCCAGCGACATCGCCGTGGAGTGGGAGAGCAATGGGCAGCC |
| | | GGAGAACAACTACAAGACCACGCCTCCCGTGCTGGACTCCGACGGCTCCTT |
| | | CTTCCTCTACAGCAAGCTCACCGTGGACAAGAGCAGGTGGCAGCAGGGGAA |
| | | CGTCTTCTCATGCTCCGTGATGCATGAGGCTCTGCACAACCACTACACGCAG |
| | | AAGAGCCTCTCCCTGTCTCCGGGTAAATGA |
| 86 | VRC01 Heavy | ATGGGATGGTCATGTATCATCCTTTTTCTAGTAGCAACTGCAACTGGTGTAC |
| | Variable Chain | ATTCCCAGGTGCAGCTGGTGCAGTCTGGGGGTCAGATGAAGAAGCCTGGCG |
| | (with antibody | AGTCGATGAGAATTTCTTGTCGGGCTTCTGGATATGAATTTATTGATTGTAC |
| | secretory | GCTAAATTGGATTCGTCTGGCCCCCGGAAAAAGGCCTGAGTGGATGGGATG |
| | signal) | GCTGAAGCCTCGGGGGGGGGCCGTCAACTACGCACGTCCACTTCAGGGCAG |
| | | AGTGACCATGACACGAGACGTTTATTCCGACACAGCCTTTTTGGAGCTGCGC |
| | | TCGTTGACAGTAGACGACACGGCCGTCTACTTTTGTACTAGGGGAAAAAAC |
| | | TGTGATTACAATTGGGACTTCGAACACTGGGGCCGGGGCACCCCGGTCATC |
| | | GTCTCATCA |
| 87 | EF1-VRC01 | CCGGTGCCTAGAGAAGGTGGCGCGGGGTAAACTGGGAAAGTGATGTCGTGT |
| | with Ab signal | ACTGGCTCCGCCTTTTTCCCGAGGGTGGGGGAGAACCGTATATAAGTGCAG |
| | sequence | TAGTCGCCGTGAACGTTCTTTTTCGCAACGGGTTTGCCGCCAGAACACAGGT |
| | (AGT114) | AAGTGCCGTGTGTGGTTCCCGCGGGCCTGGCCTCTTTACGGGTTATGGCCCT |
| | | TGCGTGCCTTGAATTACTTCCACGCCCCTGGCTGCAGTACGTGATTCTTGAT |
| | | CCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGC |
| | | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGGCCACCATGGGATGGTCATGTATCATCCTT |
| | | TTTCTAGTAGCAACTGCAACTGGTGTACATTCCCAGGTGCAGCTGGTGCAGT |
| | | CTGGGGGTCAGATGAAGAAGCCTGGCGAGTCGATGAGAATTTCTTGTCGGG |
| | | CTTCTGGATATGAATTTATTGATTGTACGCTAAATTGGATTCGTCTGGCCCC |
| | | CGGAAAAAGGCCTGAGTGGATGGGATGGCTGAAGCCTCGGGGGGGGGCCG |
| | | TCAACTACGCACGTCCACTTCAGGGCAGAGTGACCATGACACGAGACGTTT |
| | | ATTCCGACACAGCCTTTTTGGAGCTGCGCTCGTTGACAGTAGACGACACGG |
| | | CCGTCTACTTTTGTACTAGGGGAAAAAACTGTGATTACAATTGGGACTTCGA |
| | | ACACTGGGGCCGGGGCACCCCGGTCATCGTCTCATCAGCTAGCACCAAGGG |
| | | CCCATCGGTCTTCCCCCTGGCACCCTCCTCCAAGAGCACCTCTGGGGGCACA |
| | | GCGGCCCTGGGCTGCCTGGTCAAGGACTACTTCCCCGAACCGGTGACGGTG |
| | | TCGTGGAACTCAGGCGCCCTGACCAGCGGCGTGCACACCTTCCCGGCTGTC |
| | | CTACAGTCCTCAGGACTCTACTCCCTCAGCAGCGTGGTGACCGTGCCCTCCA |
| | | GCAGCTTGGGCACCCAGACCTACATCTGCAACGTGAATCACAAGCCCAGCA |
| | | ACACCAAGGTGGACAAGAAAGTTGAGCCCAAATCTTGTGACAAAACTCACA |
| | | CATGCCCACCGTGCCCAGCACCTGAACTCCTGGGGGGACCGTCAGTCTTCCT |
| | | CTTCCCCCCAAAACCCAAGGACACCCTCATGATCTCCCGGACCCCTGAGGTC |
| | | ACATGCGTGGTGGTGGACGTGAGCCACGAAGACCCTGAGGTCAAGTTCAAC |
| | | TGGTACGTGGACGGCGTGGAGGTGCATAATGCCAAGACAAAGCCGCGGGA |
| | | GGAGCAGTACAACAGCACGTACCGTGTGGTCAGCGTCCTCACCGTCCTGCA |
| | | CCAGGACTGGCTGAATGGCAAGGAGTACAAGTGCAAGGTCTCCAACAAAG |
| | | CCCTCCCAGCCCCCATCGAGAAAACCATCTCCAAAGCCAAAGGGCAGCCCC |
| | | GAGAACCACAGGTGTACACCCTGCCCCCATCCCGGGATGAGCTGACCAAGA |
| | | ACCAGGTCAGCCTGACCTGCCTGGTCAAAGGCTTCTATCCCAGCGACATCG |
| | | CCGTGGAGTGGGAGAGCAATGGGCAGCCGGAGAACAACTACAAGACCACG |
| | | CCTCCCGTGCTGGACTCCGACGGCTCCTTCTTCCTCTACAGCAAGCTCACCG |
| | | TGGACAAGAGCAGGTGGCAGCAGGGGAACGTCTTCTCATGCTCCGTGATGC |
| | | ATGAGGCTCTGCACAACCACTACACGCAGAAGAGCCTCTCCCTGTCTCCGG |
| | | GTAAACGTAGACGAAAGCGCGGAAGCGGAGAGGGCAGAGGAAGTCTGCTA |
| | | ACATGCGGTGACGTCGAGGAGAATCCTGGACCTGGATCCATGGGATGGTCA |
| | | TGTATCATCCTTTTTCTAGTAGCAACTGCAACTGGTGTACATTCCGAAATTG |
| | | TGTTGACACAGTCTCCAGGCACCCTGTCTTTGTCTCCAGGGGAAACAGCCAT |
| | | CATCTCTTGTCGGACCAGTCAGTATGGTTCCTTAGCCTGGTATCAACAGAGG |
| | | CCCGGCCAGGCCCCCAGGCTCGTCATCTATTCGGGCTCTACTCGGGCCGCTG |
| | | GCATCCCAGACAGGTTCAGCGGCAGTCGGTGGGGGCCAGACTACAATCTCA |
| | | CCATCAGCAACCTGGAGTCGGGAGATTTTGGTGTTTATTATTGCCAGCAGTA |
| | | TGAATTTTTTGGCCAGGGGACCAAGGTCCAGGTCGACATTAAGCGAGAATT |
| | | CGTGGCTGCACCATCTGTCTTCATCTTCCCGCCATCTGATGAGCAGTTGAAA |
| | | TCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGG |
| | | AGAGTGTCACAGAGCAGGACAGCAAGGACAGCACCTACAGCCTCAGCAGC |
| | | ACCCTGACGCTGAGCAAAGCAGACTACGAGAAACACAAAGTCTACGCCTGC |
| | | GAAGTCACCCATCAGGGCCTGAGCTCGCCCGTCACAAAGAGCTTCAACAGG |
| | | GGAGAGTGTTAG |
| 88 | EF-1 | CCGGTGCCTAGAGAAGGTGGCGCGGGGTAAACTGGGAAAGTGATGTCGTGT |
| | promoter, | ACTGGCTCCGCCTTTTTCCCGAGGGTGGGGGAGAACCGTATATAAGTGCAG |
| | CD4/IgG1 | CCGGTGCCTAGAGAAGGTGGCGCGGGGTAAACTGGGAAAGTGATGTCGTGT |
| | fusion protein | ACTGGCTCCGCCTTTTTCCCGAGGGTGGGGGAGAACCGTATATAAGTGCAG |
| | version 2, | TAGTCGCCGTGAACGTTCTTTTTCGCAACGGGTTTGCCGCCAGAACACAGGT |
| | antibody | AAGTGCCGTGTGTGGTTCCCGCGGGCCTGGCCTCTTTACGGGTTATGGCCCT |
| | secretion signal | TGCGTGCCTTGAATTACTTCCACGCCCCTGGCTGCAGTACGTGATTCTTGAT |
| | (AGT124) | CCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGC |
| | | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAGCCACCATGGGATGGTCATGTATCA |
| | | TCCTTTTTCTAGTAGCAACTGCAACTGGTGTACATTCCAAGAAAGTGGTGCT |
| | | GGGCAAAAAAGGGGATACAGTGGAACTGACCTGCACAGCTTCCCAGAAGA |
| | | AGAGCATACAATTCCACTGGAAAAACTCCAACCAGATAAAGATTCTGGGAA |
| | | ATCAGGGCTCCTTCTTAACTAAAGGTCCATCCAAGCTGAATGATCGCGCTGA |
| | | CTCAAGAAGAAGCCTTTGGGACCAAGGAAACTTTCCCCTGATCATCAAGAA |
| | | TCTTAAGATAGAAGACTCAGATACTTACATCTGTGAAGTGGAGGACCAGAA |
| | | GGAGGAGGTGCAATTGCTAGTGTTCGGATTGACTGCCAACTCTGACACCCA |
| | | CCTGCTTCAGGGGCAGAGCCTGACCCTGACCTTGGAGAGCCCCCCTGGTAG |
| | | TAGCCCCTCAGTGCAATGTAGGAGTCCAAGGGGTAAAAACATACAAGGTGG |
| | | TAAGACCCTCTCCGTGTCTCAGCTGGAGCTCCAGGATAGTGGCACCTGGAC |
| | | ATGCACTGTCTTGCAGAACCAGAAGAAGGTGGAGTTCAAAATAGACATCGT |
| | | GGTGCTAGCTGAGCCCAAGAGCTGCGACAAGACCCACACCTGTCCACCATG |
| | | CCCCGCACCTGAACTCCTGGGGGGACCGTCAGTCTTCCTCTTCCCCCCAAAA |
| | | CCCAAGGACACCCTCATGATCTCCCGGACCCCTGAGGTCACATGCGTGGTG |
| | | GTGGACGTGAGCCACGAAGACCCTGAGGTCAAGTTCAACTGGTACGTGGAC |
| | | GGCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGGCT |
| | | GAATGGCAAGGAGTACAAGTGCAAGGTCTCCAACAAAGCCCTCCCAGCCCC |
| | | CATCGAGAAAACCATCTCCAAAGCCAAAGGGCAGCCCCGAGAACCACAGG |
| | | TGTACACCCTGCCCCCATCCCGGGATGAGCTGACCAAGAACCAGGTCAGCC |
| | | TGACCTGCCTGGTCAAAGGCTTCTATCCCAGCGACATCGCCGTGGAGTGGG |
| | | AGAGCAATGGGCAGCCGGAGAACAACTACAAGACCACGCCTCCCGTGCTG |
| | | GACTCCGACGGCTCCTTCTTCCTCTACAGCAAGCTCACCGTGGACAAGAGC |
| | | AGGTGGCAGCAGGGGAACGTCTTCTCATGCTCCGTGATGCATGAGGCTCTG |
| | | CACAACCACTACACGCAGAAGAGCCTCTCCCTGTCTCCGGGTAAATGA |
| 89 | EF- | CCGGTGCCTAGAGAAGGTGGCGCGGGGTAAACTGGGAAAGTGATGTCGTGT |
| | 1α promoter, | ACTGGCTCCGCCTTTTTCCCGAGGGTGGGGGAGAACCGTATATAAGTGCAG |
| | CD4/IgG1 | TAGTCGCCGTGAACGTTCTTTTTCGCAACGGGTTTGCCGCCAGAACACAGGT |
| | fusion protein | AAGTGCCGTGTGTGGTTCCCGCGGGCCTGGCCTCTTTACGGGTTATGGCCCT |
| | version 3, | TGCGTGCCTTGAATTACTTCCACGCCCCTGGCTGCAGTACGTGATTCTTGAT |
| | antibody | CCCGAGCTTCGGGTTGGAAGTGGGTGGGAGAGTTCGAGGCCTTGCGCTTAA |
| | secretion signal | GGAGCCCCTTCGCCTCGTGCTTGAGTTGAGGCCTGGCCTGGGCGCTGGGGC |
| | (AGT125) | CGCCGCGTGCGAATCTGGTGGCACCTTCGCGCCTGTCTCGCTGCTTTCGATA |
| | | AGTCTCTAGCCATTTAAAATTTTTGATGACCTGCTGCGACGCTTTTTTTCTGG |
| | | CAAGATAGTCTTGTAAATGCGGGCCAAGATCTGCACACTGGTATTTCGGTTT |
| | | TTGGGGCCGCGGGCGGCGACGGGGCCCGTGCGTCCCAGCGCACATGTTCGG |
| | | CGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCC |
| | | CGCCCTGGGCGGCAAGGCTGGCCCGGTCGGCACCAGTTGCGTGAGCGGAAA |
| | | GATGGCCGCTTCCCGGCCCTGCTGCAGGGAGCTCAAAATGGAGGACGCGGC |
| | | GCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCA |
| | | GGCACCTCGATTAGTTCTCGAGCTTTTGGAGTACGTCGTCTTTAGGTTGGGG |
| | | GGAGGGGTTTTATGCGATGGAGTTTCCCCACACTGAGTGGGTGGAGACTGA |
| | | AGTTAGGCCAGCTTGGCACTTGATGTAATTCTCCTTGGAATTTGCCCTTTTTG |
| | | AGTTTGGATCTTGGTTCATTCTCAAGCCTCAGACAGTGGTTCAAAGTTTTTTT |
| | | CTTCCATTTCAGGTGTCGTGATGTACAGCCACCATGGGATGGTCATGTATCA |
| | | TCCTTTTTCTAGTAGCAACTGCAACTGGTGTACATTCCAAGAAAGTGGTGCT |
| | | GGGCAAAAAAGGGGATACAGTGGAACTGACCTGCACAGCTTCCCAGAAGA |
| | | AGAGCATACAATTCCACTGGAAAAACTCCAACCAGATAAAGATTCTGGGAA |
| | | ATCAGGGCTCCTTCTTAACTAAAGGTCCATCCAAGCTGAATGATCGCGCTGA |
| | | CTCAAGAAGAAGCCTTTGGGACCAAGGAAACTTTCCCCTGATCATCAAGAA |
| | | TCTTAAGATAGAAGACTCAGATACTTACATCTGTGAAGTGGAGGACCAGAA |
| | | GGAGGAGGTGCAATTGCTAGTGTTCGGATTGACTGCCAACTCTGACACCCA |
| | | CCTGCTTCAGGGGCAGAGCCTGACCCTGACCTTGGAGAGCCCCCCTGGTAG |
| | | TAGCCCCTCAGTGCAATGTAGGAGTCCAAGGGGTAAAAACATACAAGGTGG |
| | | TAAGACCCTCTCCGTGTCTCAGCTGGAGCTCCAGGATAGTGGCACCTGGAC |
| | | ATGCACTGTCTTGCAGAACCAGAAGAAGGTGGAGTTCAAAATAGACATCGT |
| | | GGTGCTAGCTGAGCCCAAGAGCTGCGACAAGACCCACACCTGTCCACCATG |
| | | CCCCGCACCTGAAGCTGCAGGGGGACCGTCAGTCTTCCTCTTCCCCCCAAAA |
| | | CCCAAGGACACCCTCATGATCTCCCGGACCCCTGAGGTCACATGCGTGGTG |
| | | GTGGACGTGAGCCACGAAGACCCTGAGGTCAAGTTCAACTGGTACGTGGAC |
| | | GGCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGGCT |
| | | GAATGGCAAGGAGTACAAGTGCGCTGTCTCCAACAAAGCCCTCCCAGCCCC |
| | | CATCGAGAAAACCATCTCCAAAGCCAAAGGGCAGCCCCGAGAACCACAGG |
| | | TGTACACCCTGCCCCCATCCCGGGATGAGCTGACCAAGAACCAGGTCAGCC |
| | | TGACCTGCCTGGTCAAAGGCTTCTATCCCAGCGACATCGCCGTGGAGTGGG |
| | | AGAGCAATGGGCAGCCGGAGAACAACTACAAGACCACGCCTCCCGTGCTG |
| | | GACTCCGACGGCTCCTTCTTCCTCTACAGCAAGCTCACCGTGGACAAGAGC |
| | | AGGTGGCAGCAGGGGAACGTCTTCTCATGCTCCGTGATGCATGAGGCTCTG |
| | | CACAACCACTACACGCAGAAGAGCCTCTCCCTGTCTCCGGGTAAATGA |

While certain of the preferred embodiments have been described and specifically exemplified above, it is not intended that the disclosure be limited to such preferred embodiments. Various modifications may be made thereto without departing from the scope and spirit of the present embodiments described herein.

1. A viral vector comprising a therapeutic cargo portion, wherein the therapeutic cargo portion comprises a nucleotide sequence that encodes at least one soluble exogenous factor capable of inhibiting HIV infection, and a T cell-responsive promoter that regulates expression of the nucleotide sequence.

2. The viral vector of claim 1, wherein the at least one soluble exogenous factor comprises an anti-HIV antibody.

3. The viral vector of claim 2, wherein the anti-HIV antibody is a VRC01 antibody or a 3BNC117 antibody.

4. The viral vector of claim 1, wherein the at least one soluble exogenous factor comprises a soluble CD4 protein or a fragment thereof.

5. The viral vector of claim 4, wherein the soluble CD4 or a fragment thereof comprises a dimeric soluble CD4.

6. The viral vector of claim 5, wherein the dimeric soluble CD4 comprises a sequence having at least 80%, at least 85%, at least 90%, at least 95%, or at least 98% sequence identity to SEQ ID NO: 9, SEQ ID NO: 76, or SEQ ID NO: 77.

7. The viral vector of claim 6, wherein the dimeric soluble CD4 comprises SEQ ID NO: 9, SEQ ID NO: 76, or SEQ ID NO: 77.

8. The viral vector of claim 1, wherein the T cell-responsive promoter comprises a CMV promoter, an IFN-α promoter, an IFN-β promoter, an IFN-γ promoter, an EF-1α promoter, an IL-2 promoter, a CD69 promoter, or a fragment thereof.

9. The viral vector of claim 8, wherein the T cell-responsive promoter comprises an IL-2 promoter.

10. The viral vector of claim 1, wherein the therapeutic cargo portion further comprises a secretory signal that is operably linked to the nucleotide sequence that encodes the at least one soluble exogenous factor.

11. The viral vector of claim 10, wherein the secretory signal comprises an antibody secretory signal or an IL-2 secretory signal.

12. The viral vector of claim 1, wherein the nucleotide sequence comprises a sequence having at least 80%, at least 85%, at least 90%, at least 95%, or at least 98% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87.

13. The viral vector of claim 1, wherein the nucleotide sequence comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 78, SEQ ID NO: 80, SEQ ID NO: 81, SEQ ID NO: 82, SEQ ID NO: 83, SEQ ID NO: 84, SEQ ID NO: 85, or SEQ ID NO: 87.

14. The viral vector of claim 1, wherein the therapeutic cargo portion further comprises at least one small RNA that targets any one or more of Vif, Tat, and CCR5.

15. The viral vector of claim 14, wherein the at least one small RNA comprises a sequence having at least 80%, at least 85%, at least 90%, at least 95%, or at least 98% sequence identity to SEQ ID NO: 62, SEQ ID NO: 63, or SEQ ID NO: 64.

16. The viral vector of claim 15, wherein the at least one small RNA comprises SEQ ID NO: 62, SEQ ID NO: 63, or SEQ ID NO: 64.

17. The viral vector of claim 14, wherein the at least one soluble exogenous factor comprises soluble CD4 or a fragment thereof.

18. The viral vector of claim 17, wherein the soluble CD4 or a fragment thereof comprises a dimeric soluble CD4.

19. The viral vector of claim 14, wherein the T cell-responsive promoter comprises a CMV promoter, an IFN-α promoter, an IFN-β promoter, an IFN-γ promoter, an EF-1α promoter, an IL-2 promoter, a CD69 promoter, or a fragment thereof.

20. The viral vector of claim 14, wherein the therapeutic cargo portion further comprises a secretory signal that is operably linked to the nucleotide sequence that encodes the at least one soluble exogenous factor.

21. The viral vector of claim 14, wherein the at least one small RNA comprises a sequence having at least 80%, at least 85%, at least 90%, at least 95%, or at least 98% sequence identity to SEQ ID NO: 65.

22. The viral vector of claim 21, wherein the at least one small RNA comprises SEQ ID NO: 65.

23. A lentiviral particle produced by a packaging cell and capable of infecting a target cell, the lentiviral particle comprising:
an envelope protein capable of infecting the target cell; and
the viral vector of claim 1.

24. A modified cell comprising a lymphocyte infected with a lentiviral particle, wherein the lentiviral particle comprises:
an envelope protein capable of infecting the lymphocyte; and
the viral vector of claim 1.

25. The modified cell of claim 24, wherein the lymphocyte comprises a T cell, a B cell, an NKT cell, or an NK cell.

26. The modified cell of claim 25, wherein the lymphocyte is a T cell, and wherein the T cell comprises a CD4 T cell, a CD8 T cell, or a γδ T cell.

27. The modified cell of claim 26, wherein the T cell is a CD4 T cell.

28. A viral delivery system comprising:
at least one helper plasmid comprising nucleotide sequences for expressing a functional protein derived from each of a Gag, Pol, and Rev gene;
an envelope plasmid comprising a DNA sequence for expressing an envelope protein capable of infecting a target cell; and
the viral vector of claim 1.

29. The viral delivery system of claim 28, wherein the at least one helper plasmid comprises first and second helper plasmids, wherein the first helper plasmid encodes nucleotide sequences for expressing functional proteins derived from the Gag and the Pol genes, and the second helper plasmid encodes a nucleotide sequence for expressing a protein derived from the rev gene

30. A method of treating HIV, the method comprising:
contacting peripheral blood mononuclear cells (PBMC) isolated from a subject with a therapeutically effective amount of a stimulatory agent, wherein the contacting is carried out ex vivo;
transducing the PBMC ex vivo with a lentiviral particle, wherein the lentiviral particle comprises:
an envelope protein capable of infecting the PBMC; and
the viral vector of claim 1; and
culturing the transduced PBMC for at least 1 day.

31. The method of claim 30, further comprising infusing the transduced PBMC into the subject.

32. The method of claim 30, wherein the stimulatory agent comprises a Gag peptide or an HIV vaccine.


SEQUENCE LISTING (89 sequences)

SEQ ID NO: 1 - DNA, 2181 bp, Artificial Sequence
  VRC01 (IL-2 secretory sequence)-HV-CH-T2A-LV-CL
atgtacaggatgcaactcctgtcttgcattgcactaagtcttgcacttgtcacgcaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgtacaggatgcaactcctgtcttgcattgcactaagtcttgcacttgtcacggaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 2551 bp, Artificial Sequence
  CMV- VRC01 (IL-2 secretory sequence)-HV-CH-T2A- LV-CL (AGT111)
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagatctcgaggccaccatgtacaggatgcaactcctgtcttgcattgcactaagtcttgcacttgtcacgcaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgtacaggatgcaactcctgtcttgcattgcactaagtcttgcacttgtcacggaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - DNA, 2208 bp, Artificial Sequence
  3BNC117 (IL-2 secretory sequence)-HV-CH-T2A-LV- CL
atgtacaggatgcaactcctgtcttgcattgcactaagtcttgcacttgtcacgcaggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcagcgtcgaccaagggcccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgtacaggatgcaactcctgtcttgcattgcactaagtcttgcacttgtcacggacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgtacggtggctgcaccagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - DNA, 2578 bp, Artificial Sequence
  CMV- 3BNC117 (IL-2 secretory sequence)-HV-CH- T2A-LV-CL (AGT112)
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagattctagatctcgaggccaccatgtacaggatgcaactcctgtcttgcattgcactaagtcttgcacttgtcacgcaggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcagcgtcgaccaagggcccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgtacaggatgcaactcctgtcttgcattgcactaagtcttgcacttgtcacggacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgtacggtggctgcaccagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - DNA, 2187 bp, Artificial Sequence
  VRC01 (Antibody secretory sequence)-HV-CH-T2A- LV-CL
atgggatggtcatgtatcatcctttttctagtagcaactgcaactggtgtacattcccaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgggatggtcatgtatcatcctttttctagtagcaactgcaactggtgtacattccgaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - DNA, 2557 bp, Artificial Sequence
  CMV-VRC01 (Antibody secretory sequence)-HV-CH- T2A-LV-CL (AGT113)
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagatctcgaggccaccatgggatggtcatgtatcatcctttttctagtagcaactgcaactggtgtacattcccaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgggatggtcatgtatcatcctttttctagtagcaactgcaactggtgtacattccgaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 7 - DNA, 594 bp, Artificial Sequence
  sCD4(D1+D2)
atgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagcttga

SEQ ID NO: 8 - DNA, 1710 bp, Artificial Sequence
  EF-1-sCD4(D1+D2) (AGT116)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacagccaccatgggatggtcatgtatcatcctttttctagtagcaactgcaactggtgttcattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagcttga

SEQ ID NO: 9 - DNA, 1401 bp, Artificial Sequence
  sCD4(D1+D2)-IgG1 Fc
atgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - DNA, 2517 bp, Artificial Sequence
  EF-1alpha promoter- sCD4(D1+D2)-IgG1 Fc fusion protein (truncated SEQ ID NO 9), antibody secretion signal (AGT117)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacagccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 11 - DNA, 54 bp, Artificial Sequence
  IL-2 secretory signal
atgtacaggatgcaactcctgtcttgcattgcactaagtcttgcacttgtcacg

SEQ ID NO: 12 - DNA, 57 bp, Artificial Sequence
  Antibody secretory signal
atgggatggtcatgtatcatcctttttctagtagcaactgcaactggtgtacattcc

SEQ ID NO: 13 - DNA, 350 bp, Artificial Sequence
  Cytomegalovirus (CMV) promoter
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 14 - DNA, 1104 bp, Artificial Sequence
  Human elongation factor 1 alpha (EF-1alpha) promoter
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 15 - DNA, 615 bp, Artificial Sequence
  Interferon gamma (IFNgamma) promoter
tgtatttctactgggcagtgctgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcag

SEQ ID NO: 16 - DNA, 523 bp, Artificial Sequence
  Prothrombin/Human alpha-1-anti trypsin (hAAT) enhancer/promoter
gcgagaacttgtgcctccccgtgttcctgctctttgtccctctgtcctacttagactaatatttgccttgggtactgcaaacaggaaatgggggagggacaggagtagggcggagggtagcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 17 - DNA, 228 bp, Artificial Sequence
  Rous Sarcoma virus (RSV) promoter
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 18 - DNA, 180 bp, Artificial Sequence
  5′ Long terminal repeat (LTR)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 19 - DNA, 41 bp, Artificial Sequence
  Psi Packaging signal
tacgccaaaaattttgactagcggaggctagaaggagagag

SEQ ID NO: 20 - DNA, 233 bp, Artificial Sequence
  Rev response element (RRE)
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 21 - DNA, 118 bp, Artificial Sequence
  Central polypurine tract (cPPT)
ttttaaaagaaaaggggggattggggggtacagtgcaggggaaagaatagtagacataatagcaacagacatacaaactaaagaattacaaaaacaaattacaaaattcaaaatttta

SEQ ID NO: 22 - DNA, 590 bp, Artificial Sequence
  Long WPRE sequence
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 23 - DNA, 389 bp, Artificial Sequence
  Short WPRE sequence
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 24 - DNA, 250 bp, Artificial Sequence
  3′ delta LTR
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 25 - DNA, 290 bp, Artificial Sequence
  Helper/Rev - Chicken beta actin (CAG) promoter - Transcription
gctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 26 - DNA, 1503 bp, Artificial Sequence
  Helper/Rev - HIV Gag - Viral capsid
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 27 - DNA, 1872 bp, Artificial Sequence
  Helper/Rev - HIV Pol - Protease and reverse transcriptase
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 28 - DNA, 867 bp, Artificial Sequence
  Helper Rev - HIV Integrase - Integration of viral RNA
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 29 - DNA, 234 bp, Artificial Sequence
  Helper/Rev - HIV RRE - Binds Rev element
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 30 - DNA, 351 bp, Artificial Sequence
  Helper/Rev - HIV Rev - Nuclear export and stabilize viral mRNA
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 31 - DNA, 577 bp, Artificial Sequence
  Envelope - CMV promoter
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 32 - DNA, 1519 bp, Artificial Sequence
  Envelope - VSV-G - Glycoprotein envelope-cell entry
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatga

SEQ ID NO: 33 - DNA, 352 bp, Artificial Sequence
  Helper/Rev - CMV early (CAG) enhancer - Enhance Transcription
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 34 - DNA, 960 bp, Artificial Sequence
  Helper/Rev - Chicken beta actin intron - Enhance gene expression
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 35 - DNA, 448 bp, Artificial Sequence
  Helper/Rev - Rabbit beta globin poly A - RNA stability
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 36 - DNA, 573 bp, Artificial Sequence
  Envelope - Beta globin intron - Enhance gene expression
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 37 - DNA, 450 bp, Artificial Sequence
  Envelope - Rabbit beta globin poly A - RNA stability
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 38 - DNA, 31 bp, Artificial Sequence
  Forward Primer
taagcagaattcatgaatttgccaggaagat

SEQ ID NO: 39 - DNA, 36 bp, Artificial Sequence
  Reverse Primer
ccatacaatgaatggacactaggcggccgcacgaat

SEQ ID NO: 40 - DNA, 2745 bp, Artificial Sequence
  Gag, Pol, Integrase fragment
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 41 - DNA, 1586 bp, Artificial Sequence
  DNA Fragment containing Rev, RRE and rabbit beta globin poly A
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 42 - DNA, 1614 bp, Artificial Sequence
  DNA fragment containing the CAG enhancer/promoter/intron sequence
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 43 - DNA, 1531 bp, Artificial Sequence
  DNA fragment containing VSV-G
gaattcatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgagaattc

SEQ ID NO: 44 - DNA, 1227 bp, Artificial Sequence
  Helper plasmid containing RRE and rabbit beta globin poly A
tctagaaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcacccggg

SEQ ID NO: 45 - DNA, 884 bp, Artificial Sequence
  RSV promoter and HIV Rev
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 46 - DNA, 511 bp, Artificial Sequence
  Promoter - PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 47 - DNA, 1162 bp, Artificial Sequence
  Promoter - UbC
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 48 - DNA, 642 bp, Artificial Sequence
  CAG promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 49 - DNA, 120 bp, Artificial Sequence
  Poly A - SV40
gtttattgcagcttataatggttacaaataaagcaatagcatcacaaatttcacaaataaagcatttttttcactgcattctagttgtggtttgtccaaactcatcaatgtatcttatca

SEQ ID NO: 50 - DNA, 227 bp, Artificial Sequence
  Poly A - bGH
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 51 - DNA, 1512 bp, Artificial Sequence
  HIV Gag - Bal
atgggtgcgagagcgtcagtattaagcgggggagaattagataggtgggaaaaaattcggttaaggccagggggaaagaaaaaatatagattaaaacatatagtatgggcaagcagggaactagaaagattcgcagtcaatcctggcctgttagaaacatcagaaggctgcagacaaatactgggacagctacaaccatcccttcagacaggatcagaagaacttagatcattatataatacagtagcaaccctctattgtgtacatcaaaagatagaggtaaaagacaccaaggaagctttagacaaaatagaggaagagcaaaacaaatgtaagaaaaaggcacagcaagcagcagctgacacaggaaacagcggtcaggtcagccaaaatttccctatagtgcagaacctccaggggcaaatggtacatcaggccatatcacctagaactttaaatgcatgggtaaaagtaatagaagagaaagctttcagcccagaagtaatacccatgttttcagcattatcagaaggagccaccccacaagatttaaacaccatgctaaacacagtggggggacatcaagcagccatgcaaatgttaaaagaacccatcaatgaggaagctgcaagatgggatagattgcatcccgtgcaggcagggcctgttgcaccaggccagataagagatccaaggggaagtgacatagcaggaactaccagtacccttcaggaacaaataggatggatgacaagtaatccacctatcccagtaggagaaatctataaaagatggataatcctgggattaaataaaatagtaaggatgtatagccctaccagcattttggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcagctacactagaagaaatgatgacagcatgtcagggagtgggaggacccagccataaagcaagaattttggcagaagcaatgagccaagtaacaaattcagctaccataatgatgcagaaaggcaattttaggaaccaaagaaagattgttaaatgtttcaattgtggcaaagaagggcacatagccagaaactgcagggcccctaggaaaaggggctgttggaaatgtggaaaggaaggacaccaaatgaaagactgtactgagagacaggctaattttttagggaaaatctggccttcccacaaaggaaggccagggaatttccttcagagcagaccagagccaacagccccaccagccccaccagaagagagcttcaggtttggggaagagacaacaactccctctcagaagcaggagctgatagacaaggaactgtatcctttagcttccctcagatcactctttggcaacgacccctcgtcacaataa

SEQ ID NO: 52 - DNA, 1872 bp, Artificial Sequence
  HIV Pol - Bal
atgaatttgccaggaagatggaaaccaaaaatgatagggggaattggaggttttatcaaagtaagacagtatgatcagatactcatagaaatctgtggacataaagctataggtacagtattaataggacctacacctgtcaacataattggaagaaatctgttgactcagattggttgcactttaaattttcccattagtcctattgaaactgtaccagtaaaattaaaaccaggaatggatggcccaaaagttaaacaatggccactgacagaagaaaaaataaaagcattaatggaaatctgtacagaaatggaaaaggaagggaaaatttcaaaaattgggcctgaaaatccatacaatactccagtatttgccataaagaaaaaagacagtactaaatggagaaaattagtagatttcagagaacttaataagaaaactcaagacttctgggaagtacaattaggaatacacatcccgcaggggttaaaaaagaaaaaatcagtaacagtactggatgtgggtgatgcatatttttcagttcccttagataaagaattcaggaagtatactgcatttaccatacctagtataaacaatgaaacaccagggatcagatatcagtacaatgtacttccacagggatggaaaggatcaccagcaatatttcaaagtagcatgacaagaatcttagagccttttagaaaacaaaatccagaaatagtgatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaagacagctggactgtcaatgacatacagaagttagtgggaaaattgaattgggcaagtcagatttacccaggaattaaagtaaagcaattatgtaggctccttaggggaaccaaggcattaacagaagtaataccactaacaaaagaaacagagctagaactggcagagaacagggaaattctaaaagaaccagtacatggggtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaagaaacatgggaaacatggtggacagagtattggcaagccacctggattcctgagtgggagtttgtcaatacccctcccttagtgaaattatggtaccagttagagaaagaacccataataggagcagaaacattctatgtagatggagcagctaaccgggagactaaattaggaaaagcaggatatgttactaacagaggaagacaaaaagttgtctccctaactgacacaacaaatcagaagactgagttacaagcaattcatctagctttacaagattcaggattagaagtaaacatagtaacagactcacaatatgcattaggaatcattcaagcacaaccagataaaagtgaatcagagttagtcagtcaaataatagaacagttaataaaaaaggaaaaggtctacctggcatgggtaccagcgcacaaaggaattggaggaaatgaacaagtagataaattagtcagtactggaatcaggaaagtacta

SEQ ID NO: 53 - DNA, 867 bp, Artificial Sequence
  HIV Integrase - Bal
tttttagatggaatagatatagcccaagaagaacatgagaaatatcacagtaattggagagcaatggctagtgattttaacctgccacctgtggtagcaaaagaaatagtagccagctgtgataaatgtcagctaaaaggagaagccatgcatggacaagtagactgtagtccaggaatatggcaactagattgtacacatttagaaggaaaaattatcctggtagcagttcatgtagccagtggatatatagaagcagaagttattccagcagagacagggcaggaaacagcatactttctcttaaaattagcaggaagatggccagtaaaaacaatacatacagacaatggcagcaatttcactagtactacagtcaaggccgcctgttggtgggcggggatcaagcaggaatttggcattccctacaatccccaaagtcagggagtagtagaatctataaataaagaattaaagaaaattataggacaggtaagagatcaggctgaacatcttaaaacagcagtacaaatggcagtattcatccacaattttaaaagaaaaggggggattggggggtatagtgcaggggaaagaatagtagacataatagcaacagacatacaaactaaagaattacaaaaacaaattacaaaaattcaaaattttcgggtttattacagggacagcagagatccactttggaaaggaccagcaaagcttctctggaaaggtgaaggggcagtagtaatacaagataatagtgacataaaagtagtaccaagaagaaaagcaaagatcattagggattatggaaaacagatggcaggtgatgattgtgtggcaagtagacaggatgaggattag

SEQ ID NO: 54 - DNA, 1695 bp, Artificial Sequence
  Envelope - RD114
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 55 - DNA, 2013 bp, Artificial Sequence
  Envelope - GALV
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 56 - DNA, 1530 bp, Artificial Sequence
  Envelope - FUG
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 57 - DNA, 1497 bp, Artificial Sequence
  Envelope - LCMV
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 58 - DNA, 1692 bp, Artificial Sequence
  Envelope - FPV
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 59 - DNA, 1266 bp, Artificial Sequence
  Envelope - RRV
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 60 - DNA, 2002 bp, Artificial Sequence
  Envelope - MLV 10A1
ggatccacgccgctcacgtaaaggcggcgacaacccctccggccggaacagcatcaggaccgacatggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 61 - DNA, 2030 bp, Artificial Sequence
  Envelope - Ebola
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 62 - DNA, 118 bp, Artificial Sequence
  miR30-CCR5
aggtatattgctgttgacagtgagcgactgtaaactgagcttgctctactgtgaagccacagatgggtagagcaagcacagtttaccgctgcctactgcctcggacttcaaggggctt

SEQ ID NO: 63 - DNA, 116 bp, Artificial Sequence
  miR21-Vif
catctccatggctgtaccaccttgtcgggggatgtgtacttctgaacttgtgttgaatctcatggagttcagaagaacacatccgcactgacattttggtatctttcatctgacca

SEQ ID NO: 64 - DNA, 113 bp, Artificial Sequence
  miR185-Tat
gggcctggctcgagcagggggcgagggattccgcttcttcctgccatagcgtggtcccctcccctatggcaggcagaagcggcaccttccctcccaatgaccgcgtcttcgtc

SEQ ID NO: 65 - DNA, 359 bp, Artificial Sequence
  miR30-CCR5/miR21-Vif/miR185-Tat microRNA cluster sequence
aggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtc

SEQ ID NO: 66 - DNA, 524 bp, Artificial Sequence
  IL-2 promoter
atctatcttattgtatgcaattagctcattgtgtggataaaaaggtaaaaccattctgaaacaggaaaccaatacacttcctgtttaatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctt

SEQ ID NO: 67 - DNA, 1357 bp, Artificial Sequence
  CD69 promoter (1050) + CNS2 enhancer
cagacactgaggcttgggttgggcgaggccattcagacactaaaacccagtgcagttctccctcaagtgtgaccttatatgaagaatccggagggaggtttctgaaaggaaatgatgtaatgtgaggcagatgcaaagtgcggcaggaaggcagggtgtacagtccttatcacggcagctgccttagtggtatgtgttcaaaggaaccacaaacttccgacctgaggcagtttccggtgacaacctgctcatcatatttcaaaatgatttttttctttcagtgagtgaatgaggtactggaatgtcctctagatgataacttccaacccacctatgcataaaatttaacgtctttattctaaataagtgatattaataataaaatttggggcaccaagattattaatcagagtggtattttgatttccctccttaaatcaccatacatagctttctgcattcatcttgcgttgactgtcattacttgtctgagtgagactgataccacagcgatgttttaaataataatcatacctcaaaagactgaagtctcagaggtatctgaagagaataacctagagcacagggggagaattgaaggagctgttactgaggtgacataaaagcagtctaaatgacagtaaaatgtgacaagaaaattagcaggaaacaaatgaaacagataatttaagataaacaattttagagcatagcaaggaagttccagaccacaagctttctgtttcctgcattcttacttcttactacgtgatacatctagtcaccagggaagaagcgaatgacacacttccaaaaaccaattcgtagctttctaaataaaaccctttctagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcac

SEQ ID NO: 68 - DNA, 932 bp, Artificial Sequence
  CD69 promoter (625) + CNS2 enhancer
cagacactgaggcttgggttgggcgaggccattcagacactaaaacccagtgcagttctccctcaagtgtgaccttatatgaagaatccggagggaggtttctgaaaggaaatgatgtaatgtgaggcagatgcaaagtgcggcaggaaggcagggtgtacagtccttatcacggcagctgccttagtggtatgtgttcaaaggaaccacaaacttccgacctgaggcagtttccggtgacaacctgctcatcatatttcaaaatgatttttttctttcagtgagtgaatgaggtactggaatgtccaagctttctgtttcctgcattcttacttcttactacgtgatacatctagtcaccagggaagaagcgaatgacacacttccaaaaaccaattcgtagctttctaaataaaaccctttctagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcac

SEQ ID NO: 69 - DNA, 417 bp, Artificial Sequence
  VRC01 Heavy Variable Chain (with IL-2 secretory signal)
atgtacaggatgcaactcctgtcttgcattgcactaagtcttgcacttgtcacgcaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatca

SEQ ID NO: 70 - DNA, 985 bp, Artificial Sequence
  IgG1 Heavy Constant Chain
caccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 71 - DNA, 369 bp, Artificial Sequence
  VRC01 Light Variable Chain (with antibody secretory signal)
atgggatggtcatgtatcatcctttttctagtagcaactgcaactggtgtacattccgaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcga

SEQ ID NO: 72 - DNA, 366 bp, Artificial Sequence
  VRC01 Light Variable Chain (with IL-2 secretory signal)
atgtacaggatgcaactcctgtcttgcattgcactaagtcttgcacttgtcacggaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcga

SEQ ID NO: 73 - DNA, 318 bp, Artificial Sequence
  IgG1 Light Constant Chain
gtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 74 - DNA, 441 bp, Artificial Sequence
  3BNC117 Heavy Variable Chain (with IL-2 secretory signal)
atgtacaggatgcaactcctgtcttgcattgcactaagtcttgcacttgtcacgcaggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcagcgtcgaccaagggccca

SEQ ID NO: 75 - DNA, 369 bp, Artificial Sequence
  3BNC117 Light Variable Chain (with IL-2 secretory signal)
atgtacaggatgcaactcctgtcttgcattgcactaagtcttgcacttgtcacggacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgtacggtggctgcacca

SEQ ID NO: 76 - DNA, 1290 bp, Artificial Sequence
  sCD4-IgG1 Fc (with antibody secretory signal) version 2
atgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 77 - DNA, 1290 bp, Artificial Sequence
  sCD4-IgG1 Fc (with antibody secretory signal) version 3
atgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgctgtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 78 - DNA, 1469 bp, Artificial Sequence
  AGT103
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacaaggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtc

SEQ ID NO: 79 - DNA, 2814 bp, Artificial Sequence
  IFNgamma promoter, VRC01, antibody secretion signal sequence (AGT115)
tgtatttctactgggcagtgctgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagctcgaggccaccatgggatggtcatgtatcatcctttttctagtagcaactgcaactggtgtacattcccaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgggatggtcatgtatcatcctttttctagtagcaactgcaactggtgtacattccgaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - DNA, 2904 bp, Artificial Sequence
  EF1alpha promoter, CD4/IgG1 fusion protein, antibody secretion signal, miR30-CCR5/miR21-Vif/miR185-Tat microRNA cluster sequence (AGT118)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacagccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagcggccgctcgagcatgcatctagtcaaggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtcg

SEQ ID NO: 81 - DNA, 2884 bp, Artificial Sequence
  EF1alpha promoter, miR30-CCR5/miR21- Vif/miR185-Tat microRNA cluster sequence, CD4/IgG1 fusion protein, antibody secretion signal, (AGT119)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacaaggtatattgctgttgacagtgagcgactgtaaactgagcttgctctactgtgaagccacagatgggtagagcaagcacagtttaccgctgcctactgcctcggacttcaaggggcttcccgggcatctccatggctgtaccaccttgtcgggggatgtgtacttctgaacttgtgttgaatctcatggagttcagaagaacacatccgcactgacattttggtatctttcatctgaccagctagcgggcctggctcgagcagggggcgagggattccgcttcttcctgccatagcgtggtcccctcccctatggcaggcagaagcggcaccttccctcccaatgaccgcgtcttcgtcgcggccgcgccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 82 - DNA, 1937 bp, Artificial Sequence
  IL2 promoter, CD4/IgG1 fusion protein, antibody secretion signal (AGT120)
atctatcttattgtatgcaattagctcattgtgtggataaaaaggtaaaaccattctgaaacaggaaaccaatacacttcctgtttaatcaacaaatctaaacatttattcttttcatctgtttactcttgctcttgtccaccacaatatgctattcacatgttcagtgtagttttatgacaaagaaaattttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactcttgaattcgccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 83 - DNA, 2028 bp, Artificial Sequence
  IFNgamma promoter, CD4/IgG1 fusion protein, antibody secretion signal (AGT121)
tgtatttctactgggcagtgctgatctagagcaatttgaaacttgtggtagatattttactaaccaactctgatgaaggacttcctcaccaaattgttcttttaaccgcattctttccttgctttctggtcatttgcaagaaaaattttaaaaggctgcccctttgtaaaggtttgagaggccctagaatttcgtttttcacttgttcccaaccacaagcaaatgatcaatgtgctttgtgaatgaagagtcaacattttaccagggcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcaggaattcgccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgctgcagatccggagcccaagagctgcgacaagacccacacctgtccaccatgccccgcccacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 84 - DNA, 2659 bp, Artificial Sequence
  CD69 (1050) promoter, CD4/IgG1 fusion protein, antibody secretion signal (AGT122)
cagacactgaggcttgggttgggcgaggccattcagacactaaaacccagtgcagttctccctcaagtgtgaccttatatgaagaatccggagggaggtttctgaaaggaaatgatgtaatgtgaggcagatgcaaagtgcggcaggaaggcagggtgtacagtccttatcacggcagctgccttagtggtatgtgttcaaaggaaccacaaacttccgacctgaggcagtttccggtgacaacctgctcatcatatttcaaaatgatttttttctttcagtgagtgaatgaggtactggaatgtcctctagatgataacttccaacccacctatgcataaaatttaacgtctttattctaaataagtgatattaataataaaatttggggcaccaagattattaatcagagtggtattttgatttccctccttaaatcaccatacatagctttctgcattcatcttgcgttgactgtcattacttgtctgagtgagactgataccacagcgatgttttaaataataatcatacctcaaaagactgaagtctcagaggtatctgaagagaataacctagagcacagggggagaattgaaggagctgttactgaggtgacataaaagcagtctaaatgacagtaaaatgtgacaagaaaattagcaggaaacaaatgaaacagataatttaagataaacaattttagagcatagcaaggaagttccagaccacaagctttctgtttcctgcattcttacttcttactacgtgatacatctagtcaccagggaagaagcgaatgacacacttccaaaaaccaattcgtagctttctaaataaaaccctttctagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcacgaattcgccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - DNA, 2234 bp, Artificial Sequence
  CD69 (625) promoter, CD4/IgG1 fusion protein, antibody secretion signal (AGT123)
cagacactgaggcttgggttgggcgaggccattcagacactaaaacccagtgcagttctccctcaagtgtgaccttatatgaagaatccggagggaggtttctgaaaggaaatgatgtaatgtgaggcagatgcaaagtgcggcaggaaggcagggtgtacagtccttatcacggcagctgccttagtggtatgtgttcaaaggaaccacaaacttccgacctgaggcagtttccggtgacaacctgctcatcatatttcaaaatgatttttttctttcagtgagtgaatgaggtactggaatgtccaagctttctgtttcctgcattcttacttcttactacgtgatacatctagtcaccagggaagaagcgaatgacacacttccaaaaaccaattcgtagctttctaaataaaaccctttctagctggagagagatccatgagcatagagatcttaaaattcatgttcagcaataaatcctggggccccagacagtgtcaggtgcatagggggtgttcagtaaatatcagttaaatgtatgcataaatcgataaacgggattcctggaaaatactacactctccttctccaaattatcttcatctcaaagacaggaacctctaacttttaattctttacttagattatgctgtctcctaaactgtttatgttttctagaaatttaaggcaggatgtctcagagtctgggaaaatcccactttcctcctgctacaccttacagttgtgagaaagcacatttcagacaacagggaaaacccatacttcaccacaacaacacactatacattgtctggtccactggagcataaattaaagagaaacaatgtagtcaagcaagtaggcggcaagaggaagggggcggagacatcatcagggagtataaactctgagatgcctcagagcctcacgaattcgccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - DNA, 420 bp, Artificial Sequence
  VRC01 Heavy Variable Chain (with antibody secretory signal)
atgggatggtcatgtatcatcctttttctagtagcaactgcaactggtgtacattcccaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatca

SEQ ID NO: 87 - DNA, 3089 bp, Artificial Sequence
  EF1-VRC01 with Ab signal sequence (AGT114)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaggccaccatgggatggtcatgtatcatcctttttctagtagcaactgcaactggtgtacattcccaggtgcagctggtgcagtctgggggtcagatgaagaagcctggcgagtcgatgagaatttcttgtcgggcttctggatatgaatttattgattgtacgctaaattggattcgtctggcccccggaaaaaggcctgagtggatgggatggctgaagcctcgggggggggccgtcaactacgcacgtccacttcagggcagagtgaccatgacacgagacgtttattccgacacagcctttttggagctgcgctcgttgacagtagacgacacggccgtctacttttgtactaggggaaaaaactgtgattacaattgggacttcgaacactggggccggggcaccccggtcatcgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgtagacgaaagcgcggaagcggagagggcagaggaagtctgctaacatgcggtgacgtcgaggagaatcctggacctggatccatgggatggtcatgtatcatcctttttctagtagcaactgcaactggtgtacattccgaaattgtgttgacacagtctccaggcaccctgtctttgtctccaggggaaacagccatcatctcttgtcggaccagtcagtatggttccttagcctggtatcaacagaggcccggccaggcccccaggctcgtcatctattcgggctctactcgggccgctggcatcccagacaggttcagcggcagtcggtgggggccagactacaatctcaccatcagcaacctggagtcgggagattttggtgtttattattgccagcagtatgaattttttggccaggggaccaaggtccaggtcgacattaagcgagaattcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 88 - DNA, 2508 bp, Artificial Sequence
  EF-1alpha promoter, CD4/IgG1 fusion protein version 2, antibody secretion signal (AGT124)
ccggtgcctagagaaggtggcgcggggtaaactgggaaagtgatgtcgtgtactggctccgcctttttcccgagggtgggggagaaccgtatataagtgcagccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacagccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - DNA, 2406 bp, Artificial Sequence
  EF-1alpha promoter, CD4/IgG1 fusion protein version 3, antibody secretion signal (AGT125)
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgtacagccaccatgggatggtcatgtatcatcctttttctagtagcaactgcaactggtgtacattccaagaaagtggtgctgggcaaaaaaggggatacagtggaactgacctgcacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacaaggtggtaagaccctctccgtgtctcagctggagctccaggatagtggcacctggacatgcactgtcttgcagaaccagaagaaggtggagttcaaaatagacatcgtggtgctagctgagcccaagagctgcgacaagacccacacctgtccaccatgccccgcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgctgtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga